(19) United States
(12) Patent Application Publication
(10) Pub. No.: US 2025/0340870 A1
(43) Pub. Date: Nov. 6, 2025

(54) SYSTEMS, METHODS, AND COMPOSITIONS FOR ALTERING THE EXPRESSION OF ENDOGENOUS CIRCULAR RNAS

(71) Applicants: Madhukar H. TRIVEDI, Albuquerque, NM (US); UNM Rainforest Innovations, Albuquerque, NM (US)

(72) Inventors: Nikolaos Mellios, Albuquerque, NM (US); Alexander K. Hafez, Albuquerque, NM (US)

(21) Appl. No.: 18/549,887

(22) PCT Filed: Mar. 11, 2022
(86) PCT No.: PCT/US2022/071110

§ 371 Date: Sep. 8, 2023

Related U.S. Application Data
(63) Continuation-in-part of application No. PCT/US2022/017936, filed on Feb. 25, 2022.
(60) Provisional application No. 63/159,798, filed on Mar. 11, 2021.

(51) Int. Cl. C12N 15/113; A01K 67/00; A61K 48/00; C12N 9/22

(52) U.S. Cl. CPC C12N 15/113; A01K 67/00; A61K 48/0008; A61K 48/005; C12N 9/226; A01K 2217/075; A01K 2267/03; C12N 2310/20

(57) ABSTRACT

A method for disrupting the formation of circular RNAs in vivo, by disrupting or removing one or more intronic regulatory sequences responsible for circRNA backsplicing. The systems and methods can further be used for inhibiting the expression of therapeutically relevant circular RNAs in vivo related to brain and other disorders, as well as the creation of transgenic animal having one or more circRNA mutations that can be used as models mammalian diseases related to circRNA production and regulation, and in particular human brain disorders.

### CROSS-REFERENCE TO RELATED APPLICATIONS

This application claims the benefit of the filing date of U.S. Provisional Application No. 63/159,798, filed on Mar. 11, 2021, and to PCT/2022/017936, filed on Feb. 25, 2022, the disclosures of which are incorporated by reference herein.

### SEQUENCE LISTING

The instant application contains a Sequence Listing which has been submitted electronically in ASCII format and is hereby incorporated by reference in its entirety. Said ASCII copy, created on Apr. 30, 2024, is named “1863271US1.txt” and is 530,329 bytes in size.

### TECHNICAL FIELD

The technology relates to the field of therapeutics, and in particular systems, methods, and compositions for altering the expression of endogenous circular RNAs.

### BACKGROUND

Circular RNAs (circRNAs) are a category of non-coding RNAs (ncRNAs) that are particularly enriched in the human brain and have recently been implicated in various neurological and psychiatric disorders. Despite the emerging role of circRNAs for brain development, function, and disease, as well as cancer and other disorders, methods are needed to manipulate their expression, so as to probe their function and provide for their use in therapeutic interventions. One major limitation in circRNA manipulation approaches is that circRNAs in their vast majority originate within protein-coding genes, utilizing exons that are also needed for the production of linear messenger RNAs (mRNAs) that are needed for protein production. Previously, it was shown that the use of short-hairpin RNAs (shRNAs) against the unique circRNA splice junctions can achieve moderate, but specific knockdown of circRNAs without influencing mRNA expression from their parent gene. However, such an approach might not be robust enough and could potentially result in off-target effects.

### SUMMARY

The disclosure provides systems, methods, and compositions for specifically and robustly manipulating circRNA expression. In one aspect, the systems, methods, and compositions inhibit circular RNA (circRNA) formation, also referred to a circRNA biogenesis, in in vitro and in in vivo systems. In this aspect, the systems, methods, and compositions are configured to identify and establish a nucleic acid sequence encoding a target circRNA having at least one intronic regulatory sequence that is configured to be capable of promoting circRNA backsplicing. In this embodiment, a nucleic acid editing system, such as a CRISPR/Cas9 system, or other gene editing endonuclease system, may be configured to target and disrupted the intronic regulatory sequence of the target circRNA sequence. This disruption or removal of the intronic regulatory sequence prevents or inhibits target circRNAs formation by inhibiting its ability circularize through backsplicing thereby inhibiting not only its expression, but activity.

In another aspect, the systems, methods, and compositions are configured to inhibit circHomer1 circRNA formation. In this aspect, the systems, methods, and compositions are configured to identify and establish a nucleic acid sequence encoding circHomer1 circRNA, or a fragment or variant thereof, having an intronic regulatory sequence capable of promoting circRNA backsplicing. In this embodiment, a nucleic acid editing system, such as a CRISPR/Cas9 system, or other gene editing endonuclease system, may be configured to include a pair of gRNAs complementary to the 5′ and the 3′ portion of an intronic regulatory sequence, which may include intron 5 or intron 1 of circHomer1 circRNA. In this aspect, the CRISPR/Cas9 protein endonuclease system removes or disrupts the intronic regulatory sequence inhibiting the backsplicing of said circHomer1 circRNA.

Another aspect of the methods and systems to identify is to identify an intronic regulatory sequence of a target circRNA and generate gRNAs complementary to the 5′ and the 3′ portion of the intronic regulatory sequence, that can facilitate the disruption and/or removal of the intron sequence by a CRISPR gene editing system thereby inhibiting the back-splicing of the target circRNA. In another aspect, the disclosure provides a composition comprising one or more RNA (gRNAs) that can be used to disrupt or remove all or a portion of human or mouse circHomer1 intronic complementary regions for circHomer1 biogenesis (such as intron 1 or intron 5), so as to downregulate circHomer1 expression for therapeutic or diagnostic purposes. In another aspect, a method is described that can be used to downregulate or knockdown circRNA expression for the purpose of creating treatments to brain and other disorders, such as cancer. In one embodiment, the subject is a human. In another aspect, a method is described that can be used to downregulate/knockdown or knockout circRNA expression for the purpose of generating a circRNA transgenic knockout or knockdown animal. In one embodiment, the subject is a mouse.

In another aspect, a method of treating a disease or condition in a mammal, e.g., a human, through the inhibition or knockdown circRNA expression is described. In one aspect, a method of treating a disease or condition in a mammal, such as a human, is provided through the use of CRISPR, or other gene editing endonuclease system, to remove all or portions from an exemplary intron region of a gene or genome disrupting the formation of a target circRNA.

In an aspect, the disclosure includes a method of treating a disease or condition in a mammal, e.g., a human, through the deleting the complementary intronic sequences that span the exons that are utilized for circRNA biogenesis. In one aspect a method of treating a disease or condition in a mammal is provided. for instance in a human, through the use of CRISPR, or other gene editing endonuclease systems, to remove multiple complementary repetitive sequences from an exemplary intron, in this case intron 5 or 1 of circHomer1.

### BRIEF DESCRIPTION OF THE DRAWINGS

Figures 1A-1F. CRISPR-mediated specific knockout of circHomer1. (A-B) Schematic representation of CRISPR-mediated knockout of circHomer1 in mouse targeting complementary sequences >125 bp highlighted in green (Comp Seq 1), blue (Comp Seq 3), and yellow (Comp Seq 3). (C) Mean±SEM relative to no gRNA control, circHomer1 levels after CRISPR-mediated circHomer1 knockout in a heterogeneous population of mouse Neuro2a cells. (D) Schematic illustrating genotyping strategy of circHomer1 intron deletion, whereas a 4645 bp product is expected from wild-type sequence and a 248 bp product is expected following CRISPR mediated deletion. (E) 248 bp PCR product from tail DNA of mice derived from zygotes microinjected with circHomer1-targeting sgRNAs and Cas9 protein. (F) Sanger Sequencing analysis of circHomer1KO mouse aligned to the predicted sequence following CRISPR mediated deletion in circHomer1 intron 5.

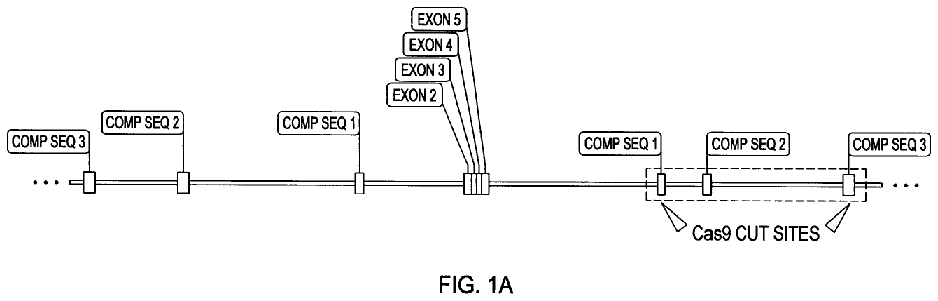

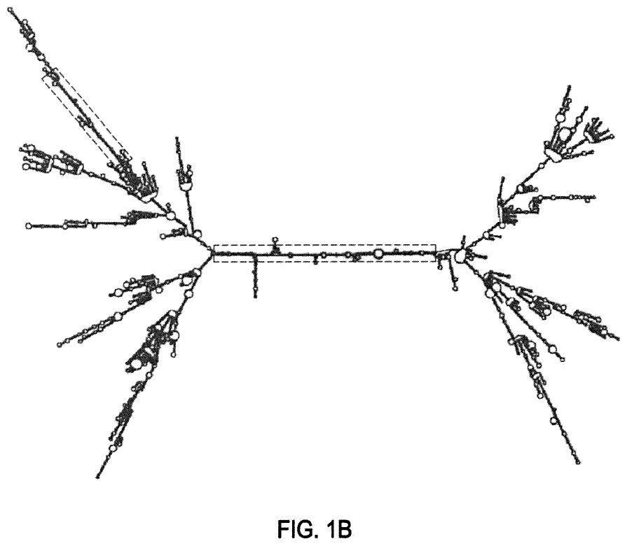

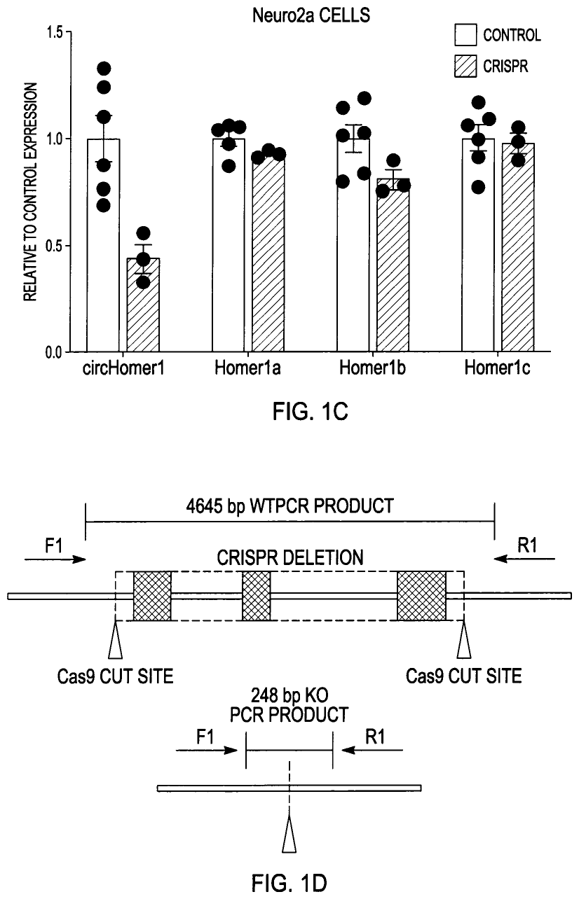

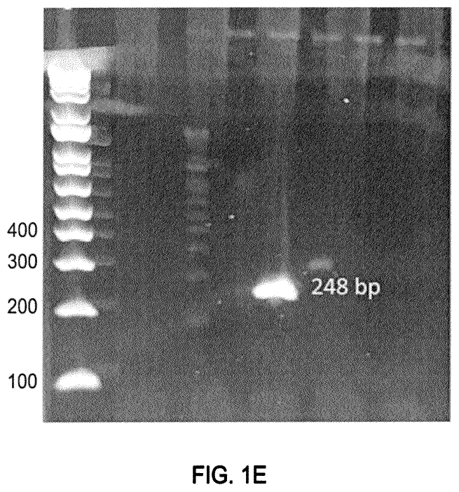

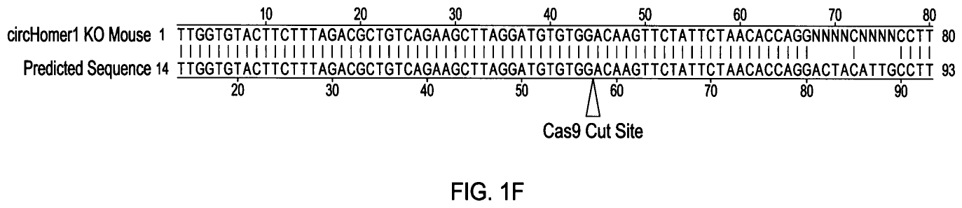

### DETAILED DESCRIPTION

As noted above, circRNAs are important regulators of gene expression within the brain that could be of relevance for the pathophysiology, pathogenesis, and treatment of numerous psychiatric and neurological disorders, as well as cancer. As a result, the disclosure provides for the use of genome editing technologies to affect circRNA expression for therapeutic and diagnostic purposes. According to one embodiment, deleting the complementary intronic sequences that span the exons that are utilized for circRNA biogenesis can significantly reduce the expression of a given circRNA. The present disclosure provides compositions, kits, assays, systems, and methods for manipulating circRNA expression, and in particular kits, assays, compositions, systems, and methods for deleting the complementary intronic sequences that span the exons that are utilized for circRNA biogenesis can significantly reduce the expression of a given circRNA, all of which can be applicable to the treatment of brain and other disorders.

With half-lives of several days, circRNAs are the most stable RNAs ever discovered, thus allowing them to serve as long-term regulators of gene expression. Numerous studies have suggested that circRNAs are enriched within the human brain and are differentially expressed in the brain of subjects with psychiatric or neurological disorders. For example, circHomer1, is a highly-expressed circRNA, derived from exons 2-5 of the HOMER1 gene (SEQ ID NO: 6), which is altered in psychiatric disorders, such as Schizophrenia and Bipolar Disorder and neurological disorders, such as Alzheimer's disease, Parkinson's disease, and frontotemporal dementia. Downregulating circHomer1 expression in the mouse brain can result in significant alterations in cognitive function and affect the expression of numerous brain genes related to psychiatric and neurological diseases. Moreover, the same circRNA has been implicated in Colorectal Cancer and Hepatocellular carcinoma. Therefore, finding methods to manipulate circHomer1 or other disease-associated circRNAs could allow for therapeutics for brain and other disorders.

According to an embodiment the present disclosure provides compositions, kits, assays, systems, and methods for circRNA-mediated therapeutics for brain disorders, cancer, and other disorders. Such methods involve the use of genome/gene editing systems, such as CRISPR, to delete parts of intronic sequences in introns for circRNA biogenesis so as to downregulate the expression of a specific circRNA without altering the levels of the mRNAs expressed from the same genes.

According to a specific embodiment, the present disclosure provides a method to manipulate circRNA expression and their use as a therapeutic for one or more huma disease or conditions. CircRNAs are a category of long non-coding RNAs (ncRNAs) that are derived from the circularization and covalent joining of backspliced exons and/or introns. CircRNAs are particularly enriched in the mammalian brain (at least 100,000 different circRNAs have been identified in the human brain), yet, with few exceptions, lack the capacity of being translated into protein. Some circRNAs are known to sequester microRNAs (miRNAs) by containing partial complementary sequences and others to associate with RNA-binding proteins (RBPs) and transcription factors. Recent work has suggested that circRNAs could be altered in neurological and psychiatric disorders and that they could be responsible for regulating brain development, maturation, plasticity, and aging. Moreover, multiple studies have linked circRNAs to various cancers. Due to their closed loop structure, circRNAs are particularly resistant to degradation thus exhibit very high stability, with half-lives ranging from a few days to a week. This unparalleled stability in conjunction with their capacity to regulate the expression of multiple protein-coding genes, allows circRNAs to accumulate in various tissues and serve a center role for tissue homeostasis and disease.

According to an embodiment, the present disclosure provides a circRNA, e.g., circHomer1 (SEQ ID NO: 7), encoded in the gene homer1 (SEQ ID NO: 6), as a target for intronic sequence genome editing-mediated therapeutics related to brain disorders and cancer. Examples of such disorders are Schizophrenia, Bipolar Disorder, Alzheimer's disease, Parkinson's disease, Frontotemporal dementia, Epilepsy, Colorectal Cancer, and Hepatocellular carcinoma.

According to an embodiment, the present disclosure provides a method for manipulating circRNA expression for the purpose of therapeutics. For the purposes of the present disclosure, the terms “patient” or “subject” refer to any animal (e.g., mammal), including, but not limited to, humans, non-human primates, equines, canines, felines, rodents (mice, rats), and the like.

According to a specific example, a method is described that can be used to inhibit, by downregulating/knocking down or knocking out circRNA expression for the purpose of generating a circRNA knockout or knockdown transgenic animal model. In one embodiment, the subject is a mouse,

According to another embodiment, the genome editing method of the disclosure could involve any form of genome editing to remove a part of intronic sequence relevant to cireRNA biogenesis, such as meganucleases, zinc finger nucleases (ZFNs), transcription activator-like effector-based nucleases (TALEN), the clustered regularly interspaced short palindromic repeats (CRISPR/Cas9) system, as well as RNA editing of the precursor mRNAs containing such intronic sequences via CRISPR/Cas12a, CRISPR/Cas13, or other related genome editing approaches. According to an embodiment, at least one of the circRNAs to be inhibited may be selected from a circRNA encoded by the nucleotide sequences according to one or SEQ ID Nos. 1-433 and 439.

In one aspect, the disclosure includes systems, methods, and compositions configured to inhibit circular RNA (circRNA) formation, also referred to as cireRNA biogenesis, in in vitro and in in vivo systems. In this aspect, the disclosure includes systems, methods, and compositions configured to identify and establish a nucleic acid sequence, which may include an isolated gene encoding sequence or fragment thereof, or a sequence that is part of a genome of a subject, such as a human or mouse. In this embodiment, the nucleic acid sequence may encode a target circRNA having at least one intronic regulatory sequence and capable of promoting circRNA backsplicing. As noted above, in certain embodiments, the systems and methods of the disclosure may be applied to any circRNA having at least one intronic regulatory sequence, which may include one or more intron sequences. For example, in one embodiment, the target circRNA of the disclosure may include a target cirRNA, e.g., one of SEQ ID Nos. 1-433 or 439, or any combination thereof. Notably, identification of circRNAs and related intronic/exonic sequences is characterized by those of ordinary sill in the art. (See Das D, et al., Identification and Characterization of Circular Intronic RNAs Derived from Insulin Gene. Int J Mol Sci. 2020;21(12):4302. Published 2020 Jun. 17; and Ragan C, et al., Insights into the biogenesis and potential functions of exonic circular RNA. Sci Rep. 2019 Feb. 14;9(1):2048. Both being incorporated herein by reference)

In this embodiment, a nucleic acid editing system, such as a CRISPR/Cas9 system, or other gene editing endonuclease system, such as a TALEN system, or a zinc-finger nuclease system may be configured to target and disrupted the intronic regulatory sequence(s) of the target circRNA sequence. This disruption or removal of the intronic regulatory sequence inhibits the target circRNA's ability circularize through back-splicing thereby inhibiting not only its expression, but activity, while continuing to allow the nucleic acid sequence of the disclosure to encode a functional messenger RNA (mRNA) that is not interrupted by disruption or removal of the intronic regulatory sequence.

In this embodiment, a nucleic acid editing system, such as a CRISPR/Cas9 system may include one or more guide RNAs (gRNAs) complementary to the 5′ and 3′ portion of an intronic regulatory sequence respectively. In this embodiment, the CRISPR/Cas9 and gRNAs may be delivered to a cell or isolated nucleic acid, disrupt or remove the intronic regulatory sequence thereby inhibiting the target circRNA's ability circularize through back-splicing. This process inhibits not only the circRNA's endogenous expression, but in vivo activity in a subject. As circRNAs have been directly linked to one or more diseases or conditions, in one embodiment, the step of inhibiting a target circRNA's ability circularize through back-splicing may treat a subject that has, or is at risk of developing. In one example, such a disease or condition that may be treated by the methods and compositions of the disclosure may include, but not be limited to: schizophrenia, bipolar disorder, Alzheimer's disease, Epilepsy, Frontotemporal Dementia, Parkinson's disease, Colorectal Cancer, and Hepatocellular carcinoma, or a combination of the same.

In another embodiment, the disclosure may include systems, methods, and compositions to inhibit the biogenesis of circHomer1 circRNA (SEQ ID NO: 7 or SEQ ID NO: 13), in vitro, or in an in vivo system. In this embodiment, a nucleic acid sequence, e.g., a genomic nucleic acid sequence or a human or mouse, encoding a circHomer1 circRNA, or fragment or variant thereof, may include an intronic regulatory sequence capable of promoting circRNA backsplicing. In this embodiment, gene editing system may be established to disrupt or remove this intronic regulatory sequence. For example, a CRISPR/Cas9 protein endonuclease system, having a pair of gRNAs (SEQ ID Nos. 1-2 and 8-9) complementary to the 5′ and the 3′ portion of circHomer1 intronic regulatory sequence respectively, generally referred to as a 5′ and 3′ cut site (SEQ ID Nos 3-4, and SEQ ID Nos 10-11), may be used to disrupt or remove the circHomer1 intronic regulatory sequence, which may include intron 5. The aforementioned methods and compositions of the disclosure inhibit the back-splicing of circHomer1 circRNA, forming a non-functional circRNA structure having its intronic regulatory sequence removed (SEQ ID NO: 5 or SEQ ID NO: 12). Being unable to circularize, the linear RNA molecule may be degraded by nuclease (RNases) enzyme.

As noted above, circHomer1 circRNAs, also referred to as circHomer1, has been directly linked to one or more diseases or conditions, in one embodiment, the step of inhibiting circHomer1's ability circularize through back-splicing may be used treat a subject that has, or is at risk of developing a disease or condition selected from the group consisting of: schizophrenia, bipolar disorder, Alzheimer's disease, Epilepsy, Frontotemporal Dementia, Parkinson's disease, Colorectal Cancer, and Hepatocellular carcinoma, or any combination thereof.

Delivery of one or more of the gene/genome editing compositions to a target nucleic acid sequence of the disclosure may be accomplished through a variety of mechanisms known within the art, including viral, as well as physical methods. See M. Behr., et al., In vivo delivery of CRISPR-Cas9 therapeutics: Progress and challenges, Acta Pharmaceutica Sinica B, Volume 11, Issue 8, 2021, Pages 2150-2171; and Xu X, Wan T, Xin H, Li D, Pan H, Wu J, Ping Y. Delivery of CRISPR/Cas9 for therapeutic genome editing. J Gene Med. 2019 July;21(7), being incorporated herein by reference).

As noted above, a “gene-editing system,” may mean one or more endonuclease-based systems configured to modify a nucleic acid sequence or genome. In one embodiment, a “gene-editing system,” may include the gene-editing CRISPR, such as CRISPR/cas-9 technology which describes an RNA-guided gene-editing platform that makes use of a bacterially derived protein (Cas9) and a synthetic guide RNA to introduce a double strand break at a specific location within the genome. Editing is achieved by transfecting a cell or a subject with the Cas9 protein along with a specially designed guide RNA (gRNA) that directs the cut through hybridization with its matching genomic sequence. By making use of this technology, it is possible to introduce specific genetic alterations in one or more target genes. In some embodiments, this CRISPR/cas-9 may be utilized to replace one or more existing wild-type genes with a modified version, while additional embodiments may include the addition of genetic elements that alter, reduce, increase or knock-out the expression of a gene, gene sequence, such as an intronic regulatory sequence.

In some embodiments, the agent for altering gene expression is a zinc finger nuclease or other equivalent. The term “zinc finger nuclease” as used herein, refers to a nuclease comprising a nucleic acid cleavage domain conjugated to a binding domain that comprises a zinc finger array. In some embodiments, the cleavage domain is the cleavage domain of the type II restriction endonuclease FokI. Zinc finger nucleases can be designed to target virtually any desired sequence in a given nucleic acid molecule for cleavage, and the possibility to design zinc finger binding domains to bind unique sites in the context of complex genomes allows for targeted cleavage of a single genomic site in living cells, for example, to achieve a targeted genomic alteration of therapeutic value. Targeting a double-strand break to a desired genomic locus can be used to introduce frame- shift mutations into the coding sequence of a gene due to the error-prone nature of the non-homologous DNA repair pathway.

Zinc finger nucleases can be generated to target a site of interest by methods well known to those of skill in the art. For example, zinc finger binding domains with a desired specificity can be designed by combining individual zinc finger motifs of known specificity. The structure of the zinc finger protein Zif268 bound to DNA has informed much of the work in this field and the concept of obtaining zinc fingers for each of the 64 possible base pair triplets and then mixing and matching these modular zinc fingers to design proteins with any desired sequence specificity has been described (Pavletich NP, Pabo Colo. (May 1991). “Zinc finger-DNA recognition: crystal structure of a Zif268-DNA complex at 2.1 A”. Science 252 (5007): 809-17, the entire contents of which are incorporated herein).

In some embodiments, separate zinc fingers that each recognizes a 3 base pair DNA sequence are combined to generate 3-, 4-, 5-, or 6-finger arrays that recognize target sites ranging from 9 base pairs to 18 base pairs in length. In some embodiments, longer arrays are contemplated. In other embodiments, 2-finger modules recognizing 6-8 nucleotides are combined to generate 4-, 6-, or 8-zinc finger arrays. In some embodiments, bacterial or phage display is employed to develop a zinc finger domain that recognizes a desired nucleic acid sequence, for example, a desired nuclease target site of 3-30 bp in length.

Zinc finger nucleases, in some embodiments, comprise a zinc finger binding domain and a cleavage domain fused or otherwise conjugated to each other via a linker, for example, a polypeptide linker. The length of the linker determines the distance of the cut from the nucleic acid sequence bound by the zinc finger domain. If a shorter linker is used, the cleavage domain will cut the nucleic acid closer to the bound nucleic acid sequence, while a longer linker will result in a greater distance between the cut and the bound nucleic acid sequence. In some embodiments, the cleavage domain of a zinc finger nuclease has to dimerize in order to cut a bound nucleic acid. In some such embodiments, the dimer is a heterodimer of two monomers, each of which comprise a different zinc finger binding domain. For example, in some embodiments, the dimer may comprise one monomer comprising zinc finger domain A conjugated to a FokI cleavage domain, and one monomer comprising zinc finger domain B conjugated to a FokI cleavage domain. In this non-limiting example, zinc finger domain A binds a nucleic acid sequence on one side of the target site, zinc finger domain B binds a nucleic acid sequence on the other side of the target site, and the dimerize FokI domain cuts the nucleic acid in between the zinc finger domain binding sites.

The term “zinc finger,” as used herein, refers to a small nucleic acid-binding protein structural motif characterized by a fold and the coordination of one or more zinc ions that stabilize the fold. Zinc fingers encompass a wide variety of differing protein structures (see, e.g., Klug A, Rhodes D (1987). “Zinc fingers: a novel protein fold for nucleic acid recognition”. Cold Spring Harb. Symp. Quant. Biol. 52:473-82, the entire contents of which are incorporated herein by reference). Zinc fingers can be designed to bind a specific sequence of nucleotides, and zinc finger arrays comprising fusions of a series of zinc fingers, can be designed to bind virtually any desired target sequence. Such zinc finger arrays can form a binding domain of a protein, for example, of a nuclease, e.g., if conjugated to a nucleic acid cleavage domain. Different types of zinc finger motifs are known to those of skill in the art, including, but not limited to, Cys2His2, Gag knuckle, Treble clef, Zinc ribbon, Zn2/Cys6, and TAZ2 domain-like motifs (see, e.g., Krishna S, Majumdar I, Grishin N V (January 2003). “Structural classification of zinc fingers: survey and summary”. Nucleic Acids Res. 31 (2): 532-50). Typically, a single zinc finger motif binds 3 or 4 nucleotides of a nucleic acid molecule. Accordingly, a zinc finger domain comprising 2 zinc finger motifs may bind 6-8 nucleotides, a zinc finger domain comprising 3 zinc finger motifs may bind 9-12 nucleotides, a zinc finger domain comprising 4 zinc finger motifs may bind 12-16 nucleotides, and so forth. Any suitable protein engineering technique can be employed to alter the DNA-binding specificity of zinc fingers and/or design zinc finger fusions to bind virtually any desired target sequence from 3-30 nucleotides in length (see, e.g., Pabo C O, Peisach E, Grant R A (2001). “Design and selection of novel cys2H is2 Zinc finger proteins”. Annual Review of Biochemistry 70:313-340; Jamieson A C, Miller J C, Pabo C O (2003). “Drug discovery with engineered zinc-finger proteins”. Nature Reviews Drug Discovery 2 (5): 361-368; and Liu Q, Segal D J, Ghiara J B, Barbas C F (May 1997). “Design of polydactyl zinc-finger proteins for unique addressing within complex genomes”. Proc. Natl. Acad. Sci. U.S.A. 94 (11); the entire contents of each of which are incorporated herein by reference).

Fusions between engineered zinc finger arrays and protein domains that cleave a nucleic acid can be used to generate a “zinc finger nuclease.” A zinc finger nuclease typically comprises a zinc finger domain that binds a specific target site within a nucleic acid molecule, and a nucleic acid cleavage domain that cuts the nucleic acid molecule within or in proximity to the target site bound by the binding domain. Typical engineered zinc finger nucleases comprise a binding domain having between 3 and 6 individual zinc finger motifs and binding target sites ranging from 9 base pairs to 18 base pairs in length. Longer target sites are particularly attractive in situations where it is desired to bind and cleave a target site that is unique in a given genome.

In some embodiments, the agent for altering the target gene is a TALEN system or its equivalent. The term TALEN or “Transcriptional Activator-Like Element Nuclease” or “TALE nuclease” as used herein, refers to an artificial nuclease comprising a transcriptional activator like effector DNA binding domain to a DNA cleavage domain, for example, a FokI domain. A number of modular assembly schemes for generating engineered TALE constructs have been reported (Zhang, Feng; et. al. (February 2011). “Efficient construction of sequence-specific TAL effectors for modulating mammalian transcription”. Nature Biotechnology 29 (2): 149-53; Geibler, R.; Scholze, H.; Hahn, S.; Streubel, J.; Bonas, U.; Behrens, S. E.; Boch, J. (2011), Shiu, Shin-Han. ed. “Transcriptional Activators of Human Genes with Programmable DNA-Specificity”. PLOS ONE 6 (5): e19509; Cermak, T.; Doyle, E. L.; Christian, M.; Wang, L.; Zhang, Y.; Schmidt, C.; Baller, J. A.; Somia, N. V. et al. (2011). “Efficient design and assembly of custom TALEN and other TAL effector-based constructs for DNA targeting”. Nucleic Acids Research; Morbitzer, R.; Elsaesser, J.; Hausner, J.; Lahaye, T. (2011). “Assembly of custom TALE-type DNA binding domains by modular cloning”. Nucleic Acids Research; Li, T.; Huang, S.; Zhao, X.; Wright, D. A.; Carpenter, S.; Spalding, M. H.; Weeks, D. P.; Yang, B. (2011). “Modularly assembled designer TAL effector nucleases for targeted gene knockout and gene replacement in eukaryotes”. Nucleic Acids Research.; Weber, E.; Gruetzner, R.; Werner, S.; Engler, C.; Marillonnet, S. (2011). Bendahmane, Mohammed. ed. “Assembly of Designer TAL Effectors by Golden Gate Cloning”. PLOS ONE 6 (5): e19722; each of which is incorporated herein by reference).

Those of skill in the art will understand that TALE nucleases can be engineered to target virtually any genomic sequence with high specificity, and that such engineered nucleases can be used in embodiments of the present technology to manipulate the genome of a cell, e.g., by delivering the respective TALEN via a method or strategy disclosed herein under circumstances suitable for the TALEN to bind and cleave its target sequence within the genome of the cell. In some embodiments, the delivered TALEN targets a gene or allele associated with a target circRNA. In some embodiments, delivery of the TALEN to a subject confers a therapeutic benefit to the subject, such as reducing, eliminating expressing a circRNA in a subject in need therein.

In some embodiments, the target gene of a cell, tissue, organ or organism is altered by a nuclease delivered to the cell via a strategy or method disclosed herein, e.g., CRISPR/cas-9, a TALEN, or a zinc-finger nuclease, or a plurality or combination of such nucleases. In some embodiments, a single-or double-strand break is introduced at a specific site within the genome by the nuclease, resulting in a disruption of the target genomic sequence, such as a intronic regulatory sequence.

In some embodiments, the target genomic sequence is a nucleic acid sequence within an intronic regulatory sequence In some embodiments, the strand break introduced by the nuclease leads to a mutation within the intronic regulatory sequence that impairs the expression of the encoded gene product, which in this case may include a circRNA. In some embodiments, a nucleic acid is co-delivered to the cell with the nuclease. In some embodiments, the nucleic acid comprises a sequence that is identical or homologous to a sequence adjacent to the nuclease target site. In some such embodiments, the strand break affected by the nuclease is repaired by the cellular DNA repair machinery to introduce all or part of the co-delivered nucleic acid into the cellular DNA at the break site, resulting in a targeted insertion of the co-delivered nucleic acid, or part thereof. In some embodiments, the insertion results in the disruption or repair of the undesired allele. In some embodiments, the nucleic acid is co-delivered by association to a supercharged protein. In some embodiments, the supercharged protein is also associated to the functional effector protein, e.g., the nuclease. In some embodiments, the delivery of a nuclease to a target cell results in a clinically or therapeutically beneficial alteration of the function of a circRNA.

In some embodiments, cells from a subject are obtained and a nuclease or other effector protein is delivered to the cells by a system or method provided herein ex vivo. In some embodiments, the treated cells are selected for those cells in which a desired nuclease-mediated genomic editing event has been affected. In some embodiments, treated cells carrying a desired genomic mutation or alteration are returned to the subject they were obtained from.

A method and compositions are provided that employ genome editing to delete complementary regulatory regions within the introns that span circRNAs. Deleting these intronic regions that are meant to base pair and facilitate circRNA production results in a robust decrease in circRNA biogenesis, without affecting linear splicing and mRNA production. The method was demonstrated by using clustered regularly interspaced short palindromic repeats (CRISPR) to remove multiple complementary repetitive sequences from intron 5 of circHomer1, a circRNA implicated in both psychiatric (e.g., Bipolar disorder, Schizophrenia, epilepsy, and alcohol and cocaine addiction) and neurological disorders (e.g., Alzheimer's and Frontotemporal Dementia). This approach can specifically and robustly manipulate circRNA expression for the purpose of probing function in animal and culture models and for use as circRNA-mediated therapeutics for neurological and psychiatric disorders, as well as cancers such as Colorectal Cancer and Hepatocellular carcinoma, e.g., the use of gRNAs specific for intron sequences in a circRNA and Cas allow for a robust regulation of gene expression.

The disclosure provides for the use of genome editing technologies to affect circRNA expression for therapeutic purposes. According to one embodiment deleting the complementary intronic sequences that span the exons that are utilized for circRNA biogenesis can significantly reduce the expression of a given circRNA. The present disclosure thus provides compositions, kits, assays, systems, and methods for manipulating circRNA expression for the treatment of brain and other disorders.

In one example, the disclosure provides a composition comprising one or more CRISPR guide RNA (gRNAs) that can be used to delete parts of human circHomer1 intronic complementary regions for circHomer1 biogenesis, e.g., (intron 1 or intron 5), so as to downregulate circHomer1 expression for therapeutic purposes.

In one embodiment, a method is described that can be used to downregulate or knockdown circRNA expression for the purpose of providing treatments to brain and other disorders, such as cancer. In one embodiment, the subject is a human.

In one embodiment, a method is described that can be used to downregulate/knockdown or knockout circRNA expression for the purpose of generating a circRNA knockout or knockdown animal. In one embodiment, the subject is a mouse.

### Exemplary Embodiments

The disclosure provides a method using genome editing to remove intronic regulatory sequences responsible for circRNA biogenesis for the purpose of reducing the expression of a specific circRNA for therapeutic purposes.

In addition, a method for performing such a deletion is provided. The method includes utilizing CRISPR-Cas9 and gRNAs or other genome editing tools to remove parts of the intronic regulatory sequences that are able to bind to each other to promote circRNA backsplicing.

In one embodiment, a composition is provided comprising gRNAs and CRISPR-Cas9 or other genome editing tools to downregulate circRNA expression. In one embodiment, at least one of the circRNAs is circHomer1. In one embodiment, gRNAs target an intronic regulatory sequence either 5′ or 3′ of the circRNA of interest that is capable of promoting cireRNA backsplicing. In one embodiment, genome editing is aimed in at reducing the expression of circHomer1 for the treatment of schizophrenia. In one embodiment, genome editing is aimed in at reducing the expression of circHomer1 for the treatment of bipolar disorder. In one embodiment, genome editing is aimed in at reducing the expression of circHomer1 for the treatment of Alzheimer's disease. In one embodiment, genome editing is aimed in at reducing the expression of circHomer for the treatment of Epilepsy. In one embodiment, genome editing is aimed in at reducing the expression of circHomer1 for the treatment of Frontotemporal Dementia. In one embodiment, genome editing is aimed in at reducing the expression of circHomer1 for the treatment of Parkinson's disease. In one embodiment, genome editing is aimed in at reducing the expression of circHomer1 for the treatment of Colorectal Cancer. In one embodiment, genome editing is aimed in at reducing the expression of circHomer1 for the treatment of Hepatocellular carcinoma. In one embodiment, the species is human or mouse. In one embodiment, genome editing is aimed in at reducing the expression of circHomer1 for the treatment of Hepatocellular carcinoma.

In one embodiment, a vector is provided encoding or one or more isolated gRNAs for genome editing of an intron in a circRNA. In one embodiment, the circRNA is a brain disorder-associated circRNA biomarker. In one embodiment, the circRNA is circHomer la. In one embodiment, the circRNA is circHomer 1b. In one embodiment, the editing deletes complementary regulatory regions within the intron(s) that span circRNAs. In one embodiment, the editing deletes one or more intronic regions that base pair and facilitate circRNA production.

In one embodiment, a method to prepare a vector for genome editing of an intron in a circRNA is provided comprising: selecting an intron present in a target circRNA of a mammalian genomic target, the expression of which target generates the circRNA; selecting nucleic acid sequences for homology arms that bind to nucleic acid sequences in the intron; and introducing to a vector the nucleic acid sequences for the homology arms.

In one embodiment, a method to decrease expression of a circRNA in a mammal is provided comprising introducing to a mammal an effective amount of a composition comprising isolated nucleic acid for a pair of gRNAs each comprising a targeting sequence, one of which is upstream and the other of which is downstream of sequences in an intron in a circRNA of a mammalian genomic target, the expression of which target yields the circRNA, or an effective amount of a composition comprising isolated nucleic acid for a pair of gRNAs each comprising a targeting sequence, one of which is upstream and the other of which is downstream of sequences in an intron in a circRNA of a mammalian genomic target, the expression of which target yields the circRNA and a composition comprising a vector comprising nucleic acid comprising homology arms that have targeting sequences for the intron, wherein the mammal expresses Cas or is administered a composition comprising isolated Cas or an isolated nucleic encoding Cas.

In one embodiment, the mammal is a human. In one embodiment, the circRNA is circHomer circRNA. In one embodiment, the circRNA is circHomer1a, circHomer1b, circCUL4A, and circADAM22. In one embodiment, the method allows for editing in the genome of the mammal to delete complementary regulatory regions within the intron(s) that span the circRNA. In one embodiment, the method allows for editing in the genome of the mammal to delete one or more intronic regions that base pair and facilitate circRNA production. In one embodiment, the effective amount deletes portions of one or more introns. In one embodiment,
the composition is systemically administered. In one embodiment, the composition is locally introduced. In one embodiment, the circRNA is a brain disorder-associated circRNA biomarker. In one embodiment, a nucleic acid sequence of interest is flanked by the homology arms in the vector. In one embodiment, the nucleic acid sequence of interest is a marker gene. In one embodiment, the nucleic acid sequence of interest is a therapeutic gene.

### Exemplary circRNAs

Human circHomerl Knockdown
Human circHomerl KO 5′ gRNA: TTGATTCATAAACGAATTCT
Human circHomer1 KO 3′ gRNA: ACTGTAGTCGTTACTGAATA
Human circHomerl KO 5′ Cut-Site:
ATTGTAGCTCAAGATGAAGAACTTGAGAATTATTGTTACACCAATTTC

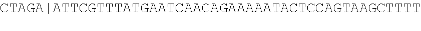

CAGAAAAATACTCCAGTAAGCTTTT
CTTATGTCAGTAAAGTAGTTTGAAT
Human circHomer1 KO 3′ Cut Site:
TAGAAGAGTTACTTTGAGTGTTCTTAACAAGTACCCGTTTAAGAAAT

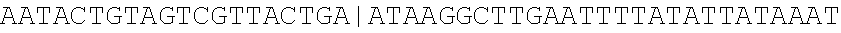

ATAATTAGTAATATCAGTAAAGATGG
Human circHomer1 KO Sequence after intronic area deletion:
ATTTTATTACTTTTTTCTCAGGGTTATCTTAACATTCTTCCAGTTTTCC
ACATCCATAGTAAGTTTTAGCATCCATCACTGTATGTAGTACTTTTAA
AGAATTGTAGCTCAAGATGAAGAACTTGAGAATTATTGTTACACCAA
TTTCCTGGCTTGAATTTTATATTATAAATATAATTAGTAATATCAGTA
AAGATGGTAAGCTATCCAGACCAACTTTCAGTGAGATATTTACCTCA
GAATTTGTCTTTTGATTCTCATAGCCATAAATATAAATCCTAGGAGAT
GTAAATATATTAC
hsa_circ_0006916 (circHomer1) circRNA whole genomic sequence:
GGAACAACCTATCTTCAGCACTCGAGCTCATGTCTTCCAAATTGACCC
AAACACAAAGAAGAACTGGGTACCCACCAGCAAGCATGCAGTTACT
GTGTCTTATTTCTATGACAGCACAAGAAATGTGTATAGGATAATCAG
TTTAGATGGCTCAAAGGTAAGCTACGTTTACTTTGAATGATTTGGCTG
GTTTTGCTTTTTTCAGTATGACATTTTAGTTCTATTCAGAACATTTTAT
CTTCATTAATATAAAATGTTTCCTTTTGAGTTCTTAAAACTTTTAAGA
AACAAAATGGATTATTAAGGTAGCAACATGTTTTGTCCACTAAATGA
CTAACCACTTTATTACATTGTTACATGCCAAAATATTCTACAAATTTG
AGGTCATTCCATTTATTTAATGTTCATTGCATATCTGTTATGTGTCAG
GCATTATGCCAGGTACTAGGAATATAGTGGTGGACAGAATAGATATG
GTCTAGAATTCTAGGAGCTCTAATTCTTTTCAAATATACTGCTACAGA
CTAGTAGCAGTTTAAAGTCCCTATATCCTATGAAGTTTGTCAGTGTCA
ATACTAAAGCTGACATTTCTTCTCTCAAGAGAAGTATAAGGACAAAA
CCTATTTTATTTTTAGGAGAGGCAATTAGTGTAACAGGAGCAAGGAC
TGCCTGGGTTCAGATCTCAACTCCACCATCTACTGAGTGTTTTCTCTTT
GGGCAGATTACTTCTCTGAGCCTCATTGTTCTCAACTCAGTACTGGTG
GTTGTAAGGATTAAATGAGTTAATATCTGCAAAGCACTGAAATCAGT
GCTTAGAGGACAGTGTGCCCTTCAGAAGAGTTGGTTGGGGATCCCTT
TTTTTTTTTTTTTTTTGAGACGGAGTCTTGCTCTGTCACCCAGCCTGGA
GTACAGCTCAGCTCACTGCAACCTCTGCCTCCTGGGTTCAAGTGATTT
TCTTGCCTCAGCCTCCCCAGTAGCTGGTATTACAGGCGTGCGCCTCCA
TGCCTGGATAATTTTTGTATTTTTAGTAGAGACAGGGTTTCACCATGT
TGGCCAGGCTGGTCTTGAACTCCTGACCTCTCGATCTGTCTGCCTCAG
CCTCCCAAAGTGCTGGGATTACAGGCATGAGCTACCGCGCTGGGACG
GGGGATCCTTTAATCATCAATGAACTTCATGGAACTCACTTAGAGAA
AGAAATTCCAAAGAAAAATCAGACTCTAAGATTCTAGGTTTGAAGTG
TGTAGAAAGATGAGGTCTTCAGTTTCTTAAAGCCATGAAGCCTTTCCA
CTTTCAGGAATTTTAGCAATTAAATGAAGAAGGGGTTAAGTGCACAC
ACTGTAGAGCCAGACTGACTGGTTTGAATTTTTGTTTCTACTACCTGC
TTGCCATCTATATTAGTCCATTCTCATACTTCTATGAAGAAATACCTG
AGACTGGGTAATTTATAAAGGAAAGAGGTTTACTTGACTCACAGTTC
CACATGGCTGGGGAAGCCTCAGGAAACTTACAATCGTGGCAGAAGGT
ACGTCTTCACAGGGCGGCAGGAGAGAGAGTGAGAGCAAGCAGGGGA
AATGCCAGATGCGTATAAAACCATCAGATCTCGTGAGAGTCACTATC
ACAAGAACAGCATTGGGGAAACTGCCCCCCATGATTCAATTACCTCC
ACCGGGTTCCACCCTTGACACATGGGGATTACAATTCAAGGTGAGAT
TTGGATGGGGACACAGAGCCAAACCATATCACCATCTGATCTTGAGC
AAAGTATTTAGCTTCTGCATGCAGGGCTGGGCACTGAGGTTTGTGCCT
GTAATTCTAGCACTTTGGGAGGCCGAGGCATGAGGATCACTTGAGAC
CAGGAGTTTGAGACCAGCCTGGGTAACACAGTGAGTTCCCATCTCAA
AAAAAAAAAAAATTAGCTAGGTGTATTGGTGCATGCCTGTAGTCCTA
GCTACTCAGGAGACTGGGGCTGGAGCTAAGGAGGTCAAAGCTGCAG
TGAGCTATGATTATATCCAGAGCGAGACCCTGTCTCTTAAGGGGAAA
AAAAAACTGTATGCCTCAGTTTTCTCATCTGTAAAAAGGAATGAATA
AAAATACCTACATCATTCCACCATAGGGTTGTTGTGAAAAGTAAGTA
AATTTAGGCAAAAGCACAGTGTATATAGTAATCACTATACAAACACT
CACTGCTATTGTTGTTAATACTATTATCAGGCGATTATTGCCTAGTGT
TTTATGGGGAATAAAAGATAATTTAATGCTAAACTTTTTGTCTGTTTT
TTCTGTGTACTCATTCCTTATCTCCTTAACTAGGTTAACTAGATGATA
AGACCCTAAGCGCTATTTGCACTTTGTGTCTTGTACATAAGTAACTAA
TATGTATTTACTTGAATAATTGACTTTTTCATTTTTACTTGGAGGATTT
AAAGGACTTGAGAAATAGAAATAGTTCATTTCCGTGAAGCTTTATTT
AGTGAAGAATAATTTTGTCATATTTTGTAACTGATTTAGTGAAGTTTG
ACAGATACTAGATATTATAACCTGAGACTCTAGAGTGTTTATTTCTTT
TCCCTTATTTGAACCTTGAATTCTCTGTAATCTGTTTTGTTTCTGGGCC
CAAACGACAGATAACGTGCCTCCTTTATTAGAAAGGAGGGTTAACAG
AGGTAACAGAAAAAGATAACAATCCTCCTTTATTAGAAAGGAGAGTT
AAATGTTGTATACAAAAAAAGGTCAACTGAATAGTCCCATGTCGGAG
AAAAGAAACCATATGGAAGTGATTAAATAGATTTCATTTGCTAACTT
AATTTTCACCAGATTAAAATACTTTGAGTTATTTTAAAGGAAGGAATT
TTAGTGATTAACACATAATCCTCATGTGTTCTTCCCACTGAAGCAATA
TGAAATGTTGCTCTTGCATTTTTCCATTTCTGAATATACGTTGCTGATT
GTAATTATTAGAATCATAAATGACTTTGGAAAAGCTTTTTGTATTTTT
TTAACAAAATCTATCTTCTGTAGCAGAGTTATATTTTCTATCACATTTT
CCATTTTCTAATTACTTTTCTAAACTGATGCTATTAACTGCTGCAGTTC
CTACAGGAGTGGTTGTTTTTTTAAGTCAGCAATAGTGGTTTATTTGAC
TCAGCTAAACAAGTGTTTTCTCCATCTCACCCCCGACTTCTGCTCTTC
ATGATTGAGTTGGAAACCCCTTAGGTGCCCTGCCTAGAAAATGTGCA
CTCGTTCTCACTTAGTGCCTCTACTGTGCATATGAAGTTTGCTTTAAG
GTTTTCTGATTATTCTACAGAGACCTCAGCATGGTCAAGGGCCAGGA
TCAGGGCACTGTGTGATCCATACATGAGGTAGTAATGACACTGCATT
TCTACCTCTTTTCCTTAAAAGGACTAGAACTCCAGGGTTGAGAGACAT
ATTTATTTTCAGAACTAAAATTTTTCTTTTAAATGCATTCTACTCAAA
GAAGCTAAGAAATAAAGCTTTTTAAAAGTGTGGGATCATCTATCATT
TTAAGGTTTTCTTCATTAAAGAACAAGTAATACAGAGACAAGGGAAA
GGTTCAGAATGGAGATTAAAATAAAACTCATAAAAGCGGAAAGAAG
TTAATTTGAAATAGTTAAAATGAGGGGGCTTAGTGACCAACTACCAG
CACGTTGGTCAGTTTTCTTAGATGCCTATAAAACCTGTTGTTGACTCA
CTTTTGAGTTATTAGGGTCACGTATAAAATGCCCTTGTCAGAAAACAC
AGAGATCCCAGTTCAGATGTGCCCCCTCAGGACCAGCAAATCTAAGG
CAGTGCCTACCAGTCTTGATTTCTTGCGTTTCACTCTGTCACATCCTG
GTCTTTATAGTACTTACTACTGTCTGAGATTACATTGGTCAGCTATTT
ATTTACTTGTATGTTTACTGACTCCTTTTATTCTAGACTGTAATTTCCA
ATTATATTGCCCCTCTTATTGTTGTAGCTCAAATTTTTAGAACTGTGCT
TGGCATTTAAAAGGTACTCAATTAAAGTTGAATAAACGAATGATTCC
TTAACCATTTGTTGATAAAGTCCTTCCTATTTAGTCTTTCATTTGATGA
CTTCATTTGATGCCATTCAAGTTAGAGCAGTTATTAGCCACATTTTAC
ACATCCCAAATGATATGCTAAGAAAAGCAGTTTTTGAACTGAAGGGT
AAGGAAACTGTATAAGTATGAATATGTAAATTTTGTCTGAGGTAAAA
ACACTTAATTGAGCATAAGGGCTCTCATATAACAGTGGACACATCTT
CTGTCTGAGAGAAAGGAAGGGACCACTCTGTTTTACTGGTTTTTTTTT
TAGTAGGACAGGTGTCATGCATTCATTTTTATGACTTCTCCAATACCT
GCATAGCTCTTAAATATTTACTTTTATACTGTTGTTCACATGGTAAAC
AAATTTAGTTTTGTTCAGAAAATGCTCACATGGTAAGCAAATTTAGTT
CAGAAAATGCTTGATGTATCATTCCACATTTAAGAGTTCAGCAGATG
ACTGCTTATGGATTTGTTAACATCTGTCTATTACTATTGGAATCACTTT
CTAATCTTAAATTTGGAGTGCGTTAGACTAAAGAAATGATTCCTCACT
TTTATGGTATTAGCAGAGAGAATGAGTTAATATTTTATGATAATTGAA
ATTTATTATGGTGATCACCAAAGTTAATATTTAGTAACATGCTAAAAT
ACTAGTTTTCTAAGAAATTATGTGTGAAACAAAAATGATAGCAATTC
TCAATATCATGAATCTATACCACTTTATGAGAAAAGAAATGAGTATG
CAGTTGACTCATGACACTGGTTTGAATTATGCAGGTTCACTTATACGT
GGATCAAAAAATACAGTATTTGTAGGATGTGAAAACCAAGTATTAGA
GAGTTGACCTCGTATATGCAGGTTCTGCAGTGCTGACTATGGGACTTG
AGTATGCATAGGTTTTTAGTATACATAGGGGTCCCGGAACCAATCTC
CCAAGTCTAGCAAGGAACACCTCTATATTTAGAGGTTTGTTTTTTCCT
TGAGTAAAGGGAAAAAGTACTAAACGATTGCAAGTCTTTTCTGTATC
ATAATGTATTTCAGGTAAATCAGCTTGAAGAAAACTCAATTATGGAT
TTTAGAAAATCAGTGTCGGCCGGGCGCAGTGGCTCACGCCTGTAATC
CCAGCACTTTGGGAGGCCGAGGCGGGCAGATCAGGAGATCGAGACC
ATCCTGGCTAACATGGTGAAACCCCGTCTCTACTAAAAAAAATACAA
AAAAGTTAGTAGGGCGTGGTAGTGGGCGCCTGTAGTCCCAGCTACTC
AGGAGGCTGAGGCAGGAGAATGGCGTGAACCCGGGAGTTGGAGCTT
GCAGTTAGCCGAGATCACGCCACTGCACTCCAGCCTGGGCGACAGAG
CAAGACTCCATCTAAAAAAAAAAAAAAAAAAAAAAAAAAAAGTGTC
ATATTTTCTATGCATATTTTCATTTTTGAAAGATTCTTTGGAGGGTGGT
ACCCTACTTTTAAATAAACCACTATTAAGGCCTACCTTCTTCTAGTGA
TTATCATCCAAAATTAATAAATATTGATTCTAAACATTCAAAAGGAA
AAAACTGACCATATTAAAAACTCTTTGACACTTTTTGGATCATAGGTA
AACTTATTTGAGTGTAAAATGTTGAAATTTTTGATAGAGTGTCATCAT
ATTTATATTTTAGTTAACATATTTCCACACTATCACTTATAATGCCAG
AAACTATTTCTGTAACTATCTTTATGTATTTAACGTTGTGGCTTATTGA
TAATCTTAAGCTTTTATGTAACTGAATGGTCTTGTATTTAAATGCCTG
TTTGCTGATTTTTTGCTGGTTGCTCATACTTGCTTTATTTTTTAGGCAA
TAATAAATAGTACCATCACCCCAAACATGACATTTACTAAAACATCT
CAGAAGTTTGGCCAGTGGGCTGATAGCCGGGCAAACACCGTTTATGG
ATTGGGATTCTCCTCTGAGCATCATCTTTCGAAAGTGAGTTAAATCAT
AAAATTTGAATGAAAATCTTCATCTTCAAGTCGTATATCTTAAATATA
TAATACCAAATGGAGAGTATAAAATATAGGAGAAATTTATTTAATAT
TCTCGAAACAGCAGTAGTGACTTCAGAGCAAGAAGTAAGAGATCATT
AGATTCTGCTGTCACTTGGAAAATGTACACATTAAAAACAGTCACCA
TTGGTTGAGAAGGATTTATGTGGTTAATTGTCTGTGTGATAGGAATTA
AAAGGTGGGGATTATCACTTCAGAATGGATTTTTGTCAGAGATTAGT
ACCCTCAATTGGAGATGGTCTACAAGATAGGCAGATACCAAGCTCTT
TTGAATTGTGAAAACTCAAGCTGACAGTGATATAGATTTCAGCATTTT
GATGTGACAGCAGGTACATATAAATTACTGTGTTTAGGGGAAATTGT
GCTTTATCTGGATGTGGAATACTCCGAGCTCTGATGCAGTGCAGACT
GAGATACTATAGACATCATGAGGAATTTTTAGATAATACTCACCATG
TGACCAGAAAGGCCAATAAAATGCAAAGCATCACCAGGCTAAATATT
GGAAATAAGACAGTTCTCCTGTCAATAAGCAAAAGCCATGTAATGTT
TATACCAGCAGCACCTTGGGTTCTTGTGGATGTTACACAGAAAAGAA
TATATAAGAAAAAACAATCTCAGTATACTAAGTGATCAAAGAGAAA
AGTAAAACTCACTTTTCTCTTCAGAGCTTGTGCCTGCCAGACCTAAAG
ATTTATTCTTAAAACCTCTGTTTGAAGCATTCAGTTAAAAGAATAACC
ATATATTTTAAATACTTTACATATGATTTGGTATATGCAGGATCTGAT
ACTGGAACTCAGGCTAGAGAGTTGAAATTATCCAAGTGCAGAGAAAT
CAGGCTCTTGTAGATATACAGTGTTTATATTTACATTAAATTTTAAGC
TGTTTTTCTTTACAGAAATTATTCTGTATTCAAATTCTTAAACTGGGTT
CTTTTTAGAAATCTTACACTTTTATGAATATATATTGAATCTATTTTAT
TTTCTTCTAATTATAAACATTTCTTAACACTGTTTTCAAGTGTTTTTGT
TATCTTTTGGATATGAATTATAGTTTAAATCTCAATTTTAAGGGTATT
TTAACTTTCGCCTTGGTGATTATAAATCATTTTCTGTAGGATGATTAA
AACAAATATTAAGGCTGGGCACAGTGGCTCATGCCTTTAATCCCAGC
ACTTTGAGAGGCCAAGGAGGGCAGATCATTTGAGGCCAGGAGTTCAA
GACCGGCCTAGTCAACATGGCAAAACCCCGTTTCTACTAAAAATACA
AAAAAATTAGCTGGGTGTGGTGGCACACACCTGTAATCCCAGCTACT
TGGGAGGCTGAGACATGAGAGTCATTTGAACCCAGGAGGCAGAGGC
TGCAGTGAGCCAGGATTGCGACACTGCACTCTAGCCTGGGTGACAGA
GCAAGACTCTGTCTCAAAAAACAACAACAACAAACAAATATCATTTT
GGTTTCTCTTGTACACATCTTATTCTTTAAATGTATTTCTTGAACTGTG
GTAAAATAAGAGTTTTACATTCAAAGTATCTCAGCATAACTAGGATA
CAGGTTTCCATTGAACATTGCCCTTCCATAAACAGTTCTGTTGAGAAA
TTATATGTTTATTCTAATACTTTTGCCATCATTCAGAACCTTTTTAGAA
CTTTGTGTTTTTTTTTATTGCCTATTAAAGACTATAGCAAAAATCTATA
AATATATCTTGCTGAATTTCTTAACTTGACAATACAAGAACTAAATCA
TGGATTATTTTCTCAAACTTGCCTTAAATGCTTTTTGGGCTGAGTAAT
TTTGTCTGTAAATTAAGTTCTTCGTTACATCTTTCAAGTTACTGAGCA
GCTAGCTAATGTAGTAAAGGAATAGTTTATTAACTTCTGTCAGTTCTA
CTCTTTTCCCTTGTTTGAACTTTTCTCTGAGAATGGCTTTTTTTTTTTAA
TTTTTTTTCTTCTTTAAAGTTAGCATATGCTTTTGACCAGCCTTACTGG
TGGTAAATCTAGTATTTGAGGGTAGGTTGAATATTGGGAAACAGCTC
TTAAGCTCTTGGAGCCAATTCTAGCTTATATACTCACAGAACTTGGCT
CCAAGAGCTTCAGATCAGTCTCTTCAGAATGTTTTGATCCAAAGCTGA
CAAAATCATAATTTATTTTTCAGGCTACCTTGTAGACTAATTTTGGTG
TTTTGCAGACTGTAGGAGGCTCAAAGGCATTTATAATATTTTACATAA
ACAAAGCAAGTACATAAACATTCTAGCTTGTTTTATCAGTTAAAAGC
CATGAGTAAGTCAAAACATTATAAGGGTTTGGAGAAGGCAATTCTAG
ACAAAGACATCTCAAAATAAGGGAAAGTACCATCGTTGAAAAAAAA
TGTGTGGTCTCTTAAGATTGAATGGTATGAGCAAAATTCCAGCTATGC
TTGGATTGTTAGAGTGCTTACTAAGCTGTATTTTGTTATTATGGGAAA
TCTAAGCTGTCACTTTTTTATTTTGCTGAAAACTAAGGATATACAAAT
TAGCAATAGTGTAGATTTTTACGAAGTTGAGAAGGCTTTTAAAGATT
AACTATTGTGGAATGTTAAATTAGTGTAAGAAAAACCTTGTATACTA
TTTCAGATTACATAAAGAGAAGTTTTGTTTTGATTTCATTTCCACCAA
TGTGAGTGAGACCCTTGTAGCTTCGTATTCAGATGTTGTACTGTTTGG
AGATACTCTGTTCAATTGGAGAAGTATAGTAGCACAAATTCACTTTCC
TCAGATACACATTATAGGTAGAAGTGACAAGGTTTAAGGTAAGAAGG
AAAAGTIGTGATAATGATGGCTTTAATTTTGACCCTCCTCCATCTCGC
CAGGATTAGAGTATCACAGGCCTTTCCTTCCAAAATTTTTCTTCCCAA
GATGGAAATGAAATCAGAGTTCGTTTATGAGGGTTTGAGAAAGGAAC
TATAATTTCATGTTTTAATTACCAGCTCTAACAGATATTTTAAGTTGC
ATAGGGATTTGGGGTTTTTACATGTAGTTATTATGTAGTCTTAACTTA
TTCTCACTGATAATAGTGATAGACTAGCTTCCACTTCCAAAACATAAA
TAGATATTCTTTGTGGCCAGGATGTTTTATAGTTTCCTATGAGTTCCT
AAGAATTAGCATTTCATGTACAATAATTTTTGTTTTGAAATTGTTATC
CATCTTAAAGACCACTGAGTATTATTCCTTTCTGCTGCTGTACATGAG
CTATTTAGGAATCTTCGGAAATAAATTTTTAGTTATGTCAGTTAGTGA
AAATAGTAAATATTACTATTTGTATTACTGAAAATGCATTGTGTATTA
AGAGAGTTTATATTTTAGTAAAATTTAAAAGCATTTTCTAAATACTGT
GATGTTTACAAGTGGATACTTGTGAAATTCAGTTTTCTCAATGATAAA
CATAGCACAGTACGCTTATGTTTACTGATTCTTTCCCCTACCTTGGTCT
CAGTAAATTATTTCTGAACAACCAGGAGTCCTTTAGGTAAGCTAGCT
GACTCTGAAGTCTGACTTAGCTATATCATGATGTTTTGAATAAGATTT
GTCAAAATACTTCATAGTGCACAGACAACTGTTTGGAATTAGGTTTC
ATTTCCTGTCATTTTCATATTAGAAGCTATAATTGGTTTGGAGGCTAT
ATTGCTTGTGTAGAGTCAGTTATTCAGAAAATGAATAACTTTACTAAG
GTAAAAGTGGGCACTGTGACCTATTTTTATTTGGTTAATGTATGTAGT
CTCTATACATTCAGTTTGCAGAAAAGTTTCAGGAATTTAAAGAAGCT
GCTCGACTAGCAAAGGAAAAATCACAAGAGAAGATGGAACTTACCA
GTACACCTTCACAGGTGGGTATATCATTTCTATTCTTAATTATGAACC
TTGTTCATTTCAGATAATATCCTTCATGCCAGAATTAAATCTCCAAAG
TACACACAGAAATGCACCCTTTAGTGTGGTGGCTCACACCTGTAATC
CCAGGACTTTGATAGGCTTAGGTGGGAGGATTACTTGAGCCCGGGAT
TTCAAGACTAGCCTGAGCAACATAGGGAGACCTCATCTCTACAAATT
AAAAAAAAAAAAAAAAAAAAAAAAATTAGCCAAGTGTGGTGGTGCA
AGCCTGTGGTCTCAGCTACTTGGGAAGCTGAGGTAGGAGGATCACTT
GACTGGGAGGTCGAGGCTGCAGTGAGCTATAATTGTGCCACTGTGCT
CCAGCCTGGGCAACAGCAAGACCCTGTCTCAAAAAGAAAAAGAGAG
AGAGAGAAATGCACCCTTTACTTAATTTGTACTGGTGGAAAAAGGTT
TTTCTATATGCCAATGCCTACAAGAATATTTGTAATCAAAATTTCAGT
TTAGTAATTCAATTCAAATTATTTAATGTGCTTGTAACTTAGCAAAAG
TACATAAAGATCATGTGATAGTCAAATGTAACCACAAGGGGAGTCAT
AGGAAAAGTTCAGGAAAACAAGTTTATTATACTTGCTGGTCCTAGAA
ACAGGGAAAGTCTTAAGGTGGTCAAGAGACAGAAAGCAGGAGTGAG
GGGAAAGCCGAGGTAGAGCTTTATTGGAATTTCCATTGGAAAGGTAA
AGCAGGGGAGGGGAAATAGTTTAGGATTGGCTGGGTTGAATAATTTC
AGGGGGCTCTAAACTGTAGGGGGGTCCCTCATTGTCTGGTAACTGG
CCCTGGGATGGATTAAGTTAGAGGAATATTGTCTCCTGGATCAGATG
TATGGATCAGATAGAGGAGGTATGGCTCTGGATTGGCTAGTTTGCAT
ATCAGAGGCATGCTCTGGACTGGGCTCTTGCTGTCCTTAAGAATTAGC
TAGCCCTAGAGGGGAGAAGCAGTCTCTTTGCAGCCAGAAAATTTTTT
TAAAAGATGTCAGAATGTCATAATATACATAAAGTTAAAATACATGC
ACGTGCGTGCACACACGCACAAACAATAGGGTTTGTTTTACATACTTT
TATTATTTATTTATTTATTTTTGAGCTAGAGTCATGCTCTGTCACCCAG
ACCGGAGTGCAGTGGCACAATCTCGGGTCGCTGCAACCTCTGCCTCC
CAGGTTCAAGTGATTCTCATGCCTCAGCCTCCCAAGTAGCTGGGATTA
TAGGCGTGCACCACCCCACCTGGCTAATTTTTGCATTTTATTAGTGGT
GGTGTTTTATCATGTTGGCCAGGCTGGTCTCCCTGGCCTCAAGTGATC
CGCCTGCCTTGGCCTCCCAAAATGCTGAGATTACAGGCGTTAGCCAC
AGCCCCAGCCTGTTTTACATACTTTTAAAGTATACTTAAATCTGACAG
TTGGAACATCTTTATGCTTAGCACTGAGACAATGGTTATAGGTTTTAC
AAAAGTATTACTTTTTCTTTTAACAAAGGACCTTTTACTTGTCCATCA
TAATCTTCCTTCAGTTGGAAGTTATATCTCCAAGTAAAGTTGATTAAA
TAGTTACATTTTGGTACTTTTATAATACAGTGGTTTTACTGTATGATTA
ATATAGAATGCCACTATTTTCAGCTTTTTAATAGTAGGCTTTTCTAAA
ATTAATTTTTATTTTAATTGTATCCTACTAGTAGGATTCAGCTTTTGGC
TGTTCTCCTCTGTAGTAATTGTTTCTGAACTGTACTCAGTAGCTTTTAT
GGAAAGGAAGAATCAGATGTTACAAATGTTTCTCTGGAATAATGTCA
GTAGTAGCAGATTTACATGGAACTTTCTTGAAGAAAATAGGAATTGC
CTTGGAATTTAATAACTAACCAAATGATTGGAACAGTTTAGAAAATT
ATTTATTCTTTATCTCCCTCTCCACTTCTATCACCCAGCTCTATTTGGA
AAACTACTGGTATAGAGAGTTGATTTTTTTTTTAATAAGAATAAAGG
AGAGCTCTGTTAAATATGCTTAAAGTGCATATTCCCTTAGACTAAAG
GTGTACTCTGGATTGAAGCCAAAGCTCCATTATACGTCTGTACATGCA
ATGACCTGGAATAACTTTGATGATCATTTGAAACTTCTCTTGAATCTG
TCTTCTCTAAGTGCTGCGAAGGTGTAGTGATACTTTTAAACATAAAAC
CCCATTTAAAATTAAGTGGCAATACATATTGGGAGCTCTAGTTCCTCT
GGTGAGTGATTCCAGTGTTATAAGAAGATCCAACAAACAGCACATTC
AGTAAAAGAGTACTCAAAGGATTTTAATAAAAGTGTGATGGATTAAG
CTTTGCCATTGTACTTAGTTCCCGTTTCTCTAATCGTAACTTCCATTTT
AGTTGAATTGGTCATGAACCATTATAAGTTGCTGCTAACACCTTGTTC
CTCTTTGGTGGAAGGAATGACTTTAAGCCTCTGCTTTGATTAAAGGAA
TATATAAAAGATGCTTATATTTGAAAAATACTATGAAATATAAAGAA
TAAGGAAATAAGTTCATCACTATTTGAGAGCCAAAATATATGGACCT
TAATTTATCTTATCAAAACATTTGAAGCCTAATTGTCAATTTAATATA
CAGAAGCCAGAGAGAATCATTTTTTAAAAAGCATTTATCAGCCAGGC
ATGGTATGCACCTATAGTCCCAGCTACTCATGAGGCTAAGGTGGGAG
GACTGCTTGAGCCCAGGGGTTTGAGGCTGCAGTGAGCTACAATCACG
TCACTGCACTGCAGCCTGGGTGACAATATCTAATGTGTCTGTTTTAGT
TTGAATTGTATGACCATTGGCCATACCTACCCTACTTGATTCTCAGTG
GTTTAACCCAATCTGCTGAATTAATGCTGCCTGAAGAATGTAACGTA
ACTCTGGAATTTAGCAAAAGAACGGTTAAGTGGGGTAATTGATAATA
GGTTAAACACGAAAATCGATAGTAGTAGAAACTTGGTTGGTCAAAGC
TTTTGTTTTTATTTGAGGGAGGTGTAAGAGACTGGTTAAAAGTTTGAG
TCAGACAAACCTAGATATAAATTACTTTTCTGTTGCCATGTAATACAC
TTGGTTGTTTAACACAAGTGTGTTTATACATTTATAACATGAGTATTA
CATTACTTATAGAATCATTGATCATCATCAATAAGTATGGTAACGAC
ACAGTGTCAGGTCATGGTTAATAATGGGAGTTCTAGAATCTGAATTT
GAAACTGGGCTCCAACCCTTAATAGTTTTCTGAGCTTAGGCAAGTCTC
TGTTTTTCTTCCTCTATAAAATGAGAATGAGAACAATATGTCCTTAAT
AGGAATATTGTGAGAATTAAATGAGGATTAGAAAGTACTTAGCATAT
AGTACTAAATGCCTAATAAATGTTAACAACTGATGTGGTTGTAATGG
GGAGGATGATGATGTAAGGCCTTAACATACTGTCTGGCACTAACAGT
TATCTTTAAAAATTCAGATGCCTTAATTGTATATTTATACAATATGTA
TTTGAAAAATACTATGCGTCAGAATTCTGCATCAGAATTCCCTGAAA
GACTTGCTAAAACTCAGATCGGTGAGCTCCACTCCCAGACTGTCTGA
TTTAGTAGGTCTAAGATGGAACCCAAGGATTTGCATTTCTAACAAGTT
TTCTAGGAGATGCTGAAGCTATTCCTATGTTAGATATTATATTATATT
GTAGAATATTATATATGCCTAATTCAGTTGTGAGTATTATTTTAATTT
TTTAGCCATTTACATTTTAGTCATTTCAAAGTATTTAGCCTGGCTTTCA
TTAAAATTACCACTCTAGTTTTGCATTCAAAATCAGTAGTTTACATAG
TACTTAATTCAGTTTTATAATTATATAAATAGAATTGGCATAAACAGG
TTTGGGACACATACAGCTGACTCTTGGTAAGCATTTGATGCAGCTGGT
GGTTGCAAAATATTCAAGCAGAGTAACAATTAACCCACTAACCCTAA
GCATCAATCAGCCAATATACTTTACATAGCGAAGGAAGAGCATAAAT
TAATCCAGAAAGTCAGATTATAAGAACTTCTGTGGTTGTCACAAAAT
GGATTTCAGAAATGGTGGCTCTGGATGGTGACTAACTATAGGAAAAA
TGTATGACTGAGGATATGAGGATCTTGTTTGAGAGCTCAACCTCATCT
TCCTTTATGTATCGTTTTGCTGTCTTCTTTCCATTCTCACTGCCATGGG
ATCTCAAGACTATTAGCTCCTCCCCAGTTTTTCTGTCAGGCCCTCTGC
TGAGAAAACAGTGGTGTTCTCATCTTTGTTTAAGTATAAAACTGGAAT
GGACTGAGTCTTCCATGAATGTTTGTTAAATGGTAAAGAGAGGGCCT
TGTTTTCCTTAAGCCAACAAGGTTGAAACTGTAATGGGAATTTAATTT
CAGGTTTTATTGGCATGAAATGGAAGGTGGTAGTGGTATTTGGTATA
GTGGGTCATGGCAGTGCTGCTGCGTACCTCCCCAGCCCATCCTTCCAA
TACACAGTAGGTCACTGAAACCACCCTAAAGGCTTGTCCTAGAGTGA
GGGTCAAGGTCAACTTTTTTCCATGTGTTCTAGAATATTTCTCATTTTT
TGGCTTTCATAGCCAAAATTTCAGTTGTGGGTTTTATTTTAATTTTCAC
TTAAGTTACCCACTTCACAAATGGGTAGGAAGAGAGAATTCTTAAGA
AGCATTTCCTGAATCTCACATCTGTGCTCAGGTGCCTTTTTTATAATA
GGTTGCATCTGTTAGTTTTGATTTCCTTGATACTCTCTCAGATTTGTTC
TTCCAAAAATAAATTTTTTCTTCAGTGTTACTGTCCTTTTTCTTACTGT
AGATTATTTGTTTTGGTGGGAGTTATAGGGAGAGTTTCTCAGAAAAA
AAATGAGATGTAAGTGGTTTTATTTTCTCAAAGCAAGGATATAAAGT
CAATAGAAAAGATTATTTCATCTGTTTTTATTTCATTTTATTTTTTGCT
TTTTTTTTTCATTTTTCTTATCAGCTTTGGGTATACACACACACACACA
CACCCCAAAAAAGAATAACTGAAGTAGGATGTTTAGGAAATTACACA
CAATTTAGATTAAAAAAGAAATAGATGCTTAGAATATGTTAAATAAA
AAACATGTTTCAAGTACTATTTTTAGCATATGCAAAATTTCTGGAAGG
TTTTGCAGAATACTTTTGGCTACCTCTGCGGTAGTTGGAGGGAAAGA
GGGTTTCATTTTCCTTTTGTGCCCTCATGTACTGTTGGAATTTTTTGTT
GTTTCTCATCAGTAAATACTAATTTTATATTTTTAAAACTGACTTTAA
AAAAACCCAAGTTCTCGATCGGTTCAGAATAAAAAATTAAATTAAAA
AAATAAAATACCATGCTTTGGGTGGGCTTTTTGTTTTTTACTTTAGGT
ATAATGACCTAGGATAACTATGCCTTTACTTAAATATAGCTAATATTC
AAGTTTTCTGAAGAAAAACTTGCTGCTTTGAGATGTTGGAATTGCTAC
AACCTCTATCCCTTTCTTTCTCCCCTTCTTAACTTGCCCCTGGGAACAG
TCTTTCTACTTTAGTAGCATTAACATTTGTAGCCTCCCCAAGTTCCAT
GCCTCAAACCCTACTCCCTCTTATATTACCCTCAAAGTGACCGGAGCA
TTTATCCCAGAAGATGGTAACAACCATGAGTCAAACTGCTACTGCTC
TTCCTCTCTCTCTTTTGATCCTAATGTACTTTTGCACACTCTTGTCAAC
CAGTTCCAGCAAATCCCTGAGTTAATCCTTAATTGCTAACCCTAGTTA
TGCCCTTTAAGTTTAATAAGGTAATTTATTTTTATTTTTATTTTTTATTT
TGAGATGGAGTCTCACTCTGTTGCCCAGGCTGGAGTGCAGTGATCTC
GGCTCGCTGGAACCTCCACCTCCCAGGTTCAAGCGATTCTTCCACCTC
AGCCTCCCGAATAGCTGGGACTACAGGCACCTGCCATCATGCCCAGC
TAATTTTGTTTGTATTTTTGTAGAGATTGGGTTTCATCATGTCAGCCA
GGCTGGTCTTGAACTCCTGACCTCAGGTGATCCGCCTGCCTTGGCCTC
CCAAGGATTACAGGCGTGAGGCACCACGTCCCGGCCGGTAACTTGTT
TCTGTTATCATTTCTACTGAATTGAATCTTCACCAATTTAACTGTTTAG
CAGGTTGCACATTCAACCTTTCATTATTTTAGTTTGGCTTGGCCTGTCT
TGGAGAGAAATAAGCCTGATTTGTTAATACCAGAGATGATTATTTTA
TGATATCTTACTGGCTGGTTAAATTTTTTCTGACTAATTAATATGGTC
CATAACTGACCTAGAAGTAGTCACTTTGTGATTGCTATTCCCATTATT
CACTTAATAGGATTTGACTTTGTTGTAAAACATTTTTATATTGTCACA
TTATACTGTAGATTTTATTCGTGCATTGACTGTCAGCTAAAGGAAACA
GCTGATTTAGGAGAAATATTTAATAAAAATGAAATTAAAGGTCAAAA
TTAGAAGTTTTACTGAAAATTGAAAAATAGTTAAGTGATAATGTGGA
ATTAAGAAAGTATCCTTTTGATCTTTAACAGCTTGTATAGTTTATAGC
ATTATGGAACCATTAGTTAAAATACTTAAATGTGAAATAAAATTCAT
ACAGCAGTATAAAACCAGAAGTCTCAAAAACAAAAGTCTCACCACGT
TAATATTTACATATTTTTTCTAGCACTTCATGTGTAACATTCTAAATGT
GAAGGATATAAATATAACCCAGTTACTGTTTTACATTCTGATTTTTAA
AAATTATATGCTACATTGCTCATATTTGTCTTATTTTTATTAGAGGAA
ATCTTGTATTTTTTTTAAGTTGACAAATTATGATCATTCCTAACCACTC
CCCTGTTGGACACTTATTTTTTTCTCCCAAATATAGACAGATATTTCA
ATTGTAACATTTTTTTGCCCAGGGATCATAAGAGGTCCACCAAGCTGC
TCTTTAACAGTCTGGTTTTAGCACCTCCCCTAGTTAATAGGTCTATCA
AATGACTCCTCATTCTGATTTCCAATATCAAATTTGGATAATCTGATG
GTGGCAGACATAAGTGTCAGCTTGAGCTTCATTTTAAATTCAATTTAT
TTAAAATGACATAAATTTTAATGTTATTTAAATAAATTTACTTTGAAA
GGAAAATCTTAATGAAAAGTAGAAATAAGACAGGAAATTTTGTAAA
GAAAAGTAATCCCACTATGAGTACCAGACAAGATAAGTTGAAATGCA
CTAACATCATTATAGATAATAGGTTATAGAGGCATTTCTTCTTGCTTT
TCTAAGTAGCTCTATAGGGATTGAAAAGTAGGGCAGATAAAACGTGT
TGCAAATTTCATTCACCCCTTTGCCCTCCCCCTCTCCTCATCCACTTCT
TCTCCTGTACCCCTTCCATTATCTACTTCTTTCACCAAAATCACATCTT
CTTGCATTTTTGTGCATCTTTTCTTCTGAAGTATAGTAATTCTTTGAAT
ATCTTTTATTCTGACCAATTAGTTAAATAAACCAAACATTTTTAAATT
ATTCGAAACTGTTTCTAAATTGTGTTATATTTCCGTTTCTAAATTGTAT
GTTTCAGAGAGTTTTCTTGTGAGTAGCCCAGCTTCAAAAAACTTTTTC
TCCTAGCTTGCCATGAATAAAAAATAAAAATTTCCATGTTTTATGTTT
TTAAGTCTTTGACTCAAATTTCAGTTGAAATTTGGTTCATCAGTAAGC
ATACATCAAAGGCAGTTTTAACCTCAAGATGATATTGTGTACTTTAAC
AAAAGTGTAACTATTTTTTATCCCCCACCCCTTTTTTTAAAGGAATCC
GCAGGCGGGGATCTTCAGTCTCCTTTAACACCGGAAAGTATCAACGG
GACAGATGATGAAAGAACACCTGATGTGACACAGAACTCAGAGCCA
AGGGCTGAACCAACTCAGAATGCATTGCCATTTTCACATAG
Length: 18009 nt
Human circHomer1 (hsa_circ_0006916) mature circRNA sequence
GGAACAACCTATCTTCAGCACTCGAGCTCATGTCTTCCAAATTGACCC
AAACACAAAGAAGAACTGGGTACCCACCAGCAAGCATGCAGTTACT
GTGTCTTATTTCTATGACAGCACAAGAAATGTGTATAGGATAATCAG
TTTAGATGGCTCAAAGGCAATAATAAATAGTACCATCACCCCAAACA
TGACATTTACTAAAACATCTCAGAAGTTTGGCCAGTGGGCTGATAGC
CGGGCAAACACCGTTTATGGATTGGGATTCTCCTCTGAGCATCATCTT
TCGAAATTTGCAGAAAAGTTTCAGGAATTTAAAGAAGCTGCTCGACT
AGCAAAGGAAAAATCACAAGAGAAGATGGAACTTACCAGTACACCT
TCACAGGAATCCGCAGGCGGGGATCTTCAGTCTCCTTTAACACCGGA
AAGTATCAACGGGACAGATGATGAAAGAACACCTGATGTGACACAG
AACTCAGAGCCAAGGGCTGAACCAACTCAGAATGCATTGCCATTTTC
ACATAG

### Mouse Intron Deletion (circHomer1; circbase allias: mmu_circ_0000491)

CircHomer1 KO 5′ gRNA: AAGCTTAGGATGTGTGGAAC
CircHomer1 KO 3′ gRNA: GTTAGAATAGAACTTGTCAC
Mouse circHomer1 KO 5′ Cut-Site:
ACATTTCAAGGAGTGATCACTCTTTGGTGTACTTCTTTAGACGCTGTC

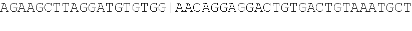

TACTAATCGCTTTGAGAGAGTGTTAT
Mouse circHomer1 KO 3′ Cut-Site:
AACAAAAACTAAAAATGGAGCTTTACTTCTGAACTCTCAGTTCTGGC

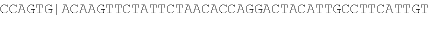

TTTAACTTGGCATTAATATTTAAAA
Mouse circHomer1 KO Sequence after intronic area
deletion:
TGGGTTCGTATATACTTTCAGTTAAGAGCTGTAGTACTGTTTTATGTC
CTCGTCATAGCTATGCAATAATAGTCTCATCAGATACATTTCAAGGA

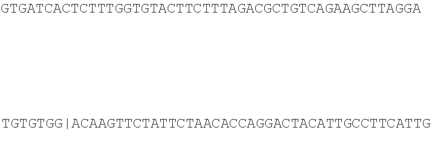

TTTTAACTTGGCATTAATATTTAAAATTGGGAAGTATGATTTGTATAA
CCTTTTCCTTTTTTTTTTTTAAGATCGAGTCTTGAGTCCTTACAATTTC
TAAGCAAAGTTT
Mouse circHomer1 (mmu_circ_0000491) mature
circRNA sequence
GGAGCAACCTATCTTCAGCACTCGAGCTCATGTCTTCCAGATTGACCC
GAACACAAAGAAGAACTGGGTACCCACCAGCAAGCATGCAGTTACT
GTATCTTATTTTTATGACAGCACAAGAAATGTGTATAGGATAATCAGT
TTAGATGGCTCAAAGGCAATAATAAATAGCACCATCACACCAAACAT
GACATTTACTAAAACATCTCAAAAGTTTGGCCAATGGGCTGATAGCC
GGGCAAACACTGTTTATGGACTGGGATTCTCCTCTGAGCATCATCTTT
CAAAATTCGCAGAAAAGTTTCAGGAATTTAAGGAAGCTGCTCGGCTT
GCAAAGGAGAAGTCGCAGGAGAAGATGGAGCTGACCAGTACCCCTT
CACAGGAATCAGCAGGAGGAGATCTTCAGTCTCCTTTGACACCAGAA
AGTATCAATGGGACAGACGATGAGAGAACACCCGATGTGACACAGA
ACTCAGAGCCAAGGGCTGAGCCAACTCAGAATGCATTGCCATTTCCA
CATAG.

### DEFINITIONS

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein, “circular RNA” has to be understood as a circular polynucleotide that can encode at least one protein as define herein. The production of circRNAs can be performed using various methods provided in the art. For example, U.S. Pat. No. 6,210,931 teaches a method of synthesizing circRNAs by inserting DNA fragments into a plasmid containing sequences having the capability of spontaneous cleavage and self-circularization. U.S. Pat. No. 5,773,244 teaches producing circRNAs by making a DNA construct encoding an RNA cyclase ribozyme, expressing the DNA construct as an RNA, and then allowing the RNA to self-splice, which produces a circRNA free from intron in vitro. WO1992001813 teaches a process of making single strand circular nucleic acids by synthesizing a linear polynucleotide, combining the linear nucleotide with a complementary linking oligonucleotide under hybridization conditions, and ligating the linear polynucleotide. The person skilled in the art may also use methods provided in WO2015034925 or WO2016011222 to produce circular RNA. Accordingly, methods for producing circular RNA as provided in U.S. Pat. Nos. 6,210,931, 5,773,244, WO1992001813, WO2015034925 and WO2016011222 are incorporated herewith by reference.

As referred to herein, the terms “nucleic acid”, “nucleic acid molecules” “oligonucleotide”, “polynucleotide”, and “nucleotides” may interchangeably be used. The terms are directed to polymers of deoxyribonucleotides (DNA), ribonucleotides (RNA), and modified forms thereof in the form of a separate fragment or as a component of a larger construct, linear or branched, single stranded, double stranded, triple stranded, or hybrids thereof. The term also encompasses RNA/DNA hybrids. The polynucleotides may include sense and antisense oligonucleotide or polynucleotide sequences of DNA or RNA. The DNA molecules may be, for example, but not limited to: complementary DNA (cDNA), genomic DNA, synthesized DNA, recombinant DNA, or a hybrid thereof. The RNA molecules may be, for example, but not limited to: ssRNA or dsRNA and the like. The terms further include oligonucleotides composed of naturally occurring bases, sugars, and covalent internucleoside linkages, as well as oligonucleotides having non-naturally occurring portions, which function similarly to respective naturally occurring portions. The terms “nucleic acid segment” and “nucleotide sequence segment,” or more generally “segment,” will be understood by those in the art as a functional term that includes both genomic sequences, ribosomal RNA sequences, transfer RNA sequences, messenger RNA sequences, operon sequences, and smaller engineered nucleotide sequences that are encoded or may be adapted to encode, peptides, polypeptides, or proteins. All nucleic acid primers, such as SEQ IN NOs. 1-446, are presented in the 5′ to 3′ prime direction unless otherwise noted.

As used herein, “complementary” refers to the ability of a single strand of a polynucleotide (or portion thereof) to hybridize to an anti-parallel polynucleotide strand (or portion thereof) by contiguous base-pairing between the nucleotides (that is not interrupted by any unpaired nucleotides) of the anti-parallel polynucleotide single strands, thereby forming a double-stranded polynucleotide between the complementary strands. A first polynucleotide is said to be “completely complementary” to a second polynucleotide strand if each and every nucleotide of the first polynucleotide forms base-paring with nucleotides within the complementary region of the second polynucleotide. A first polynucleotide is not completely complementary (i.e., partially complementary) to the second polynucleotide if one nucleotide in the first polynucleotide does not base pair with the corresponding nucleotide in the second polynucleotide. The degree of complementarity between polynucleotide strands has significant effects on the efficiency and strength of annealing or hybridization between polynucleotide strands. This is of particular importance in amplification reactions, which depend upon binding between polynucleotide strands. An oligonucleotide primer is “complementary” to a target polynucleotide if at least 50%, 60%, 70%, 80%, 90% or more nucleotides of the primer form base-pairs with nucleotides on the target polynucleotide.

The term “sequence identity” or “identity,” as used herein in the context of two nucleic acid or polypeptide sequences, refers to the residues in the two sequences that are the same when aligned for maximum correspondence over a specified comparison window. As used herein, the term “sequence identity” may refer to the value determined by comparing two optimally aligned sequences (e.g., nucleic acid sequences) over a comparison window, wherein the portion of the sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleotide or amino acid residue occurs in both sequences to yield the number of matched positions, dividing the number of matched positions by the total number of positions in the comparison window, and multiplying the result by 100 to yield the percentage of sequence identity. A sequence that is identical at every position in comparison to a reference sequence is said to be 100% identical to the reference sequence, and vice-versa.

The term “expression,” as used herein, or “expression of a coding sequence” (for example, a gene or a transgene) refers to the process by which the coded information of a nucleic acid transcriptional unit (including, e.g., genomic DNA or cDNA) is converted into an operational, non-operational, or structural part of a cell, often including the synthesis of a protein. Gene expression can be influenced by external signals; for example, exposure of a cell, tissue, or organism to an agent that increases or decreases gene expression. Expression of a gene can also be regulated anywhere in the pathway from DNA to RNA to protein. Regulation of gene expression occurs, for example, through controls acting on transcription, translation, RNA transport and processing, degradation of intermediary molecules such as mRNA, or through activation, inactivation, compartmentalization, or degradation of specific protein molecules after they have been made, or by combinations thereof. Gene expression can be measured at the RNA level or the protein level by any method known in the art, including, without limitation, Northern blot, RT-PCR, Western blot, or in vitro, in situ, or in vivo protein activity assay(s).

An “expression vector” is a nucleic acid capable of replicating in a selected host cell or organism or in vitro. An expression vector can replicate as an autonomous structure, or alternatively can integrate, in whole or in part, into the host cell chromosomes or the nucleic acids of an organelle, or it may be used as a shuttle for delivering foreign DNA to cells, and thus replicate along with the host cell genome. Thus, expression vectors are polynucleotides capable of replicating in a selected in vitro system, host cell, organelle, or organism, e.g., a plasmid, virus, artificial chromosome, nucleic acid fragment, and for which certain genes on the expression vector (including genes of interest) are transcribed and translated into a polypeptide or protein within the cell, organelle or organism; or any suitable construct known in the art, which comprises an “expression cassette”. In contrast, as described in the examples herein, a “cassette” is a polynucleotide containing a section of an expression vector of this disclosure. The use of the cassette assists in the assembly of the expression vectors. An expression vector is a replicon, such as plasmid, phage, virus, chimeric virus, or cosmid, and which contains the desired polynucleotide sequence operably linked to the expression control sequence(s).

A polynucleotide sequence is “operably linked to an expression control sequence(s)” (e.g., a promoter and, optionally, an enhancer) when the expression control sequence controls and regulates the transcription and/or translation of that polynucleotide sequence. As used herein, the phrase “gene product” refers to an RNA molecule, such as a circRNA or a protein. Moreover, the term “gene” may sometime refer to the genetic sequence, the transcribed and possibly modified mRNA of that gene, or the translated protein of that mRNA.

Notably, all DNA sequences provided may encompass all RNA and amino acid sequences, as well as primers and probes for detection of the same and vice versa as would be ascertainable by those of ordinary skill in the art, for example through Uracil substitutions as well as redundant codons. Additionally, all sequences include codon-optimized embodiments as would be ascertainable by those of ordinary skill in the art. As such, the term “encoding” or “coding sequence” or “coding” means both encoding a nucleotide and/or amino acid sequence and vice versa.

The term “transformation” means introducing an exogenous nucleic acid into an organism so that the nucleic acid is replicable, either as an extrachromosomal element or by chromosomal integration. The terms “transgenic,” or “genetically engineered,” or “genetically modified,” or “recombinant” as used herein with reference to a host cell, in particular a mammalian host-cell, denote a non-naturally occurring host cell, as well as its recombinant progeny, that has at least one genetic alteration not found in a naturally occurring strain of the referenced species, including wild-type strains of the referenced species.

The term “gene” or “sequence” refers to a coding region operably joined to appropriate regulatory sequences capable of regulating the expression of the gene product (e.g., a polypeptide or a functional RNA) in some manner. A gene includes untranslated regulatory regions of DNA (e.g., promoters, enhancers, repressors, etc.) preceding (up-stream) and following (down-stream) the coding region (open reading frame, ORF) as well as, where applicable, intervening sequences (i.e., introns) between individual coding regions (i.e., exons). The term “structural gene” as used herein is intended to mean a DNA sequence that is transcribed into mRNA which is then translated into a sequence of amino acids characteristic of a specific polypeptide.

A nucleic acid molecule may include either or both naturally occurring and modified nucleotides linked together by naturally occurring and/or non-naturally occurring nucleotide linkages. Nucleic acid molecules may be modified chemically or biochemically, or may contain non-natural or derivatized nucleotide bases, as will be readily appreciated by those of skill in the art. Such modifications include, for example, labels, methylation, substitution of one or more of the naturally occurring nucleotides with an analog, internucleotide modifications (e.g., uncharged linkages: for example, methyl phosphonates, phosphotriesters, phosphoramidates, carbamates, etc.; charged linkages: for example, phosphorothioates, phosphorodithioates, etc.; pendent moieties: for example, peptides; intercalators: for example, acridine, psoralen, etc.; chelators; alkylators; and modified linkages: for example, alpha anomeric nucleic acids, etc.). The term “nucleic acid molecule” also includes any topological conformation, including single-stranded, double-stranded, partially duplexed, triplexed, hair-pinned, circular, and padlocked conformations.
modification is typically achieved by technical means (i.e., non-naturally) through human intervention and may include, e.g., the introduction of an exogenous nucleic acid and/or the modification, over-expression, or deletion of an endogenous nucleic acid.

As used herein, “intron” is broadly defined as a nucleotide sequence that can be removed by RNA splicing. “RNA splicing” means cutting out introns from pre-mRNA to form mature mRNA. As used herein, the term “messenger RNA” (mRNA) means a polynucleotide which encodes a polypeptide of interest and which is capable of being translated to produce the encoded polypeptide of interest in vitro, in vivo, in situ or ex vivo.

As used herein, “heterologous” or “exogenous” in reference to a nucleic acid is a nucleic acid that originates from a foreign species, or is synthetically designed, or, if from the same species, is substantially modified from its native form in composition and/or genomic locus by deliberate human intervention. A heterologous protein may originate from a foreign species or, if from the same species, is substantially modified from its original form by deliberate human intervention. By “host cell” is meant a cell which contains an introduced nucleic acid construct and supports the replication and/or expression of the construct.

As used herein, the term “genome” refers to chromosomal DNA found within the nucleus of a cell, and also refers to organelle DNA found within subcellular components of the cell. The term “genome” as it applies to bacteria refers to both the chromosome and plasmids within the bacterial cell. In some embodiments of the disclosure, a DNA molecule may be introduced into a bacterium such that the DNA molecule is integrated into the genome of the bacterium. In these and further embodiments, the DNA molecule may be either chromosomally-integrated or located as or in a stable plasmid.

As used herein, the term “promoter” refers to a region of DNA that may be upstream from the start of transcription, and that may be involved in recognition and binding of RNA polymerase and other proteins to initiate transcription. A promoter may be operably linked to a coding sequence for expression in a cell, or a promoter may be operably linked to a nucleotide sequence encoding a signal sequence which may be operably linked to a coding sequence for expression in a cell.

A “cell type-specific” promoter primarily drives expression in certain cell types in one or more organs, for example, vascular cells in roots or leaves. An “inducible” promoter may be a promoter that may be under environmental control. Examples of environmental conditions that may initiate transcription by inducible promoters include anaerobic conditions and the presence of light. Tissue-specific, tissue-preferred, cell type specific, and inducible promoters constitute the class of “non-constitutive” promoters. A “constitutive” promoter is a promoter that may be active under most environmental conditions or in most cell or tissue types.

The term “vector” refers to some means by which DNA, RNA, a protein, or polypeptide can be introduced into a host. The polynucleotides, protein, and polypeptide which are to be introduced into a host can be therapeutic or prophylactic in nature; can encode or be an antigen; can be regulatory in nature, etc. There are various types of vectors including virus, plasmid, bacteriophages, cosmids, and bacteria.

A “variant,” or “isoform,” or “protein variant” is a member of a set of similar proteins that perform the same or similar biological roles. For example, fragments and variants of the disclosed polynucleotides and amino acid sequences of the disclosure encoded thereby are also encompassed by the present disclosure.

A “variant” nucleotide sequence (a nucleic acid sequence or polynucleotide sequence) is one having at least 80%, 82%, 85%, 88%, 90%, 92%, 94%, 95%, 97%, 98% or 99% sequence identity to a reference nucleotide sequence. A variant nucleotide sequence may encode a variant peptide.

By “fragment” is intended a portion of the polynucleotide or a portion of the amino acid sequence. For polynucleotides, a fragment comprises a polynucleotide having deletions (i.e., truncations) at the 5′ and/or 3′ end relative to a reference sequence; and a variant may include a deletion and/or addition of one or more nucleotides at one or more internal sites relative to a reference polynucleotide; and/or substitution of one or more nucleotides at one or more sites relative to the reference polynucleotide.

The terms “inhibit” and “reduce” or grammatical variations thereof as used herein refer to a decrease or diminishment in the specified level or activity or presence of a circRNA, of at least about less than 15%, 15%, 25%, 35%, 40%, 50%, 60%, 75%, 80%, 90%, 95% or more. In particular embodiments, the inhibition or reduction results in little or essentially no detectible presence or activity (at most, an insignificant amount, e.g., less than about 10% or even 5%) of a target circRNA.

The terms “approximately” and “about” refer to a quantity, level, value or amount that varies by as much as 30%, or in another embodiment by as much as 20%, and in a third embodiment by as much as 10% to a reference quantity, level, value or amount. As used herein, the singular form “a,” “an,” and “the” include plural references unless the context clearly dictates otherwise.

The terms and expressions that have been employed are used as terms of description and not of limitation, and there is no intent in the use of such terms and expressions to exclude any equivalent of the features shown and described or portions thereof, but it is recognized that various modifications are possible.

All patents and publications referenced below and/or mentioned herein are indicative of the levels of skill of those skilled in the art to which the invention pertains, and each such referenced patent or publication is hereby incorporated by reference to the same extent as if it had been incorporated by reference in its entirety individually or set forth herein in its entirety. Applicants reserve the right to physically incorporate into this specification any and all materials and information from any such cited patents or publications.

1-15. (canceled)

16. A method of inhibiting circular RNA (circRNA) formation comprising:
providing a nucleic acid sequence encoding a target circRNA having at least one intronic regulatory sequence either 5′ or 3′ of the circRNA that is capable of promoting circRNA backsplicing;
contacting the nucleic acid sequence with a nucleic acid editing system configured to target said intronic regulatory sequence in said target circRNA;
removing or disrupting said intronic regulatory sequence inhibiting the back-splicing of said target circRNA.

17. The method of claim 16, wherein said nucleic acid sequence further encodes a functional messenger RNA (mRNA) that is not interrupted by disruption or removal of the intronic regulatory sequence.

18. The method of claim 16, wherein said target circRNA comprises a target cirRNA selected from the group consisting of: SEQ ID NOs: 1-433, and 439, a fragment thereof and a variant thereof.

19. The method of claim 16, wherein said nucleic acid editing system is selected from: a CRISPR system, a TALEN system, or a zinc-finger nuclease system.

20. The method of claim 19, wherein said CRISPR system further comprises one or more guide RNAs (gRNAs) complementary to a 5′ and/or 3′ portion of said intronic regulatory sequence.

21. The method of claim 16, further comprising endogenously expressing said target circRNA in a mammal.

22. The method of claim 21, wherein said mammal comprises a human or a mouse.

23. The method of claim 22, wherein said human or mouse has, or is at risk of developing, a disease or condition selected from: schizophrenia, bipolar disorder, Alzheimer's disease, Epilepsy, Frontotemporal Dementia, Parkinson's disease, Colorectal Cancer, or Hepatocellular carcinoma, or a combination thereof.

24. The method of claim 16 to 24, wherein said nucleic acid sequence encoding a target circRNA sequence is expressed in vitro.

25. The method of claim 16, wherein said target circRNA comprises circHomer1 circRNA.

26. The method of any of claim 25, wherein said circHomer1 circRNA is selected from the nucleotide sequences according to: SEQ ID NO: 440, SEQ ID NO: 446, or a fragment or variant thereof.

27. The method of claim 25, wherein said nucleic acid editing system comprises a CRISPR/Cas9 protein system and one or more gRNA targeting at least one intronic regulatory sequence of said circHomer1 circRNA.

28. The method of claim 27, wherein said one or more gRNA targeting at least one intronic regulatory sequence of said circHomer1 circRNA are configured to target intron 5 of said circHomer1 circRNA.

29. The method of claim 27, wherein said one or more gRNAs targeting at least one intronic regulatory sequence of said circHomer1 circRNA are selected from: SEQ ID Nos. 434-435, and 441-442, or a fragment or variant thereof.

30. The method of any of claim 25, wherein the circHomer1 circRNA is disrupted, wherein the distributed circHomer1 circRNA is selected from: SEQ ID NO: 438, SEQ ID NO: 445, or a fragment or variant thereof.

31. A method of treating a disease or condition in a subject in need thereof, comprising inhibiting circular RNA (circRNA) formation according to the methods of claim 16.

32. The method of claim 31, wherein said disease or condition selected from schizophrenia, bipolar disorder, Alzheimer's disease, Epilepsy, Frontotemporal Dementia, Parkinson's disease, Colorectal Cancer, or Hepatocellular carcinoma, or any combination thereof.

33. The method of claim 31, wherein said subject is a mammal.

34. The method of claims 31, wherein said subject is a human.

35-62. (canceled)

63. A transgenic, non-human animal whose genome comprises a circHomer1 circRNA mutant wherein one or more introns of said circHomer1 circRNA has been disrupted or removed.

64. The transgenic, non-human animal of claim 63, wherein the disrupted or removed intron comprises intron 5 of said circHomer1 circRNA.

65-68. (canceled)

69. An isolated nucleotide sequence selected from the group consisting of: SEQ ID NO: 438, SEQ ID NO. 445, and a fragment or variant thereof.

70. An isolated nucleotide sequence selected from the group consisting of: SEQ ID Nos. 434-435, SEQ ID Nos. 441-442, and a fragment or variant thereof.

71-72. (canceled)


SEQUENCE LISTING (446 sequences)

SEQ ID NO: 1 - DNA, 228 bp, Homo Sapiens
gaatggaagttggatacactttgtgacttgtacgagacactgaccattacacaggctgttatttttctcaatacgaggcgcaaggtggactggctgactgagaagatgcatgccagagacttcacagtttctgctctgcatggtgacatggaccagaaggagagagatgttatcatgagggaattccggtcagggtcaagtcgtgttctgatcactactgacttgttg

SEQ ID NO: 2 - DNA, 137 bp, Homo Sapiens
tggaagcattgacatttcctccttcttctggaaagtcattcatcatgggagcagatgaagcccttgaaagtgaactgggacttggagaactagcaggccttacggtggccaatgaagcagactcactaacttatgat

SEQ ID NO: 3 - DNA, 189 bp, Homo Sapiens
tgtgatttggtgtacaccttcttccacccactgccccgggatgagaaggctatgggcaccagcccagctcctaagtcctcagatggcacatgggcccggcccgtcggaaaggtgggaggggaggtgaagctgtccatctcctacaaaaacaataaactcttcatcatggtgatgcatattcggggcttg

SEQ ID NO: 4 - DNA, 327 bp, Homo Sapiens
gatcgcgtcttcgccaggaggactttcccccgcggattgtggagcatccttccgatgtcatcgtctctaagggcgagcccacgactctgaactgcaaggcggagggccggccaacgcccaccattgagtggtacaaagatggggagcgagtggagactgacaaggacgatccccggtcccacaggatgcttctgcccagcggatccttattcttcttgcgcatcgtgcacgggcgcaggagtaaacctgatgaaggaagctacgtttgtgttgcgaggaactatcttggtgaagcagtgagtcgaaatgcgtctctggaagtggcat

SEQ ID NO: 5 - DNA, 748 bp, Homo Sapiens
gtggagttggcatgaacttaacagcagcagatactgtgatttttgttgacagtgactttaatcctcagaatgacttgcaagcagctgccagggctcatcgcattggccaaaacaagtctgttaaagttattcggctgattggtcgagacactgtggaagaaatagtctataggaaagcagcctccaaactgcagctcaccaacatgatcatagaaggaggccattttactctgggagcccagaaacccgctgccgatgctgacctccagttgagtgagatactcaaatttggtttggataaactgctggcctctgaggggagcaccatggatgaaatagacctggagtccatactgggagaaacaaaagatggccagtgggtctctgatgccttgcctgcagcagaaggagggagcagagatcaagaggaaggaaaaaatcatatgtacttatttgaaggtaaagattattctaaagagcccagtaaggaagacagaaaatcatttgaacaactggtaaaccttcagaaaacccttttggagaaagctagtcaagagggccgatcactccgaaataaaggcagtgttctcatcccaggccttgtggagggatctaccaaaaggaagcgggttctgagtccagaagagctggaggacagacagaagaaaagacaagaagcagctgccaagagaaggagactcatagaggagaagaagaggcaaaaggaagaggctgaacataagaaaaa

SEQ ID NO: 6 - DNA, 373 bp, Homo Sapiens
gaggctgaacagtacccagggggagattcgtgtcggtcctagtcatcaggccaaacttccagatctgcaaccatttccttctccagatggtgatacagtgacccaacatgaggaactggtctggatgcctggagttaacgactgtgacctccttatgtacttgagggcagcaaggagcatggcggcatttgcaggaatgtgtgatggaggctctacagaggacggctgtgtcgcagcctctcgggatgacaccactctgaatgcactgaacacactgcatgaaagcggttacgatgctggcaaagccctgcagcgcctggtgaagaagcctgtgcccaagctcatcgagaagtgctggaccgaggacgaagtg

SEQ ID NO: 7 - DNA, 1501 bp, Homo Sapiens
ccccccggtatgagattgtcatcatcactgtggtagcagccgcagtcataatgggcactgcaggcctcagcacgtacctctataaccgccagcggaagatcaagaaatacagactacaacaggcccaaaaagggacccccatgaaaccgaacacacaagccacgcctccctgaacctatcccgggacagggcctcttcctcggccttcccatattggtggcagtggtgccacactgaacagagtggaagacatatgccatgcagctacacctaccggccctgggacgccggaggacagggcattgtcctcagtcagatacaacagcatttggggccatggtacctgcacacctaaaacactaggccacgcatctgatctgtagtcacatgactaagccaagaggaaggagcaagactcaagacatgattgatggatgttaaagtctagcctgatgagaggggaagtggtgggggagacatagccccaccatgaggacatacaactgggaaatactgaaacttgctgcctattgggtatgctgaggccccacagacttacagaagaagtggccctccatagacatgtgtagcatcaaaacacaaaggcccacacttcctgacggatgccagcttgggcactgctgtctactgaccccaacccttgatgatatgtatttattcatttgttattttaccagctatttattgagtgtcttttatgtaggctaaatgaacataggtctctggcctcacggagctcccagtcctaatcacattcaaggtcaccaggtacagttgtacaggttgtacactgcaggagagtgcctggcaaaaagatcaaatggggctgggacttctcattggccaacctgcctttccccagaaggagtgatttttctatcggcacaaaagcactatatggactggtaatggttacaggttcagagattacccagtgaggccttattcctcccttccccccaaaactgacacctttgttagccacctccccacccacatacatttctgccagtgttcacaatgacactcagcggtcatgtctggacatgagtgcccagggaatatgcccaagctatgccttgtcctcttgtcctgtttgcatttcactgggagcttgcactatgcagctccagtttcctgcagtgatcagggtcctgcaagcagtggggaagggggccaaggtattggaggactccctcccagctttggaagcctcatccgcgtgtgtgtgtgtgtgtatgtgtagacaagctctcgctctgtcacccaggctggagtgcagtggtgcaatcatggttcactgcagtcttgaccttttgggctcaagtgatcctcccacctcagcctcctgagtagctgggaccataggctcacaacaccacacctggcaaatttgattttttttttttttccagagacggggtctcgcaacattgcccagacttcctttgtgttagttaataaagctttctcaactgcc

SEQ ID NO: 8 - DNA, 2089 bp, Homo Sapiens
gcccaaggtcccggaggctatggcagcggctaccatcgtgcacgacacgtctgaggccgtggagctctgccctgcttacggcttgtacctgaagcccatcaccaagatgaccatcagcgtggcactcccgcagctgaagcagccggggaagtccatctccaactgggaggtgatggagaggctgaagggcatggtgcagaaccaccagttctccacgctgcgtatttccaagagcaccatggacttcatccgcttcgagggggaggtggagaacaagagcctggtcaagtcttttctggcctgcctggacggcaagaccatcaagctcagcggcttctccgacatcctgaaggtgcgcgcggccgagttcaagatcgacttccccacccgccacgactgggactccttcttccgcgacgccaaggacatgaacgagaccctgccgggggagcggccggacaccatccacctggaggggctgccctgcaagtggttcgccctgaaggagtcgggctccgagaagcccagcgaggacgtcctggtcaaggtgtttgagaagttcggggagatccggaatgtggacatccccatgctggacccctaccgggaggagatgacgggccgcaacttccacaccttcagtttcggggggcacttgaacttcgaggcctatgtgcagtaccgcgagtacatgggcttcatccaggccatgagcgccctgcgcgggatgaaactcatgtacaagggcgaggacggcaaggccgtggcctgcaacatcaaggtgagtcctgggcaccgagagagccacgcgcttcctccctctggcgacttccttccagcagggtcagcagaacgctcccagccacacccctgaggctgtggggacctcccctaagtaaaatggcagcaacagtcctgtgcctgtccaatttcagaggcacaggcgtgaggtggagtaggggtcccgtgctgtggttttggaaatttggtatcttaaggaagatgtgggggagacacaggtcatatagtaggtgagggatggagcctcaggaactctgacagggatttgaaccgtgtcagaaccaagagaacccggccttcccagaatgcaccttgaaaccagggctgaacgttccctttagcctcatgtgcttttttattttttttattttttttatttttatttttttttcgaggcagagtctcgatctgtttcctggactggagtgcagtggcgcaatctttgctcagtgcatcctccgcctcctgggttcgagcgattcttctgcctcagcctcccgagtagctgggactacaggcgcgtgccatcacacctggctaatttttgtatttttagtagaaacagggttttgccgtattggccaggctggtctcgaactcctgacctcatgaactgcctgcctcgccctcccaaagtgctgggattacaggcgtgagccactgcgctcagcctagcctcgtgttttgaccgtgttggcttcacagtgacaaatgagccagcctcctggagggggttttgtggccttaagatgtgccctgtgggtgtgggcaggcggcctgctcagtgactgtggccacagggaatggtggagccgggtagcggaaggggagctgtgtctcgcagcggtggcgtgtcacttgtgttcagtaagtggcgagggctagttggtgttcctcgctgagtcgtaactccgtgggatgagctaagtcttgagccactgatggtgacttgaagggattggtttggcttctgggctccactgtctgggtctgcaccggacatgagtggtgagcggtgagcggtgagcgggcgctcaggctctgaggccactcggccctacggcccaggagagggcgcctggccgtgtgtctggggggttttgggggaaggccatctgacactgtttttgcttttaaaggtttcttttgattcgaccaaacacctgagtgatgcctcaattaagaagcggcagctggagaggcagaagcttcaggaactggagcagcaaagagaagaacaaaagcgcagagagaaggaagcggaggagaggcagcgagcggaggaaag

SEQ ID NO: 9 - DNA, 1089 bp, Homo Sapiens
gttatcctacgcggagagccaggtaccaatgggtgcgctgcaatccagacagtaattctgcaaactgccttgaagaaaaaggaccaatgttcgaactacttccaggtgaatccaacaagatcccccgtctgaggactgacctttttccaaagacgagaatccaggacttgaatcgtatcttcccactttctgaggactactctggatcaggcttcggctccggctccggctctggatcaggatctgggagtggcttcctaacggaaatggaacaggattaccaactagtagacgaaagtgatgctttccatgacaaccttaggtctcttgacaggaatctgccctcagacagccaggacttgggtcaacatggattagaagaggattttatgttataaaagaggattttcccaccttgacaccaggcaatgtagttagcatattttatgtaccatggttatatgattaatcttgggacaaagaattttatagaaatttttaaacatctgaaaaagaagcttaagttttatcatccttttttttctcatgaattcttaaaggattatgctttaatgctgttatctattttattgttcttgaaaatacctgcattttttggtatcatgttcaaccaacatcattatgaaattaattagattcccatggccataaaatggctttaaagaatatatatatatttttaaagtagcttgagaagcaaattggcaggtaatatttcatacctaaattaagactctgacttggattgtgaattataatgatatgccccttttcttataaaaacaaaaaaaaaaataatgaaacacagtgaatttgtagagtgggggtatttgacatattttacagggtggagtgtactatatactattacctttgaatgtgtttgcagagctagtggatgtgtttgtctacaagtatgattgctgttacataacaccccaaattaactcccaaattaaaacacagttgtgctgtcaatacctcatactgctttacctttttttcctggatatctgtgtattttcaaatgttactatatattaaagcagaaatataaccaaaggttaa

SEQ ID NO: 10 - DNA, 842 bp, Homo Sapiens
tacgtagagaaatcgacactcttaatgaacgtttagctgctgaaggacaagcgattgatggagcagagctgagtaagggccaactcaaaacaaaagccagtcacagcaccagccagctctcccagaaactgaagaccacttacaaggcttccaccagcaagattgtctccagctttaagaccacgacatcgagagctgcctgccagctcgtgaaggagtacatcggccaccgggacggcatctgggatgtcagcgtggccaagacacagccagtggtgctcgggactgcatcagccgatcacacggctttgctgtggagcatagagacagggaagtgcctagtcaagtacgcaggccacgtgggctcagtaaattctatcaaatttcatccctcagagcagttggctctcactgcttctggagatcagactgctcatatctggagatacgcggtgcagctgccgacaccccagcctgttgctgacactagtatatctggggaagatgaagtagagtgctctgacaaggacgagcccgacctcgatggggatgtgtccagcgactgccccaccatccgcgtcccactgacatccctcaagagccaccagggcgtggtcatcgcctccgactggctggttggggggaagcaggctgtgactgcctcctgggaccggacggcaaacctgtacgacgtggagacgtccgagctcgttcactctctgacagggcacgaccaggagttgacgcactgctgcacacaccccacccagcggctcgtggtgacctcctcccgtgacacgactttccgcctctgggatttcagggacccctccatccactcg

SEQ ID NO: 11 - DNA, 681 bp, Homo Sapiens
acagatgcttgctctggcctgcagggcttcctgattttccacagttttggtgggggcactggctccggcttcacttctctgctgatggaacgcctctccctggattatggcaagaaatccaagctggagtttgccatctacccagccccccaggtctctactgcagtggtggagccctacaactccatcctgaccacccacaccacactggaacattcagattgtgctttcatggtggacaacgaagccatctatgacatctgccgcaggaaccttgacattgagcgccctacctataccaacctcaaccgcctcatcagtcagattgtgtcctcaatcactgcttctctccgctttgacggggccctcaatgtggacctcactgagttccagaccaacctggtgccctacccccgcatccacttcccgctggtcacctacgcgcccatcatctctgccgagaaagcctatcacgaacagctctctgtggccgagataaccagctcctgctttgagcccaacagccagatggtgaagtgcgacccgagacatggcaagtacatggcctgctgcatgctctaccggggcgacgtggtgcccaaggatgtgaatgtcgctattgctgccatcaagaccaagaggaccatccagtttgtagactggtgtcccacaggcttcaag

SEQ ID NO: 12 - DNA, 1627 bp, Homo Sapiens
gtgtgtagctgggacggtgctggtctgagctggaccttgtctgatggcttcctccaaccctcctccacagcctgccataggagatcagctggttccaggagtcccaggcccctcctctgaggcagaggacgacccaggagaggcgtttgagtttgatgacagtgatgatgaagaggacaccagcgcagccctgggcgtccccagccttgctcctgagagggacacagaccccccactgatccacttggactccatccctgtcactgacccagacccagcagctgctccacccggcacaggggtgccagcctgggtgagcaatggggatgcagcggacgcagccttctccggggcccggcactccagctggaagcggaagagttcccgtcgcattgaccggttcactttccccgccctggaagaggatgtgatttatgacgacgtcccctgcgagagcccagatgcgcatcagcccggggcagagaggaacctgctctacgaggatgcgcaccgggctggggcccctcggcaggcggaggacctaggctggagctccagtgagttcgagagctacagcgaggactcgggggaggaggccaagccggaggtcgaggtcgagcccgccaagcaccgagtgtccttccagcccaagctttctccagacctgactaggctaaaggagagatacgccaggactaagagagacatcttggctttgagagttggggggagagacatgcaggagctgaagcacaagtacgattgtaagatgacccagctcatgaaggccgccaagagcgggaccaaggatgggctggagaagacacggatggccgtgatgcgcaaagtctccttcctgcacaggaaggacgtcctcggtgactcggaggaggaggacatggggctcctggaggtcagcgtttcggacatcaagcccccagccccagagctgggccccatgccagagggcctgagccctcagcaggtggtccggaggcatatcctgggctccatcgtgcagagcgaaggcagctacgtggagtctctgaagcggatactccaggactaccgcaaccccctgatggagatggagcccaaggcgctgagcgcccgcaagtgccaggtggtgttcttccgcgtgaaggagatcctgcactgccactccatgttccagatcgccctgtcctcccgcgtggctgagtgggattccaccgagaagatcggggacctcttcgtggcctcgttttccaagtccatggtgctagatgtgtacagtgactacgtgaacaacttcaccagtgccatgtccatcatcaagaaggcctgcctcaccaagcctgccttcctcgagttcctcaagcgacggcaggtgtgcagcccagaccgtgtcaccctctacgggctgatggtcaagcccatccagaggttcccacagttcatactcctgcttcaggacatgctgaagaacacccccaggggccatccggacaggctgtcgctgcagctggccctcacagagctggagacgctggctgagaagctgaacgagcagaagcggctggctgaccaggtggctgagatccagcagctgaccaagagcgtcagtgaccgcagcagcctcaacaag

SEQ ID NO: 13 - DNA, 593 bp, Homo Sapiens
gcagcggaggaggaaatggcaggtcctaatcaactctgcattcgccgctggactaccaagcatgtagctgtgtggctgaaggatgaaggcttttttgaatatgtggacattttatgcaataagcaccgacttgatggaatcacattgctaacattgactgaatatgatctccggtctcctcctctggaaatcaaagtcttaggggacattaaaaggttaatgctctcagtccgaaaattgcagaaaatacatattgatgttttagaagagatgggctacaacagtgacagtcccatgggttccatgacccctttcatcagtgctcttcagagtacagactggctctgtaatggggagctttcccatgactgtgacggacccataactgacttgaattctgatcagtaccagtacatgaatggtaaaaacaaacattctgttcgaagattggacccagaatactggaagactatactgagttgtatatatgtttttatagtatttggatttacatctttcattatggttatagtccatgagcgagtgcctgacatgcagacctatccaccactcccagatatattcttagacag

SEQ ID NO: 14 - DNA, 726 bp, Homo Sapiens
actttgaagattatccaatgagaattttatatgaccttcattcagaagttcagactctaaaggatgatgttaatattcttcttgataaagcaagattggaaaatcaagaaggcattgatttcataaaggcaacaaaagtactaatggaaaaaaattcaatggatattatgaaaataagagagtatttccagaagtatggatatagtccacgtgtcaagaaaaattcagtacacgagcaagaagccattaactctgacccagagttgtctaattgtgaaaattttcagaagactgatgtgaaagatgatctgtctgatcctcctgttgcaagcagttgtatttctgagaagtctccacgtagtccacaactttcagattttggacttgagcggtacatcgtatcccaagttctaccaaaccctccacaggcagtgaacaactataaggaagagcccgtaattgtaaccccacctaccaaacaatcactagtaaaagtactaaaaactccaaaatgtgcactaaaaatggatgattttgagtgtgtaactcctaaattagaacactttggtatctctgaatatactatgtgtttaaatgaagattacacaatgggacttaaaaatgcgaggaataataaaagtgaggaggccatagatacagaatccaggctcaatgataatgtttttgccactcccagccccatcatccagcagttggaaaaaagtg

SEQ ID NO: 15 - DNA, 280 bp, Homo Sapiens
gttgccatgtcaaatcttagcgaggctgtgcaggatgcagacctgctggtgtttgtcattccccaccagttcattcacagaatctgtgatgagatcactgggagagtgcccaagaaagcgctgggaatcaccctcatcaagggcatagacgagggccccgaggggctgaagctcatttctgacatcatccgtgagaagatgggtattgacatcagtgtgctgatgggagccaacattgccaatgaggtggctgcagagaagttctgtgagaccaccatcg

SEQ ID NO: 16 - DNA, 586 bp, Homo Sapiens
tttgtccacactagaaattgaaaatagagcccaagaccttcatctctttgagactttgaagactgatccagaagcctttcaccagcacatggtcaagtatatttaccctactattggtggctttgatcacgaaaggctgcagtattatttcactcttctggaaaactgtggctgtgcagatttggggaactgtgccattaaaccagaaacccacattcgactgctgaagaagtttaaggttgttgcatcaggtcttaattacaaaaagctgacagatgaaaacatgagtcctcttgaagcattggagccagttctttcaagtcaaaatatcttgtctatttccaaacttgttcccaaaatccctgaaaaggatggacagatgctttccccaagctctctgtacaccatctggttacagaagttgttctggactggagaccctcatctcattaaacaagtcccaggctcttcaccggagtggcttcatgcctatgatgtctgcatgaagtactttgatcgtctccacccaggtgacctcatcactgtggtagatgcagttacattttctccaaaagctgtgaccaag

SEQ ID NO: 17 - DNA, 1298 bp, Homo Sapiens
tctgcagatctccaggggagcccaccagcctagtcaacatggcctcggaagacattgccaagctggcagagacacttgccaagactcaggtggccgggggacagctgagtttcaaaggcaagagcctcaaactcaacactgcagaagatgctaaagatgtgattaaagagattgaagactttgacagcttggaggctctgcgtctggaaggcaacacagtgggcgtggaagcagccagggtcatcgccaaggccttagagaagaagtcggagttgaagcgctgccactggagtgacatgttcacgggaaggctgcggaccgagatcccaccagccctgatctcactaggggaaggactcatcacagctggggctcagctggtggagctggacttaagcgacaacgcattcgggcccgacggtgtgcaaggcttcgaggccctgctcaagagctcagcctgcttcaccctgcaggaactcaagctcaacaactgtggcatgggcattggcggcggcaagatcctggctgcagctctgaccgaatgtcaccggaaatccagtgcccaaggcaagcctctggccctgaaggtctttgtggctggcagaaaccgtctggagaatgatggcgccactgccttggcagaagcttttagggtcatcgggaccctggaggaggtccacatgccacagaatgggatcaaccaccctggcatcactgccctggcccaggctttcgctgtcaaccccctgctgcgggtcatcaacctgaatgacaacaccttcactgagaagggcgccgtggccatggccgagaccttgaagaccttgcggcaggtggaggtgattaattttggggactgcctggtgcgctccaagggtgcagttgccattgcagatgccatccgcggcggcctgcccaagctaaaggagctgaacttgtcattctgtgaaatcaagagggatgctgccctggctgttgctgaggccatggcagacaaagctgagctggagaagctggacctgaatggcaacaccctgggagaagaaggctgtgaacagcttcaggaggtgctggagggcttcaacatggccaaggtgctggcgtccctcagtgatgacgaggacgaggaggaggaggaggaaggagaagaggaagaagaggaagcagaagaagaggaggaggaagatgaggaagaggaggaagaagaggaggaggaggaggaagaagagcctcagcagcgagggcagggagagaagtcagccacgccctcacggaagattctggaccctaacactggg

SEQ ID NO: 18 - DNA, 365 bp, Homo Sapiens
gcctgtctccactaactctggaccctaaaacagctcacccaaatctggtgctctccaaaagccaaaccagcgtctggcatggtgacattaagaagataatgcctgatgatcctgagaggtttgactcaagtgtggctgtactgggctcaagaggcttcacctctggaaagtggtactgggaagtagaagtagcaaagaagacaaaatggacagttggagttgtcagagaatccatcattcggaagggcagctgtcctctaactcctgagcaaggattctggcttttaagactaaggaaccaaactgatctaaaggctctggatttgccttctttcagtctgacactgactaacaacctcgacaag

SEQ ID NO: 19 - DNA, 7273 bp, Homo Sapiens
gctgaactcacgggaatcaaatggcgtaggtacaattttggagggcatggggactgtggacccataatttcagccccagcccaagatgatccaattctgttaagtttcatccgctgtctgcaagctaacctgctttgtgtatggcgtcgtgatgtcaaaccagattgcaaagagttatggatattctggtggggagatgaacccaacctagtgggtgtaatacatcatgaactgcagggtaaggtttctgtttttttcttaaaaattatttgcatcatatttttttgaacattgataaatttcaaacagattattaagacttgtctaaaagaacgatcttaagtgtttcatggatgtctctctcaaaaggaacattctcacccctcttcctcccatccctgtttttttttcctaactcttgtccttttccagttatgctgagaatcagaaccttgtgaaccgctgctgttaaatgagagtattgtgtctcctgggtgcggtggggtggaaaccaggctaagatccactgtttttaaatgatagtgaattgtgagtaactaacaagacagtgaattgaacagttactgtttttttccttggactgtggttgttatggttcactgccagctgaactctttggtggaaaattggagagtgatacagaaatttagttttgagcctaattggacaaaattgtgagattacattgaatagtattttgaggaaagatgaatatatgggatctggtctgaattgggtgtagcaaattttcttttgcttttatttctgtttttcatgtaatttggtgattccaggaaacattggctccggtgttgcctgatttttagcttcagattgcatctgatatctggaaactgaattaatgtaagagtttcatttttctgtgctgctttatatatttaccacagttttatgagcaggaacttttatcagttgtctttgaatttctggtaaaaatttttacctttttatctaagtgtttctatttagtatgcaaagggattttctcaagcaagactaattctaatctttagttagggtattaactctttttgacttagtctcttagaaccatcttttattatctccaaactatattgtgttaatctgtgtctgaaaaatttcagatcgtgtttgaaatgttttgttacttgatgtttttaatgttttttttttttaaaggaaggagtaaaactaactgtaatgtatgttcgaggctctgttctaaagcttttacatttgcctgaaggatttcctgccaacttttgcttgacaccttgctagtttttcttggcagccattttttatctgtgatatctcctgccacagcagccagagggcggtaaaccacaatggactaattgaactgtttactttatggtccatttgtctgttcagctcaaacctttatcctgatattttttagggtatttggtctataaataagagaaccattgtgggtgacattgcttgcttagctgggtaatcagcaaagtcccagaacttaacattcatctcaaagcctgttctatgtagttagcagttaacttaggtaaaattagagtttcagaacatctggattctgtttatcagaattttaaaaataagatgtcacctagaagataattacaacatttttgtttgtccattaaaatatgaaaacatatttttactttttttccctatgtagtagagaggtataaagctttttaaaaagtgtctttcttatctttcatgattgtttctgactgtcatcttctgtttctctgtattaatctcttacgttttagctttttggtctgctctttgttagtgtggtacatataattttgtaatgatgaggaaaccaaacccaactgctaatgagggttatttagttgataatatagaataaatttatttatctatgccaacggagaaaatcgtcagtgttcttagaaatagcttattaaattattgtgttatttttcatgggctttgttactgagttaacaaaaaggagtagggcatttaaattttgggatagttgctttagattgcatgtgtaaattaatatcttaagtattttttggtaaagagttaagcaaagattgaattatttttagatatttttaaaattacagaaacactacatttaagttagaaaattaggaaaacagtcatcatatggagagaacaaagaaataaaaatactgaagtctgaaagagttttagatgttttatgtgtaaatgaaattggtaccatacagtttttctaattttctaattaaatgtagtattgtgaatattttctcttgggaatgattacttttctttggagtttgatagtggtataaactgtttatattttgtattctatcaggaaagtattacagtaatggaataattataatttcattgagtgcgtctcctctttttaatatgaattttgagcaagtttttagattcatttatatgaagtgtccatttttgtaggtaaaagtagttgaaaaaggtaatgtatggttcaacctaataacagaaaatatctagccttaattagaaaaataagtcataaagacaactttgtttcaaataaggggaaatacgatagcagtcatgaaaaactgaaggatacaaagagaatattttaattggtagccatacaaagtggagatattttagaatctatttcattctctaatatatttgagtcattttattttgggtaaaataaattataggcggagcatggtggctcacgctggttatcccaacactttgggaggctgagggggcccaaatgcttgaggccaggaatttgagaccagcctgaccaatatggtgaaaccctgtgtttacaaaaaataaaaaaaaaaaattagccaggtgtggtgacgtgcacctgtaattccaactactcgggtgactggggctggaggatcacttaaacccaggaggccaaggttgcagtgagcagagataatgccactgcactccatcctgggcaacaaagtgacacctgtctcaaaaaataaaataaataaatagtagcagaatccttttagcaggctttaatgttaagcatgagattaccattatttgtaacattttttaaatagggacattttactgtttcttacattgctgcttatttatgttatttggcatttcctggctggtcacggtggctcacgcctgtaatccctgcactttgggaggcctaggcaggaggatcacctgagttcaggagttggagaccagcctggccaacatagtgaaaccccctctctagtaaaaataaacaaaaattagctgggcatggtggtgggtgcctgtaatcccagctactggagaggctgagacaggacagtcgctggaaaccgggaggcagaggttacagtgggctgagatcacgccacagcactctgctgggcaacacagcaagactccatctcaataaataaataaataaataaataaataaatagcagaatcctgttagctggcttaaattaagcatgacattaccattatttaacattttttttaatagaggcattttattgtttcttaaattgctttttagttatttggcatttccctttgtcttcatagcgtactccaaatagcgtcctaccaagaaacctcacggtgagatgtaataccaaagcaaccatcttgcatttaaatgtataaagtatcccataaaatgagaataatcctacagggtacacacacaagttagttttatgatattatactttttgtttaattgcttgacatatctctgaaatagtctgagagaaggctataacacatttataatctgtccttgtcacttggataaacacttgggttcctgtctggggaattgttaatgcgtgtggtgtagtgattcagagcacgggctctgggttgcttgggtttgaatgcttgcatagtcactagctgtgtattcttgatctaattatttaacttaaactgtgcctcagttttctcactgtgaattaggttatgaggttaagaaaaattaaaagtacttagagcagcatatatagcagttaatctataattgtaactattgctactgctactgtgcaaagacctacaggctgcgaatgaattgtggcaagaacaggttacctggggcttagggtaggagtgaaatagtctcccctctaaaaggctgagtaggtgggtcatagtatggagtacacagaaccaaggataagacatttagacttaatttgatagattttgaggaattaaacatttctgatcaggaaagcgatataattaggaagattaacttagtaataatacaggattaatgtgtacagggaaaccataatattgtgattttaaaattctaaattggaatataaacttaaatattctgttcaggatacagatgtatttaaattaaaaatactaatgattatatactataattcatattaatagtctttaacacttttattatacctttgctgttttggcttgactaaattctacattttaatttaagttttagagattaatacagtgtgctgagtggacttctcctaatctaccaaacaaatacttcttacttttttttttttttttttttcctttagacagagtcttgctctgttgcccaggctggagtgcagtgatgcaatcttggctcactgcaacctctgcctcccgggttcaaatgattctcctgcctcagccttctgagtagctgggattgcaggtgtatgccaccacacctggccaatttttgtatttttagtagagatggggtttcaccatgttggccaggttgatctcaaactcccaacctcaatcgatccgcccgcctcggcctcccaaagtgctgggattacaggcgtgagccactgggcctggcaacaaatgctttttaaataaacacaatattctcgccaattcactgcctgttagttagtggtgggaaagacatgcaatctggaaaacccaaaatagtacagagagcaagcacagaactgccaattgcttgccccaaagcattacaaatctctggtccagaaggagaagtatcagattcagcctagtatgtgcttaagaatgtttgtcgaatcagttaatgaaacgtattttatgaggttctaatgtagcgcagtattgcactcccggcagattgacttataataaatttgtgaaaatttttaatgatttgataatgcgaggatattgaagcctagttaaaataaagcatattatttctggttttaattcccaaggggatagaaatagccaatttttggggtatattttctaccacaaatatttagcactaaatatgcactgtctacaattttaaaatttatgttgtctctttttttgattattagacatataaattaaggatacatctgtacaactctaaaaatgattgtccctataacagtatcagaagtcttctgaattggttttattgtcagtgcaggggtagattggcagttattaccctttgttgtgaatttagagacataaatattgctttttctatttttaagagtgctgccacctgtgccaggaggtgtgggcgattgtaaaaacattagacataagagagcctggaaaaatggatctgttttctgacagtgaccacggtgtaagataatttaagatactagtactattgtttattgaactcattgcaagtgggcattttgtaggagtgtgggaaggctggatttttttagttttgtaatctgtatctatagattacagtctacaattccaaatggtgggattgataaaacctttgtttagataactccaagcaagaacctagcttctccactcctcctagctttttatttttaaatatatttacacacttatatgcatatgcctctttttccacctaaactatttgaggcaattttcagacttcacggacctttgcccctaaatacttcagcatttaagtcctgtgaatgagaatgtgaagcactcttttacgtaaccagagcacttttggcatgctcaagaaatttaatatcgatataattaaattatctaatatttagtccatattcaaatttcccaattgtcttagcaataccttttatagttttctctctctctgatccagaatgcaatcaggatcacacatttttagatggcttgtgtcctctcatttagaatattcctgaagttgtcttggggatcaatgatattgatgttatttgaagggagcagtttagttgtcctgtaaaatatcccacattttgaatttgtctaattgttttcttacagttagagtcaggataaacatatttggcaagaatactgagtaggtgatgttgtgtcctttccacggcatcacatcagaaacaggtcatgtcagtttgtctcaatattgatggtactgagcttgatcccttgattaaggtagtgtctgccagatttctccagtgtaaagatatcctttccttttgtaattgttaagtaaatttgggatgaaactttgagattgtgtgaatatctctttcctcaaaagagcctaattttgagcctgtttaaaccaaccgaaaaattccggaatcacctgcttttagaaaatcagttgagatcacatgaagactgcatttttaagtcacagcagagttcaagaggagaagagaataaaatattgaagatctaccaaaggcaaattataatggcaatgtcacactatgaaaatgaatagcccctattgcttagcatacctaaaaagagaagtgttgacattgaattttccaaaatcatagacaataatgtatgtaggaatatattactgtgtatcttagagtctgtgaactttcaattgatttttgatggtttctggttatccttaactaaatggtaaaaataatttcatcggtattatacatgaatttattatcagttgaaattaaaaaattgctttctggaaaagtaatgactattttatatataaattgacaggaataagattatctcttgtcattttgaaaatttctttcaaggagaaaatctgatctttgactcattttcactctgtttttctatctttttttttagtggtattaaatttgtgagtcattgaaattaatccctgactgaattggaaaggaattgtatttgcagtatttggatttatttatttttcaggtatggaattctggtgattttgaaaacatgaatgataccattttgcagcagcattgtagatttgtagtattttagattggtatcacagtgcacctgaaaa

SEQ ID NO: 20 - DNA, 584 bp, Homo Sapiens
aaaagatgttggtttaaagcgattttttcctaagagtttactggattctgtcaaggccaaaacactaagaaaactgatccaacaaacatttagacaatttgccaaccttaatagagaagaaagtattctgaaattctttgagatcctgtctccagtctacagatttgataaggaatgcttcaagtgtgctcttggttcaagctggattatttcagtggaactggcaatcggcccagaagaaggaatcagttacctaacggacaagggctgcaatcccacacatcttgctgacttcactcaagtgcaaaccattcagtattcaaacagtgaagacaaggacagaaaaggaatgctacaactaaaaatagcaggtgcacccgagcctctgacagtgacggcaccatccctaaccattgcggagaatatggctgacctaatagatgggtactgccggctggtgaatggaacctcgcagtcatttatcatcagacctcagaaagaaggtgaacgggctttgccatcaataccaaagttggccaacagcgaaaagcaaggcatgcggacacacgccgtctctgtgtcag

SEQ ID NO: 21 - DNA, 1246 bp, Homo Sapiens
ctgactacacagacttagtcttctccactccgtgttcctgcagctagagacatgacctaacaccctgatgaccactctcagggaccttgagtgactggccggtgcaccatggaacttaaagtatgggtggatggagttcagaggattgtttgtggagtcactgaagtcacaacttgccaggaggttgtcatagccttagctcaagcaataggtcgaactggaaggtacacccttatagagaaatggagagatactgaaagacacttagcacctcatgaaaatcctatcatatccttaaacaaatgggggcagtatgctagtgatgtgcagctcattctacgacgaactgggccgtctctcagtgagcgacccacttcagacagtgtggctcgaattcctgaaagaactttatacaggcagagtctgccccccttagctaaactgaggcctcagattgacaaatcaatcaaaaggagggaaccgaaaaggaaatcactgacatttacaggaggtgccaaaggattaatggacatttttggaaaaggtaaagaaactgagtttaagcaaaaggtgctgaataactgcaaaacaacagcagatgagttgaagaagctaatccgtctgcagacagagaagcttcaatccattgagaaacagctggaatctaatgaaatagaaataagattttgggagcaaaagtataattccaaccttgaagaggaaattgtccgtctagagcaaaagatcaaaagaaacgatgtagaaattgaggaggaagaattctgggaaaatgaattacagattgaacaggaaaatgaaaaacagctgaaggatcaacttcaagaaataagacagaaaataacagaatgtgaaaacaaattaaaggactatttggcacagatccggactatggaaagtggtcttgaagcagaaaaattgcaacgggaagttcaagaggcacaggtcaatgaggaagaggttaaaggaaagatcggtaaggtcaaaggggagattgacattcaaggccagcagagtctgaggttggaaaatggcatcaaagctgtggaaagatctcttggacaagccaccaaacgcttacaggacaaagaacaggaactggagcagttgactaaggagttgcggcaagtcaatctccagcagttcatccagcagacagggacaaaagttaccgttttgccagcggagcccattgaaatagaggcctcacatgcagacattgaaaggg

SEQ ID NO: 22 - DNA, 1308 bp, Homo Sapiens
aagcagtggacaggggagaatccgctccaacgttgtccacctccccttcaccctcctccccttcacccacttccccttcacctactctgggcagacgaaggcctgaaataggaacgtttcttagaaagaagaaaactagtgacatctactttgtgtctctagtttgggccattgtcgtcatgcagatctggttgaacctgtggattgtgcagttgctgccagtgccgattgcagtctggatactcaaaaagcttgtcattcactttggagttgtggatttcctagagaaacgctaccatgtgtggtggggcattatagaaagcttcctgaaggagcggcagggagctctcgcgccttggcccattgtcgggcttggaaaattcttgttaaaagttgatagcaagctctggcactggctaaataagaagatgatcatctggttggagaagatgttagataaaataattagcattttcatcatatttttgttagtgataggaactcttcttttagccctactcctgactgcaaaggtacatcaagagagtgtacacatgattgaagtcacaagtaatttgattaatgaaactctagcaaatcaccctgagtgggcaaattggcttcctgaggctcaggtagtccaaagagccctgaattctgcggctaacaacgtgtatcagtatggacgagaatggataactcacaagctccataaaattctaggagataaggtgaacaatactgctgtaattgaaaagcaagtactagaactttgggacagactgtatcactcttggtttgtaaagaatgtaacacactctggaaggcacaaaggacagaagttgcatgtcagtcgtcagaatagctggctgggagacattctggactggcaggatattgtttcctttgttcacgagaacattgagacatttctttcgatcttggagtctctgtggatcgttatgagccggaatgtgagcctgctgttcaccactgtcactacactcttgaccatcctcttctacagcgggacagcccttctcaattttgtactctctctgataattttcctgaccacactattttatctattaagttccagtgatgagtactacaagccagtgaagtgggtgataagcctgactccactatctcagccaggtccttcttctaatattattggccagtctgtggaagaagctatcagaggggtgtttgatgcttccctcaaaatggctggcttctatggattgtatacctggctgactcatactatgtttggcatcaatattgtcttcataccatcag

SEQ ID NO: 23 - DNA, 1072 bp, Homo Sapiens
gtgtgtttgataactgctcccacactgtcagtgacaaaggctgggccctggggatccgctcagggaaggacaagggaaagcgggatgctcgcttcttcttctccctctgcaccgaccgcgtgaagaaagccaccatcttgattagccacagtcgctaccaaccaggcacatggacccatgtggcagccacttacgatggacggcacatggccctgtatgtggatggcactcaggtggctagcagtctagaccagtctggtcccctgaacagccccttcatggcatcttgccgctctttgctcctggggggagacagctctgaggatgggcactatttccgtggacacctgggcacactggttttctggtcgaccgccctgccacaaagccattttcagcacagttctcagcattcaagtggggaggaggaagcgactgacttggtcctgacagcgagctttgagcctgtgaacacagagtgggttccctttagagatgagaagtacccacgacttgaggttctccagggctttgagccagagcctgagattctgtcgcctttgcagcccccactctgtgggcaaacagtctgtgacaatgtggaattgatctcccagtacaatggatactggccccttcggggagagaaggtgatacgctaccaggtggtgaacatctgtgatgatgagggcctaaaccccattgtgagtgaggagcagattcgtctgcagcacgaggcactgaatgaggccttcagccgctacaacatcagctggcagctgagcgtccaccaggtccacaattccaccctgcgacaccgggttgtgcttgtgaactgtgagcccagcaagattggcaatgaccattgtgaccccgagtgtgagcacccactcacaggctatgatgggggtgactgccgcctgcagggccgctgctactcctggaaccgcagggatgggctctgtcacgtggagtgtaacaacatgctgaacgactttgacgacggagactgctgcgacccccaggtggctgatgtgcgcaagacctgctttgaccctgactcacccaagag

SEQ ID NO: 24 - DNA, 646 bp, Homo Sapiens
gtttgggatgtacattccgttcctgcagctgaattgcgacctccgcaagacaagcctcttcaaccacatggcctccatggggccccgggaggcggtcagtggcctggcaaagagccgggactacctcctgacactgcgggagacgtggaagcagcacacaagacagctgtatggcccggacgccatgcccacccatgcctgctgcctgtcacccagcctcatccgcagtgaggtggagttcctcaagatggacttcaactggcgcatgaaggaagtgctcgtcagctccatgctgagcgcctactatgtggcctttgtgcctgtctggttcgtgaagaacacacattactatgacaagcgctggtcctgtgaactcttcctgctggtgtccatcagcacctccgtgatcctcatgcagcacctgctgcctgccagctactgtgacctgctgcacaaggccgccgcccatctgggctgttggcagaaggtggacccagcgctgtgctccaacgtgctgcagcacccgtggactgaagaatgcatgtggccgcagggcgtgctggtgaagcacagcaagaacgtctacaaagccgtaggccactacaacgtggctatcccctctgacgtctcccacttccgcttccat

SEQ ID NO: 25 - DNA, 945 bp, Homo Sapiens
ctggttggtggagaatttgacttggagatgaactttattatccaggatgctgagagtataacatgtatgacagagcttttggagcactgtgatgtaacatgtcaagcagaaatatggagcatgtttacagccattctacgaaaaagtgttcggaatttacagactagcacagaagttgggctaattgaacaagtattgctgaaaatgagtgctgtagatgacatgatagcagatcttctagttgatatgttgggggttcttgccagctacagcatcactgtcaaggagttgaagcttttgttcagcatgcttcgaggagaaagtggaatctggccaagacatgcagtaaaattattatcagttcttaatcagatgccacagagacacggtcctgatacttttttcaatttccctggttgtagcgctgcggcaattgccttgcctcctattgcaaagtggccttatcagaatggcttcaccttaaacacttggtttcgtatggatccattaaataatattaatgttgataaggataaaccttatctttattgttttcgtactagcaaaggagttggttactctgctcattttgttggcaactgtttaatagtcacatcattgaagtccaaaggaaaaggttttcagcattgtgtgaaatatgattttcaaccacgcaagtggtacatgatcagcattgtccacatttacaatcgatggaggaacagtgaaattcggtgttatgttaatggacaactggtatcttatggtgatatggcttggcatgttaacacaaatgatagctatgacaagtgctttcttggatcatcagaaactgctgatgcaaatagggtattctgtggtcaacttggtgccgtgtatgtgttcagtgaagcactcaacccagcacagatatttgcaattcatcagttaggacctggatataag

SEQ ID NO: 26 - DNA, 342 bp, Homo Sapiens
agagtatgggagccagaggaacatcattatctaataagtcacctaagggaaagagaaatgaaaagatgttgccaggcagggagaaggaagaggagaaagagtgtggggtaatgaatgccaaggaaacaaagaaaggggaacggtttcaaacattgccaagagaaccagtaagtatcccctggatgcaacaagggtgtcagtggggagactatttgcagtgcagacatcaagacagaggtcagacggccaagcattgagcattactgaggatgagaaagtagagacagaacaagcagtcatctcttttaaaatgtttggctgtgaaggagaagtggggcaggg

SEQ ID NO: 27 - DNA, 190 bp, Homo Sapiens
gcgggacctaggccaggtgtctgtcggcagttttgagatcctgccagggcggcagttccgggtcgtggtgctcagcactgtgcacacctgccagagcctgctcagccctggggcactggcccctgagttcttcaccgacgcccgcgtgctcaacaccgtcctgacccgcgcccagtcccagctggtggta

SEQ ID NO: 28 - DNA, 466 bp, Homo Sapiens
gtagtgtcgcctgcctctggtgggaccctattcagcggttactcttctcaggagcatctgacaacagcatcatcatgtgggacatcggaggaaggaaaggccggacgctgttacttcagggccatcatgacaaggtgcagtcgctgtgctaccttcagctcaccaggcagctcgtctcctgttcctcggacggcggaattgcagtgtggaacatggatgttagcagagaagaggctcctcagtggttggaaagtgattcttgtcagaaatgtgagcagccatttttctggaacataaagcagatgtgggacaccaagacgctggggctaagacaacatcactgcaggaaatgcgggcaggctgtctgcgggaagtgcagcagcaagcgctcaagttacccagtcatgggcttcgagttccaagtccgggtttgtgattcttgttacgactccatcaaagatgaaga

SEQ ID NO: 29 - DNA, 889 bp, Homo Sapiens
ccgatttattttgcatattcccagcgaggaaagagacaatgcaatccgagtgtgttttcagattgaacttgcccattggttttacttggatttctacatgcagaacacaccaggattacctcagtgtgggataagagactttgctaaagctgtcttcagtcattgtccgtttttgctgcctcaaggtgaagatgtggaaaaagttttggatgaatggaaggaatataaaatgggagtaccaacatatggtgcaattattcttgatgagacacttgaaaatgtactactagttcaggggtacctagcaaaatcaggctggggatttccaaaaggaaaagtaaataaagaagaagctcctcatgattgtgctgctagagaggtctttgaagaaactggttttgatatcaaagactatatttgtaaggatgattacattgaacttcgaatcaatgaccagcttgctcgtttgtacatcattccaggaattccaaaagacacaaaatttaacccaaaaactagaagagaaattcggaacattgagtggttctctattgagaaattgccttgtcatagaaatgatatgacccccaaatccaaacttggtttggcacctaacaaattttttatggccattccctttatcagaccattaagggactggctttctcgaagatttggcgattcctcagacagtgacaatggattttcctcaactggtagcacgccggctaaacccactgtggaaaaattgagtcgaaccaaattccgccacagtcagcagttatttcctgacggttctcctggtgaccagtgggtaaagcacaggcaaccactgcagcaaaagccatataataatcattctgaaatgtctgaccttttaaaaggaaag

SEQ ID NO: 30 - DNA, 532 bp, Homo Sapiens
catgttggaacaggattccaggagaatgaaccctagtgtgagccctgcaaatcaaaggaggcaccttctaggattttctgctaaagaggcacaggacacatcagatggtattatccagaaaaacagctacagatatgatcattctgtttcaaatgacaaagccctgatggtgctaactgaagaaccattgctttacattcctccacctccttgtcagcccctaattaacacaacagagtctctcaggttaaatcatgaacttcgaggatgggttcatagacatgaagtagaaaggaccaagtcaagaagaatgacaaataatcaacagaaaacccgtattcttcagggtgctctggaacagggctcaaattctcagctgatggctgttcaatacacagaaaccactagtagtatcagcaggaactcagggagtgagctacaagtgtattatgcttcacccagaagttatcaagacttttttgaagccatccgcagaaggggagacacattttatgttgtgtcatttcgaagg

SEQ ID NO: 31 - DNA, 512 bp, Homo Sapiens
ccttatttagtgtctctgacaaaaccggccttgtggaatttgcaagaaacctgaccgctcttggtttgaatctggtcgcttccggagggactgcaaaagctctcagggatgctggtctggcagtcagagatgtctctgagttgacgggatttcctgaaatgttggggggacgtgtgaaaactttgcatcctgcagtccatgctggaatcctagctcgtaatattccagaagataatgctgacatggccagacttgatttcaatcttataagagttgttgcctgcaatctctatccctttgtaaagacagtggcttctccaggtgtaactgttgaggaggctgtggagcaaattgacattggtggagtaaccttactgagagctgcagccaaaaaccacgctcgagtgacagtggtgtgtgaaccagaggactatgtggtggtgtccacggagatgcagagctccgagagtaaggacacctccttggagactagacgccagttagccttgaag

SEQ ID NO: 32 - DNA, 340 bp, Homo Sapiens
cggtcacagcctcataccaagggctggagagacgatcatgcaggtcccaggaggctggaactgtgggagcagcacaggggaatgggcttgtgggtctggggtggcgttctgggtgtggcccgcctcgcagtgaccacactgaagtgggcaccgtcctgcaggcagaaggtgtgccgtgagggcagaggcttcctcccatggacccttcctggcggttaccgcccttttgctcccctccactgaggactccctgttcctttgctcccggtgccctcatgcacaccagctgtggccagtctttcctgagggccagccctgtgcctagggtgccctgccccac

SEQ ID NO: 33 - DNA, 192 bp, Homo Sapiens
caagtttatttctgatcgtgaaagtagaagaagtctcacaaacagccatttggaaaaaaagaagtgtgatgagtatattccaggtacaacctccttaggcatgtctgtttttaacctaagcaacgccattatgggcagtgggattttgggactcgcctttgccctggcaaacactggaatcctactttttct

SEQ ID NO: 34 - DNA, 309 bp, Homo Sapiens
cctcagggtgctttcctgaagtcctgccccgagccccaacccaaccctgaggagggggccctctggagctcgggtgcccccggccccgcagggagagatgattgacaggatcgagtacaatgtggaacacgcggtagactatgtggagagggccgtgtctgacaccaagaaggccgtcaagtaccagagcaaggcgcgccggaagaaaatcatgatcatcatctgctgtgtgatcctgggcatcgtcatcgcctccactgttgggggcatcttcgcctagaagccacccaaactgccactccactccag

SEQ ID NO: 35 - DNA, 330 bp, Homo Sapiens
gaggacagtcagtgacccacaccggcctgcccatcatggcctccctggccaacacagctatctccttcagctgcaggatcacctatccatacactccccaattcaaggttttcacagtcagctactttcatgaagatctccagggacagaggagccctaagaagccaacaaactgccaccctggactgggcacagagaaccagagccacaccctggactgccaggtcacccttgtgctgccgggagcatcggccactggcacctactactgctctgtccactggccacactccacggtgagaggcagcggcaccttcatcctggtcagag

SEQ ID NO: 36 - DNA, 266 bp, Homo Sapiens
gcgccatcttctccatcagactcccctgagccctgggcctcttcctcgtcctcctcttcctcatcatcatcaaactgctggatgcggtccttgtagcatatctcaagtaggttggcgttgggctgcagggcacaggggtgggaggatgggccacactgcccacagccccacacccctccacccgccacaaccaggggacagggcagtactcacgttctcatcgtcagcattgagggagaaggtgatgttggctgtcttgtcaaaag

SEQ ID NO: 37 - DNA, 1227 bp, Homo Sapiens
gaaaaatagaaatgaaggtacatatgcacacaaaattttgcctcatttgtttgctgacatttatttttcatcattgcaaccattgccatgaagaacatgaccatggccctgaagcgcttcacagacagcatcgtggaatgacagaattggagccaagcaaattttcaaagcaagctgctgaaaatgaaaaaaaatactatattgaaaaactttttgagcgttatggtgaaaatggaagattatccttttttggtttggagaaacttttaacaaacttgggccttggagagagaaaagtagttgagattaatcatgaggatcttggccacgatcatgtttctcatttagatattttggcagttcaagagggaaagcattttcactcacataaccaccagcattcccataatcatttaaattcagaaaatcaaactgtgaccagtgtatccacaaaaagaaaccataaatgtgatccagagaaagagacagttgaagtgtctgtaaaatctgatgataaacatatgcatgaccataatcaccgcctacgtcatcaccatcgtttgcatcatcatcttgatcataacaacactcaccattttcataatgattccattactcccagtgagcgtggggagcctagcaatgaaccttcaacagagaccaataaaacccaggaacaatctgatgttaaactaccgaaaggaaagaggaagaaaaaagggaggaaaagtaatgaaaattctgaggttattacaccaggttttccccctaaccatgatcagggtgaacagtatgagcataatcgggtccacaaacctgatcgtgtacataacccaggtcattctcatgtacatcttccagaacgtaatggtcatgatcctggtcgtggacaccaagatcttgatcctgataatgaaggtgaacttcgacatactagaaagagagaagcaccacatgttaaaaataatgcaataatttctttgagaaaagatctaaatgaagatgaccatcatcatgaatgtttgaacgtcactcagttattaaaatactatggtcatggtgccaactctcccatctcaactgatttatttacatacctttgccctgcattgttatatcaaatcgacagcagactttgtattgagcattttgacaaacttttagttgaagatataaataaggataaaaacctggttcctgaagatgaggcaaatataggggcatcag

SEQ ID NO: 38 - DNA, 20725 bp, Homo Sapiens
ataaagaaactgaggctcaatgaacttgtaacgctttagtgtcagagctctagaatgggtgaaggccagattcagacccaggcatcgtgactcccaagtctgcttgtggatccttgaaagcaagggtgagccataagcattgcacctttgagagaactggtaagttagaaccgcagggacttctgagagcttccttcatcattttctgcatgcctcccagtgtgcatagtctctgctccttctcccgttagcatgagccgtgtgtgtgatacagtgtgagcactggtgtggagggttcagatccatgtgggtggcccccacactctagccaagtgagcatgtaaagatgtttttatgtagcttcatcgataattaacttccactctctgcctcattccatgcctccccacttgccttcctccctccctccagcctgggtgcttgggggcctaggctggggacctcactgtgactagcttagcaattctctagctccaggaggctgccaggacccaggcgggagtgcaggaagttttcaaggtctgtgctccagggtgtgtgatgacttgactgctccctgaagagctcgctgcttataatcgcatagtttttggcatgtgctctctgacaaaagaacagatgtttcctacaagaacaaaagaacagggcataactgttggcctaacgctttgccactaataacattgaaagtggaagcaacaagtcctaacgagaaggtccctacaggatcatgaagccagtgccagggaattggagtcaaaatgaaagaactcacggctgcttttacgttttaataggcagtgtgctggatgttttttaacctctttggataaatagcaacaggctattctttctagctgtgttcatgaattttgtagctgtacagtaatgatggcttcacaagttcccttacaagatcccaagcccgtgtccatttcagagacacactgagagggttatgacaggctcggccaaccctgcagcatcagcctacagtccatatatgggggaagccttagctaaatcgcagctctcagccaaactaaaaacaccagagcaagtttttctctttgttgctggctggaacttcaaagcacggagagcccacacacaaaaatgaaaaacaaagaaccctaaaaaagggcccagcctcagagcccacccccaaaagtttagtatgcctcttgtgttaaaaacctttcaccccatatgtcacatgtcacccagggtttcagtcgcgttcctagagtcattcctctgatttttcatctctgaagaatatcttctttgaggttgtccttggctttcgcccaaaggaaagacatttattttttctcattctttcccccaaccacctaccctgactatttcttcaaactaacagacacagtgaaaatattatttaaatgtttttaacttcttttaccatgaactgttgtcaccaaaaaggttcaatgttcgggtttagtaagtcatggttagcaaagaacattcacagcatctgatgtcaacaggatctttacaaatctctcctcctatttcccttggactttattgagagggatggatactatatcccaatgtggcaactggacatcctcatttatatttgtggtcccagcttatacctttttcctgatgttctgtttgctgatttgcatgattcattatatgatcacctcttctttaagaaggggttttgaaaatatttcttatgattctagcaatttatatcaaattttatggtgctatcattataatgcgaatgtgggaaatgcagaaccaaaaccttgaagcgacttgaaattttcataaggcaaatccattcatcacatatacatttgagtccatgttatttttctgaaaatgtggttgagctaatgcaattatgaaaatgttgtttcttggaaatgaaaaggttttgtgttcagcagggtacataaagtaggtgtaagcactctcatatttgggctccaaatatgggttacatgttgagactgagtgccagttgaggagcacattaaaaaaacacccaatcatatgattgtgttattcatagttatacattctgtgtcccagagactaaatatcaacagcagattccttcaaagattggatgggtatgagtgacaaagtgacggatcttaacatcttgaaatggtggcagaattgagttagctgtttttataaaaaaaaataggccaggacggaagttagacacattagtgctgctgaatttgttttgaatcaactctgtggctctaggcaaccagatttctctccatgtgtcggtttcctgatctgcaaaatgggcataaatgtgttttctctaggacctctctcaaaattctcacgaagataaaacaggcctttatttgggatagtgctttgaaagtaacgcatattacacatgtttctcaagctgtacttaaaacagcaggctgtgattttgaggcagccacatcagaaggtgtctggagctctcggcctctgtggggcatgctcagagtgaattgaaatttttggtcagtgaatcctggtggctaaatgactattttccatatttaattcatctatttttagcttattttcatgacttgattcagaacttaatagaaacaaatgtgcatctattacaattcaaaattgaacttcctgggtggtgttgggtgggagaaacagagattgaaatgagagagagagagaggaaaacttaacatttctatggtaataaaaaagtagtttttacaaaactgaaaacactaaaaattgaacccacaaaaatggtatgatgaaaccaactgaatttctgcatttggaagcaaaccagaactcggtgactgtactcaggtctctctcttcttttcaaactgttggatgcctttgcttcatcgctgtgttgttgatgtttgcattttaattataacaatgacacttacctgtccctatttaagccatcatgaaaactctgtgttgaaaatctgcttgactggatgaaaagtctaaaaaacgcaggaggatagcactgctgtccccaagccatatgggcaagtgatttgaggtcattttccagcaggagtgacaaatttaactcaagtggtctccccttctacctcccatttcttgtgggtaaggtctgggtcaatgattatagtcagtgactacgaatgagtggatttctagggagaggcacatgtatcctgttaggctgtcagtatgccgtggtttgcctgagagagtgcaggtttacccctgtggcccagcccgatgattaattgtacctcttttcattcctaaggtgtttggatttgggtgagaagttgtgtgatcatactattcatgctgggtgctgagcacacaacctgggcattagggtctctgtgtcctcatcaagtgggaggtctggcccagataacataaaccagttgtcattgcatgggatcagggtagacctaggtgctttgatgacaatctgttggtctgtgctcttccctttaatatatattcccaaactaattgtctcactaaattcactaagtgcagtttctggctgcaacatttctacttgggaaagacgccagaattgtcagccagtaaagacttcagacctttcttggccaatttgccaaaaaaacatcagagggagttattgatcttttcccttgtactcattcagctaatttttttttctgtttcctttcttttcttcttcttaagctgacatccaataaaagctatttctttgaggtgccagcaacatgtcagcaccaataccattgacaatatgaattgtagtccccccacctccatttctctacaaatgtccttttcttggttctgttgacaacacaggtatttgtcaagctttgctttcctcttgcatttattcatgggtgactttgaacctggactggttcatgggactaaattccatttggatactgtgtttgaatactactgcaagttgcatgcttaaaaatttattaaacagtatacaccacttatcagagcaatcttattttatacttaatttatagaagaaagcccagtagattagtcagggacatctcaatagctagtttctcagcaggagcaaattttggatttggccttcagaaatatgaactattctttcagaaatttctggcactgaatagtgctacacttccccccgtgagagctgttcacttaccaccttttcttagctggaggcccaggctcatactagcctatgaggtacaattaaagaggaggcctagagaattgcaggcataattaccacagagatgggcaaggtagttatggtgacaataaatatgtaagatatctgggaagcttttttttttttttttttttgagacagagtctcactctgttgcccaggttggagtgcagtggtgtgatcttggctcactgcaacctccacctcccaagttcaagtgattctcctgcttcagcctcccaagtagctgagattacaggcatgcaccactacaagcagctaatttgttttgtatttttagtagagacggggtttcaccatgttgttcaggctggtcctgaactcctgacctcaaattatccacctgcctcagcctcccaaagtgctgggattacaggcatgagccaccgtgcctggcctctgaaaagttttttttttttttttttttctgagatggagttttgctcttcttgcctaggctggagtgcaatggtgcgatcttggcacactgcaacctccgccacctctggatagtgacaactatgaacagattaatatgcaaactgatggttgggacagtcatttgacaaatttgcaactgatttgcctgaattatcctgaaaatatctcaccctttcacttgaatgatgctgtgcagaacactctgctgatgcttgaaggtatttcctgtgtagagtcccccagtgagtacaaaaatatgggccaagtgtagcccagacatctgagtaactctcactcaattgtggatccacatgagaagaagtaaaaccaatgtcccatttcctacattgacgcagaggatgctgcttatctcagcacctggcttgtcaagcagccacgctcaaacctctgatcctctttgatcctgcctgatactgcttcatagaatttcaattccagaagaactcaaatcagcaatcttttctttatattccatttcttccctgcttctgcccactcaccttttctcgtggtcctaggatcagggcgtttgaggttatataacttaatatgagtaggaatcacttggagttaggaagagagaagtgttgatatagtggaatataagtaatttacaaattgatatatgcattctaaatatatgcctgtgaaatttctgtaattctaaaatgggaatagaaccaaaatgtttagtcagacatgataattatagctaacatttattgaaggcttataacgggccagctactgttctaattgctttacatttattagctaatgttctcacaatagcccaatgagacaattcttatcaccattttacaaagcaagaaataggcgcagagaggttgtgacttgttcaaagtcaccactgggtgatggagtcaatattgggacccgtgtcttgtgactcccccagtcacagtgccatgctgccggtacagttccaaaatgatattaacacttgccccttaactttgaaagagttttgcaaagcctggtcttataatatgtaaataacagtgcttgaatttcttggctgataagtattaataaccagaaagtaatattttagaatgcggtatccctggatattaattagcttgcttatatatgttagtcctctagggctgctgcgataaaggaccacagactgggtagcttaacacagcagcaatttcttctctctcagttttggaggctggaagtctaaagtggaggtgctggcagggacgtgctctctcagaaggttctaggggaggatccctcctgtctcttccagcctctgctggctctccttggccttccttgccttgtgaacgcagctcccttgtgaacgcagctccctgccttcatcacatggaattctccctgggtgtgtgtctgtgtctacatttttctcttctcaaaaggacaccagtggcctggtgcagtggctcacgcctataatccgaccactttgggaggccgaggcaggtggatcacctgaggtcaggagttccagaccagcctgaccaatatggtgaaaccctatctctactaaaaacacaaaaattagccacacgtggtggcatgcgcctgtaatccaagctacttgggaggctgaggcgggagaatcacttgaacctgggaggtgtaggttgcagtgagacaagatcacaccattgcactccagcctggggaacaagagcaaaaccctatctcaaaaaataataataaaaaaattaaattaaagatttaaatttaaatttaaaaaaaggacaccagtcattcccctcatcctggtataacttcatcttaacttgatgacgtctgcagaaaccctatttccaaataaggtcacccaaggtcttggagtttaggacttaaacatgactttttggggcacacaatcaacaacaatatacatatctgcttaggctatattatcacctatgttttaattttgagaataaaacaagaataatgtttcccccatgcaatttctttctttttcctccaacacacgtgcataattcttttatttcttccttaaaacagcatctgatctaccagcagatgtttcccccataataacaggcacataataccccacttcagaggtctgggatcgggtggagtggttctcatgatagctttttattttatagcagacctctttttattttgtcttctaaattgcttttccattacttgtttataatattaatagttaaaaccatagtatgggagtgatcatatagttgacatttacaattaacattgtacggccaggatttctccatgttattgcaaaaacttagctgatttgttaacattccttataatatttcagcaaattaaaaatatacaattaaatctttctcatagttggcacttaggttgtttccatttttttcttttgtgttagtactgcagtgaatatttatgcttatatcttttttcttcctttgttcttttttccaagaggaatttctaaaagaatataatattttggtgtctcttgatagcctatcgtttgatattgattgctgtcagaaattgcactggaatcaccccaatattgtttcacaagctgatcatataaaattttgttttaaatagccttttattgcttacattttaacttcattgttattatggttgaacacttttcaaatgtatttcttctaaacattaaaatcatctgggaactgaaaaaaaaaaaagacagcttatctttctttcgcagttgttaaaaacatgtgagaaggaagttagattgaattagttgcataacccaatatttaaggtttcctgagttgatacggccacatattttttttgttcctaatgctcaaatttccaaatttcatacttaaaagagccaacattaacacacagacacacacacacacacacacacacacacacacgtacatttgctttattaagataaatttgttagaacaaaagacctagtatatataacttgttttgcttaaataacattatttaaatgtatactgtgttcagtggtgcattccccctagtttagtatggtgaacacatgtcctaaatattcagtataaacttgtagaatggaagatgattcaccccttaatgaatttataagtatctgcttaaattatctttagtaaataaaattagcagcttatcagtcacttttctaaaatggctccttgctaaattaatttgcaagtttatcaaattacatttatgactttaaaatatatagtaatatgcattcattaggaatttatctcagatttctgaaatgtatgcatttaagattctatggatgattagaggagacatattttaaaaagtgtatatcttcaatgatatgtctttgaagtcatatctactttatatcaattattcgtaattgaatacaagagggtgcaattgagggctggcaaaaggggattttagttaaccaatatgattatcctttatttaaagttaattatattttttcacttctcctgttttacagcaagtgtggagttggctccaaactcagagtctgtctgggtactcagtcctacatctgtttaatgtcggtgtccttcttggcctcttcccatagacattgggttgctttagacttatgagtcttggccttggcggatgggacagtgagctcagtcttcattcactgacttgaggatttagtagatcttgattctggctctcttctgatccaagaaccacactacagagctaagtaatcaggcttcctgctttatgagctaaacatgtatatgtatataatatgcatatattatacatgtatgtgtatatatatatatataactgtatatatatatatatataactgtatatatatatatatatatatatataactgtatatatatatatatatatatatatataactgtatatatatatagctgaacaacgtgagtttgagctatgcaggcccatgtatgtgtggattttcttccacttttgctacctatgagatggcagaccaaatcctcctcttcttcctcctttcagcctgctcaacatgaagatgatgaggatgaaggcctttatgatgatccacttccacttaatgaagagtaaatatattttctcttccttataatttttaacaacatcttttatctagcttactttattgtaagaatacagaatataatacacataacatataaaatatgtgctcatcaactgtttatgttattactaaggcttccagtcaattagtagttaagtttttggaaagtcaaaaatcttacataaatttactgctatgtagggggtcagccccactaacccccatgttgttcaagggtcagctagacacacatgcacacatatatgcacacacatatctatctataaattttcatcttatgtcttttgtattctgggcttggtttgcaattctaatgtgaatggagggccaactatttgtcacagaaaaggagaaagatgctactaaaatcaacttcaactgctttagccccaaagagagtagcattacttattcttagactgaaatctttttatttaaataaaatatgttgatagagaactgttggtgcagtgtttgagtgctcaagagactggactggagagaaacaaagaacctaagagatagctgatgtcctctgcaggaggaatgaagaggggtaaggaggagaaaaagtgagaatgagcctggttttctgaaagtctaatgagacgtgctagccaattaggatttcctgttagtggtggtgactgaaagaaatgaaaaaaagaaggaatgtctaacatttaattttaaattgtttattcatgttgttactatttaatttccttcatctctctagtcctttcaaaggaaagaggccatgttccctgtctgggtagacaaggggtcagtgaaaagagaacaggtaggaaagtcattctacttcttggaagtaatatccttggtttgattgtccaaaacaccatccttgaattctctttgctagaaagagctcctgtatcagctgagaaggcctccatttgagccaacactattcccaatcagccttgtctgatcccagaatggctttctgagaacaggaaggttttcaattcttcctaccacaccaaccagtctcggataggctgtaggacctggaacatcatagcaagtgatatttggggttcacattgattatagtctgccacaaagggctacagaatcttccaaaggctgtggtgcaaacatctgcctgcttattccttattcttggggctgatcctggccacctggagtctgctggtcaccaagatccccagcttggacagactaattcctggactctgctctccctttcctgatatttcttctttatactttggctgctttcaactcgaggtttttgacctacctccatctttgtctctagaataataggtgcccattcactttcccatgataagtcttttgcctttgttcacagggagggcattccctacatttccagtcttagcttttcccagaaggactagcagagtctcaatcctaatcactatttaaagtagtacccaaaccagactagcacaacccaccaagccccagccctccctcctgagctactacaggttcaactggttgactggtcccctagcaccttgcaaagaacatgcactcagtgtatatttcttaataagtggatgaatgttttattctattggggagacagaagaagagtaacaagagagacattctagtagaaaagtaatattaatgaggctaaatgtattcatttgtctttttacctaatagacaataaaagattgccctattggaagagaagctcaaggctagatgaaatgccaaagggtcttttgtaagaagtttccaggtatccttaagcttgtggtagtcacccctggagtatcttttttaaagttcaaagatatccattatagacgcagggggaaaagcacaaaaagaaaaacaaaaacatttagagttacattaggcaggatttaggtacatacagaagttcgttagcgaggtgcctaacttttgtgcaggtttgtgtttctgtaaataaatcacactcggaattatctacttggcagagaggggagagacctgagatttagaaacaaacaatcaaatggagaaccgaggtttgagtttggtgcattctctataaattatataaattatcttaagcaatcaccagccccttgaagttttatttgcctcatctgcatattgaaaataataattgttgccctgcttgcttctagactttttttttttttaggatagagatgaggaaaatagtaactaattttatacatatatatatatatatatatatatatattttaacagagctgtttttagctaaaaatcaagtgtggtctctccccaaacccagggagtagctgagattctctgtggagggagaacctaccggaggagaatgttagggcctgtgcagaaaataggattgaaagaaacatggtcaatctcatgtttaggaaaaattcttgtaaagatttttgtttctacttgactaggtcaataaagataaggcactaaagataggcaatctaaatgccattttacacatatggtggatggataactctgtgtcctaatatttctgaaccttagttaccttatatgtaaagtaaaaaaaagtaccaatccatacatccatttcctttttgccatctcttgtcgactaggcacttttcaggattttccaaggagatcattgaaactttgttttgcttttctgcaaagatgtgtatatgaagttgtgtttgctgtctggccttatgagagactcagtgcttttctgacactaacattctggggctctttgtccagtccttcctccttggtcttccatggttctctgagatgcaggtaattgaatgcttgagctgggaaaaagcttttgttctatgatgattctaagtcttactctctcattgcaaacactaaagtgtagttctaagcgcaagtacagttttcctcatttttatttatctttcccaagattttatagcaaagtttttgttttctgcaccacctctaccctgctacccctccttaagcccctacgcagctgaaagctggaagctgaaacgttttcaacttgagcagacaggttgaagatgctgccaaaccatgtggaaatacattggtatagactggaactttgtgtaagaaaaattcacatttttagaatctttactatatttggcttacctgggttaagcaattgcatgttttcatggggagaggcccaaggcctagtagcttaatgtgcaaatcacaaggctggggctctggaggaaatcctagcctttgaggagtggcagctgtgtaaaaactcaacagaggaaggacaaactgtgttctctccttctcaccattgccctctgtcttcctcttcttcactctcagcctatgtgaaaaaatcagatccaggctgggcacagtggctcatgcctgtaatcccagcactttgggagcccaaggcgggcggatcacgaggtcaggagttcgagaccagcctggccaatatggtgaaatgctgtctctagtaaaaatacaaaaaaaaaaaaaaaaaaaaattagccaggtgtggtggtgctcacctgtagtcccagctactcgagaggatgaggcagaagaattgcttgaacccaggaggcggaggttgcagtgagccaagatcatgccactgcacttcagcctgggcgacagagtgagaggccatttcaaaaaaaatcggatccattttcctaaagcaactatcttatccctaagttaatttcaaacctacatatcatcttccagaccatatttttaatattgacaattttatactgacttggtaaaattggtgacaaagattatttcatatttttggttcaaatataatatagtcaatgtccagtcagtgtcaagacagttttcacccaaattatgaaggctaagagaaacattcacctcaagcgggaatgtcccttctgcttggtggtcatttctagttatcttagagaccttcagagttcacacctgaaggacaagtgagggcaacttgggcttctttatttcaattttactgaattttgatcattaatcagattagatctctacatttatggcatacactacacatacatgcactcatatttacctatatgaataattgcaataaatatatttcttctcaaagtaacaaaattttaactaaaaaacaaatctaacaagcacttaaattgggactgatcattcatatacaaaatgctccttttgtttttgttttttggctttagagaagaaccaaatcaaggatctgtcaaactcctaccatatattagaagtatgtcacatgtaatacaatgatctaatattagatagcaatggatcttatataagaactgaaaaaaagttagaattgaaacaatgtcatgtgaacatgagggggttggaaatatttttaaacgaatgtttctcattgttgcatgggtagtgagagtaactgccaaaatgtgggcaaaactggagaattatgcaatgggaggaaagaaatcaaaacatgcaatataatttcgactctattgttatcagaattttttttgaattaataataaaattcagcaggtggaatgagtcagttgatgactcaggaaggcccataggaaacttaaaactgttaagcagaacacttcttccttcaccacgaatttcagttttcttcaaatggcctttcaacagaaaattgcatctgtaggctctccatcccccacccccacaacccacatttgactttgagcctttatgaaaatgatagattgccactacagattgttcgcatatttttttttcagttttaggaaaacaaatgaaacaagtgttttgaagatgtctccagaggtgcccagtacaagggggtgggaatggagacgtaagtgaggaaatggtctgttttgtgacttggaataagcagagtactcctagttctggccttgactcccacctgtatccttgaaatggttttctaacttgtctccctggtagaatctctccagtgtttccatgacactgctggaggagtgatctttctgaatcacagtggtgatcatgatgattttctgatgcgaattaggcagtatctccccctgcggccaccctacgtccttccactgtgagcttctttcatctactttttggctctgtgaaacccctgcacccgagtgcacatttgtctctctcttaaggatgtcgactccatgtggatgctgactttgtttttcacttatctctgccttttctttctttttgtttttttttttttgagacggagtctcgctctgtcgcgcaggctggagtgcagtggtgcgatctgggctcactgcaaactccgtctcccgggttcacaccattctcctgcctcagcctcccactgggactacaggcgcccgccaccacgcccagctaattttttgtatttctagtagagataaggtttcaccgtgttagccaggatggtctcgatctcctgacctcgtgatacacccgcctcggcctcccaaagtgctgggcacttatctctgctttcaaatgttgcctcctcctggaggcctcccccatctcatgccttgcgtgttgtccattgtatcttctcctctgttctcagaggatttggtctctgcctcacttagcacttcatagtgctctgtcatgattcacatgttttctccactgtgtgttcccatcatagttgactgaacccttcatatttggtgaccttaatagtcccttacaacatcatattccttactaggtttcagaggacagcgtgcgctaacagcatgaacatagatttttctggttgacctttgtattcctgactgagtcttgtcctgatatttcttggacctgtggcaatgccttctttttgaggaagtcccctcatcaccagtcttcctgccgatactctcatggagacagtgccattgcttagggtgctatatcctggggcttccaatcttcaaggtcctcacatgtttgcactatcctgagttacatgtgccagaagcttcctgtgttctgtgtagctataaggacaatgcctttttcttctcaggattcaccattggtccccagggagggacaggccgagaagcaggtaggcaagagactgaggcagatcaccatcaactggtttttcaaattcagtcacggtgtgtatccagtctcaatctcagctgcaataccctggtacctttctctttcctttcatcacttaggcagtcactaacgtttagtaggtgctgtctgaaacttcagaacgctctgggactctctgacgaaggcctttgtgttcctgcaaacctccctcttgtccctgaccagtttggtcactgttgtttggtacccaaccctgccctatttttctctaggctggtcaggctctgacgccttgtagctgaattcccaggctgagccacacttctggtacacttaactgaaagcctggagagaacctacaaaaaatattacaaagcagggaacaaaaatcttaaaactggacactgaaaatggacgccttataagagcatctcagaatttcaaattcaactgcctctgaggcccccagagaatacaatattctactctaaataaaaaccacatacgtcatggtgcctttttttcttttcttttcttttctttttgagatggggtcttgctttgttgcccaggctggagtgcagtggcacaatctcagctcattgcaacctccgcctcctgggctcaagtgattctcctgcctcagcctcccgagtagctgggattacagatgccaaccaccatgcctggctaatttttttatttttagcagagatggggttttactatgttggccaggctggtcttgaactcctgacctcaggtgatccacccgcctcggcctcccaaagtgctggaattacaggcgtgaaccaccgcacccggcccattgtgccttttattaacattattaaaatatagctctttattgacacctcagctctctgtaccaggaaagaatgtctactttattttggtaaaattgaagggaatctctaaaatattcccatctggaacacaaggatattgtgttttcaaatctcaatctccttttaagaagatatacttttagttgtcgaatttttcttactactgggggatttatactcaaattactggtggcacactcaatagcctgattgcaatgattttcagtgcgtgatggacaccatatttgagaggtgttgatctgaaaggaagttagagacgtcatacctattaccacgatgcataatttgaaagtggagtggaaagatctagatagtagaacaatagataaccttagtaataatcttatccttagataccgctagacttctatgaaaatttgtcaataccattccactctcgcttttctgaaaaacttggtgtttcggacttcagcatttttctgattttataatttatgtcacctttctagtacagcgtggcagcactccctagtagaccacttaattgtgatatttcacaattgccaatcagggatcgtggccaatggcaggaaagcaaaagtatccacagcaggttttgaaaaacagtcatgagagacagctggcatgcacctttgctctcttcctctccatcactgcctctgacttgctgccttgaagatggatttagccatcttggattctgaaggtgaggcatgaggaaggtgagctggaaagaccctgggtgctaaataagcccagacagctcacctctgggcctcattcacatgacagagaaaaaaactttctatcttgtttaagccactgttttcaatctattttttttcctctgtcacacatagccaaaccaaaccctaaatgattcaggtattatggagaatgaagatcctagattcggtaagatttttttttttttttttttttttttttgagacagagtcttgctctgtcgcccaggctggagtgcagtggtgcgatctcagctcactgcaagctccgcctcccgggttcacgccattctcctgcctcagcctccccagtagctaggactacgggcacccgccaccatgcctggctaattgtttttgtattttttttagtagagatggggtttcactgtgttagccaggatggtctcgatctcctgaccttgtgatccgcccgcctcggcctcccagagtgctgggattacaggcatgagccaccgcgcccggcctcggtaagatttttaattactgcactgacctagtcagtcgatgtctcaatttgtgtatgagaaagccacatataaatgtattatattttagtgtgcactgtgcagctgattagattttctgcatgaaaatatgaaagtactacaacagaattcaaaataaagtagaatttatgagcttggggtagatcctcttaactttcctcctttatgtttggaataatttttttttaagtttacacaaaattatctcatcatgggagaggaaacttcttcctcatcccaatagtaagtttaacaagtaccgtagtttgttttgcctcaatattcctgattcacatagcacagtgtttaatattgaccaaaagatgccttcagctcacatcaggcaaaggaagtggttacctgatgctgtggtctgtaagtctgtttctcccctggattcatatattgaaacctaatcttcaatatgatgatattaggaggtgaggccttttggaggtggttaggtcatgagggtcgagctctcatagaaggaattggtgtccttataaatgaagctccagagagctccctcaccccttccaccatgtgagaatacagccagaaggcatcatctatgaagcagaaagggggacctcaccagacaccaaatctgccagcaccttgatcttagacttctcggcctccaaaactgtgagaaataaatttctgttgcttataagccacccagtctatggtattttgtgatagcagctgcaataaacgaagatgcctgactacattgactaaagagaagataaaacctaatcttagaaaagtttcttctcatttatttagggcttgattcttcctgtaccttggctgtattccaatggcaaatcaggacttttctgtgtatctaaacactggctgcctatagagcaggaattggtccctcagaacaactgtttccacactgacttgtgggaatctttttgagtgttttgagagttcaagggttggcagagcctgggatggcaggactctaaagagctctgagtcaggatatgcctgtgctggtattggtcattcatttgaatgggttttctgactatattattagcaaaatattttctctccagcaaaggctcaaattacattgcaatcttattttttttttaaacggtgtctcactctgtcgcccaggctggagtgcaatggtgtgatctcagctcattgcaacctccgccacctcctgggttcaagtgattctcctgcctcggcctccgtagtagctgggattacaggtggccaccaccacacccagctaattttttttttttttttcatttttcttagtagagacggggtttcaccatgttggccaggctggtctcaaactcctgaccttgtgatctgcccgcctcagcctcccaaagtgctgggattacaggcatgagccaccgcaccccacctgcagccttattttataattaacatatatgctctttttacaggtttccttgactagggattaagatggttaatttgtggctctcatgagatacaagcagtgtataataattattattttttccatgggactactcccctaagtttgctcatctccagtgcagatatttgcctgttttcactgtggcctggacaaatgagtatgaaggcatttgtgcagaggttttcaaactatgttcttagatcttgtaggatcccacagaggtgtctcaagctttggagaagagagtaagatggttcttgatcctcttaccggcttgctttactcacaggtggagactggtgagatgggaggaggaggcaaaatttaaggaaattcaatcagactatcattttcctatacaaccattaggtgcctacacatctgttattttattttattttatttttgagacggagtcttgccctgtcacccagactggagtgcaatggcatgatctcggctcactgcaacctccgcctcctgggtttaagcaattctcctgcctcggtctcctgagtagctgggattacaggtgtgcaccaccaggcctggctaattttttgtacctttagtagagatgggggtggggggtttcaccatgttggccaggctggtctcgaactcctgacctcgtgatccaccctcctcagcctcccaaagtggtggcattacaggcatgcgccaccgcacctggccagtttattcttttaacctcaaaaatgcatgagcaaaggtgaaggaaatagcactaatgacattgacaggtgagaaaactgagggtaagtggattttagaacgtcacctcatcagttaatggtgtctcttgggtctgagtcttaagatttttccaccaagttgaatgaggacagagctacttcatgattgtctatgtagacattgccaccacaataacaccttgtgagagagctaatcaaggcaacttgcttttggccctgttcaattctaggctgtgttatcctttactataaagcacattgacccaccatgaatggaggccttaagattgacctcccttcattccaccctccggtattgatggagctcctgagttaacacagtccaatggggaaaacagacgaacaattaagatggaatgtcaagagtgccacaagggagatgtgagtcgtatgcaagaaagcgagtggggggaacacgtaacagctctggttcaggaatctgataaggtttcgtagagaagacagcatttcagtgggcttggtggagatgggcaggagtttgtcgggcacagaaaagagcaaatggcatccaggcaggctcattacatacaaaagcatcttcatgggaaggcacgtggcacgtttggaatcagtaggaatccatcacaggtagagccaaagtaagttgagagaagaggcagatgagacttaagcaagtcacttgagagaagtggcacatcttcagtttcatatatttaagattgtaacatctgggagaagaatcaaagtgaaaatgtcacctatgggtatgaaattaggtccatttatttatttatttagacaagagtctcgctctattgcccaggctggagtgcagtagtgagatctcactgcaacctctgcctcctggcttcaagtcactctcatgcttcagtctcccgtgtagctgggattgcaggtatgcaccaccatgcctggctaatttttgtaattttttttttttattagagatggggttttgctatgttggccaggctggtctcgaactcctgacctcaggtgatccacatgcctcggcctcccaaactgctgggcgtgagccaccatgcctggccttgagtccttttagtgtctcttcctgtctcttgggtttttgagtgaacctgcagcacctcttcattcccgggcccttccttttctcccacaacctcccctccctctcttttgcaccctgcaggtgcatgcatacattgcctcctcttttccttctccctgcttttctgacgaaagttctggtccttctgttgagagctggctccttggatatagcacagatacctacttatttgtgtttcagttcccttgcctgccaaaatctcagatggagctttaggactcatggaagaccttcctcaagggggacttgaagtgctctgggaaaaatatgcgagtgtgaccctttccagggggcagcacgaggatggccaaacccgctactctccctggggctgg

SEQ ID NO: 39 - DNA, 1825 bp, Homo Sapiens
agaaagagttttgaagaaaactgggcataggctcagcaaaaccaaacagaagaggaacagaaaaagaaacaaaaagcagaacagtcagaatagaatcatggaggaaaactcattagaattcttaagtgatcttacaccgggagatcaggacccatctcagagtgaagaggaagacattgaaaagaccagaagagaatcagaatatcccttcattgatggtctacaaaatgaagtcggagattttgtgactggatataaagaaaaaagatggaaaaataaagatcctaaagacagtttccaaaacgttatgtctatagttgaattagacaacacaccaaagaattacctctctaaggaaggtgataacttgtttgtaagtttgttactgaggccaaatgaaatctccgttacttgtccaatactgactcaaaacctttcctgtgtaacaactgatgactgctctggcatgaaggtagaaaagcatattagaaataggcataccatagcattagacacccaggacctttctgcggaaacttcatgcttatttatgaagaagagagaaatagtagataaaaatctctcacatgaacccattctgtgccatcaacatggaatcagaatgtcagataaagttttaagagaggaacaagtgtatacaactaaaatcaatcactgggcttttttcacaaccaatttatctgatgaagatttacagctgggctctgacagacagccctattttggtagctggcctgcaggacctcataagtttatatgtgaacagagaccaaagaaagatagagcatgtaagttggctggtcctgacagcagggggcaatggattcaaatgatcttcacttcggtggcagcatcagaaccaggaaacaatccagaaatattgacagacaaactactgataggaaatgaagatttttcacctccacctgaaactatggattcattcatagaaacaaacctcttcagaagctgcttacctcaaccggatataccaaagaatgccttagaatcaacaaaaaataagaaaaggaggaagaaaaggattttcaatttggtaccaaattttgacttattaggacagagtcgtatcggtgtaaaagaaagggagaaatgtgacctgttaacaaaaaaccatggactaaaaattactttgggagaagaaaaagatagaatttcagaaaggaacagtgaagaggagaataaacaaaaacttatgacctttgatcatcatccattgtggttttaccttgatattatcaaagctacccctttaaatattgatggacagcgttattctcattgcctgtcatttaacagactaaggtgctctgcatctttatacaaaaattatattccttcttttgtgctacataatttatctagtatttggaagccatcttttacaaacaagaaactgtttttgactttcgaatctcagacaagagtaggtaataaactaaatgatgcagggtttatttctccagaaattttacatagtcatcctgatacttcgtgctctttgggagtcacttctgattttcactttttaaatgaaaggtttgatagaaagctgaaaagatgggaagaacctaaggaattaccagctgaggacagccaagacttaacaagcactgactaccgttcccttgagctaccattatcacaagggtttgcctttcaattagtaaagctttttggatctccaggcgttccaatggaatccttgttgcctgatgactatgtggttccccttgactggaagacactaaagatgatctacttgcaatggaagatgtcagtggag

SEQ ID NO: 40 - DNA, 570 bp, Homo Sapiens
tgagattacgtactataggtgaacttttagctcctgcagctccttttgacaagaaatgtggtcgtgaaaattggactgttgcttttgctccagatggttcatactttgcttggtcacaaggacatcgcacagtaaagcttgttccgtggtcccagtgccttcagaactttctcttgcatggcaccaagaatgttaccaattcaagcagtttaagattgccaagacaaaatagtgatggtggtcagaaaaataagcctcgtgaacatattatagactgtggagatatagtctggagtcttgcttttgggtcatcagttccagaaaaacagagtcgctgtgtaaatatagaatggcatcgcttcagatttggacaagatcagctacttcttgctacagggttgaacaatgggcgtatcaaaatatgggatgtatatacaggaaaactcctccttaacttggtagatcatactgaagtggtcagagatttaacttttgctccagatggaagcttgatcctggtgtcagcttcaagagacaaaactctcagagtatgggacctgaaagatgatg

SEQ ID NO: 41 - DNA, 1151 bp, Homo Sapiens
gtttttggcgcattctcacagacttagatgaaactgtgatggccaccgcagggggcaggtgctgacatcgtccccagccctgtggctgttcatccggacatcatttccaacctcaatatctaaatgccacagtgctcttggagcaagttgggctggggaccactgttgccttttaagaccataaaaccatgggaaacgcagaaagtcaacatgtagagcacgagttttatggagaaaagcatgccagcctggggcgcaagcacacttcccgctccctgcgcctctcgcacaagacgcggaggaccaggcacgcttcctcggggaaggtgatccacaggaactccgaagtgagcacccgatccagcagcacccccagcatcccccagtccctggctgaaaatggcctggagcccttctcccaagatggtaccctagaagacttcgggagccccatctgggtggaccgagtggacatgggcttgagacctgtgtcttacactgactcttctgtcactcccagcgtagacagcagcatcgtcctcacagcagcctctgtgcagagcatgccagacactgaggagagcaggctttacggggatgacgctacatatttggctgagggaggcaggaggcagcattcctatacatccaatgggcccactttcatggagacggcgagctttaagaagaaacgctccaaatctgcagacatctggcgggaggacagcctggaattctcactctctgatctgagccaagaacatttaacaagcaacgaagaaatcttgggttccgccgaagagaaggactgcgaggaggctcgggggatggaaacgcgggcgagtccgcggcagctcagcacctgtcagagagccaattccttgggtgacttgtatgctcagaaaaactctggagtgacagcaaacggggggccggggagcaaatttgcaggctactgtcggaatttggtgtctgatattcccaatcttgcaaaccataagatgccaccagctgctgctgaagagactcctccgtacagtaattataacacacttccctgtaggaaatctcactgtctctctgaaggtgccaccaacccacaaattagccatagcaacagcatgcaaggcagaagagctaaaacaactcag

SEQ ID NO: 42 - DNA, 531 bp, Homo Sapiens
gtttgatgattcctgtcttttgtgtcgtggagcagttggacggctctcttgaatatgacaacagagaagaacacgccgagtttgtcctggtgcggaaagatgtgctttttagccagctggtggagactgcgctcctggccctggggtattctcacagctctgcggcccaggcccaaggaataatcaagctgggaaggtggaaccctctccccctcagttatgtgacagatgcacccgacgcgacagtggccgacatgctacaagatgtctatcatgttgtgacgttgaaaatccaattacaaagttgttcaaagttggaagacttgcctgcggagcagtggaaccatgccacagtccgcaatgccttaaaggaactgctcaaagagatgaaccagagcacattagccaaagaatgccctctctcccagagtatgatttcatccattgtaaatagcacatattatgccaatgtgtcagcaaccaagtgccaggagtttgggagatggtataaaaagtacaagaagattaaag

SEQ ID NO: 43 - DNA, 278 bp, Homo Sapiens
ggaagagacttccaccttaggatagtgttgcctgaagatttacaactgaagaatgcaagattattatgtagttggcagctgagaacaatacttagtggataccatcgaatagtacaacagagaatgcagcactctcctgatctaatgagctttatgatggagttgaagatgcttttggaagttgccttaaagaatagacaagagctgtatgcactacctcctcctccccagttctactcaagccttattgaagagataggaactcttggttgggataa

SEQ ID NO: 44 - DNA, 361 bp, Homo Sapiens
aatgaattaccagatggattaaacaaaaagcgtttacaaatcttattaaaaggctatggtgaatatccaacaaaatacagaatgttcatttggcgctctctgctacaactgcctgaaaatcatactgcgtttagtaccctcatagataaggggactcatgtggcatttctcaaccttcagaagaaataccccatcaaaagtaggaagctactcagagtattacagagaaccttatctgcattagctcactggtctgtcatttttagtgacacaccatatcttccactcttggcatttccatttgtaaaattattccagaacaaccaactcatctgttttgaagttattgctactctcataa

SEQ ID NO: 45 - DNA, 158 bp, Homo Sapiens
aaatcttgcagcagctccaccagcagctggttgaagctgaacgtaggtcagtgacatacctcaaacggtacaataagatccaggccgactaccacaatctcattggagtcacagcagagctggtggattctctagaggccacagtcagcggcaagatg

SEQ ID NO: 46 - DNA, 1114 bp, Homo Sapiens
gtgaatgtaaatgcgaggacacgtggtccgggtgcataatgggagacactggctattatcttcctaaaaagttcacccagtgtaatattgaagagtatcatgacttcctgaatagtggaggtggtgcctgccttttcaacaaaccttctaagcttcttgatcctcctgagtgtggcaatggcttcattgaaactggagaggagtgtgattgtggaaccccggccgaatgtgtccttgaaggagcagagtgttgtaagaaatgcaccttgactcaagactctcaatgcagtgacggtctttgctgtaaaaagtgcaagtttcagcctatgggcactgtgtgccgagaagcagtaaatgattgtgatattcgtgaaacgtgctcaggaaattcaagccagtgtgcccctaatattcataaaatggatggatattcatgtgatggtgttcagggaatttgctttggaggaagatgcaaaaccagagatagacaatgcaaatacatttgggggcaaaaggtgacagcatcagacaaatattgctatgagaaactgaatattgaagggacggagaagggtaactgtgggaaagacaaagacacatggatacagtgcaacaaacgggatgtgctttgtggttaccttttgtgtaccaatattggcaatatcccaaggcttggagaactcgatggtgaaatcacatctactttagttgtgcagcaaggaagaacattaaactgcagtggtgggcatgttaagcttgaagaagatgtagatcttggctatgtggaagatgggacaccttgtggtccccaaatgatgtgcttagaacacaggtgtcttcctgtggcttctttcaactttagtacttgcttgagcagtaaagaaggcactatttgctcaggaaatggagtttgcagtaatgagctgaagtgtgtgtgtaacagacactggataggttctgattgcaacacttacttccctcacaatgatgatgcaaagactggtatcactctgtctggcaatggtgttgctggcaccaatatcataataggcataattgctggcaccattttagtgctggccctcatattaggaataactgcgtggggttataa

SEQ ID NO: 47 - DNA, 446 bp, Homo Sapiens
ggcaagtgtggtcttggggagatggtgactatgggaaattgggcagaggtggtagtgatggctgcaaaaccccaaagctgattgaaaagcttcaagacttggatgtggtcaaagtccgctgtggaagtcagttttccattgctttgacgaaagatggccaagtttattcatggggaaaaggtgacaaccagagacttggacatggaacagaggaacatgttcgttatccaaaactcttagaaggcttgcaagggaagaaggtgattgatgtggctgcaggctccacccactgcctggctctgactgaggacagcgaggtccacagctgggggagcaacgaccagtgccagcactttgacaccttgcgcgtgaccaagccagaacctgcagcattgccaggactggacaccaaacacatagtgggaattgcctgtgggcctgcccag

SEQ ID NO: 48 - DNA, 672 bp, Homo Sapiens
gtttccacgctttgagaaggactgaatgagatcatgcgcatggcagcttctctaggatagcaccctgcgtgaacacagtagctgctcagttaagtctttgatgggtgtgatcctgattcagcttgatcaattctgcagtcatactggattgtgtttgaaaaggtgtccagaaaaacctggccatctgcctcctgtcttcccagtaccccaaccccatggataaaaatgtcatctcagtaaaaatcatggaggaaaaaagagatgaatcaacaggggtcatctacagaaagaggattgcaatctgtcagaacgtggttccagaaattttaaggaaggtgagcattttgaaagtacctaatatccaattagaagaggagtcatggctcaatcctcgggaaagaaacatggccatacggagtcactgccttacgtggacacagtatgcatccatgaaggaagagtctgtcttccgggaaagtatggaaaacccaaattggacagagttcattcaaagaggcaggatttcaatcacaggggttggatttctcaactgtgttttagaaacttttgccagcacattcttacgacagggagcccagaagggaattagaatcatggagatgctgctaaaggaacagtgtggtgcccccttagctgaataaagaatcatca

SEQ ID NO: 49 - DNA, 294 bp, Homo Sapiens
gttacgtggtgcttctgtaatcattaggtttgtgaccaatacaaccaaagagagcaagcaagacctgttagaaaggttgagaaaattggaatttgatatctctgaagatgaaatattcacatctctgactgcagccagaagtttactagagcggaaacaagtcagacccatgctgctagttgatgatcgggcactacctgatttcaaaggaatacaaacaagtgatcctaatgctgtggtcatgggattggcaccagaacattttcattatcaaattctgaatcaagcattccg

SEQ ID NO: 50 - DNA, 8279 bp, Homo Sapiens
atttctacatcgtactaaggaagaatgatttcctatttgatatagctgaaccatgcaaaaatcagatccttgaaaaggtcagaacctgtatttatacatagtagaaatagagagttatcctctattaggctgtgtatgaatatatgtgtcacaaaaaacagagcaaatgaaaaaacttcccctttcttgacattttattctgcagtttttacaaattatgtgtgcctttataatgtgcaaactacactacttaaaagtatgtgtagagtactgttattaatcctgttttatagaaacaataatgggctaagagacttattcaaaatcctgtaactaatagctgtaagataaagcccagatcttttgcctctaagtcttaacctttccactacattgtaagttcgtaattagtatcagaatttagtagacatgtagtatgcaagctgtatgaacattggaaataatgtatataaaagtctatgaaaaagtgttaaaacccctttatggatgttaagtatcataaataattcagatcttgaccttcaatagctaacatataatcttgggatgataccagagtcctcaatatataatataccattggcttagtatagcaagcaaaattctttacaaggtgcctcatttctacttttctagcctcagctcattcagtttctgtcatctgtccctgtagcttactttgtcccttttcatctgtaataaatggctagcattgtccaaacacattgtccctttccatttctatgttcaattccacataattaggaagaatgcctgaaatgcctatttgtctttcttcggataatcctagctcaacttcatctcctccatgaagctttcctcaacccccccccaacccctggcaaagttaatcattcccttctgttttcccagagcactttttatagcaaatctctgtatagcaaaaatcacacgatgattgtatatttgaattgctatctttcctactaatctgtgagttctttgtgagcatgaacagggacttacacttatttgtctttgtgtctttaacatgttactactaggcttagattttgttactcagctcagatgtattttgtttaatgaataaaataagagctacagttctttccttataaggatctgtatgtacagtattctaagtgggagactgaccacacaaaaatacctaaaaacacatagatatatgtaaaacgatatagtgtatgcttgaggtaaaataatggaatgatcagagccaggttgagttatttagggaaggattcctctaggttagtagttggcaatctatggcccacaggccaaatctatctgccaccagttttttgtaaataaagttttactggaacatagccatgttcatttgttagtgtattatatgtctgccttaatgctatgataacagagttgagcagttgcaacagagaccatatgtcctgtaaaactgaaaatatttgccatctggccctttacagaaaaagtttgccaacccctgctctaggagtgggctttggatatgaaaagaagaggaatgtgaattggtgaagcataagtgtgcatcataatgcaatgaaaacaagccatagtagtaggtatgctaggtaagaaagctccactccaattcagtttgtcagagagacctatgctgaagagattaaatgtcaagaatgtttggattgagaaagagggaacacaaatctaatggaaagaaattttgaagtaaacactccgggcctgcagcaaaatagtttcctaaatagtagcaaaggatgatgatgaaggaaaaggaagcagcaacataaattaacattgactgagtgtttactatgtgccaggtactgttctaagtggtgtgtgtgtgtgtgtgtgtgtgtgtgtgtctcctatttaatcctctcaacaactaaatgagataattgctattattgtctttattttgctgcggaggaaactcaggcacaggaggctaagttaacttggtcaagttaatacaactggtaagtgatagaaccaagtctgaacccaggctgtcagtctgcagagtcttcataatagagaaggggaaatctggagatatttggtaaaaatacttttaagcttggagtaaggtacttagtccaagtaattgggaaatgtactatcccaacatggtaaagagaaaatgaacagacaataaaagagtaatgatggaaggcagggtttccaattagaagagtatactatggattaggcagaatgggcagggcaaggaagcagatacttaagacatgttatatagtcatacagtaagatacattatatagtcagtcattgcgtttaaccttgatgagaccagaaagcaattaacttttccttgcacacatgaaagctgtcctaaaatacttgaagagctggtatatccaattgagatatagattagcagtgactttcaaggaggtaaggggcattcaaccttcccacaaaaatatacacaccttagtatcccaagtgtattataattggtggaggtgtcaagtcatagcacacaccattctctctcattctgtctccctaagaggactggttcctgtccaaccctgtatgattgctatggtaaatatttacccctttggacaatttttttaaaaaaatacatatatattgtatgcatgttaaagtaatacatatgcactatgaaaattttgccaatagaagtataccaaatgcaaacattggctagagatggtaggattggagatggaaggaggaggagacattaaactaaatgactttcaacatttcttccaaatctgtagttctatgtttataccagcagagtggtaagcacaaaggaaaagaccacaatgtgactttgacatggttggcttgaatagaggataaaatggtataggagaaaatccaatatatgaaatctaaatttatagagcagattactttgcagtgaaccttggacttatttttataaagattacttttaactagccattttgatatgttcaaatatacttaattttaataaaaaatttcatttacacttaacttgtcagtagtatatttactgtgtaaaataaagcagacacacattgttcattatttcaaagaaggaagagatttcagccaagttatgttgtagtagctacagtggaaataattgagctgggccttgcccataaacctgcaatatgatagaacttaaaaggggaccaggcatggtaactcacacctgtaatcccagaactttgggaggccaagacaggaggattgcttgaggccaggagttcaagaccagcctggataacatagtgagacctcgcctctacaaaaaataaaaaagttagccaggcgcattgatgcacaccagtaatcctagccactcagaaggctgaggtgggaggattgcgtgagtccaggagtttgaggctgcagtgaactatgatcatgccactgtactccagcctgagcaacagggtgagaccctgtctcttaaaaactaactaacttaaaagataccttaggtatgtaaggtatgtaaagtgcctaccacagtgccagtaaatggtagatggagacgcagaagagaaaaaagatgatggtggtgatgatgctgttgttatgtcttctgtccccatatcacacttctgtaaggcttcagaaagaaagttggtattgtatatatttgtatacacacatacatacatgcacacacacactgtccatgctacaaaacctaaacttgcacagtgttgtttataaaaaccaggactcagatttgaagtctcagatttggaaggaactttaaaggttacccagtccagcctccctcttcctcctaatgatttagtatcttcaccaataggttattttacctctgctttcacatccttagtgatggcaaattcatgcctctccttcccagagagcttatcccacttttggatacagagctctgactggcagaaagttcttcctaataatatgctgaagtctatctacctatagcttccacccactggttctaattctctcccttggggtaatattgaagaagtagagcaaatctcatctctctcctacatgaaatccctccagatatttgcaaacagctctcatgtttcccctgagtctttccttctagttaaccttccctagtttcttaaattctcactcttcttgttgctttactttaaatgagattgtgcatttgtatctcattcaaaatataggacctagaatgaaacataaaatcctacttctgatatgtttggactggcaagaatggagcatccaggactgtcatcgtctttattccaaatattgactggaataatgatgatgctgatgcattgcttatttaatgacatcagatccatatatgtctgctaggaactatgaagtggcactataggaaccactgaacttcttctctgttcaagaagcatgagccagaagcattccaaaaagcattagcttctcttacgctctgcctcacacctctgcaagtttctggaaagggtaaacattatacatagttaaagaatactaggactgcctgtaggacaaaggaagtcatggcattttcattagccacttattgctagctgcattacttgttaaaggaaatactggtgttctcatgggttcttttctgtgcatacacaaagatgtatagttgctacaaagcaagtggagcagggggacaccaaagagatatgctccatcttctttgttcacctctgctacatggaggagataggatttattgctttctttgagaaagagattggccagtttccttcctcccccttcttctggtggatcattccagaagaatattgcatctcatcatagttatctcctaataaagtaaaagtaaatgaaggagagctggagacagagaatcagctgagatgtgctggaaatactcggctgggcgcagtggctcgtgccttgtaatcccatcactttgggaggccgaggcgggtggatcacctgaggtcaggagttcaagaccaacctggccaacatgatgaaaccctgtctctactaaaaatacaaaaaattagccaggcttggtgacaggcacctctcatcccagctactcaggaggctgaggcaggagaatcgctggaacccgcgaggcagaggttgcagtgagccaacatcatgccactgcactccagcctgagcaaccagagtgaaactccgtctcaaaacaaagtaacaacaacaacaaaaaaaacttgttaatgagagagtggctgatcagtggcacatcacagatgaatcctctgtctcctgcctccctgtagcttgtccttccatcaccacagtgagtagatgttctgctcacagcttttccattttgatttctctgcatattggcatagtattatgatttatctaaaactgagagttctgtcttttcttattgaattttcagaagtatgtttgatgcatcttttttctgttttccagtttccatttctaggggaagtattcttctcttcctgttctttgaaatacttaacacaatatctggcatgtagtaaacacttaaaatgataattatatattaataatttgacttattactatacaggtgataatttgtaactaagtttttaattgtatcttaatatatgaagaattctacttttccttgcttcaaggggtggcctgttgtgattgttttattacattttgaaaggatactaccatagccttacagattcttctactacctattatacttctttcaacgacttccagaaatacggagtcattacattctccagatctgcactatgcaatgagtagccacaaccatgtatgtctaattaaatgtaaatttaataaaaattaaatacaattaaaaatgcactcagtttgcactagctacatttcaagtcctaaatagccacatgtggctagtggctacccattggacaacaggaatattgaacatttccatcattgcattaaattttgagaggctgaggctggcagattcttgaggtcaggagttcgagaccagcctggccaacatggtgaaaccccatctctactaaaaatacaaaaattagccaagcatggcggcacatgcctataatcccagctactctggaggcactgctttggatattcgtagtctatgctcttgtgctcttttcttagaaattgaagaggatatattttctagggtgttcctaagactaatctcttaatgtttcagggctctgaacatgcattttgaggattcacgttactgttcttattccagccccttagtgccattactatcttttggaataaatacaagaactttcacatcctccaagaagattatactaccataaattttattttgtttatttttatttatttatttattttgagacagtgtttctgttgcccaggctggagtgcagtggtatgaccatagctcactgcagccttgacctcctgagctcaggtgatcctcctgccttaacctcccaagtagctggaactataggtgcacacaccaccacacctggctaatttttatattttttttttttttgtagagatggggtttcaccatgtttctgagtctcaaactcctagagatgggggtctcactgtgttgtccaggctggtctcgaactcttgagttcaggcgattcccccctacttcagcctcctaaaatactgggattacaggtggtcctcctgccttaacctctttacaggtgtgagccaccacacccagccattaagcactttcaaagccattttttcatttgttatttacagcagcccagtgaagcaagtagcattgggattattattcccattttactaataagaaaactgagattcagttaagttacagcctgaagtctttttgttttttggggtttttttttttttttttgaccaagtctcactctgtcgcctaggttggagtgcagtggcatgatctcagctcactgcaacctccgcctcctgggttcaagtgattctgatgcctcagcctctcgagtagccgggattacaagcatgtgccaccatgcttggctaatttttgtatttttagtagagatggggtttcgccatgttggccaggctggtctcgaactcctgacctcaagcatccgtcagcctcagcctttcaaagtgctgggactacagacgtgagccaccgtgtctggccatgttacctgcctaacgtcttaaagggaaaataaacattaaggcaaaatagaggagattcagatcagggtaaaattttaacgtcaaattcagcattttttgttttgtttttactttgtttttgttctactttactcccactccgactgtaataaaagagtgaaagttctttaggggaactgaggtactcccaaagaattgctacagtcccatttgtcctgtgatcatgtgcttggggtgccataacagaataccacagactgagtggcttaaacaatagaaatttatatcctcaaagttctgggggctggaagttcagtgtcaggatgctagcggcagaggctagagatgaggtctttctccttagcttatagatgacaccttctctctgagtcctcatgtagcatttttctctatgtgagagaaagagatatctctcatgtctcatccttttgttttaaagaaaccaattatatcagattagggtctcacctgtatgactttatttaaccttaattacctccttaaatgccctgtctccaaatgtagtcacattgggggttagggctttaacgtatgaatttgggggagacacagttcagcccataacaccatgttaatgggtttttagtgtcttttgatattctggcttaacctggttagctggaaactaaatttagtatttgaatgggaccaaatgtagcaaatatctgtattctttatttctgtgaatatagaggcttaattaaattcaacctgtggtacttgctgtaaggtactttgcaactccaagatattctaatagcaaaaaatctgtatctaaatttcctacttttcttataatgtagcctaattgttaataattagagatctcagtactttacaggtaataaatgttcttctccttcctccccacctaccttttttacagatggagaaattagcatacaaagaaactgacttgtcagaagtcagagcaaggtattggtggatccagggataaatcccaaacttcttaacccctagaccggtttttagtccattgactatgcagcctaatg

SEQ ID NO: 51 - DNA, 1231 bp, Homo Sapiens
gaaggtgggagtcaatcattttgacaagtctcctgaaaggaacagctagcaggaactgaaacctttttccatttggtctcgtggcaaaggcagagattgctccagcagctccacacaaaatgatgtgctcacgggtgccctctgaacagtcttctggtacctctctcttgcctaaagacggtgccccattttcttgggattccttggatgaggatggattggatgactccttgctggagctgtcagagggagaagaagatgatggtgatgtaaattacacagaggaagagattgatgcactgttgaaggaagatgacccatcatatgagcagtcttctggggaagatgatggtgggcatgttgagaagggagaaagagggagtcaaattctacttgatactccccgagagaaaaattcatcgtacagcctgggaccagtagctgagactcctgacctcttcaaactacctcagctaagtacatcaagtggtcatggaccagctcatactaaaccattaaacagacgctctgtactagaaaagaatcttataaaagtaactgttgcaccatttaatccaacagtttgtgatgctctgcttgataaggacgagactgattcgtccaaagatactgaaaaactctcttcccttggagaagagatgagagaagatggtcttagcccaaatgaaagcaaactttgtactgaatctgaagggatcagccccaataactctgcctggaatgggccccagctctcttcttcaaacaataactttcaacagactgtctctgataaaaatatgcctgacagtgagaaccctacgtctgtattctctcggatctcagaccattcagagactcctaatatggagttatcctgcagaaatggtggttcacacaagtcaagttgtgaaatgagatctctggttgtttccacctcatcaaacaaacaggatgttcttaacaaggattctgggaagatgaaaggccatgagagaagactaggcaaagtcattcctgttctacaaactaagaccaggactaatgttccgacgttttcacagtcaaatctagaacagcagaagcagctttatctcaggagtgtcattgctcatatagaagacccagaggacactaaccaaggtatctcgggggagctttgtgccttgatggatcaagttcatcatatgcagcactcaaaatggcagcatccttcggacctcaccacgcg

SEQ ID NO: 52 - DNA, 1539 bp, Homo Sapiens
tccgcacgactgttacagaggtctccagagccttctctctcctggtaggtttcggctcaggaccctgaatcctggcccacaggcaagcctgatgggacgtgcggggtagtacttgtatggggagacgcgggaaggaggaagaagcacatctgtcctccgcggcctttagctctgagcatccggccgcgggccctgactttgccgctcagtttggggcgcagggggggccgtgccctggcggaggagcagaaggcagagggtagcagctgcggctcagcggagagacttgttgcatttgcagctaaaaccaggccctacttgtccttgtgcaaaatggcaactcttaaggaaaaactcattgcaccagttgcggaagaagaggcaacagttccaaacaataagatcactgtagtgggtgttggacaagttggtatggcgtgtgctatcagcattctgggaaagtctctggctgatgaacttgctcttgtggatgttttggaagataagcttaaaggagaaatgatggatctgcagcatgggagcttatttcttcagacacctaaaattgtggcagataaagattattctgtgactgccaattctaagattgtagtggtaactgcaggagtccgtcagcaagaaggggagagtcggctcaatctggtgcagagaaatgttaatgtcttcaaattcattattcctcagatcgtcaagtacagtcctgattgcatcataattgtggtttccaacccagtggacattcttacgtatgttacctggaaactaagtggattacccaaacaccgcgtgattggaagtggatgtaatctggattctgctagatttcgctaccttatggctgaaaaacttggcattcatcccagcagctgccatggatggattttgggggaacatggcgactcaagtgtggctgtgtggagtggtgtgaatgtggcaggtgtttctctccaggaattgaatccagaaatgggaactgacaatgatagtgaaaattggaaggaagtgcataagatggtggttgaaagtgcctatgaagtcatcaagctaaaaggatataccaactgggctattggattaagtgtggctgatcttattgaatccatgttgaaaaatctatccaggattcatcccgtgtcaacaatggtaaaggggatgtatggcattgagaatgaagtcttcctgagccttccatgtatcctcaatgcccggggattaaccagcgttatcaaccagaagctaaaggatgatgaggttgctcagctcaagaaaagtgcagataccctgtgggacatccagaaggacctaaaagacctgtgactagtgagctctaggctgtagaaatttaaaaactacaatgtgattaactcgagcctttagttttcatccatgtacatggatcacagtttgctttgatcttcttcaatatgtgaatttgggctcacagaatcaaagcctatgcttggtttaatgcttgcaatctgagctcttgaacaaataaaattaactattgtagtgtgc

SEQ ID NO: 53 - DNA, 518 bp, Homo Sapiens
atgcacaaagcagggtcatcagaagcctctcgattcaaaagatgataataccgaaaaacactgcccagtgacagtgaatccttggcatatgaagaaagctttcaaagtcatgaacgaattaagaagtcaaaatttgctgtgcgatgtcacaattgtggcagaagacatggaaatttctgctcatagagtggtgctggccgcctgtagtccttattttcatgccatgtttacaggtgagatgagtgagagccgagcaaagagagttagaataaaagaggtagatggctggaccctgaggatgctaattgattatgtttacactgcagaaattcaggttacagaagaaaatgtacaggtacttctcccagcagctggtctcttacagttacaggatgtgaagaagacttgttgtgaatttttggaatcccagcttcaccctgtcaactgcttaggaatccgggcttttgctgatatgcatgcatgtaccgaccttctgaacaaggccaacacctatgcag

SEQ ID NO: 54 - DNA, 685 bp, Homo Sapiens
aagtgatgccaccaaaaagaaatgaaaaatacaaacttcctattccatttccagaaggcaaggttctggatgatatggaaggcaatcagtgggtactgggcaagaagattggctctggaggatttggattgatatatttagagacactggccatttttctctctccaatgcttagtgtggagtaaaggcagcttcccgcaaatttatttcaagcaagagcaatcggaagcttcgacatgtgtgaaagaggcgcatagagaacatagcttccatttcagctttccccacaaataaaccagagaaagatgcaagacatgtagtaaaagtggaatatcaagaaaatggcccgttattttcagaacttaaattttatcagagagttgcaaaaaaagactgtatcaaaaagtggatagaacgcaaacaacttgattatttaggaattcctctgttttatggatctggtctgactgaattcaagggaagaagttacagatttatggtaatggaaagactaggaatagatttacagaagatctcaggccagaatggtacctttaaaaagtcaactgtcctgcaattaggtatccgaatgttggatgtactggaatatatacatgaaaatgaatatgttcatggtgatataaaagcagcaaatctacttttgggttacaaaaatccagaccag

SEQ ID NO: 55 - DNA, 1953 bp, Homo Sapiens
gacaggaggaaacgcagcgccagcagcatctcatctaccctccttgacacctccccgtggctccagccagaccctagaggtcagccttgcggaccaacaggaggactcccagctttcccttttcaagaggtccccagacaccggccaccctcttccagcccctgcggccagtgcaaggaggcaccaatgctctgaggctgtcgcgtggtgcagcgtcgagcatcctcgccgaggtcctttctgctgcctgtcccgcctcaccccgctccatcacaccagctggccctctttgcttccttttcccagaatcgttaagccccgactcccactagcacctcgtaccaacctcgccccaccccatcctcctgccttcccgcgctccggtgtcccccgctgccatgagctcccccatcagcaagagccgctcgcttgccgccttcctgcagcagctgcgcagtccgaggcagcccccgagactggtgacatctacggcgtacacgtcccctcagccgcgagaggtgccagtctgcccgctgacagctggtggcgagactcagaacgcggccgccctgccgggccccaccagctggccactgctgggcagcctgctgcagattctctggaaagggggtctcaagaaacagcacgacaccctggtggagtaccacaagaagtatggcaagattttccgcatgaagttgggttcctttgagtcggtgcacctgggctcgccatgcctgctggaagcgctgtaccgcaccgagagcgcgtacccgcagcggctggagatcaaaccgtggaaggcctatcgcgactaccgcaaagaaggctacgggctgctgatcctggaaggggaagactggcagcgggtccggagtgcctttcaaaagaaactaatgaaaccaggggaagtgatgaagctggacaacaaaatcaatgaggtcttggccgattttatgggcagaatagatgagctctgtgatgaaagaggccacgttgaagacttgtacagcgaactgaacaaatggtcgtttgaaagtatctgcctcgtgttgtatgagaagagatttgggcttctccagaagaatgcaggggatgaagctgtgaacttcatcatggccatcaaaacaatgatgagcacgtttgggaggatgatggtcactccagtcgagctgcacaagagcctcaacaccaaggtctggcaggaccacactctggcctgggacaccattttcaaatcagtcaaagcttgtatcgacaaccggttagagaagtattctcagcagcctagtgcagatttcctttgtgacatttatcaccagaatcggctttcaaagaaagaattgtatgctgctgtcacagagctccagctggctgcggtggaaacgacagcaaacagtctaatgtggattctctacaatttatcccgtaatccccaagtgcaacaaaagcttcttaaggaaattcaaagtgtattacctgagaatcaggtgccacgggcagaagatttgaggaatatgccgtatttaaaagcctgtctgaaagaatctatgaggcttacgccgagtgtaccatttacaactcggactcttgacaaggcaacagttctgggtgaatatgctttacccaaaggaacagtgctcatgctaaatacccaggtgttgggatccagtgaagacaattttgaagattcaagtcagtttagacctgaacgttggcttcaggagaaggaaaaaattaatccttttgcgcatcttccatttggcgttggaaaaagaatgtgcattggtcgccgattagcagagcttcaactgcatttggctctttgttggattgtccgcaaatacgacatccaggccacagacaatgagcctgttgagatgctacactcaggcaccctggtgcccagccgggaactccccatcgcgttttgccagcgataatacgcctcag

SEQ ID NO: 56 - DNA, 251 bp, Homo Sapiens
acaaagaaacagccttggtataatagcacgttagcatcaagacgaaaacgacttactgctcattttgaagacttggagcagtgttacttttctacaaggatgtctcgtatctcagatgacagtcgaactgcaagccagttggatgaatttcaggaatgcttgtccaagtttactcgatataattcagtacgacctttagccacattgtcatatgctagtgatctctataatggttccagtatagtctctag

SEQ ID NO: 57 - DNA, 339 bp, Homo Sapiens
aaattttagtttactttcatttggagaggaagctgaggaagaagaggaggaagtaaatcgagttagtcagagcatgaagggcaaaagcaaaagtagtcatgacttgcttaaggatgatccacatctcagttctgttccagttgtagaaagtgaaaaaggtgatgcaccagatttagttgatgatggagaagatgaaagtgcagagcatgatgaatatattgatggtgatgaaaagaacctgatgagagaaagaattgccaaaaaattaaaaaaggacacaagtgcgaatgttaaatcagctggagaaggagaagtggagaagaaatcagtcagccgcag

SEQ ID NO: 58 - DNA, 753 bp, Homo Sapiens
gcaacatggcgtcttcgggagctggtgaccctctggattctaagcgtggagaggccccgttcgctcagcgtatcgacccgactcgggagaagctgacacccgagcaactgcattccatgcggcaggcggagcttgcccagtggcagaaggtcctaccacggcggcgaacccggaacatcgtgaccggcctaggcatcggggccctggtgttggctatttatggttacaccttctactcgatttcccaggagcgtttcctagatgagctagaagacgaggccaaagctgcccgagcccgagctctggcaagggcgtcagggtcctaatctggatgggtattgatcatgtccaacctgctggagccccttcacatggtggatgatgccccatgaccctgtagaaattgaatcctgctcacaacattgttggccttcttactaaccttggaccgtgattgagcccaagaaaccagggacttacgcatttggccaatgtcaaaagaacagaactttgcccactgcacacttgctgtgtacaatgactgagccctttcttgtagtttgtttccttgtttgagaggtgtgcatgcgaccgtggcttttcccaaagtttctgactttgtggtttacccccttcaccttccagggacgcagttgttacgaggttagacgtggcagctctgtgcagtgtttgagcctacagtgggatacatagggtcaaattgagaataataaactgagtcattctcctgga

SEQ ID NO: 59 - DNA, 491 bp, Homo Sapiens
ctcctccatttacaacctacaaagataaaagaaagtggaagccaaagttttggagaaaacctatttcagataatagcttcagtagtgatgaggaacagtctacaggaccaattaagtatgctttccagccagagaaccaaataaatgttccagctctggatacagttgtcactccagatgatgtcagatactttacaaatgagactgatgacatcgctaatttagaagcaagtgtgcttgaaaatccttctcatgtacaactttggctcaagcttgcgtacaagtacttgaatcaaaatgagggggagtgctcagaatccttggattctgctttaaatgttctggcgcgagcattggaaaataacaaagacaatccagaaatttggtgccattacctcagattgttctcaaaaagaggaaccaaggacgaggtgcaggaaatgtgtgaaacagctgttgaatatgctccagattatcaaagcttttggact

SEQ ID NO: 60 - DNA, 324 bp, Homo Sapiens
gttacgagaagtttctgagaagctgaacaaatataatttaaacagccacccccctttgaatgtattggaacaggctactattaaacagtgtgtggtgggaccaaatcatgctgcctttcttcttgaggatggtagagtttgcaggattggtttttcagtacagccagacagattggaattgggtaaacctgataataatgatgggtcaaagttgaacagcaactcgggggcagggaggacgtcaaggcctggtaggacaagcgactctccatggtttctctcaggttctgagactctaggcaggctggcaggcaacaccttagg

SEQ ID NO: 61 - DNA, 167 bp, Homo Sapiens
gttacctagggtatgaattaatacagacttggaaactctgaaagaacttagaatcagcattttgagagcagaagcttgggcatgctgtgattttccaataaactgctatcacaatgtcaaaatgcagttcagacaagagcaacacagagatctcaaacattaaaacg

SEQ ID NO: 62 - DNA, 138 bp, Homo Sapiens
gaaccaaccggtgagccctctcctaagagacccaggggaagacccaaaggcagcaaaaacaagagtccctctaaagcagctcaaaagaaagcagaagccactggagaaaaacggccaagaggcagacctaggaaatgg

SEQ ID NO: 63 - DNA, 138 bp, Homo Sapiens
gttgttaaaaaagcagacatgatcaacaaaaatatgactcatcaggtccaagctgagagagatgcactggcactaagcaaaagcccattcattgtccatttgtattattcactgcagtctgcaaacaatgtctacttg

SEQ ID NO: 64 - DNA, 209 bp, Homo Sapiens
ggtatttctcaagctgccagatacgtttaccaacgactcatcaacgacgggattttgagccaagccttcagcattgctcctgagtaccggctggtcatagtgggccacagcctcgggggcggggcggccgccctgctggccaccatgctcagagccgcctacccgcaggtcaggtgctacgccttctccccaccccgggggctgtggag

SEQ ID NO: 65 - DNA, 1346 bp, Homo Sapiens
aatatcgataacttgatgaccccagaaggagttggccttaccactgccttacgtgttctctgtaatgttgcatgcccaccacctcctgttgaaggtcaacagaaagatctgaaatggaatcttgccgttattcagcttttttctgctgaaggaatggacacgtttattcgagttctgcaaaaattgaacagtattctgactcagccttggaggctccatgtcaacatggggactacccttcacagagttactactatttcaatggctcgctgcacactcactcttcttaaaactatgttaacggaactcctgagaggtggatcctttgagtttaaggacatgcgtgttccttcagcgcttgttactttacatatgctcctgtgctctatccccctctcaggtcgtttggatagtgatgaacagaaaattcagaatgatatcattgatattttactgacttttacacaaggagttaatgaaaaactcacaatctcagaagagactctggccaataatacttggtctttaatgttaaaagaagttctttcttcaatcttgaaggttcctgaaggatttttttctggactcatactcctttcagagctgctgcctcttccattgcccatgcaaacaactcaggttattgagccacatgatatatcagtggcactcaacacccgaaaattgtggagcatgcaccttcatgttcaagcaaagttgctccaagaaatagttcgctctttctctggcacaacctgccagcccattcaacatatgttacggcgtatttgtgttcaattgtgtgaccttgcctcaccaactgcacttctgattatgagaactgtgttggatttgattgtagaagacttgcaaagcacttcagaagataaagaaaaacagtatactagccaaaccaccaggttgcttgctcttcttgatgctctggcttcacacaaagcttgtaaattagctattttgcatctaattaatggaactattaaaggtgatgaaagatatgcagagatattccaggatcttttagctttggtgcggtctcctggagacagtgttattcgccaacagtgtgttgaatatgtcacatccattttgcagtctctctgtgatcaggacattgcacttatcttaccaagctcttctgaaggttctatttctgaactggagcagctctccaattctctaccaaataaagaattgatgacctcaatctgtgactgtctgttggctacgctagctaactctgagagcagttacaactgtttactgacatgtgtcagaacaatgatgtttcttgcagagcatgattatggattatttcatttaaaaag

SEQ ID NO: 66 - DNA, 3193 bp, Homo Sapiens
gaaggaccatcgagacctgttcttgaatacatcgatctggtctgtggtgatgatgaaaaccctagcgcctattatagtgatattctgtttcctaaaatgccaaaacgacagggtgattttttgcattttttaaatgtgaagaaggtgaaaacagacacagaaaataatgaagtgagcaaaaatcactgcagattgtctaaggcaaaggaaccacatttcgagtatattgaacaaccaatcattgaagaaaagccatcactttcatcaaagaaagaaatagataatcttgtgcttccagattgttggaatgaaaaacaagcatttatgtttacagaacaatacaaatggcttgaaataaaagaaggtaaattaggatgtaaggattgttcagcagttcggcatttgggatcgaaagcagaaaagcatgtccatgtgtccaaggaatggattgcatatttagtaacccctaatggcagtaataaaactactaggcaagcttctctacgaaaaaaaattagggaacatgatgtttctaaagcccatggtaaaattcaggatttgttaaaggaatcaactaatgattcaatttgtaatttagtgcataaacaaaataataaaaatattgatgctactgtaaaagttttcaatactgtttacagtttagtaaaacataacagacctttatctgatattgagggggcaagagaattacaggaaaaaaatggagaggtaaattgtttaaatacacgttacagtgcaacaagaatagcagaacatattgcaaaagaaatgaagatgaagatatttaagaatattatagaagagaatgccaaaatctgtatcataattgatgaggcatctacagtttcaaagaaaaccaccctagtgatttatctccagtgcacaattcagtcagctcctgcacctgttatgttatttgtggctttaaaagaattggtgtcaactatagcagagtgtattgtcaatacattattgactactttaaatgattgtggttttacaaatgaatatttgaaagcaaatttaattgcattttgttctgatggtgctaatacaatcctgggaagaaagtctggagtagctacaaaattgttagaaaattttcctgaaatcatcatttggaactgtttaaatcatcgattacaattgtcacttgatgattctatatccgaaataaaacaaattaatcatttaaaaatatttattgataaaatttattctatttatcatcaacctaataaaaatcaaaccaagcttctaggaactgtagctaaagaacttgaaactgaaattattaaaattggtcgagtaatgggaccaagatgggcggcatgtagtttacaagctgctactgctgtatggcatgcatatcctatattatatatgcatttttctcattcttactctggtttggcgaagagattagctaacattaatttcttacaagaccttgctttaatgattgacattcttgaagaattttcagtactttcaactgcattacagtcaagatcaactaatattaagaaagcacaaaaattgatcaaacgtaccataagagctttggaaaatttaaaaattggtactggaaagtatgaatctcaaattgaagatttgatcaagtcagataagtttaaagatattccatttaataaaaacaataaatttaatgctcttcctaggagtatattactagacaatataattcagcacatgaacctacgccttttatctgacagaaaccatgaagatatttttaattactttgatttgctggaaccttccacatggccttatgaagaaataacttcaccatggatagctggtgaaaaaacattatttcatttgtgtaaaattttaaaatatgaagttgatttgaatgattttcgggaatttgtaaataataatataaaatcaaacaatgtttcaattcctacaactatatacaaagctaaaaagatagttagcaccattgcaatcaatagtgctgaagctgaaaggggtttcaatttaatgaacataatttgtacaagggtgagaaatagtttaacaatagatcatgtatcagatttaatgacaataaatttactggggaaagaattagcagattgggatgcaacaccgtttgtaaaatcttggtcaaattgcaaccacaggttggctacagatacaagagttcggcaaaagtcaacaaaagtcttccatgagaatcaattggctatatggaacttaaaatagaatattgtatacgttttttgtcatctgtaaattatgtactacacatcctttatatacataaaggtcttttttttttttggaaagccagttaaacttttatcagcatgttgctgtttaaaaggcgttctttaagaagataatcttgaagattggttttagaagctatagttttttagagattggcccatgtttgctagagtgggtcataatacatattccatgaagttctgtacagaacaaacaccgtttataattttgtactgttttactttaagtaaggatgcaaaaaatagcaggactcagctaagttctaagccctggaggttatattaaataaaagagaaatggaataaaaactgtaagccttttggctcttgtctttctcatttccatatctgtgctatctagtatggtagccactagctacatgtggctgatcagcttagggctaaaagtaaaatttttatttatttacttttacttaaatatgaaaactgaaacaatgtgaaatatttttctgcttagctttactgtttttcagtaatacattttactctgttaccttgcataagatatggttgttgtagtgtgcctgtcaaacttgtgtgtcatttccaatattgaatataaacatattgctgatagatatgatactgctgtcagcagatggattcaatgcaagttattttttctacaaattgatgtaacatcatgtttatctgaatattttatgcagacaacaggaattacagtgattcctgtgtaaattataactggtaattgaaatacttattttaatcatgattaaattatttttctagtttacacatgaatatgagcaaatttttaaatttaaaatgaaggcatactgaaaaagaatcaagtggcgatgcagaacctagtacaactggaacagtaaacagactcaaagaag

SEQ ID NO: 67 - DNA, 433 bp, Homo Sapiens
gtctgtaaacagatttggctaggattatttgatgatagatcttcagcaattccagacttcagggatccacaaaaagtgaaagagtttctacaagaaaagtatgaaaagaaaagatggtatgtcccgccagaacaagccaaagtcgtggcatcagttcatgcatctatttcagggtcctctgccagtagcacaagcagcacacctgaggtcaaaccactgaaatctcttttaggggattctgcaccaacactgcacttaaataagggcacacctagtcagtccccagttgtaggtcgttctcaagggcagcagcaggagaagaagcaatttgaccttttaagtgatctcggctcagacatctttgctgctccagctcctcagtcaacagctacagccaattttgctaactttgcacatttcaacagtcatgcag

SEQ ID NO: 68 - DNA, 679 bp, Homo Sapiens
agaaatggtcaatgcatggtttgctgagagagttcacaccatccctgtgtgcaaggaaggtatcagaggccacaccgaatcttgctcttgtcccttgcagcagagtcctcgtgcagataacagtgcccctggaacaccaaccaggaaaatctctgcctctgaatttgaccggcctcttagacccattgttgtcaaggattctgagggaactgtgagcttcctctctgactcagaaaagaaggaacagatgcctctaacccctccaaggtttgatcatgatgaaggggaccagtgctcaagactcttggaattagtgaaggatatttctagtcatttggatgtcacagccttatgtcacaaaattttcttgcatatccatggactgatatctgctgaccgctattccctgttccttgtctgtgaagacagctccaatgacaagtttcttatcagccgcctctttgatgttgctgaaggttcaacactggaagaagtttcaaataactgtatccgcttagaatggaacaaaggcattgtgggacatgtggcagcgcttggtgagcccttgaacatcaaagatgcatatgaggatcctcggttcaatgcagaagttgaccaaattacaggctacaagacacaaagcattctttgtatgccaattaagaatcatagggaagag

SEQ ID NO: 69 - DNA, 239 bp, Homo Sapiens
ctcctaagccactgcctgctggtgaccctggccgcccacctccccgccgagttcacccctgcggtgcacgcctccctggacaagttcctggcttctgtgagcaccgtgctgacctccaaataccgttaagctggagcctcggtagccgttcctcctgcccgctgggcctcccaacgggccctcctcccctccttgcaccggcccttcctggtctttgaataaagtctgagtgggcagca

SEQ ID NO: 70 - DNA, 451 bp, Homo Sapiens
atcccaagtgtccttcaagatatcactgtactcatgaagctggtgtacatagtgttgggctaacttggattcataaacttcacaaatttcttggatcagatgaagaagataaggatagtttacaggaactctctacagaacagaaatgctttgttgaacacatcctttgtacgaagccattgccctgcaggcagccagctccaattcgaggattttggattgtacctgacattctgggacccacgatgatctgcatcaccagtacctatgaatgcctcatatggccgttattaagtacagtccatccagcgtctcctcccctgctttgtactcgagaagatgttgaagtggcagagtctcccctccgtgttctggctgaaaccccagattcctttgaaaagcatattagaagcattttgcaacgtagtgttgccaatccagcatttttgaa

SEQ ID NO: 71 - DNA, 351 bp, Homo Sapiens
ggtatttctcaagctgccagatacgtttaccaacgactcatcaacgacgggattttgagccaagccttcagcattgctcctgagtaccggctggtcatagtgggccacagcctcgggggcggggcggccgccctgctggccaccatgctcagagccgcctacccgcaggtcaggtgctacgccttctccccaccccgggggctgtggagcaaagctctgcaggaatattctcagagcttcatcgtgtcactcgtcctggggaaggatgtgattcccaggctcagtgtgaccaacttggaagatctgaagagaagaatcttgcgagtggtcgcgcactgcaataaacccaag

SEQ ID NO: 72 - DNA, 85 bp, Homo Sapiens
acacagcagccagggggtacccagtgaccttcccctgaacagaaagaccattccattctggctacagtgtaatggaggagcccag

SEQ ID NO: 73 - DNA, 951 bp, Homo Sapiens
gtgcacaaaaagctttaaacaagttaaaatctaggcaacagactccctgaagtgttgactgcccaacttaatcatgactgtgattcaaaaatgttaataaataaaccaaggtgtagatcatggggaattcatatgctgggcaactgaaatctgctcgatttgaggaagctctccacaactccatagaagcctccctcagatgtagtagtgtggtaccacggccaattttttcccagctatacctggaccctgaccagcatcctttctcatctgcagatgtcaaacccaaggtggaggatctggacaaagatttggtaaaccgctacactcaaaatggaagtctggatttttctaacaatctaacagttaatgaaatggaagatgatgaagacgatgaagaaatgtctgattcaaacagcccaccaattccctattcacaaaaacctgccccagaaggatcttgcactacagatggtttttgtcaagcaggaaaggatttgcgtttggtatcactgtgtatggaacaaattgacatcccagcaggattcctcctggtgggggccaagtctcccaatctgcctgaacacatcctagtttgtgctgtggacaagcgatttctaccagatgatcatggaaaaaatgcacttttagggttttctggaaattgtatcggctgtggagaaagaggatttcgatatttcacggaattttccaaccacattaacttgaagctcaccactcagccaaagaagcagaagcacttaaagtactacctagtcagaagctcccagggtgtactgtctaaaggacctcttatctgctggaaagaatgtagaagccgacaatcctctgcttcttgccactctattaagccaagctcttcagtgtcgtcaactgtgaccccagaaaatgggacaactaatggatacaaatcaggattcactcagacag

SEQ ID NO: 74 - DNA, 706 bp, Homo Sapiens
aaataacatctgctttgctgccgagctcagaggagaccccagacccctcccgcagccagagggctggagcctgctcagaggtgctttgaagatgccggaggccccgcctctgctgttggcagctgtgttgctgggcctggtgctgctggtggtgctgctgctgcttctgaggcactggggctggggcctgtgccttatcggctggaacgagttcatcctgcagcccatccacaacctgctcatgggtgacaccaaggagcagcgcatcctgaaccacgtgctgcagcatgcggagcccgggaacgcacagagcgtgctggaggccattgacacctactgcgagcagaaggagtgggccatgaacgtgggcgacaagaaaggcaagatcgtggacgccgtgattcaggagcaccagccctccgtgctgctggagctgggggcctactgtggctactcagctgtgcgcatggcccgcctgctgtcaccaggggcgaggctcatcaccatcgagatcaaccccgactgtgccgccatcacccagcggatggtggatttcgctggcgtgaaggacaaggtcacccttgtggttggagcgtcccaggacatcatcccccagctgaagaagaagtatgatgtggacacactggacatggtcttcctcgaccactggaaggaccggtacctgccggacacgcttctcttggag

SEQ ID NO: 75 - DNA, 62046 bp, Homo Sapiens
ggaaaagaaaccaagtctccttgaatcagtttatcccaatgtcttctggttttctatgttggctacagtcaagataacctattactaaggtaggttgtgtgtttctagaccaccaaatctgttcaatagacctttttgcatctgagtggacaggtcctagatgacactggagacatagaggtgaactccctccctggccttcaggtactcacattgcatttgggaacctaggcattaagcaaacaaccatgatacaatgtgctaagtgctatcactaaagtataaacaaagtggtttgaggggcgttatgttcccccatattgacctgaagctgtagtattttgactgcttctagagtacctgtatgtctctgcatgtccaggcagtctgatttattaacagggccctcttttaatcttggagtctctatgtggatgataaactgtatggtctttctatttataaatcaaaaaccaatataggaggctagtttaatttgtgagcaaatatgtttctatttattaagaattcttgagttgcctgcctctacattagtggttgaggcagtggagtgagttcctgatctgactcacctttctcaccaacagtgatacttgtactttagcatatacatgagagactgagttgattctatagcctaactggaatttattcattccttcatccatctactcaatgaatcaattctacatttactgagcactgattgtgtgtcagccaatctactaggcacgtgggatacagtgatgatctatatagatatatctctgcccccacagaacttacagagtggagcctcaaaaagtgatttcaattttatccatctgctttgtgtcttctctttaaatccttgctacccaaagtaggctctcaatattagcagcatcagtgtcacccagaagcttgttagaaatggagaacttaggcccacctcacacctattgaatctgaatctgcagtttcaaaagatcctcaataatctgagcagattctgattcagttggtctagggtcttcacttctgacaaactcgcaggcaatgtcaatgctgttggtccatggatcgcactttaagggacttccccagttctttacaaaatgtgggttgcaacccaatagtgggtcatgaaatcaatatagtgagttgtaactagcacattcaaaaatattttaaaataggatagagtacacaaacaattcagagtgcatttcatgtcatgatgaaggcattgttgcatgaaacttgaatcagctttttgtacatctgcacacagacacaaacacagatgtatgtagactgagttctgatgaaaacgtatttcttaatacgggtcgcagctgataacagtttgaaagttattgttctagacaagcttatctaggccagaaagaagtcctcagaagtgattccagtctagaacagagagctcagatcaaaggggttctgttaatattagaactatccagaatcccagtgaggacatccaggaaaaagtagaacagtggggctagaaagatcctctactaggggtgacagctgtgtgctgattggatgatgagatgcccatgattagtggcttgtaccagttggcattaggtttttgcatgacccaagattttggactgtgagctccttgaaggtgggacctgtatgttactcctagaggctaaaacagtgcctttcccgcagggggagtttccgcagtgtactgtgactttttatatctttcatactactcaatataccacatgtaacaaatgaggtcttttctgtctttccactgggaatcatgtattattcatctttgcttttttctagggcctactccaatatctagagatggcagatattcagtaagtttattcacttaacaaatattttatttgttattcagcaaagaagattttctctcagctatattacatcagttttagaaggcacacagatgaacaggcttgataaagagcaaatctgtgaaaaacaaacacttgtggactaaatggtatcacatttacagagctgtaagagaggtataagtcaccctgggcaccgagttttatgctgtattgattaactttccatttcccttggttcaattttagtttctctcttagaattatgtttctaccctttgcattctcaccgccaatcatttattgctgcacgtgtctttccccctccatatactgtgctccttaattcactaagggactgtactaacatagcctaaatacccaccttccagccacccttcaggagggtattagctgtatttgtgcacagcgagagtgccttcaaggcagagggcttcctcccctatacttcacctccaaatctctttcctttccacaaatgctagtgctaggcagctgtgctcactattattcgtcttctctgtagttcttgactttgcctgtacgttataaacacttggtattttaatatttcccatgtccaggttgcagttcaaaccaatgaagtcagaatctctggaggtgggacaaaggtatttgtagtttttaggcttctcagatgattccaatgtgcagtgaagattgaatgctattgcttagaggaaaattcagtcacctgatatttgaagtttaggtaatttccttagccctcaaaatatgttcatttcttttctagtccttagtatccctgcccaagtctgaaaatatttatattgctatttaatcagaagtaaagcaattaaaatgtacatgttactcaagaagaagaaacattagaacattgctcaacttatcacctgtgtggttattaagctattgtctctatttcaaaagcacctttctgtattctactttgtgactctgggtttttttcaccatctagctcttaggtggcactagaagaggaccacaagcctgccagagggaggagagtattgcttcttcctgttcccattagtgtcacctcagcagtgacttttcaccctggagtggcaggcaagtttctagattatttgagtactccccaaatcagtcttgctgcaccccctgagagataccagcagcaatgaagtgtgagtcccagctctgggctcccccctccaagattctgggttttcgtaatcccttgtgcttattaatgctgttaccttagggtcctttttttttttttttttaattttcaatgcttgtccagcaagtttcctatactaaattgctatatggaaacatctattcttctctcctttgactgtacctaactcaaaatgccttctcatttaacaaccagtgctctgcaacaaggtgagctttctaggaaggcaaataacaagaaggctcaggttggcttgtctccattttggggcaatgggagtgtgctggaagtcttccaaggaacaagagtgagaagaagatgctagaatgaagaggtggacagacataggggaggaaagcgcactggatacaaactttattaccttcattattttgctaggtctgtcttagctgagagactctgagagtattaatcagagataaatgggaagtaagtaatagtctatagtaatccatagtggcatttattaataatacaaatcttctggcctccatctcttcttcttcccataagcccacagtttgtccacacataagcatatgtggttttgtccttaataatagggtagtgcaaatagttaattcactactcctagtgccacgagtggcacgttggagaccatcctgtctgttcttatgtaacctgtcataaatccacgtggatatgtatctcattaagacagccatttaggatttgctttttcagaattgtctactttggtcagtagtattttaactgtaaaataaggatggtttcacttggtccaagtgtccctactgtacctaatacatttaactttacaaacagttttatacaagcaatacattaaccaatgagctgtaatgagtagaatttggatgttcatgagggagatttaaattcacacaccattttcctagactccttcactgtcttgacactttcctattattagctctctttttggagatattttgtgttaaaattccaaaaagttctcagtcaccatctgagaatttttgcgtatacaacaatgtaaagaaaaactaggccttgctaagatgaagtacccaggatcaactggtagcctaggagtttgactatggtccacgattcaaattggtgggccaaacaataacaaaggtggacttttgggggttacttttgtacatttgctttttgtatcctcgctaaagaggtgaagtaactagtagccagtgtgttgaggggaaattcactactgcctggaggctctactgtgtattctacacctcctggtctccaatgagatctttatttcttgttttgaatacttatattaactccagaaaaaatttagttctagaatctggatactgagcaacactgcagctctctttcttagttcaaatagtctcatctgcttttctttgatgaatctttttctttgtaggatctgtttctctgtgggatcaacattttctctctgtgtgtcagaaagaatgtcttttaccgtccttgacagggagagtcactttctgttgcatgtgagcaatcagagcaatcatccttctctcacgttgaatgagtaggtcaaacagtattagtaatattgcctagtttactctaatgttgattatattagtttttttgcattttttgtgatctagagcttcagttgaacctttcattctgaccatgattatttagtaagaccacagatccatccactaacttaaaggagaatgaatgaccaactgtaaccccgccatctgattaggaaaacaaggaaacaaacaaaggcaataaagcccttcaaaaatgtttttaaccagaaaacactgaatagtagaaatatcactgtaaatggagtcaggcgatgtggattctagtcaaagctctaccacttattaactgcgtgaccatagaatgtcgcttaacctctctgaaccttagtttcctattccataatgcaaaacgtaagttataatatgcgttttgtccaccttacagacaaggatcttttacagaggctggaaattcctccaactcgtctgttcaggttgactttctggaaaagtgttttattctcagaggatttgattattgagttatctttctattctgctgctgtcagtagatttgccataaatgtttcctgagagtctctctcttgaagagattcaggccccagttaagttcatcccaattaaaaatagaaccctgagatgcagcagctgtagcacctcttaaacctcttcaaataacattgaacgattaagtggctgctactgtggagatagtgcccacaaaaccagcagcaggccctctcggaaaatgataacactctaagtggctgtcagtccttagtcatttctagtcattcaggggatgttgccttatcttctacaagttctaaaagttcctgggaagagaagaatcacataaattctttatttaaaaaaatcccccttcccacagcaaaattctctaaaacaatactaaacaagcaggcatgcgcaattctaatgaagtctatcatttgagtggaagagaattgctatggttaccaatgtttctgtctggcagttttgggataaaagtctgatgaagtgatattgtctggaaaatgagcattgattcctagggagatggaagaatttactttttttcttcctatttcttgaatacatggctagctttcaaatatatgactcaacaattacattcggcttgaatggatttttttctgacattgtttcttattttcttctgatttatcaaggagagtttaaaaataaactttccttatcccatccataatagaaatttcagctattaagaacttaccaagtgaaaaattatagaaatcactaaagttcagagggagatctctttattatatttcaacagctatttgctttggagacatttcttttccccctttgtaaagctaaattgcatatatcactatttattaataagcagaatgacaattatttaggctgccaaatctcttttataaacatttcgttagctaggtaggatttttatatactttcccaaagggccaagttgacagacatgggctttcactttatttccatagagatctggatccaaatccctgggtccaaatatatgccattcacttcccatatttagtcctgttatctctctgaagactggcccaagagaggcctttctgatgagacatttagcacagcgattaatcactagataaacatgtgaatgggtccatttgcaaagtgaaagctccctaaacagaggcttgtcctttacaaaaagagcaggtccaattttccttaaaagttcatggttgcctatgtggtgtgatttgagaaatcctgaaccttctgttcccattactttccctgttgagctctacatttcatagtgtatcactcctgtggatgaaacctggcctctccagggcagtttctctaataaatggccatagtcctgggaaaggtcctgatgccttagtgtgcagtgtgcttgacaccataagatcctttcttctttgataaaaaaaaaaaaaaaaaagactaagacaagaaaactggtggctgttggggaaaggtgtggaaagaatgaaagaagaaaaccgatgcatggaaagatggtaaagtaccaatggaccggtgtgagtcaggttctgaatttaggagtgaggactcaaatatgcctgtttactgggttagcttttggaacgatcctatggaacatatatcctatgaacattatagaagtcactaaagctgactgcaaattgagtgtaagttgagagtatctttctggaaaaacaaattggaaatatttatcaaagggataaagaacttacatatatttagaaccaaaaatttcaattgcaggactctatcctaagaaaaacaattagcaatgaacacgtttcaacccaaagatgttcactgggacattatttaaaatagcaaaaatattagaacaagttacatgtttattaatagagaaatgaatactatggaaataaatttatgctgtaaaagtgaccaaataaagcttggctcaggggctcatgcctgtaatcccagcactttgggaggctgaggtgggccgatcatctgaggtcaggagttcgaaaccagcctgaccaacatggtgaaatttcatcactactaaaaataaaaaaaattagccaggtgtggtggcgcatgcctgtaatcccagctactctggtgagtgaggcaggagaatcacgtgaacctgggaagaggaggttgtagtgagctgagatcacgccactgcactcaccctgggtgacagagcaagattccatctcaaaatatagtatataaaatatgctaataatatgatctcaaacatataaaataggtaaaagaaaggaattttaatagtggttgtccatggatgattgctatttttatttattccttttcatatcttctaaatttttcagaatggtcatttattacttttaggtacaaaaaagttatttaaaaattcatccaaaagcaaatgtttgtgtcaacgttacaaacctaaaatatggatgtcctctaaggaggttgacttcagctgtatagctatttcccagtggataattaaagacctgtcagactttctctcatatatgagtcctttatgtttaagggctgtaattctatataaaatgtctgcatagaataacatttggcacctgatgtttgattctggatagtattgtagttctctattgccaaatgtgagactaaaaagttagatggaggttagcatcaagaaatgttttctaagcttttcccctggaactggacaatggtagaaatgtgccagaagcaaaggaagcctgtggaataagtctctttcagcccaaagttatattaaatagtttcatctattcactggacatagatttgagtcatcaaacttcttccctaagtttggtttgcttcccatgtttcagatatatctgaagagcaggtctggcctcactgccttggtaccattttggtgatcttcccttcacctggaatgcatcttaaacctctcctatctcaggtttcttaggcccacgttaggtaatagctctttgaaacttctactattgcccccaattggtgtgatctccccatcctttgaacttccatggcacacttatatggtgttttcatattaactctattagattatcaatttcttaagcacaaggtctgctgtgccttactcaactttgtattccccatgtcttgcccatcttagcagaactgaatttgtttatttaatgaatcaggtactagtttggctggcccaaagtctgactgtctcacttcatgccatttagatatgatgcccatttattagctctaaaaaatcacaagtaagaataacaatgaatttttcaaaacttcttgatgaaatatctttctagaataaactattaagaaaacaaaaacatagagcacagagctacaaacttaagttaccagggtgctctaccatggtttattgatctttagtagtgtggaagaacaacctgtgctttcgtgttagaaagaactgggtttgaagctaatcttgacaattattggcagtgaaattttgggttaatgttctatcttcttcaagtttagtttccttactttcaaaatgtggaattagttaattaatggctgtctcctagtgctgttgtgagggttatataaaatgatgtatgaaagcacttagcaaagtatctggcacacagatttaagtctctatgctcatgaggacagagaatccatcatgtccaggacaatttatgttctcttggaaagtcagggttatatcccctagactttgcagggtagttaatttctgggtctattattctgaaatcaccaatctactctcataaccaccccccactccgccacacagacacacaaacagctcatctgaaaattttgtaggaatactgatgttgggctctgatatggtttggcagtgtccccacccaaatctcattttgaattgtagctcccataattcccatgtgtcataggagggacctgatgggagataattgaatcatggagggaggttttcccatgctgttcttgtgatagtgaataagtctcatgagatctgatcgttttataaaggggcgttcccctgcacatgctgtcttgcctgccatgtaagactttgctccccattcatcttctgccatgattgtgaggcctccctagccatgtggaactgggagcccattaaacctctttcctttataaattacccagtctcgggtatgtctttattagcagcatgagaacagactaatacaggctcagaaaataatacctccaaatgagagcctccacagcagccttagaagaaaaagtttttctctgaccttctcttgccctcctatctctcagtcccatttttcctcaaggctaggcatagaaaccagaatccttccccaaggaaggtcatagaaagcagaacctcttttcccaaaagccagccataaatcctgaaaatattatatgtaagaactgaccataaagaaattatctgacccacattgtttgattctaggacataagactgccatttcagagagggctgtgccccacaccagaaggaaggaatgcatgttcagagaagccaagaagaatctagactggctggccctgctgggcttccccactcattctcttagctttagatcttaccctttttgtccaggcatatttctacatgactgctcatactttgttgaatctaagcataaaaatggacaatttcccctatatctttgggttttaattatgaaggctctcatgtatacacattagataaatttatatgccatttcttttgttaatcagtctgcttcatatcagtgattttcagcaaacctctaggggccagagccctggttctcacattgagctggaatattgaaggaatttctgcagtgaaatcgtatttggctgtacatatataatctgtgaacctcttaaataattaaagcataacttactaataaagcaggaaagtttagtgggctccagaaccgaaaccagttggatttaaaatcctgctgctatacttaacctctctgtttctgagttttcttatctgtaatgtgcgatgataatatctgccttgtagccttgctttaaggataaaatgagctaatgtctaacaagcacttaaaacagtgcctagtatatagtaagccctcaacaaaagctagcaattcccaattaattgatttaaaagctggagctcttaaaggactatgttgagccaaagtattccaataactaaatgaagctcctgtagtagagttggagccatcttttgggggcaggaagggagtgtatggcgatttgtgccttatagtggtttggatttgttaacagcactagtcaaggaacacagtgcgctgaaaaagttgccagacgccagatgagataaggagcgtcctgtgagtaaggacagacttgaacactcccagcacatagacaaaggaagtgatggctctgcctaagaaatcttgtccttcaaccttgacttcactggatttttttttcagacactgctaatcatgtaaataaacataagaacttcctgatagtggactatctctgttttcagatgtaattcctacctctatttgcaacatgggtatttaataaaagaggaataaagaggcatgcttgaatggataatcaagtaaacagattttctgatattaaaaaaataaaggaagtatataatttgctctggttatctggtctatattttatggctaagtcattttaacatttcagggactttcttcttgtataatacctagacctttactttcatatattgaaacttctctactgattgattaggtttgttgttgttgttctgtctcacttatttccatttattgtctgttgtactttttgagtttttttaaaaacttaagcttttgattgaatatctgggtttttaaaataatatttttaatttataatttaagagatgatttacttagtttaaaaattttttttctgcttcctgcactgttttctctgagtctctttctaggttttttggttttgtcttttgattgtggctcatgcattttttgttaggggatttcctcaaatgtctggagaacctagactgtccattcataattataaatgatgtactaaaaaggtattggtaataggcttaatagcatattaacaaagagtatgcaaactttcttatgggaggagatccccaaaatgtctgtcttagttcttttctcttgggtccgttagtttctctggagaggaattttattctatctggctggtcttaggttggatggcagtattctcagagctgaatgtgggcatggaatgagtggtctcactcttcagcattcagactttcacttaattgccctcttttttagtgcaggtgtcatagattgcaataacagcaccaattctgcatgcgtccttgtattcacatcctttgctgtgagactgctgtttctctccctgaataggtggggtctttttatggggtcccgagtctattctactttgggcaataaaatatgacaggattgacactgtatcacttccaaggttaggcttcaaaaagtgtgcatttttgtctgttctctcaggcctgtcactggcataagaacattttgaacatggctgccagatgatgagaggcttgtagatcagagtggagtccacatagtcaccctagccaaagccagcctagatcaaccaaccaatgttccagcatgtgagcatgcgcagacaggatccacctcctagccaatcttcagctaacaccatattcctaagcaatactgttttatggttgtataccgctggagttatgtgattgtttgctataaagcattattggcataacagataattacagggtttcaccaccatcctcagatgcaaatctctttagaaattaacctcctgtcttttgcccagactggggagaagtcaccaagctataggggtagggagggatcaggacatcaaactgcaccttatattaatctttaattaatctgggctccatactcgatctctgatgtgacaattcctgagcttttccagggccctgtggctcaagtggggtcatttctttttgaatctctctctgcaggcgccaggttttgacctggtctatgctcagacactttccatttttagaggttttatttttcactcttctttgcgttgataccctccttggttcactttgcccgtggggtttgggctctttagtttttttcctcttatatgaagaaattttaggaggaagtgggggtaaacatgtgcttttacattccatgtttagccagaaatgtctccaggttttaatttttcaccttgactttaaaactttttttttcacttgtaaatgttcatctggtaggtgtgatttacttcagggtgttaaatggcatgattataaacatctctctgaataatttaatataataataattggctcctatccaatgctttttctcttcttcattgaatctcatatataaatttatttcatggatatttatcaaggttctattatgggccacgtatagtgccaacattagtgaaacagtgggaaacacaacagacatggtccctgtccttaaggaacttatgatatagtggtttatcactagtcttcttgaagtacttctttctgccttgtcttagtaaatatcctaagatcacccatcacctacctccgttgttcactggagtttatagtgtggtgagccagcgtaagagtggaatcacagagttgtcaatttaaatattgggcagtaacttacccctttttaagccttgggatcttcagtgtaggatgaaatgttaaaaaaaattagactatattgtcaacatttaaaaattaagagctctctcttctttctccgaaacacacatgcatgcacattctgatttctcgcttctttcaaaaaatcaagatactgcaatattgggccactttcctacatgatattaagcagaactcagtatgggctggcctccttagatgggcatggtctgtgtaatttgcctaggacaccaccacttcccattatagtaagaaagttttatacatttgtgttatttgtctggcctttatgggcattgaagttttccacctgctttagatattaggaaatggcagtgctatttaaaaaaattcctaaaggaatactaatctgttaacctgtagctttcaaagacatgagacattatatttttcccagatattatagatcagaagttcttaaatattaatgtatttactcatatacacatttagtcatagattttaaatatatttcaacgtttagttgttgaaaactaaatgaagtaacaacagtacttgataatgactgggttgcagattctcaactagctttgaccttcttagatgtttcattttgtgaaatccataatttatttgatttctggccctttgtgttaactcactggactcatttgctctcttggtctacagcctcctctgaattgcttctgccctgttttcactttgatccagtttccacacagttctgaacccatggctccatttatcaaacctcacgctgtgccttcagaagcagtcttcatgttttctctccctggcatttgggctatgggcatcccttaaagatcccagcctggttcattcttctagtcccataagcctctgattaactttagtggttcaacagaactgcatctctgcaggctcttgcctggtccagccttgcttaaaccacccctcctaagatcaaaaaagagggttcaaatatcttgttaaccctagggtcctgtgatggttatccaagttgagcagaatactgttgagatattaagattgatttgaacactgaagtgaacttcccacccatgaaaatacatgaataaggattttcctctcttccttaggaaaaaaataaatctttgttcctctgataatcctattgcccttgtattttgcctaatttcttcaagagatatacactcaacaaaaaatcaaatataattcaagggtgttgtagtcgagaacaatattagaagaaggaggttgatgaccggtctccttctctgcagtgttcagtgcacttctgagaattgtgctttgccatgcataccaccaaaggagggagggagatgcagataaactagaggttgtcctcaggagagcctttaggatggattgtgggaacccaaagggcacccatgtgaggagtagttgagaagatagggaagagtatgcttggagaagaggagactttggggtcgggagtggtagtttgagagagggcaggaacatagctgtcatcagaactatcaaattgtcacatggaagaaagggtaaatgactccaaaggattgaagttgactcaatgggtagaaacctcagaaagatatatttgggttctggttttctttttctttctcttttttatttaaagaagagccatccaaatcttttttttttaattatactttaagttttttattatactttaagttttagggtacatgtgcacaacgtgcaggtttgttacatatgtacacatgtaccatgttggtgtgctgcacccattaactcttcatttaacattaggtatatctcctaatgctatccctcccccctccccccaccccacgacaggccccggtgtgtgatgttccccttcctgtgtccatgtgttctcattgttcaattcccacctatgagtgagaacacgtggtgtttgctgaacgcgcccgatctcgtccaaatcttaagttttctgtgagggagaatagtctggacctctatgcaactaagcataaagtggatgaccacttggtggggatattgtagaagggattgactcattgacagggtgattggaccagatgatctttaagatttccctctatcctgggattctacagtgtttaaaaattgtcatgacaggagattcctgcacattttaaaaattaaaatcacaatattgtacgtgctttagaaagcctagcggaatggatttaattctgcctattgtagtgataataatgcaatattaatatgtggatatgatctgtttatggattactcatatagacaaattagacatatttattggtcaacaagtgccactacacattgttggaatacatgtggctgcttttttcataaattaatcattggagtgaattaagatgagaacataacaaatgtcagctcataacaaatgtcatctaattctagaccccaatttatagatgaaaataatatatattcattttcttccttctctctttccaaattggtagagaaaagatataaatattcctcttatatcatcacttagagataagaatatgctaaatctggaaaattctgttaggaggacctataatggaataattctgcctcatttctttcacatgagataaacttcacttttattagtatttctccccagagtcttctctaatcttctcaatttgggttcttcccattttctcccatgtccttggaccacttactgtgttttaactagagcatggttatatgtaaatatgtgtgttttccccagtggatggcaaaaaggaatcttccttgcacatttttgtaatccccacaatacctactgcctttcctgcgtggggtgattctcaatagacgtttgtggtttgaaatcatttcaaatcacaaataagcaccttcacttatgcactacatatttggtccagcagttaatatgacagtactcattaaatatcccatgtgatattgtgatgaacttgctttggaagttattgatcatgaccattatatgagaagaagaaaaacctgattactctgaacacgtaggagcacagcagaaaagaaaatgcacctaaaaggatttttgttttctgatttttttttttttttttgcacagctttcaagtgcagacaactttttcttgttgtctagagccattgcttccagagcttatgaggagctataaaagaggtcaggacacctgggtctagtctcagctctaccacgaactagcaatgtggccaagcaccaagcctttctggtcttagctttctcatttataaaatgaagaagccaaagttaactggatttctagcccctgccaactctcatatctctaacagtaagttgagagatttttctctctctgacattgtcccctttctttccacctctcacaatgtgcgcaacttgagataagttacttcacctctgtgtggctcagctttcccatgtatacaatgggagatacctactttttagtgttgtgattaggattaaatgagttaatgcatgtgaagtgttggaacagtgtctgatgtggaatactcactaatgttaactttttattttactactttgctgaaaatgttagtgtatttccagttgttaaaattacaaaccaacccctcacatggtgcctgactcaggaactttaccgactatgggttttcaatctattttatatcattcctctttattttttaatttttagtttttgatgcagtgtctcccactcttgcccaggctagagtgcagtgatgcaatcatagctcactgcagccttgatatcccagcctcaggtgatcctcctgcttcagcctcctaagtagctgggatgacaggcatgtgctaccgtgactggatttaaaaaaaattttttttatgtagaacgaggtattactatgttgcccaggctacattcctctttaaagtagcattatataaaattgtatttccgtctgttctcagaatatctctgatcattcacacactgtagtctataattttctatctgtatcccctaccctgtgttgctcagtgtgtttgtttttacacaactaatctttggcttctggctcactgtcctgtgttgagggaccatgtagagacttcggtgtttgccatttccatgctcatagaactcattagtacaatagatggagacacctggtgcatagagatggaggactcagcatgagaatgtcccattttccatagccctcatttttgcttggagcatgatgaatgttacactggcatagttaagaggtccatcagttcagttcagtatattataagtaaaattaaatatattgagcatttcatctgtgataagctctgttctagttttgtatatatgagaaaatatatccaactggaggtagaagagagaaagatatgaagagaacaacagcggggtctttgttctcaaggagtctgcactgttgctcgatgtggtgctctagatatcagggagaagtatgccttggaagataataattgacacctacatagaaattcaatggatacaaaatccttttatatatgtaatatcgtttaatccttcatcactattatttgatttaatccaaagtttaactaagttttgcagaggttaaataacgtgccagcaggctgcccagccagaacgggaagtcagaatgttcttctgcctgtagggtgtgtgctccaatgtgcctggagaagaagaatgtgataatggataaaacgcagaggttccttgtatgttttactgtttttttttgttagtctgtagtacacttctgattctccaaagtggtacacaattttccaactttgaatttttgacagagtcatgatttaaagcttcaagcatatgattatgagatggtggttctcaacatttaccacaacctcccagcaaaccacacagcagtcaactctcaagatttcttccaggagggtaaagggatttttctcttcacatgccatccaactccatgggaaaacattagcagtgacaaatgactctggatgaatgcttgaatccgaggtaaatatgagcctttgctcctccacataagaaaggtgccatcagagtattctctatgaatacatttgaaagacaacatctgaacaattttggttattatctattagccttcaggagaatttctataattacgaagcacgcagtgggaagtgttattgcttggagccaagtctatttatgttgatatttgtagatatttacatttattttctttggcagacatagacaaacacatttctgaaaactactttgtctgtggaaaaacgaagcagaatatactttaaaaaaatctaccacagaaaggataataatagctttcatgtctgaagacaacagacaggcatgaaatataagaattattttatcctggacaagagaattggtgttagagcaaattttgaccatgtcacagtctggataactcttaggtttaaaacttgcacccccatagacctcagtccacagaggagataactgatctggggcttagcagtattggcagggtggtgcattatgggatttagctatagagtgtttgctccccaaagagagaactgcaggaattttaagaacttgcatcaattcccctgtgtagaacagtatgtctaagtatatttcaagtaaatcctcaactcaacacataaagctgcagttacaccacgcctttaaaaggaaattagagcacagaaacgaggccagactgcccctcctggagacaaatcccctgttcatctcttcaactgctgtcagtccttaagtatgtttacctcctgtcccacccaccccactaccacatcagtttcaggctcttgattttttcttccaggcctcaaaaagcatgtagcaatttatataatttttttagacattgtttttctagcaaattaattcagatcacaatgctttttgtgatgaattttaaaacattgattatcttttttttttaagaaagtcctcaaaaatggtctttgaaggggagatttgctctgatgagtatgatataaaacctcagcctttgaataaatacaatatgccttgctggcatagtaactaggttgcttggatgaggatgcatactaattagggcaacttaatggcataatgactagggtcatttattcatggttggttgttcttcaaatgtaatttgggagagagtgccaatgcatacccattctaccttcaatagagagagtggaattccacaggtttatggggacaaataggacaacacaggtaatttcacagatttgcagttagcagttttctatttgatacccacagtttttaacagttttagctccaaacatgttagaattaaacctcattgcctgtagagtttaaaagacagcattatcatctgtatgtgtatgaggtggcaacaactttgaagacaaacataaggcataggtggcacaggttcaaggacctgtcactctgtggactcacttctgaattgttcagttgcttttgctcttagtcacctgcatctagtagatgcattttggaatatcccaagaaagtgaaaatgacctaacattattcaagctgtgctttggtgtttctcctgttgacctttgcctgttacttgactttggttgagtaaagagctatgacatggatatcctatcatttcatattctggcctagtcctatatctatctcaatgtataattttcctgcagtcatactgtctaatgagtgctggtgagaattatgtatcataggtttgaaataagatctattttaatttttgaagagtgatgaccaatatgccattccgtctatttgactgaaactgattgcagtagtagattatattctctccaagacagtaatgacaccacgactgtttttaatgacttccaacataggaagaaaaacttagaactcagaactcagggtgggtgtggtagaagaattatcgtataaaatccatatgttttagagttggttgtactgtagtagaagagctcaggtatgttttgggttagtatagtccctcaaacaatttctttcaaatgttttctttcaaccaaatctgaaacacattttgccaaaactttttttcccttaatttttaaaggcatgtgtatttaaagggacatttaatccatatgttttggctcatttacacttaactcatcaggatattgtttttaagagttatttgagctccacaaaacccattttataagtcttttcttccgcagtacaaggaagttgtgttttgctggaaacagaaatggaacccacaaaataattaagttcggatgaaaattgagaatgtacaaggtttccgaaagccactaaacttaacttagaaaaacatgttattctcttgcagttttttagttacttctagatatcaatatgctttagccaaggtgctaaattagaacatcttttaaatctttatttcgcagttaagtgtctaaatgcttatttcccaaaataccatctttagtgtaatggataaaagcaaggttaaaaaaataaggagcatatacaaaaaagtgagctctaaatttcttttgaggttgatgcagagctatgatcttgaaccttcattgtgccttttctctccttgccagtcttttcatggaattatgcctgctatgcccataaaagttccttctgctgtgtttcatgaatgtaggtagctactgctacacaggatctcactgtgtctgcatcctcagcaccttcagagccctcagatatttcctgctgggtagaaggatggatctagggaatttattagacaatatagtgggatttcccttctgtcttgtatttctcaggaagattggtatctgaggatattcctggagagaactcaagaaaatcaacatatgctttagatagggtatatacagacaatgtccctgaagagttgggtgagagataaagcccttcagatcagcattgacctgaaattatattctcatcattttagagagaagttgagcacctctgaaataaattttgtctattgaacctcaatatctaatgaaaagaactaccctttctcaataaaaaaaaaattactacaacattttccataggccccagaattccttgagcaaaccttctgtaatgagttgagttgttgatactccccttttcttctcatatttgggtagatatctgagtaaaaatcaatttcaattagagaaggtaagagcaaaaaattaataagatttttatatttcatagtaaaatataaaaatgtgtattagacaacaattagtaaacaagtaacaaaatggcagtagtaaggcctaatgtataaataataacattaaatgtaaatgaactaaattagtcacttaaaagacagtatggctgaatggattaaacaataagacccaattatatgttgcctacaagaaagtcacttcacctgaaaagacatacatagactgaaagtgaagggatgggaaaagatatttcatgcaaatataaaccaaaaagaacaggagtgtctatacttaaatcagataaaatagagtttaagtcaaaaactgaaaagagagacaaagaagaggattctataatgataaaacgtcaatttagcaagaggacataccatagtaaatacatatgcaaccaacactgaagcactcatatatataagaaaatattaatagatctaaagagaaagacagattacaatacaatagaagtaggatacttcaacaccccgcttttggcaaggacagatcagccagaaaatcaacaaacaaacattgctgtttgactgtattgtagattaaatggacttaacatacctttagagaacatttcatccagttggatgtgaacacacatttttttgattaccacatgaaacattcttcaggacagaccatatgttagaccacaaaacaagtctcaacaaattataaaaaggtctaaatcatatcaagtatattttctgaacacaatgaaataaaacaagaaatcaatagctagaggaacttcagaaaccatacaaatacatggaaattaggcaacgtgctcttgaacaaccagtggatcaatgaagaaattaagaaggacaataaaaaatttcgagaatcaaataaagacagaacacaacacacctgaacctatgagatacagaaaaagcagcactgagggaattttataccagtaaatgcctacatccaaataataaaaaaaatttaaataattaacctaacaattcacttccaggttactagaaaagcaagaacaaatcaaaccccaaaatagtagaaggaaagaaataacaaagatctgggcagaaataaatgaaattgagactaaaagaaaaatacaaaatattaacaaaattaaactggttttgaaaaaataaacaaatggacaaacctttagctagatcaactaagaaaaaaagagataagacccaaataaataaaatcagaaatgagaaaggaacattatgatggttagcacagaaatacaaaggatcattagagaccattatgaacaactatatgccaaaaaattagaaaacttagaagaaatggatatattctgaacacatacaactcaccaagattgaaccatgaaaaaatagaaaacctaaacaaaccaatcatgaataatgagattaaagcagtagtctaaatttggtcatcaaagaaaaaccaaggacctgaaggattcactgctgaattctaccaaaacatttgaagaagtactaataccaattatactaaaatcatttaaaaattaaagaggaaggaatacttccaaattcattgtatacagccagcattaagctaataccaaaaccagacaaagacacacacacaaagtaaaactacaagccagtatccctgatgaacacagaagcaaaaatgcccccaaaaatactagcaaacagaattcaacaacccgttaaaaaaatcattctcaatgatcaagtgggattcacctcatagatgcaaagacagttcaacatacacaaatcaataaacacaatacattatgtcaacagaatcaaggacaaaatccatgtgataatttttataaatgctgaaaaagcacttgataaaattcaacatccctttatgataaaaaccttcaacaaattgggtatagaaggaacatatctcaacacaatcaaggccatatattacaagctcacagttaaaatcatacagaaagagtaaacattgaaagcctttctacaaagatctgtaataagataaggaagcccactttcaccatatgtattcaacagagtactgggaatcctagccaaagcaattaggcaagagaaagaaataaaggacatccaaattggaaagcaggaagtcaaattattattatttttttttgcagaagacttgatcttatatttagaaaaacctgaagactctgccaaaaaacttttagaactgaaaatttgtaaaattttaagatacaaataacatcttagtaaaattacaagatacaaataacatataaacatcactagtgtttctatatgccaacagagatctatctgaaaaagtaatcaagaaaacaatttcatttacaatagctacaaaaatatcaaaatacaaatttaaccaaagatgtgaaaaatctctgtgaggcaactaaaacactgatgaaagaagttgaagaggatatgaaaaatggaaaatatcccatgccatggatgataaaaattaatattgttaaaatgtctatactaccctaagtgaactacaatcaaaacaccagtgacattcttcacagaaatagaaaaacaatcctagaattcacatggaattacaaaagatctggcacagctaaggcaatcctgagcaaaaagaacaaagctggaaacatcataccacctgatttcaaattacatgacaaaactataataaacataacagcatagtactggcataaaaacagagacatagaccaatggaagaggatagagaccccagaagtaagtgcatgtatttacagtcaactcatatttgacaaaggcaccaagaatatacgctgggggaaaaggacagtctctttaataaatgatgctgggaaaacaggatatccatatgtagaaaaatgaaaaaaaaaagacccctacctttcaccatgtacaaaaattaactcaaattggattaaagacttaaatgtaaaatctgaaactataaaactagtagaaaacattggggacatacaggacattggacttagcaaagatttttttggataagacttcaaaagcacaggcaacaaaaataaaaacagacacatggtattacataaagctaaaaagcttctgaacagcaaaggaaataattaacaaagtgaagagacaacctacagaacaggagaaaatattttcaaactatccatctgaaaaggaattaataaccagagtatgtaaagaactcaaacaacttattaacaaaaaagcaagctagcaagcaagaaaaaaaatctgatttaaaaatgagcaaaagagcttaatagacatttctcaaaagtagacatacaaatggccacaaatgctcaatatcactaatcatcagggaaatgcaaatcaaaaccacaatcagatattatctgattggctttatcaaaaaagaaaaaataacgaatgctggcaaggacgcagagaaaggagaacactggtaaactgttggtgagaatgtcaaatcattatggaaaacagtaggaggttcctcagaaaaacaaagatagtactactatatgattaatcattccattgctgggtatatgtccaaaagagaggaaataagtatgtcaaagggatatctgcagtttcacgtttattgcagtagtatttgcaatagccaagatatggaatcaacctaagtgtccatctacagatgaatagataaagcaaatatagtgtatatacacaacggaatatcattcagccataaaaaagttaatgaaatcctgtcatttgcagcaacatggataggactggaggtcattataagtgaaataagccagtcacagaaggacaaataatggatgttttgaatcataggtaggggcaaataaaagtagatctcatggatatagaatagaatagtagttaccagaggctgtcaagagaaggtggaagaggataaagttggttaatgagtacaaaaatatagttaatagaaggaatgtgttctattatttgaaagtacagtaggaaaattatagttaacaataatttattgtatatttcaaaatagctagaaaagaagaattataatgttcccatcacaaagaaaagataaatatttgagttgatgaatagttcaattactctgacttgatcactatatgttgtatatgtatattaaaataccatgttcacaaaaatgtgtacaactgtgatatagcaataaaaatacaataaaagtgtgttatgaatagctgaatatctggtaatataaataaaaatctttaaaatcttaacagtctgaatactagagagtaaattttatttcttaaaagacttttactacagtcaattttgtcatgcaatattaatggagtgtaatgagtttgatagggcagttctatgtatgtgctgtcttccctttgagaatgaatatgaattcttttctgtcaacattgcatatagataatattaatcatttgaagcacacactgtggctatacctgaggtaaggtgttctggtgttgataaatgaatgaatcatcctcagagatttagatgaaattcttgaaaagtttgctattatttttaaggcaaatatttaatttgggaacagaggtccatctttctgaatatttgaggcttctaaaatgtacaaaaaggaatctgtttgacatttctgagaaatagaggaatccaatgatataagggtcttttctgaaagcattaaaaaatttagttataatcttgaatgataactttcagtgttttatcacaatgaaagtatttcattgacccaaattaatggactgtttatttttttatactgtaatgctagtgaagttgtagggaaaattatatacctagatatgtatttgtataaatatataaaccttctcaatatctggcttcagtagtcttacattttatttattgaaatttaaaaactttagtaagtcagttctcataaatattaacctaggaaaacaataatgcatacttagcagcaaggttaatttatggacctgcatttgatgtgcccttttattttcctggttcaattcctggattaagtaataattctaattttaagaaataatatgaagttatccaaataaatatagaagtagctgaaactattttttgcatattttaaattttattaaatttaaatagtaaaactaatacatatatccagactataaaataaaaatattataggtatggctacaatctcctttgatcattaccatgaatcttgatccttttttgagaagtagcaacagcatttaatttgatgtatatcttttcagaagattttatttttatttatgtaacaaatatatataaatattttttcaagactgagtcttgctctattgcccaggtgggagtgcagtggtgtgatcttggctcactgcaatctctgcctcctgggttcaagcaattctcctgcctcagcctcccaagtagctgggattacaggcgcctgctaccacacctggctaatttttgtatttttagtagagatggggtttcaccatgttggccaatctggtcttgaactcttgacctcaaatgatccacccacctcactctcccaaagtgctgggattacaggtgtgagccatcatgcccagcctatgtaacaaatatttatggccacatactatctatatagttttttttgatgtgtgtgttttaaaatgtaaatagtattttaatgtatgtggttattttgtaacttctatttggctaccttgctaaactttctaatgtgaactctaacaattcacaagttaatttcttagaggtttttttggtaattattatatcatttgcaaataatgataattttgtctgtttttattctgattcttatatctgatttgttattgtcttggatagacaaggatggtgcagaagtgctggatagataatgatagtattttgcattattccttactttaagaaattacttctaatattttatccattaagtatggtacttattacatatttccactagtgacatttattaaaagggttttcttctatttctaatttcagaagattaaaaatgaggtattaaattttagtcaatacctttacattacttattttttgtcttttgtcttttaatatggagaattggtaggttctctagtattgaaccatccttgtattcctgggataagccctatttggtcctgatgtgtaattaaaaaaaaatccctgtatctgatttgctaatatgtaattcaaggtgtatatatcttcatttataaaagttatttgtccatgatttcctttcttgtgctttctcttgttttggtattagtgctatatttgcttggtaaaatggatcagataatttcttatctttgtctttactttgaaaagtttatataacacaagaattatgtgttttttaaggatgtgataaaattcacaagcaaaatcttgagttgggtgccatttcaagtatagtcttttctgttagcttaatgtattcagtggttactgatgtgcgtgttttaaaaattctttgagctgattttgatgatttttctagaaatttgaccattttagctaagtgttcatataggtatgctaaaatttccaccttaaaatggaagattcattattgctttctcacttgccaggggcagagaaattgttctacttgacaagttaattttagctttccaccaacaaaacaacctacagagtatttgtactatatgctgctttagtttttgtggtctctcagtgaagttagttattttgcaatcaaataattatgggaaattttgcttttaaaaaaatcaagtgacaatgtattttcataatgacaccttcgattccagaattcaggaatcatagaattaaaaaatgcttcgaattttcttaaactccatgtttcctcaccagtgaaatagaaaataacaaatagctatctcatgagggtagttatattaaagtacataatgactctactggtcttcatacaacatttgtgtgtgttaagttatagggataagtttgtattaatatatgccctttgctttagagaaaacgtacttagcaaactgtgaatttgaataagagagttttcctctatgtgtaagtaattataattgaatctggggcttggactatttctaatcatcttaattaattagcaggataaagcataaatggactattttgtctaattttggttatcaaacaagtagacctgtcagcttaactaaaaataaaatgaacaaataattaagtgattgcatgtagctagttacagctttaatcaatgtaatcagtcaaataagttaaatagtcaaaagtatttgtgttttcacacacatgttaaatcagtgagaatgattttgttgtcttactgtaattgtaggaaagtagcaaatttgaatatatcattcaagcatgaatatataggttgtactgctgttgcaaaaattaaaattgacagaaagataaatttatctactccaagtccaccaagcctggcaggccagtgaaggtgatgtgagaagaatggtcaggagatgaggtcaagaacctgaactatatattctgttaagtttgggacacagttaaccaacttaaatccgttattttggaaaaggagattatatatggtgtatattcagtatcagctgaacaacttctctttagcaacatttgctcagttctggcctttatctttgaccttctttgcaagatattaataaatgctttttattctaagcgtaagtgaagtcaagtctagttaattaaataatattttctttctcatagttttgcattaagacttcactatttcaagactttgtctcaactgtccttgccattttcttgtcaattttaaagtatttccttctctaccagggaaagtgcttccaatttctcctcataagaacatttgtcttactgtcttaaaatttcaaatcgaagacacagattaagtggctagaggtttgtgtcctttttatacttccttgcattattctcctcaataaagttaaactgtgtcacacagcaataaatgtacatttttactgatggagtttccagactttttgtgattaggagattttctgtcttttacctaactcttcatccctggagaagaagatgtgtgatacatagtgagaataaataatatactggagagaggaggggaggagtgattaagaaagtcatatttgcattgctttatagagcaaggagcagacctttcagcccaacaattccacatccttgagagatgcttctaccagaggggggcacaaatatcagatatattgtgatcactttcatggcatttgagtttgatttgtatttgtattataatttctgaacatcactatcacatattttatttttaaataaatgagtggagttgtctaaatgacttaattgtaacttttgatttgtaggtagaaattgcgttaagaaataacagaaagaacatgaatttatagtcagaaaacccaggttagaatttgagctgtgctatattttagctgtattaatttgatgaaggcactcacaatttctgaattttagtagacacatcaacatgaatgataatattaatctcatcaggctgttgtgaagataaaataaaatgtttttgtaaagcactagtaagctgtctgacacctaaaagaactttaatgtctattaattcctcttttgctcaatagctatattaacagtgctgagagcagggagactcctgttatgtccccagctccatttctcagaaaatatctgaccttggtagttcccataaatgttctgagtctcaattttctaaactgtagaatgagaagaagtatctgttcaaataaaaggtccgtcttagagaaattttgtgactattaaatgagaaaataatatgcaataaactttgaaaattatagagtgctaaagatatataagccatttcctttaaaagaaaatattagttcaggcactaatgttgtatacactgtattttatctcaaaatcagaataaatagacattcaggaaaaaaaagttcttaatattttaacacatatttccttgtcatgtttcaccacatgtatccttggtcatttttctaagtccctgtgcaaataggactccaaaaaataatttttttctgagtattaacatttgttcttttacaaatataattgtgggtcactgaaaggcccttagcttcctatgaaacaactaagagtagcttgaatcttctcctagaagccatatatacataagggtatggagaatattgggagttttattttgaatgaagataaatggaaaattttaggttgaaagaaaaatgagattacttattaagatcaaataaaattaatattgttagaagttgttttttgaaagctaatacttatctaaaagaaaggtcccttgacagagtaaatttgcttgagctgcagattttctttttcttttatttttggataatttcatcagatttggaaaagaattaagaattgtctctgttattgtcatttagactttttctgaacaatagaactaatttaccaacacggaagaaacatgccaaatcactgaccatagcccttgaaaggaagggtttggtttttaaaaaaattaaattagatctataagtttgagaaatttcagcatgttttttataattctttattattgtgaaattggaaaaactcatgacattgctttatgtaaccaagtgttgcttggctgcatacataagctttgtgctgataagtggggcatctgttataatgcttagaaatacttggagttagtgtctgggctctaggatggggccgcttgactcactgtgatctgaattggaacacactgctttattccacagccatctagacagaaagtgaggacaacaatttaccagttaaagcaacaaagcaattgaggtagacagaaagtaattagtttttaatgatcttttggaagtcacatgaatagattgatttctgttttgatcttgtttgaattaatagctgcttttgagtcttcaatcccacttgagtatgcattgtttctccatttcacggtctggtgctcctgaagtttaggttttgcttggccaattgttccgttttgtaaagctcagaccagtgtttttgtgttccttagctttaaacattgttgtggtgttttccttttatttctatagctgtttcctcttgggaatttgatggggctattatcacttccacagggctaacatccaaattggaggttcttatttggaagctaaaattggaaaaaattggcgtgaattcatttctcactaaaaccgtaacccaggctccgtgttccttgggaagagcattttctgctcttgtcctggttgctgcccagactttttcttctctcctttttggattatgtgagttacttagaaccagctaatgagggtgcaggaagcagtttgtatggccggtgccactgttatgactaaaaagcttttccccaaaataaatagtagtttgcctctccccaaagctttgcagacaatgaatcacaatatccctcacttttgcccaaacattgtccctttttttcttatcagacagatgtctacatcccttcactctgcttccctgttgcccatttgattctcagacactcttccttgtcaggaaggtactaaaggcttaatatctttttaattctctcttgtttatttgtttttgatgaatcaagctatgcatgtatgtttttataaaattattttttgagaagatgtggaagatgaaaaaagccaaattcctacagagagattttcaagatttttaaacagacctcctgatttttctatacaattaagttctttaatatatgactttttaatactgttgtgctttaaaataatttttcacttaaaatgatcacctttccatgccaataaatatagatctccatcctactttttaaagactgccagtgaaagttttatatctctatggtcctagacattagggtaactataggaagaaaataggtgtgatttgaaaatagagaagatcaaagaaatatcatttactaagtctttctagaaagcagaaatgagaagaaatggtttgataaggtcaaaagaaattgcaacctggtgtgaatcaacagattttagaacactaaccaaacactacacttacattaagtgtgaatgagcattttggaactttaatagattggtgctgggatgctgaatctcttcaccatccctcactccctaggacagaggagggaagtagaaaaagaaagagagcacagagaaatggtagaaaataagatgcaggagccaaattggaaaagaacgacagagaacgctggactagcaggttgggagtagtcttaagagagagaaaaggagagggggctatgggatatttacagccaactttccatttagcgccaggcacccagtggagaaaagacaatttatccttgtcatagccctggctgcactgctgctcctcctgccaggcattttgttcccagaaggcactccccagtaatgtaaggagaaggtgggagagggggagagaaggtcagtaacgacttccatggtcagcaccctagtcgttttcagcagccgtttcagggttcttcaacagccttgtcactttatctactgtgtctgtttacttctctctggatttccattttatgttttgtataattgggtatctctagctctctatttctcccattcagctgagctaaaattaaattttaaatttttgtttaagctccttttttatgttttatattatcattttgccctcaaccttttactttctacaggttaaaaatctgatattttaaaaataaatctccaggtggaccaaaggttcatattaatccatcatctttgtgcttttagtatttcattggcaaatatcccacaggattattttatctcaaacattttatttcatgaagagatatgaatttggtccttttgcagaatcctgggagaacttaaccttcaggaaaaagacaaaatgattgccttcttccaagccagtgactcagtttgaatatacatgatgggaagtgtgcatctgccagtgacctttgggataatttggacttttggcctttttccaaaataaggaagcagtagaacagttgcgtcttgttttatgaattcagtctcattcataatccagtatttggaaatttcccaccttttccagcagaagttgcctagctctaatatggacactgagtatcctcatcatgagacatgttttgtgtgacactctggcctatttgctccagaggaaaaagtagcagcaggattaggcgatggattcaaattccaactctcttcctctatcgaactgatattctaactgtagtttggttccttgatatgtttaatgaggttttaatagcatctacctcataggattgttgtgatgactaaatgaaattttttttttttttttgagacagagtttcactcttcttgcccaggttgcagtgcaatagcgtgatctcggctcactgcaacctccacctcccaggttcaagcaattgtcctgcctcagctcccgagtagctgggattacaggtgtctgccacagtgccccgctaatttcttgtatttttagtagaggcggggtttcaccatgctggccaggctggtcttgaactcttaacctcaggtgatccacctgcctcagcctcccaaagtgttgggattacaggcatgagccactgcgcccagcctaaatgacattttatttgtgcttaagttctccataagtagtaactactgatattactaaattatttatattgtttctactaaaagtgattattagacataatgtcaagatgctcatattctgcttaggggttaaaatacttaggcattaattacctagaggatactgaagtgctaaactctatggtgatggtcatattttcaattgtaggctctcaggttaaggaaagatttaagcagttcagcaagtggggataagatgaccttcctgaagaggtgaggattgaattgggcctggaagagtaggtagaatttaaaagacacaaaagggaatttaaggttcaaaagaggaggtcaggggagaaaaagtatgagtaagcatataacaaaagactatagagttttagagctatttttgtggattttttcgttctctttccccccttttattggtatccttttatggaatctcatttcattttttagctgttacctgtgccaaaataataaacatgctcatgatgtcatgtcactttaaatctctaagaatcttggtttactattcagctgttgctttgaaagtgcacaattctggaattaattctggggagctgtgtttgcagaagtgcttatatttaggagaattgagagtttatattcctagagacaacatcaaattgctaaggaaaagctgcattccacgaaccaggaaacacaatctagtgttcatgatggctgttcgtccatgtattaaggaatttgtactgagtggtcaactgcagtaagtgctactggaatgcttcgtccctgcggtaccttacctcatgtcctcactctccctcatagtcacagactgtgaaggaagctgtggaggtggggatttaaaaagaagaggaaatcactggagagccagagggctgtttaaaattctacaagtgaagcaggttaaaaacataacccagcatttctctctttgcaactagatgcaagattaggaaggcatttaaaaaaaaaagtgggagttgaacaatgagaacacatggacacagggagggaaacatcacacactggggcctgtcgtgggggtagggggctagggaagggatagcatcaggagaaatacctaatgtagatgacgggttgacgggtgcagcaaaccaccatggcacatgtatacttatgtaacaaacctgcatgttctgcacatgtaccccagaacttaaagtataataattaaaaaaaaattcctgagattgtatgatgctctatataaaatttagaaagaggttaaattaaataatatttagctatttgttcatttaaatctttctaccactcctggtattgccctgtttacctctcagttagtctggtccatggctggagcctggctagccaggccacaggtctctgaccagaagaccgtcttacctggcaaaggacacatggcttccttcaaggccgagcatatcaacccaatgcagaaatgacagtgaatatcacaaatgcattttaggctttaaaagtttaattaaatgtaggggcagtttatgtgatttatttaattacatgcgttctttctcaaagcaccttattttccatgaaagcccctataatgaaggttcccctggtcactgtatctgggcatcacttagaaggattttaagcaaggcaatacagtgcagcatgtttgaaattaaacagatgtgggttgaagttctgtctccaccacatattagctgaatgatctggggtaagttatttaacctctctaagcttattttcccccctttaaagtaaaactaataatagtattactgcaaagtttttattactaataagactaataatagtattattccaaagttttttaagaagtaaaaactaaaagagagtcatttagtcgagtgattaatataaccactagattagattgataatagtgaatattttttaaaaatgtagtggttatcaatatcttaacattattaatgtatgacatagtaaatgtaacaattaacattaaaacagtcaatgttagctattattataattgtcataagacttgattcagttaacttttgtaagaatgtttcctcacctgcaacatgaggataatattaatattacctacagacagatgctataaactacatccaggtgtgctgtgagaattaaatgagctgctgggtgtggctgtgatttacaaaccatagtgtaattattgtgtcaaagttgaaagcaagtagatagtatctggtaggacttttgacctcaatgtaatgaccagaatctgcaagacagcattttggtgagtgctaatcttttgatttagggccagtgtttctggaaagccagaaggatggtttgtaatgaagcagaatgttttgcccagaacggctggctaagcagttgcaaattctgttttctttgcctgttaaagccattttagcttaagtgttgttgtagctactatggagtcctagctagtgtcttactgtacagaattattcatcattttctaagagaatcattttgataaggaacattctctttattttgtatctctattggggtcatcttatggggcatccaccttgggatacacaatttgtgagacattttcagtgatattatttaatattgtccttacaatcaccctctgaagtatgcattatcatctatatttacagacaagaaaagtgaaactcagtaacttgcctaacattaaatgagagagctgagattaaccctcttaccaaactcaagttaggtatatgctcttaccactgcattggttcactggttttcatattactttagtgtaagcacagaaaaagggaaggcaataggagttctcaacccagagcagtgctaccccactaaggagtgtttgaagagggtattgattgcctaactgagttaaggggctgctggatttgttgggcagggatcagcagtgccaaatattctgcagtgctcaggactatacaactgaagaattttcccacctgagcagccagcagtgattcatgaatgtctctcttgaccttttctgaaaagataaaagtggactgtgtctgtattagtatgactatattttgtagttttcacttggatgctttttgtgatataggaaaacccaacccttacttcccaagtgatatgacttgaattctttttccctctaaaacttgtgttgaaatttaatccctaatgttgcggtattgagaggtgaggcctttaagaagtgagtgggtcagagctctcataaatagattagtccattcatggattaatggactaatggactaatagattaatgaattatcatgggtgaagaactggtggctttataggaagaggaagagagacctgagctagcacatcagtacacttagccccctcactatgtgataccctgtgcagctttgggatcctttagagagtcctcactagcaagaaagattttaccagatgtgtctcctcaaccttggatggtccagcctccagaactagaagaaacaaatgtcagtttcttggaattacccagtttcagggattctgttataaacaatagaaaatgaaccaagacacaaagcaagatgaaattgtttcatctttaggtgtttgtataaaccttatcacagtacctcaatcgctgtatccataagcatgagcaccattttattcattactggctttgtggtgcctagcattgagtctagctagtagcaggtgctcaagtaatacttgtgaatacgagggcatagtggtttctctatcatctatgaagctcaggagacaggcctgaaggcatggattgtctgtgcttactgctcttcctccctgccctccaaataaacagaaccagttatccaacttggtttaaaaacattagtgtttcagcggccattaaaaatacattgcatattgctcttcctgaattcttgctccttttctgactaacatgtgatctgatagcttagggtctcatttggtcaccagcacccttctaggaaggaatgttctattccttgaatctggctgagtgatggttgccttacaaagattatttttcacgtcagcttccttaagttgtagatccattttttttccccttgtgtactttgagtgatagagctattactagagctggggtgtgttttctaaatggttggctagagcaaaagcatcatattacatttcttaagaaaggactttaatttattagaattattagttctcattttctacctgttaacctttgaaatattatttacttttccttctggcatttgcatataccttggggttatttttctctttttatatcgatcaaatatactttcatatattgtaaatgtatgtaaagcacttcaggttggcactagtccttttcaattactagttaaaacgctatgatgccctttgagaaagaactatataaatttcatattttaatttatgagctctcagttatgttacttgcttaagttcccttagcaattaagtagaggaattaaaataagaactaaacatgcctgattcctagttatttggcttcaatattttttaagagtttcccttcctcttccctccatttgtgatttcttccttcctctctttctcccaaggaactccattactgactttcttctacatcttacataaaattgttaatttccttactatacttataaccttacacttagagctcagggtagacccaaggaacgattgttaacttaggtcagacctgagcaccgtgggacctgcccaacctctctctcaccttgccaaatgcctcccactttcatagtatccccatcaccaatttcttgacaaaagtagactatccccactgtctcccacccttttattcaccccttctctttccctccttccctccctcccttccatccttccttccctttccttccttccttcccttcctttcacccttttccttccttccttcctctctgtcttttctttctttctttctttctttctttctttctttctttctttctttctttctttctttccttctttctccttccttccttgtctttttctttcttttctctttctttctttctttctttctttctttctttctttcttttctttctttcttttctttctttcctctctctctcccctccctccctcccttcctcccttccttccttcctttttctctctttctctgtttctctctctctcttttctttcttttttttctgttacaggcagcatgtattctgatgttgttttatttagttctctctccttttttggggattgggtaagtatggtagtgggtatgtgtcctcacacagaaaagtttaaactgcaatttgaatgaaattttgtaacttcctgaaatgcagattaggttgtggcatgctagttcactttggattcatgtgatgtcaccaaatggctaaaaaaaaaaaaaaaaatcaaacatgatcagcacagttattactttttaaagccttttgagaaaaacatgtgtgttgaagaacaggatagtggacacaaaaataacttcagtctctaagtgggtagagaattgttttaacttgataatgtgtttgaaaagaagataagacagtggtttgggaagtcactgccctctgccctgtgtggaatccaaggattattagcattacaagaagaaattgcagatgatcattactaaatgaagagattactgggagacacatgcctcaggtcaaagaagacaatggggattaagagattacaactaatacagagatgatattaactgttttcccaaggttgaatacaaaggaaataagaacaactatttctcaggctggagtagggagtggtgctggagctgaggttgcttttaaatattctttctaaatttggccccaaattctttaggttagttgtattactcaggaagaaagattgaagccatgttgatttggatactgtgtcaaaactcagtcttgattcactgtgcttgctgtccttcctcagtcactgctgttgcaattcaagtcctgaagacagggtaggaatccaacctgaggcagttatgggagagactgtaaattcccacactggttttcagagacccggagtctaaacccatttttgaccagaagaatgtgatggaatttcttacacgctcagatacttcatctgcaaaattgagggaataatgctagattattttcacaattccagataattgctaaacatgtaggtacctctcaaagatccacagcattagaagcactgaaaatctatgcggttgttcaataatattatatcttggaaatatcattccaggatgaaagttcatgaatgttttcccttcaagaaggctataacatttatgacataatttatacagacattatttatacagttttggaataatttatttcattttaaaagggctaataactttttatttaaggataataaaatgaatttacatacaaaacacttaaacatttccctaaagtgtgttttggtctaaacaatggtgaaaaccattaggcaaaatttgacattgaagtaaactgaaaacaagttgtctgacttagttttaacctgagacatttaatttagtttcaagatttttataaaaataaagccaagtttcaatgtgtacgttcacttgacataagactatcacatattgtaaagtaaattatagtgtaaaatatgagtatatgactaagaagatactgtcactttaagccattctttaaaaaattctgccaaacagaaatttagaggttgcgctttccttatttagctgggttaatctcttagttgccaagggcagattgatttgtttataaacttgttaaaccaaatctgcacataaccacatcacagataataaagtaggaaaagaccactcagattgtcattctgtatccactcaccacatcagtcttgttaatgtctaatttaactgtttaagcaatgtatgctgcattgcatgttttgcacattgtatttttaagtgatgtgttgatgattgttaaccatgtgtgtgggtgtgcctgaaaagatccaatgtgcagccagaattcctttttcattgacatacccaggctgggctactgtcagtgagtcattctcactgcattgacactgaggcccccatgtgcggtcttacgtgtttgagaccagatgccaggcatatttattccacgttggtaccactagcatagggaagactgtatgccactcattggttactggcagttaaaacttttttcttgacatctgccactttttaaaagccccctaattttgtaataaataagttctttccactaccacttagcaaataagctttcagtctttatagtgtattgctacctaatacatggaattataaaccttgtgagatttaaattttctttgaacgtgacatttagttttcttagcaatttttatgatggtgttgtcctgttagtgttaggtatcagtaataaagatatgtgtgtgtgtgtatgtgtgtgtgtgtgtgtgtgtgtgtctgtagcagaagtttaaagaagatgaagtgactttctgacgggtgatagcatgaacccttgtatggaaaagttttactgtagcacgtagaaatttacacatcacaacttaagaagagagaaatgtggttttcttttctagaatctaacttatactcttgaggttctgttatgcttagaggagataatgtcatttcttctagcccagtagcaatttgatccagttgatttttcttagccaaaattgaatattttagttattgttcatttcaaagcccggccacttctcagtaaagggcatctggaagaatttctttccattttcctcagtggcatctgggaacactgggaaaagtcatttccaatcaggcagtgaggcagggacatgactttgatttttttgttgtcctcctggttcctgcaggtcttggcccagtggtgcctgggcttgcctggtccaagcatggactgatcttttgagcatcctactaatttctataactgatgctcgtgcacccttgattttgagattcctgatcaatgttgatggactgttttagacctaggttctgagcacctttgcatcacccatgacagtccatggggccttccattttccatacctcactggtgtgtgtgaaccttgtggggttggtctctaaactcattgtattaggctgtttgagttgttataaagaaatacctgagactaggtaatttaaaaagaaaagatatttaattggcttactattctgcagcctatacaagcatggctccagcatcttcttatggggcccgaggaatcttacaatcataggagaagttgaagggggagcaggcattttatatggcaagagcgggagtaagactggcagggaggtgccacacactttcaaagaactcgatctcgtgagaactcactcactatcacaaggacagcaccaagccattcatgaaggatcagcccccatggccaaaacactttccaccaggccccacctccaacattcagtattacattttaacatgagatctggaggggacaaacatccaaactatatcattctgcctctggcccctcaaatctcatatccttcttacactgcaaaataaaatcatcccctgccaatagtccccgcaaatcttaactcattacagcatcaagtccaaagtcctaagtctcatctgagactcatcttccacctatgagcctgtaaaatcaaaacaagttatttgctcccaagatacaatggtaatacagtcatggagtacacattcttatctcaaaagggacaaactggccaaaagaaagggacagtaggctccatgcaagcctgaaacccagcaggcaagttattaaatctctttttctttttgagacggagtctcgctctgtcacccaggctggagtgcagtggcgcagtctcagctcactgcaagctctgcctcccaggttcacgccattctcctgcctcagccttccaagtagctgggactacacgtgcccgccaccatgcccgggtaatttttttttttaatatttttagtagagatggggtttcaacgtgttagccaggatggtctcaatctccttacctcatgatccgccggcctcagcctcccaaagtgctgggattacaggtgtgagccaccgtgcccagccttttggaagttattaaatcttaaagctccaaaataatcccctttgattccatgtccctcatctaggtcacactggtgcaagggtgggctcccaagtccttgggcagctctgcccttgtggctttgtagggtgtagtttctgttgctgctctcacaggttggagttgagtgtgtgtggattttgcacactgaggttgcaagctgctggtgactctactattctccggtctgagggggtggaggaccccctcccacagctccactaggcagtgtcattgtggggactctgtgtgggggctccaccccacatttcccctggcactgccctcgtagagtttctctgtgggggcaacacccatatagcaggcttctgcctcagctcccaggctttcctatccatcctctggaatataggtgggagctgtcaagcctccttcactcctgcagtctgtgcacctgcaggcttatcacaagaacagactaacattcgtcagtactagttttatgtattagtacattcttgtgttgctataaagaaataccagagactgggtcatttataaagaaaagaagtttaaatggctcatgattctgcaggctgtacaagcatggctccagcatctgcttctggtgagggccgtcatggtggaaggtgaaggggaagcaagcatctcacatgaccatagtgggagcaagagagagagggaggaggtgccacaaacttttaaacaaccgaatctagcaagaatttgcttactatcacgaggctagcaccaagccattcatgagagatctgcccccaggatccaatctcctcccaacaggccccatctccaacattggagattaaattttaacatgagatttggatggacaaacatccaaactatatcactcatacacaggcatcttcctcagcggtggaatctgcctcttcagctgggtccactcctgctgccattgtgaggttgtaccattccaaatgcagagtttctctgaaaggtttttgatagggccagtctctgcccaggaaagcagaggatcaacaatggtccctcagatgcccccaaacctacatcactgcttctcctgtccatagactgctctctgtccctgaggtttgacctctgggtagggaggcaggaagtgggctgcccacctctgatacctcgtccacacttcctcttcttctttctgccagcatagaaatggcttttcatttcagttcttatcttctagaattgacaatagattattgtctttcttttctactctgggaagcatgtaattattgatctgagtccttcaagtttggcttttatatacaaatacttagctttggagaatttcaatagaacccttttcctcctcaagaaagcctggctcctgctaaaccaaaagcctttgcattcagggaatagccttgctatggatgtaaaatttcatattataaagctgtaaactgattgatgactgctgctcttggctgtgtgttgctcttttcctgagtcaatgaaccccgctggattaagagggtgcagggaagataaggagaactataggaaagttacaatttgaagtttacctgtgatgaaatgacattggcttaatatcacaaaaagattaatcatccagtacatcagcatcatctgtaagccaccaattgactgttgggattttttagatgggtctgatgaaaaggaattaaatttacatttggatttctattaatgactgtatagtttgttcaaaagagagttactctagttttctttaccttgctgttattcagctattttttctctcttttataatttagtctttacatttttaaattgtaaaataaaatgcagatacagaatgctctcccatgaatttgtgtacatgttaatgaattgcttagtaaattttaaggcaaaagcctttgtaaccaacactagatcaaatagaactttgtcagccactcataagcccctctatatcctctattccaatcataatcccctcccttctgccatatgtaataattatgctatataacagaataatagtaataagcttattactattatttttgataatcacttccttgtatgtgtgtgtatgtttagtttgaacatttatataagtccactcctacacattgtaatttagtcttttccattatttaaaatttgatcagtcttttgaaaccttcagtctatggttcttctctctacctcaatcttttccctgtgctttttctgtcgaacacctggactgtttgacctgtaaagtttcccatggtctggactttgctgaatgcatgctcatggtatagtttggtatgttcctttgtccttttgtattttctgcaaattagcagctggatccagacactggatcagattcaagttcaatatctttggcaatactagaaggagtgtttgggaagactataggaggaagctcatgtctatttctttttatgaggccaacaattgttgatgtttagtgtctaaatctattatataataggttctttagtgctactttcatgctctggatccttatggtatatgaaagattcaaaaaatgtcaggcaaataggagacaagcttccccaaattgtactgtatttattttttacattctaaatgccttttacttattgcatttggtataaaccaggttgcaaaggccacttcaagcttggggcaatgtcgagccaaattttagggaaaaaagcaatttctgtggctctatctcccacctgcttttgtggaacttctccctcctacaaaggcctctctctctcaccagttactgtgggaagttctagatggtgtcttttggatgctcttgaaatggtattctcagctaccacgtggcatctttctgtcacttccctcgttgcccatccagctctgacttcctttggtgatgtcttctttaaagcctctgggctctgtgaaacattacttaggtggccccttgcagttgctactgctactgtgcagatgtgggtgacatttcccactgatgccaaagggcagagtgtgctaatttttcttctaaatgtctgttttctcctcctcttgggacacctgtaatacacttctcaacctctcttgcaatgtgatgtgaccgtgtaactaatttctagacaatgctagatatttgcaagtgatatgtgccactttgtggatagctggtttgtcccctccaaactttctttcccctttgtgctggctagatccaaatggtgatggcactctgggcggaggtctgcaaataccttaggctttagtgtcatatggtctctgtcatgactatggtactcagttcagctgttattgtgcaaaactagccatagacaatgcgtaaacaaatatgtatggttttgttcccataaaactttatttacaaaaagaatctctgagctatattttgctggtaccttccctagaggatagcagagccacaagacagaaagaacttggccactgatcactacatagaggaaagctgcttgtcaaccaggaacagccatcttccactattataatacggacaaataagtttctattgtatttcattcatgttttagtttcctatgtctgccataacacagtattattacaaagtaagaggcttaaaacagtggaattttattgtcttgcagttctagaggccagaaatccaaaatcaaggactgtggtccctctgaaacctctagaggagaatccttccctgcctcttcctagctttgggtggcatcggtcagtcctggtcactacaatctgcctctgtcctcacatagcattctccctgtgtgtttgtctctgtgtctcttctcctctctctcttgtttttcaaataataacattgtacatagttttattttcatgaagtcaacaggaattggaaggaaactacaaaatataagtgataaaaaaagtgtgaaataatacaattttacagcaggactatcctgtctgatctcttacctcgggtgttttaacatactcttccctcacttctggacatattcatggctcttctcttataaagacaccagtcatgttggatcaagaaaccagcttgctccagtatgacctcatagtcatgtatatcttaattacatctgcaaagaccctatttccttataaggttacactcagatatattaaagattagaactttaaacatgtctttttggaggtcacaattcacctcacaaagtgattatatgctcttagggagacttattactgaagcccactggactatgaccatgctgtacttcactgatactggaaaaatttatttttcacacttttttcctcacccaaagtgtctcagagttgttgctattgcttcttttttttttctttttcaaaaaatatatttacgtgaaagaagagtcaccatctttctagacctcatgctcaagtccatggagactagaaggaaggcagagatgattctggagaggtaagaaggacagcgatctcaacaataggaagacttcatgctgaagcattgttctactcgctcctcctggtgactgtgtagctgtgtttctaactctccttccctcagcataagctcttgccaggggtctctttagtcagatatactcttgaccagcattacttccttttctcttgaccctcctgcttaccaccctcacagacatgacgagacctgtttaatcccaaactgaaaacagcagacactctgacctcatatgcttttactgagagctataaaacgtgttaaagactaatgataattagaacttcatctccatctccaaacaataatcttcttttaggtgaagctaacaaatgattggttgacaggagcctgtcagaaaactcctatttagacaattttcttcaagcaagaagaaagaaataaaagtacagaaagtttagaaataaaagtacagaaagtttagaaatcgaagtacaaatcggggtgagtcaaaaggtcaaaacttaatctgtggcacacattggcagccaccacctggtacatctggtattatggttctttttattattattatttttaaatattttattatactttaagttctgggatacatgtgcagaatgtgcaggtttgttacataggtatacatgtgccatggtggtttgctgcacccatcaacctgtcatctacatcaggtatttctcctaatgctaaccctccccttgtcccccaacccccctacaggccctggcatatgatgttcccctccctgtgcccatatgttctcattgttcaactcccacttataagtgagaacatgtggtgtttggttttctgttcctatgatagttttctgagaaagacagtttccagcttcatccacgtccctgcaaaggacatgaactcattctttattatagctgcataatattccatggtatatatgtgccacattttctttatccagtctattattgatgggcatttgggttggtttcaagtctttgctattgtgaatagtgctgcaataaacatatgtgtgcatgtgtctttatattagaatgatttataatcctttgggtatatacccagtaatgggatggctgggtcaaatggtatttctagttctagatccttgaggaattgccactctgtcttccacaatggttgaactattatttacactcccaccaacagtgtaaaagcattcctatttctccacattctctccagcatctgttgtttcttgactttttaatgatcgccattctaactggggtgagatagtatctcatttgtggttttgatttgcatttctctaatgaacagtgatgatgagcttttttttcatatgtttgttggctgcataaatgtcttcttttgagaagtgcctgttaatatccttcacccactttttgatggatatcaaaagtggatatccatctggatattagccctttctcagatggatagattgcaaaaattttctcccattctgtgggttgcctattcactttgatgatagtttcttttgctgtgcagaagctctttagtttaattagatcctatttatcaattttggcttttgttgccgttgcttttggtgttttagtcatgaagtctttgcccatgcctatgtcctgaatggtattgcctaggttttcttctaggttttttatggttttaggtcttaagtttaagcctttaatccatcttgagttaatttttgtataaggtgtaagtaaagggtccagtttcagttttctgcatatggcaaagccagatttcccaacaccatttattaaacagggaattctttccccatttcttgtttttgtcaagtttgtcaaagatcagatggttgtagatgtgtggtgttattttcgaggcctctgttctgttccattggtctatatatctgttttgttaccagtaccattctgttttggttactgtagccttgtagtatagtttgaagtcagctagtgtgatgcctcaagctttgttctttttgcttaggattgtcttggctatgcagcctcttttttggttccatataaaatttaaagaagttttttgtaattcggtgaagaaagtcaatggtagcttgatgggactagcattgaatctataaattacttcaggcagcatggccatttttatgatattgattcttcctatccatgagcatggaatgtttttccatttatttgtgtcctctcttatttccttgagcagtggtttgtagtattccttgaagaggtccttcacatcccttgtaagttggattcctaggtatttcattctctttgtagcaattgtgaataggagttcactcatgatttggctctctgtatattatttgtgtataggaatgcttgtggtttttgcacattgattttgtatcctgagactttgctgaaattgcttatcagcttaaggagattttgggctgagatgatggggttttctgaatatacaatcatgttatctgcaaacagagacaattttacttcctctcttcctatttgaataccctttattttgttctcttgcctgtttgccctgaccagaacttccaatactaggttgagtaggagtggtgagagagggcatccttgccttgtgctggttttcaaaaggaatgcttccagtttttgcccattcagtatgacattggctgtgggtttttcataaatagctcttattattttgagatactttccatcaataccttgtttattgagagtttttagcgtgaaggggtgttgaattttatcgaaggccttttctgcatctattgagataatcatgtggtttttgtcattagttctgtttatgtgatggattactgatttgcatatgttgaaccagccttgcatcccagggatgaagccgacttgatcatggtggataagctttttaatgtgctgctggatttggtttgacagtattttactgaggatttttgtatcaatgttcctcagggatatcggcctgaaattttctctttttgttgtgtctctgccaggttttggtatcaggatgatgctggcctcataaaatgatttagggaggagtccctcttttctattgtttggaatagtttcgtaaggaatggtaccagctcctctttgtacctctggtagaattcggctgtgaatcggtctggtcctgggctttttttggttggtaggttattaattactccctcaatttcagaaattgttattggtctattcacttctatgcatagaggtgtttatagtattctctgatggtagtttgtatttctgtgggatcagtggtgatattccctttatcattttttattctgtctatttgattcttctctcttttctgttttattagtctggctagcagtctacctattttgttaatcttttcaaaaagcctgctcctggattcattgatttttcgaagggtttttcatgttgctgtctccttcagttctgctctgatattagttatttcttgtcttctgctagcttttgaatttgtttgctcttgcttctctagttattttaattgtgatgttagggtgttgcttttagatctttctcactttctcctgtgggcatttagtgctataaatttccctctaaacactgctttagctgcatcccagagattctggcatgttgtgtctttgttctcattggtttcaaataacttatttatttctgccttcgttttgttatttatgcagtagtcattcaggagcaggttgttcagtttccatgtagttgcacctgtgttgagtgagtttcttaatcctgagttctaatttgattgcactgtggtctgagtaactgtttgttatgatttctgttcttttgcagttgctaaggagtgttttacttccaattatgtggtcagttttagaataagtgctatgtggtgctgagaagaatgtatattctgctgatttgaggtggagagttttgtagatgtgtattaggtccacttgatccagagctgagctcaagtcctgaatatcctttttaattttctgtctcattgatctgtctaatattgacagtggagtgttaaattctcccactattattgtatgcgagttgaagtcttttgtaggtctctaagaacttgctttatgaatctgggtgcttctgtattgggtgcatatatatttaggatagttagttcttcttgttgcattgatccctttatcattctgtaatgttcttctttgtatttttcttttaatctttgttggtttaaagtctgttttatcagagactaggattgcaacccctgcttttttttgcgttccatttccttggtaagtcttcctccatccctttattttgagcctatgtgtgtctttgcacatgagatgtgtctcctgaatacagcacactgatgggtcttgactctttatccaatgtgctagtctgtgtcttttaactggggcacttagtccatttacatgtaaatttaatatcgttggctgggtgcagtggctcacgcctgtaatcccagcactttgggaggcttaggtgggcgaatcacaaggtcaggagttcaagaccagtctggccaacatggtgaaaccccgtctctactaaaaataaagaaaattagctgggcgtagtggtgggtgcctataatcccagctactcaggaggctgaggcaggagaatcacttgaacctgggagacggaggttgcactgagccgagatgggtgccactgtactccagcccagtgatagagtgagaccgtgcctcaaaaaaaaaaaaaaaattgctatgtgtgaatttgatcctgtcattatgatgctagctggttattttgcccattagttgatgcagtattttcatagtgttgatggtctttacatttcagtatgtttttgcagtggctgataatggtttttcctttccatatttagtgcttccttcaggagttcttgtaaggcaggccggcttgtgacaaaaatccctcagcatttgcttgtctttaaaggattttatttctcctttgcttatgaagcttagtttgggtggatatgaaattctgggttgaaaattcttttctttaagaatgttgaatattggtccccactctcttctggcttctagggtttctgcagagagatctgctgttagtctgatgggcttccctttgtgggtaacccaacctttctctctggctgcccttaacatttgctccttcatttcaaccttggtgaatctgatgattatgtgtcttggggttgctcttctcagggagtatctttgtggtattctctgtatttcctgaatttgtatattggcctgtcttgctaggttggagaagttctcctggataatatcctgaagtgtgttttccaagttggttcctttctccctgtcattttgaggtacatcaatcaaatgtaggtttggtcttttcacatagtcccatatttcttggaggctttgttcattccttttaattcttttttctctaatcttgtcttcatgctttatttcattaagttcaccttcagtctttgatatcctttcttccgcttgatcgatttggctattcatacttgtgtaagcttcatgaagttcttgtgctgtgtttttcagctccatcaggtcatttattttcttctctaaactggttattctaattagcagttcctgtaaccttttatcaaggttcttagcttccttccattgggtttttggaattttcagcctttttgctctatttttaattcatcttcatggctttatctacctttggtctttggtgttggaggtctttggatggggtttttgcatgggcatcctttttgctgatgttgatgctattcctttctgttttttttccttctaatagtcagggccctcttctacaggtctgctggagtttgctggagttccactccagaccctgtttgtctgggtatcaccagcggaggctgcagaacaacaaagattgctgcctgttccttcctctggaagcttcgtcccagaggggcacccaccagatgccagccggagctctcctgtataaggtgtctcccagacaggatgcacgggggtcaaggacccacttgaggaggcagtctgtcccttagcagagctcgagcgctatgctgggagatccactgctctcttcagagccagcaggcaggagcatttaattctgctgaagctgcacccagagctgccccttcccccaggtgctctgtcccagggagatgggagttttatctataagcccctgactggggctgctgcctttctttcagagatggcctgcccagagaggaggaatctagagaggcagtgtagctacagtggcttcatggtgctgcggtgggttctgcacccagttcgaacttcctggaggctttgtttacagtgtgaggagaaaactgcctactcaagcctcaataatgaccagtgtcccaccccccaccaagctcaagtgtcccaggttgacttcagagtgctgtgctggcagcaagaatttcaagccagtgcatcttagcctgtcaggcttcattgggggtgggatccactgagcaagacctcttggctccctgacttcagccccctttccaggggagtgaatggttctgtctcgctggcattccatacaccactggggtatgaaaaaaactcctgcagctagctcggtgtctgcccaaatggctgcccagttttgtgcttgaaacccagggccctggtggtgtaggcacccgagggaatctcctggtctgtgggttgcgaaaaccatgggaaaagcatagtgtctgggccagatagcactgttcctcatggcacagtccctaacagcttcccttggctaggggaggaagttctctgaccccttgcacttcccaggtgaggtgatgcctcaccctgcttctgctcaccctccatgggctgcacccactgtctaaccagtcccaatgagacgaagtgggtacctcaattggaaatgcagaaatcacctgccttcttctttgatctcactgggagctgcagaccagagctcttcctagccatcttgccagccatctgttatggttcttaaaacattaatttgctattgtgtgttcagcttatctaaaaaataaagtatgggaatcagtgaattttgaggagaaggcaatgctcttatgtgtgtgtatatggtgtgtgtctgtatttgtgcctatggataatcattagtttctcaaatgtatgcaagtgatacagcacatatgtgtatttcccagtcttcatacgctcaacagaccagccttctttttccacctcggacatatgtttctgggtaggtttcctgatttgtaatcagtcagcctctgatattcactgtgactttggtttccagccctgtctggaagaactccagcctgggagaatgtaaacattttattcagaagcctgtgatgttggaaagaggaggttttggatgatttttgtttttgtttggacttgggtcagtgtctcccatttatagctggaatgtgaactgcatggcttcagatgtgttttggaagattgatgtgggcctcatgaaactcagctacagtggagcctcaggagactaggagctggttatcatattaagaaacacatttctttttatcagtgaatattaatgctgaacagtgaagctgtggaactccttctcttacaccggcaacctcagccctcctagaaaagctgtgtagtcagaaccagtgttagggggagttctttatggtttctccaacagtcgttgatactgagtaaaaagggactacccaggggatttcaagtcaaaatctaagaactaaacttatttctgttaatgatatgaatgacatgtcattggtgaatggaaagaaaatgctctcaatatatggagctataggaataaatgctagaagtgaggctaagtttttgccagctgtatcctactgatcatattggccattatgttagaattgccactttccctctccttggatgaaaagaggtcttattttatttttccaagaaggagtcttgctctgtcacccaggctggagtgcagtggcgcgatcttggctcaccgcaacctctgcctcctgggttcaagcgattctcctgcctcagcctcccaagtagctgggattacaggagtgtgccacctcgcccagctaatttttatatttttagtagagaaggagtttcaccctgttggccaggctggtctcaaattcctgaccttgtgatctgccagcctcggcctcccaaagtgctgggattacaggtgcgagccaccatgccctgccatgaaaagaaatgttataaaagtacaaaccacttgtgctttattttatgggtgagtctacttgtggtggatccacttgtctcataccattatgtctacttctttcctttttgattgctaagaaaattttccatcttttccaacgaaaatgcaggatctaaaacagaattgcaaagttattccaaaatcaaaccaaacaaacgaacaaactaatcattaacatttaaaaatcaggagatcgcacctaagaatctgcatttcttcagtctcctggaatgcaggtgcatgtggtagcactgagtctgcatttctgcatactggaactgggtagatggagctaccttttaatgtgacacgtgctcccagtcttattaaggcaggccagctttacccatttgtgtgagctgccctgtccttgtaggcatttgaatttttaacgtttggtctataggaaagaaaacttctgcaatatttttttttcttccttgagactctaagtgatgttttggaattggaaatgagctatggttagtgaatggaaatggtcagtggaaatatgtaattaaagggacttccctagagccacgatctaaatgtatttgggcaacaatggtgcttagtgttacccatgtcgaaaagactcatcagtctgaacttagttttcacagatacatgctcaggggaaacgtttcctcctgaagatgattcctcttggttttgactgcctggatcagttcataaagctggagttatggcttcttcaaaaaagcctgtctctcatgctgtggtaaaatatgtggtcacctggctggtgaagagtttctgggtcccataatattttacataggacacaaaaaatggaatgcttttggacttaaacctgttttcttttcaactccttctgcctagaggagatggtagcctcagaagagttcttacttccccttcctagaactgtgcaaatctttcaggtcccagctggtaatggagaaattactttttgttatttaaaaaatataaacaggaaaaataaaaaccatgatatttcatgaaatacaacttcttttctgttttctactaaataagacaaatttttttttatatgtgttcaggttcctgactgtctttagtactttaccttctcatggtctctactctcttgcaatttattttatatagtctttttccaggaatcctcccgtctcattagatttttctttttgttcctttccatgctgctgtattttccttttattttagaacctgtgtctaattttccagctctctacttttattcttattttccttttgtcagcctggtgtcttttattttctattgtttcattttttccctctctgtgccctttttcatgtgctattatacagggtttgcttatttttcacttctgttcagtttgttttattctcttaccatgcattcatacctcctgcttcaaaaccaaaaaacccgaaactgattcttacttttttaattgtaaccctctccctagtttatttacactaactttacactatgagttttttatacttgaaattgccaaaatttccttatcacttagttttctcccagtttcccatagaaaattcagtggatgttctttccatatgtcagctgctgtggtaacccaagacagaggtcattatgttcagttctgatcacattttgttctttcgctggctgtattacctgaaggaagccacagtctctgtctgggtctcatttttttttatctagtaaagttaacagggagaacaaggtgtggttctcagatgaggcacacctgtgagctcgccagaactgtgctacctcaggtctcactccacacttcctgacgtggtatctgcatttttacaagatctcccgctgatctgtgccctcatggaagtttgagaagcacaatctcgatgctctttgggggtttttccaggtttaactttgctgaaaataagggaaaacctattttactaagtgaaaataagtgaaatattttaataagtgaaaataagggaaaaacataaagtgaaaactgctttggttacaaggcacagtagaataacctgggaagcatcaataaaatgtcaatgccttagaggtccctcctcagggcagctgattcagaaactctggggatggtattttttactgtaccaggtgattttaatgagcaggttgatccccacttaaatatgctatttaactttgacctcctttactgatgccctcagtgctgccctcttttggttgtgaggtaagtaattactatggaaagaggaacttcgaagtatttggtttcatctacttaaacccaccagaatttattctggggaggccataactggggagatagataactatcgaaggaggctatatgtatttttatagcgttatgaaagcaaaagcaggattcgcttacttttttttttttctcaagactgattaaggcttttgctggcaatgggagtgtttgtggtaacagaaggaagtaaccctggcaagtgatagatagacattgtatcctaagagtcttgggggtggtgggtgccggggatatgtcagtgggatggttttatttcatcagaacttggtgctggtgttgactggatactcacaattagccacatgctatgattgggttactgaacattaggttctaatttatttttaattatttcaggttgttgaactgtagctgatttaagaaaacttgaaaccaagatttttttttttaactgctcttatagtattcttagtgtgagaactggggagggaatagtcataaaacaatttgtcacaagagtggagggtgtgacaatactattaggtggtgatactttaatgttgtagttttaaaatcggatcttgagaaactgtgtctttaaagattttttctttttttaagagagagagtttcgctcttgtcgcccaggctggagtgcaatggtgtgatctcggctcactgcaacttctgcctcccggcttcaaacgattctcctgcctcagcctcccgagtacctaggattacgggcagcctgctaccatgcccagctaatttttgtatttttagtagagaaaggtttcatcatattggctaggctggtctcaaactcctgatctcaggtgatccacccgcctcagcctcccaaacggctgggattacaggcaagagccaccacacctggcctttaaagggctttttaaaaaaaggttaaatgtttttcaccagtgctcaatgagaaacacaaacatgattacattgtttggagtggctgaattacttgatcaaaatttaaaaaaaaaggtattttccctaagtggtttttgcttagtacagcacctttttttcccctcagttttcatgtcctgaattatttctgcatcattaagtttagctgaggagccaaaattctgtcattgtggggggattgctgaggtgtcacatcgacaaagggaggtacttagaataagatgtcattttggtctgttcttgagtgttactccaagaagttatgtgagcagagaaacactaatcctgtgggccatctgtctttcccatcatgtggcagaacccccatgcagaatctctagtcaaggttggaccatggctgcatttcattacatctgaaaagagtccccggtagttatgtgatttttacctcacagattgctcagaatcacataaagcctcaacaaatatttcctcacagatttattctagccctggtcaaaagtgtgaagtaggacattttcttcaagtgtatttttttttctgtcttattgaaacactttcaatactgttagcgtgactttgtgtcccatccgtccaatatttgctgtactcacagctgttacctctgcaggaaatctttgcaaactagagtcttcattctttcatggttctaggcagtaagcttagaagggacagtggagctgtaacactcactgtttcataagtttttaaacttatgctgtaaaagtctctgatacatgcaagatcagagaaaatgtagaacaaaggaggcatatagatgaggtgaggatactgggaagataataaatttgcacagaagggaagggagtttataaaatgtgggaagagtcaggaaaggctggcaggaggtgaaccggggtgtaagaaggatgtgtacaatatgtaggtagagaagatatgtcatgaggcctcgcaatgccctcattaccattatggttaggtagttgccaacacatgttttcggagcacctactgtgtgccaaacattattctggtgctcagggtatggcagtaaatgagcctagcagaatttctgtcttcaaggagctaacattctattctagtaaaaaagttctcttgcccttgctaagtttgaaatgcctgtttatggcaaaggagtagaaatggtagatgctttatataaaaatatatgatttgtcttggcaaagcatccttgaggagagattagtacagtgcaaaaaagtattgagtaacattctccccgtttccacctttctgtcaggtcatagatataaattacaattctggccctcattcaccctcacatagctttctggcatcctttcccattctctgaacatgagaggaatgagaaggttatttggaaggtcgctgatcttaggagttgtcctgctacagttaccggagaatcttgaatttcatctgaaaagactgcaaggcagtcggggcaagcatgctatgagaagtcaactggtaagcagaccccagagatgagaagcatatgggaagccaaccccatgatttgcttccttaaaaagtatgggctgtggatggattttaaatcctactctaaatacaagactggtgttagcggttgtttgcaggaaaaccaaaggaacttagcatccttcagtgtgttacagagactgtgtggcctcaagcagtgaagtgaagaattctttttgtctttttcctcatatgcccaacctggaaaccagcagaatttttgatggtgcatctggttcctggtgtgctgcagaagtgattagtgtgtcatgtcactgtggcctctttctctgccccaggagaatgtctggggaacagaagagcagagtcctgcctgggatcatgtgggtgggggagctattccttctcaacagatagtgtgagaagcactgatccaaggtcctggctgctcaaagtgtgttcctaggagcttgctggacatgcagaatctcaagttccatcccagaacctgatgtttttatttggagcactggccttggcaacacatgttccttcctcacctttctgcagcctctcactccccactgtcgcccactcctcataggctatcctcaggaatggcattggtggagtgttggtgaaggtgtagctgcaagcattctgatctttattttacaaactctaaacccagaagactccaaacccagaaaagtctaagtcacagaaagcaaaggaacagtcttatttcagacacatctacagtctttgctggttaaatgtcaggaagcccacaaaaagctgccagacatttcagggtatgaatttcgtatctgtacttcttgacatctgctgggctaccatgcccttcaatttaccaaaatatggaaatttccttatggcattctacccaaataatgaggaaaatgttgatgcttctctcagcctcaggaccttaaagccacactgttcctaaccacgcttgcctatatggcacttagcactttacacaaaaaacaaacagaggtgtttattccctggcagttaatataaatgctttctgttttgtaaaataggaacagtatttgattccattatcacagatctcttgtggacaagtcaaaattcttcaattaaggttatatctagttaaaggaatttttctctttggtctacatccccaccctgaatgtccttgcctgttagttccttgcaggtgaaaggtaaaaaacctttgcaacttcatgctataattgtgcctttgaattttcagaggaaaagacagtgagagagcaagagcagattgagaagcagaagagtgagtcgcaaaaagagaaaatgacagaaatggagcaccctg

SEQ ID NO: 76 - DNA, 103 bp, Homo Sapiens
gtagatggaggcaggtgcatgtgtgatgggaagtgtggtgacgggttgtgtgggcacacgggatgaggcgcagatggctgggggtttgggaggtgaatggatg

SEQ ID NO: 77 - DNA, 2584 bp, Homo Sapiens
ctcactccatcactcgatgagataatatgagatttctacttcggagaggccaagtctaatgaagagaaaaaaaggaaaagaagttgcaagactcgaataaaatctgctgcaccttgtaaatgctctaactggacatgaaggaaaggggcgagggaggggggtgggatttttggtgcaagtagcacatggtttaaatatgaatgaacaaacctgtgatctagtccttgtcttgtaattgtggattaatgtcaatgttaatcagcccctcaaagggagagaaaagctggaccttttcccttgctgtaccatattcagcatttgatttccatgggccccaccatttatgtgtagaatttgaaatggttgtcacctctctctgaggacagagcttgaagcctccacaccagctgctgctggagattcaaagcccaactgtgggtccgagagggaagctggctgggctggctgaagaatgaagaccactggactctccgttaatctctaaggggtctgctccccaggaacgtttctgaacaatggggactttgttggtagccatttggtagatgttcttttctatttataagtgactttaaactttcccttggctgttaagaagtttgttatagatttagctatttattgttcgatgcctgcatgctgaaacaatgcctacagctgtcttcacatgtatggacgtgtgtgaatggttgtacgttttgcacattttgtggctgttgagatgtgctttgctgcacaaacatgaaaatttttgagttacaatttggagcataactggagggtgggttggggaggggtggatttttaaaatgtcaagacagggaaggatgacaaaatggaaatttaaatgacatcctagaggtagagaaaccgtggagatcgcttttctcagactcaccaacttttaatgggatttcatggggtttggttgtgctgatagggtaaggggaggctgctttctgcccttctccccactcccatctgatttacttaattcagtctcagctgctgaaatttggaaaggaccaaattgctttacagtttttttctttgtgtagtatcttgaaatcctggaaaattctatggaatagttctgtatatagggcacaagtaaaggcattgtccaaagtttatttatttatttattaccctaagaatgctttgccataaccacatttaatgggaaaaacggcagtatcacagatgtaaattaactcaccagatttactgggcctgaactcattctcttcttgctatatgatttagcaagttctagaaggtctccaagacaataattacattggcacaatgtatacttcagtgctcacccgtaggcaaatctctttttaaaaaactctttggtgcacaagtaacacatttggccacaaaacaccaaagaattgtaggcagtggcccctattgagaagttttccggtagagttggaaatcagttgtgaatacattctttgctagttggagtgcttgtttactaagcatgtgccgtcgtaggtattagtgctagtctcagataggtgcttcccctgaggtgcaggggaagaccaaagtttgcaactcgaactgctttcgtccatgtttctcacattgctgtattttagaaaataggggttaagactgataacaaccttttacattgtgactgtgtttgcattgtctaatgacagataaatccttaacatttctctccaccttagtactttagactaattgtgtttgtccgtccatgccatgaatgagtgggctgtagttgggcctaaataaatgagctgttggaagaaaagaatcacagtactttccagcagtcagtccctggttcctagatgtgttctaagcaatgcaaatgtctaattgtcccccagtgggcatagtcagtgtcgtttatattgtagcagttacagctctgtagtttatgatgcaaatctgccaagagagatgtatgtgtcactgcatggcttctgaaagcaggatgaattttctgcagctgtttcaaagttggggtctgttcttgaatcctctattaattactgtgtgtgagccagagggagctgtggtaagggttgggcccccagcctgtagggaactttctggactcccactctttgaatcgatataggcatttggtctcactacttgaccattctcaccctgtgaaacgtcccacactttgaagcaaatacaattcacagcacagtacacacaaaaaccttggcataagacagagaaggttcttcttattttgtgggctggttgctgtagaaacacataacaaagggcagccctccacttctggtataattgtgtagccccttttctttgggcttgacacctgtcttgaataagagtgattagagctgcataatgtccctctcttggctattgaccatgtggttcacgtacaaaactctgtataagttgaaggaaaatgttcatgttcatatgtacttgtttgctatgactacattttgaggttttgtaaaactgttatttttttttttttcacaatgtgaaactgaaggtcaataaattattagagattttctcttca

SEQ ID NO: 78 - DNA, 693 bp, Homo Sapiens
gaacttggcattagaattcctcgaccactaggacagggaccaagcagattcatcccagaaaaggagatcctccaagtggggagtgaagacgcacagatgcatgctttatttgcagattcttttgctgctttgggccgtttggataacattacgttagtgatggttttccacccacaatatttagaaagtttcttaaaaactcagcactatctactgcaaatggatgggccgttacccctacattatcgtcactacattggaataatggctgcggcaagacatcagtgctcctacttagtgaacctgcatgtaaatgatttccttcatgttggtggggaccccaagtggctcaatggtttagagaatgctcctcaaaaactacagaatttaggagaacttaacaaagtgttagcccatagaccttggcttattaccaaagaacacattgagggacttttaaaagctgaagagcacagctggtcccttgcggaattggtacatgcagtagttttactcacacactatcattctcttgcctcattcacattcggctgtggaatcagtccagaaattcattgtgatggtggccacacattcagacctccttctgttagcaactactgcatctgtgacattacaaatggcaatcacagtgtggatgagatgccggtcaactcagcagaaaatgtttct

SEQ ID NO: 79 - DNA, 856 bp, Homo Sapiens
gcctaatttgtagatatcctcaagaagattatgagtcattctcactaccggaatctgttcctctattttgtttaccaatgggtgcaaccattgaatgttggccatcaaatagcaaataccctctgcctgtattttctacttttgttttaactggagcctcagctgaaaaggtttatggtgctgctattcagttttatgaaccatactctgaggagaatctcacagaaaagcagagacttcttttgggtttaacatcagcagatgggaagtctgatagttccaaaacaattcatactaacaaatgcatctgtcttctttctcactggcctttttttgatgcattcaggaagtttctgacttttctgtatcgttattccatctctgggcctcatgtccttccaattgagaagcatatttctcattttatgcataaagttccttttccatctcctcagagaccacggattttagttcagttatcaccacatgataatctgattctcagtcagcctgtgtcttcacctcttccactaagtggtggcaagttttcaacactactgcagaatttaggccctgaaaatgctgtgacactactggtgtttgcagtaacagaacataaaattcttatccattccctacggccatccgtgcttactagtgtgacagaagcattagtgtctatgattttccctttccactggccatgcccgtatgttcctctctgcccactggctttagcagatgtcttgagtgcaccatgtccattcatagtagggattgattcaagatactttgatctctatgatcctccaccagatgtcagttgtgttgatgtagataccaacactatttctca

SEQ ID NO: 80 - DNA, 213 bp, Homo Sapiens
atcacaatcgaagcaatgaaaagtgacattgatgaggtggctttacaagggatagaattctggtccaatgtctgtgatgaggaaatggatttggccattgaagcttcagaggcagcagaacaaggacggccccctgagcacaccagcaagttttatgcgaagggagcactacagtatctggttccaatcctcacacagacactaactaaacag

SEQ ID NO: 81 - DNA, 1480 bp, Homo Sapiens
agaaatggtcaatgcatggtttgctgagagagttcacaccatccctgtgtgcaaggaaggtatcagaggccacaccgaatcttgctcttgtcccttgcagcagagtcctcgtgcagataacagtgcccctggaacaccaaccaggaaaatctctgcctctgaatttgaccggcctcttagacccattgttgtcaaggattctgagggaactgtgagcttcctctctgactcagaaaagaaggaacagatgcctctaacccctccaaggtttgatcatgatgaaggggaccagtgctcaagactcttggaattagtgaaggatatttctagtcatttggatgtcacagccttatgtcacaaaattttcttgcatatccatggactgatatctgctgaccgctattccctgttccttgtctgtgaagacagctccaatgacaagtttcttatcagccgcctctttgatgttgctgaaggttcaacactggaagaagtttcaaataactgtatccgcttagaatggaacaaaggcattgtgggacatgtggcagcgcttggtgagcccttgaacatcaaagatgcatatgaggatcctcggttcaatgcagaagttgaccaaattacaggctacaagacacaaagcattctttgtatgccaattaagaatcatagggaagaggttgttggtgtagcccaggccatcaacaagaaatcaggaaacggtgggacatttactgaaaaagatgaaaaggactttgctgcttatttggcattttgtggtattgttcttcataatgctcagctctatgagacttcactgctggagaacaagagaaatcaggtgctgcttgaccttgctagtttaatttttgaagaacaacaatcattagaagtaattttgaagaaaatagctgccactattatctctttcatgcaagtgcagaaatgcaccattttcatagtggatgaagattgctccgattctttttctagtgtgtttcacatggagtgtgaggaattagaaaaatcatctgatacattaacaagggaacatgatgcaaacaaaatcaattacatgtatgctcagtatgtcaaaaatactatggaaccacttaatatcccagatgtcagtaaggataaaagatttccctggacaactgaaaatacaggaaatgtaaaccagcagtgcattagaagtttgctttgtacacctataaaaaatggaaagaagaataaagttataggggtttgccaacttgttaataagatggaggagaatactggcaaggttaagcctttcaaccgaaatgacgaacagtttctggaagcttttgtcatcttttgtggcttggggatccagaacacgcagatgtatgaagcagtggagagagccatggccaagcaaatggtcacattggaggttctgtcgtatcatgcttcagcagcagaggaagaaacaagagagctacagtcgttagcg

SEQ ID NO: 82 - DNA, 482 bp, Homo Sapiens
cccaattttgacatgctttataaccatgtgaagcaactcctctccaatgagctactcctgacacaaatggagaagtgtgccctcatggaagccctggttctcattagcaaccaatttaagaactacgagcgtcagaaggtgttcctagaggagctgatggcaccagtggccagcatctggctttctcaagacatgcacagagtgctgtcagatgttgatgctttcattgcgtatgtgggtacagatcagaagagctgtgacccaggcctggaggatccgtgtggcttaaaccgtgcacgaatgagcttttgtgtatacagcattctgggtgtggtgaaacgaacttgctggcccactgacctagaagaggccaaagctgggggatttgtggtgggttatacatccagtggaaatccaatcttccgtaacccctgcacagagcagattctgaaacttcttgacaatttgcttgcgcttataag

SEQ ID NO: 83 - DNA, 1031 bp, Homo Sapiens
ataagcaacgatgtgctgagctgaagaaagccttgtgtactgcccatgagaagttctgtttctggccagacagcccatccccagaccgatttgggatgttgcccctggatgagcctgctattcttgttagtgaattcctagatcgttttcaaagcctttgtcacttggacctccagcttccttccctaaggccggaggacttgaaaactatgtgcttgacagaagacaagatcagtcttctcctacacttgcttgaagatgaacttgatcaccgaactgatgagagaaaaactacaatcaaattaggctcagacatccaagtccacgtcactgcctgtattctctctgtgtgtggctgggcgtgtagttcctctttggaatccatgcagctctccctgataacatgttcgcaatgtatgaggaaggtggggctctggggcttccagcagattgaatcgtccatgactgacctggatgcatcctttggcctgaccagctccccaatcccaggccttgaggggcgaccagagcgcttacctctggtgcctgaatctcctcggaggatgatgacccggagccaggatgccactttctccccaggctcagagcaggctgaaaagagccctggtcccattgtctctcgaactcggagctgggactcttccagtcctgttgaccgtcctgagccagaggctgctagccccaccaccagaactcgcccagtgacccgaagcatgggaacaggagacacccctggcctggaggtaccatctagccctctgcggaaagccaagcgagctcgcctctgctcctccagcagttcggacacatcttcccgaagcttctttgatcccacctctcagcatagagactggtgcccttgggtgaatatcacacttggcaaagaaagcagggagaatggtggaactgaaccagatgccagcgccccagcagagccaggctggaaagcagtgctgaccatcctcttggcgcacaaacagtctagccagccagctgaaacggactccatg

SEQ ID NO: 84 - DNA, 186 bp, Homo Sapiens
attgaacaggagggtgtcacagtgaagagcagctcccacttcaacccagaccctgatgcagagaccctctacaaagccatgaaggggatcgggaccaacgagcaggctatcatcgatgtgctcaccaagagaagcaacacgcagcggcagcagatcgccaagtccttcaaggctcagttcggcaag

SEQ ID NO: 85 - DNA, 674 bp, Homo Sapiens
gaatgtatcctgtcaggtataatgtcagtgaatggcaagaaagttcttcatatggatcgaaacccttactacggaggagagagtgcatctataacaccattggaagatttatacaaaagatttaaaataccaggatcaccacccgagtcaatggggagaggaagagactggaatgttgacttgattcccaagttccttatggctaatggtcagctggttaagatgctgctttatacagaggtaactcgctatctggattttaaagtgactgaagggagctttgtctataagggtggaaaaatctacaaggttccttccactgaagcagaagccctggcatctagcctaatgggattgtttgaaaaacgtcgcttcaggaaattcctagtgtatgttgccaacttcgatgaaaaagatccaagaacttttgaaggcattgatcctaagaagaccacaatgcgagatgtgtataagaaatttgatttgggtcaagacgttatagattttactggtcatgctcttgcactttacagaactgatgattacttagatcaaccgtgttatgaaaccattaatagaattaaactttacagtgaatctttggcaagatatggcaaaagcccatacctttatccactctatggccttggagaactgccccaaggatttgcaag

SEQ ID NO: 86 - DNA, 903 bp, Homo Sapiens
gaggtatatcgaattccaaagaaaagtcaaactgaaaaggaaaacacaacaactgaacgaggaagggatgctgttggcttcagagatcaaacacctgccccgaagactcctaataggtcaagagagagagacccagacaagcaaactcaaaataaagagaaaaggaaacgaagaagctccctctcaccaccctcttctgcctatgagcggggaacaaaaaggccagatgacagatatgatacaccaacttctaaaaagaaagtacgaattaaagaccgcaataaactttctacagaggaacgccggaagttgtttgagcaagaggtggctcaacgggaggctcagaaacaacagcaacagatgcagaacctgggaatgacatcaccactgccctatgactctcttggttataatgccccgcatcatccctttgctggttacccaccaggttatcccatgcaggcctatgtggatcccagcaaccctaatgctggaaaggtgctcctgcccacacccagcatggacccagtgtgttctcctgctccttatgatcatgctcagcccttggtgggacattctacagaacccctttctgcccctccaccagtaccagtggtgccacatgtggcagctcctgtggaagtttccagttcccagtatgtggcccagagtgatggtgtagtacaccaagactccagcgttgctgtcttgccagtgccggcccccggcccagttcagggacagaattatagtgtttgggattcaaaccaacagtctgtcagtgtacagcagcagtactctcctgcacagtctcaagcaaccatatattatcaaggacagacatgtccaacagtctatggtgtgacatcaccttattcacagacaactccaccaattgtacag

SEQ ID NO: 87 - DNA, 432 bp, Homo Sapiens
gtcttctccaagatcttcagccacgaggccctggagagttacctgcccaagatccagctggtgatccaggacacactgcgcgcctggagcagccaccccgaggccatcaacgtgtaccaggaggcgcagaagctgaccttccgcatggccatccgggtgctgctgggcttcagcatccctgaggaggaccttgggcacctctttgaggtctaccagcagtttgtggacaatgtcttctccctgcctgtcgacctgcccttcagtggctaccggcggggcattcaggctcggcagatcctgcagaaggggctggagaaggccatccgggagaagctgcagtgcacacagggcaaggactacttggacgccctggacctcctcattgagagcagcaaggagcacgggaaggagatgaccatgcaggagctgaag

SEQ ID NO: 88 - DNA, 1736 bp, Homo Sapiens
gcataaatgagacatttttgagagttggtaagagttacaaggaactaaaaggaagcagatagtttaacataaaactgtcttataatgagtgctgcccacaatgaaaaaggatgctgtccatgaaagagagtttcccgtcatcagaaatgttcaagacttaaagccacttgctggggatgaggtaaagggggttagataaacgctaaggccctttttggttgtgggggtatctaacaatttggtctgtagctgcctgcttagtctgagtgaacttgtatatctaatttttcagtggttgaaggcctttcactgttggacttgggagtatctccgtattctggagcagtatttcatgaaactccattaataatatacctctttcatttcctaattgactatgctgaattggtgtttatgataactgatgcactcactgctattgccctgtattttgcaatccaggacttcaataaagttgtgtttaaaaagcagaaactcctcctagaactggaccagtatgccccagatgtggccgaactcatccggacccctatggaaatgcgttacatccctttgaaagtggccctgttctatctcttaaatccttacacgattttgtcttgtgttgccaagtctacctgtgccatcaacaacaccctcattgctttcttcattttgactacgataaaaggcagtgctttcctcagtgctatttttcttgccttagcgacataccagtctctgtacccactcaccttgtttgtcccaggactcctctatctcctccagcggcagtacatacctgtgaaaatgaagagcaaagccttctggatcttttcttgggagtatgccatgatgtatgtgggaagcctagtggtaatcatttgcctctccttcttccttctcagctcttgggatttcatccccgcagtctatggctttatactttctgttccagatctcactccaaacattggtcttttctggtacttctttgcagagatgtttgagcacttcagcctcttctttgtatgtgtgtttcagatcaacgtcttcttctacaccatccccttagccataaagctaaaggagcaccccatcttcttcatgtttatccagatcgctgtcatcgccatctttaagtcctacccgacagtgggggacgtggcgctctacatggccttcttccccgtgtggaaccatctctacagattcctgagaaacatctttgtcctcacctgcatcatcatcgtctgttccctgctcttccctgtcctgtggcacctctggatttatgcaggaagtgccaactctaatttcttttatgccatcacactgaccttcaacgttgggcagatcctgctcatctctgattacttctatgccttcctgcggcgggagtactacctcacacatggcctctacttgaccgccaaggatggcacagaggccatgctcgtgctcaagtaggcctggctggcacagggctgcatggacctcagggggctgtggggccagaagctgggccaagccctccagccagagttgccagcaggcgagtgcttgggcagaagaggttcgagtccagggtcacaagtctctggtaccaaaagggacccatggctgactgacagcaaggcctatggggaagaactgggagctccccaacttggacccccaccttgtggctctgcacaccaaggagccccctcccagacaggaaggagaagaggcag

SEQ ID NO: 89 - DNA, 404 bp, Homo Sapiens
gttcaagagctgcaggattccctcttgaggctggagccctgcccacatctttcccacaaccaagcaggtggctcaggcagtggttctagcagctctgaggcagacagggaaccctgggagactcaggactccttctccctggctcaccccctgcttcggcgcctccgcagccattccagcacccagatccttgggtctcttcccaaccagcccctcagtcctgagatgcacatcatggaagcccagatggagcaactccgggggagcattgagaagctcaaatgctttaatcgtctgctatcagctgtgctacagggatacaagggccgctgtgaaggcctcagcatgcagctaggccagcgggaggctgaggccacagcattgcatctggccttgcagtacag

SEQ ID NO: 90 - DNA, 478 bp, Homo Sapiens
gccagaagcggactcctagaagaggggagcagcaaggatggaatgacagccgtgggccggaggggatgctcgaaagagctgagcggagatcctacagggaataccgaccctatgagacagagaggcaggcagacttcacagctgagaagtttccagatgaaaaaccaggtgataggtttgatcgagacagaccgttgagaggacgtggaggcccgagagggggtatgcgcggcagaggcagaggtggccctgggaacagagtttttgacgcttttgaccagagaggaaagcgagaatttgaaagatatggtgggaatgacaaaatagcagtcagaactgaagacaacatgggtggatgtggagttcgaacctggggatcgggtaaagataccagtgatgtggagccaactgcaccgatggaggaacccacagtggtggaggagtcccagggcaccccggaagaggagtctccagccaa

SEQ ID NO: 91 - DNA, 328 bp, Homo Sapiens
gtgaaaaaagccaagatgcaggaatcaggagagcaaactataagtcaagtaagcaatccagatgtcagtgatcagaagcctgaaacatcaagccttgcttcaaaccttcccatgtcagaggaaattatgacatgcaccgattacatccctcgctcatccaatgattatacctcacaaatgtattctgcaaaaccttatgcacatattctctcagttcctgtttcggaaactgcttaccctggacagactcaataccagacactacagcagactcaaccctatgctgtctaccctcaggcaacccaaacgtatggactacctccttttg

SEQ ID NO: 92 - DNA, 4225 bp, Homo Sapiens
cccctcttcctcgccctctctcgcgggtcggggttacatggcggcgactgcggcaaagcgagagcctcggagacgccgctgccgccagcacagccggagacctgagccgacactgggggcagtccgcgagccccgcactctctcgatgagtcggagaagtcccgttgtatcagagtaagatggacggtagctttgattgtgattgtggtgagctggagccacctgatcactaacaaaagacatcttctgttaaccaacagccgccagggcttcctgttgaaataaatatatagcaacaaaggaaaaaaagaagcaaaacggaaatagtgcttaccagcaccttagaatgatgctgctcaggaccagtccaacactgaatgtatctgcactgtgaggagaatgttcatagaagcctgttgtgtgcatatttattcacatttttgttaaatgttaaatcgtttagcacggtaatctgagtgcacagtatgtcatttcattccgtttgagtttcttgttttcgttaaatgtctgcagagttgctgcccctttcttgaactatgagtactgcaatctttttaattctcaatatgaatagagctttttgagctttaaatctaaggggaactcgacaggcctgtttggcatatgcaatgaacatcaagaaaccatcttgctgtggaagcataattatttttcttctccctttttgaaagatctttccttttgatgccagttttcttccttgtttacacaagttcaatttgaaaggaaaaggcaatagtaagggtttcaaaatggcagagaaatttgaaagtctcatgaacattcatggttttgatctgggttctaggtatatggacttaaaaccattgggttgtggaggcaatggcttggttttttctgctgtagacaatgactgtgacaaaagagtagccatcaagaaaattgtccttactgatccccagagtgtcaaacatgctctacgtgaaatcaaaattattagaagacttgaccatgataacattgtgaaagtgtttgagattcttggtcccagtggaagccaattaacagacgatgtgggctctcttacggaactgaacagtgtttacattgttcaggagtacatggagacagacttggctaatgtgctggagcagggccctttactggaagagcatgccaggcttttcatgtatcagctgctacgggggctcaagtatattcactctgcaaatgtactgcacagagatctcaaaccagctaatcttttcattaatacggaagacttggtgctgaagataggtgactttggtcttgcacggatcatggatcctcattattcccataagggtcatctttctgaaggattggttactaaatggtacagatctccacgtcttttactttctcctaataattatactaaagccattgacatgtgggctgcaggctgcatctttgctgaaatgctgactggtaaaaccctttttgcaggtgcacatgaacttgaacagatgcagctgattttagaatctattcctgttgtacatgaggaagatcgtcaggagcttctcagcgtaattccagtttacattagaaatgacatgactgagccacacaaacctttaactcagctgcttccaggaattagtcgagaagcactggatttcctggaacaaattttgacatttagccccatggatcggttaacagcagaagaagcactctcccatccttacatgagcatatattcttttccaatggatgagccaatttcaagccatccttttcatattgaagatgaagttgatgatattttgcttatggatgaaactcacagtcacatttataactgggaaaggtatcatgattgtcagttttcagagcatgattggcctgtacataacaactttgatattgatgaagttcagcttgatccaagagctctgtccgatgtcactgatgaagaagaagtacaagttgatccccgaaaatatttggatggagatcgggaaaagtatctggaggatcctgcttttgataccaattactctactgagccttgttggcaatactcagatcatcatgaaaacaaatattgtgatctggagtgtagccatacttgtaactacaaaacgaggtcatcatcatatttagataacttagtttggagagagagtgaagttaaccattactatgaacccaagcttattatagatctttccaattggaaagaacaaagcaaagaaaaatctgataagaaaggcaaatcaaaatgtgaaaggaatggattggttaaagcccagatagcgctagaggaagcatcacagcaactggctggaaaagaaagggaaaagaatcagggatttgattttgattcctttattgcaggaactattcagcttagttcccagcatgagcctactgatgttgttgataaattaaatgacttgaatagctcagtgtcccaactagaattgaaaagtttgatatcaaagtcagtaagccaagaaaaacaggaaaaaggaatggcaaatctggctcaattagaagccttgtaccagtcttcttgggacagccagtttgtgagtggtggggaggactgttttttcataaatcagttttgtgaggtaaggaaggatgaacaagttgagaaggaaaacacttacactagttacttggacaagttctttagcaggaaagaagatactgaaatgctagaaactgagccagtagaggatgggaagcttggggagagaggacatgaggaaggatttctgaacaacagtggggagttcctctttaacaagcagctcgagtccataggcatcccacagtttcacagtccagttgggtcaccacttaagtcaatacaggccacattaacaccttctgctatgaaatcttcccctcaaattcctcatcaaacatacagcagcattctgaaacatctgaactaaaacactcagcagacatttatctttgtattcttcatgaaatgtgttttgtctttttttattactagtgtttaagtcattttttacttgaatcagatggtgtcatttagtaaggattttatgagttcttgttttttaaaatccagactttctttttctacatgtgagatagttttcattttaactggcatgtcatttgcacacaaaaataaagactagagcaaaataatgcaacgcaggaggagaaaagaaatgcactaagacaagaacattctctcatagaacattgatctgttttacaggaaacaaaccttgccttgaaatttacacagtgagactgtacataattgcatgaaaatagctatttttttcctaagacatttttcattcatgaatattttcaagtttttcatactgtacacatttcttaaaacacatgataccagcagcaactgaaaatgaatgccgaatttggtacacatgtgttatctacctcaaggtaacaagagtatgtggcaaaacatataccacccatagtgcttcacaaaatgcacttctatttagccagcgtttattgtagtaaactattcttaataaaactcactcactgtttataaatgttctggtatgcattctttatagtgaagtgttaatacatcacatcttatttattttagcaaatcagtatattttctgtatttaattataaaaaattaacttagtttttaaaatttatttgcaaatatactttactttttccatttggcactatggtttgttgcctacctagctgcatctataatgtcagcttatcctaaggctgtccacgtacttaatttacttaagtgttcattttaagtaacgtgctcactgtgtataggaatttgtattttggaggtgcttgatctatctacaaagaaaaattaattaggaattactttattataaaatgctcctagaagtcttaattgtgtttattttttaaaaaaacaaatgttagacttgtgtgcatggaagtaattaaggtacatcattattgtagtttgaaagttgtacatgataagacattttgtttttactgtatgtttttactgaatgatctattccccatcccaaggcaagcatgaataaaattaggttaaacgtagcatgtggcatcgcagtctcttagaatttgtttcatctattttattttattgaatactgtctgtatctttggttatcctgtttgaagaaaaaggacaaataaaacatggccagcaaataca

SEQ ID NO: 93 - DNA, 195 bp, Homo Sapiens
gggcaatgttaaatattgttcaagattcagcacttttggaagccattggttgccagatggagatgggtggtggagaaaataacctgaagagtcatagtcgcaccaatagtggtattagttcagcaagtggtggaagcacggagcccacgactcccgatagtgagagacctgctcaagctctcttaagagattatg

SEQ ID NO: 94 - DNA, 656 bp, Homo Sapiens
cctgctcaggaagaactcagagaagatccagattttgaaattaaggaaggagaaggacttgaagagtgctccaaaatagaagtaaaagaagaaagccctcaatcaaaagcagaaacagaattaaaagcttcccaaaaaccaatcagaaaacacaggaacatgcactgcaaggtttctttgttggatgacacagtttatgaatgtgttgtggagaaacatgctaagggacaagatttgcttaaacgagtatgtgagcatctcaatcttttggaagaagactattttggtctagccatttgggataacgcaacctctaagacatggctggattccgccaaagaaataaaaaagcaggttcgtggtgtcccttggaattttacatttaatgtaaagttttatccacctgacccagcacagttaacagaagacataacaagatattatttatgtcttcagcttcggcaggacatagttgcaggacgtctgccctgttcctttgcaaccttagcattattaggttcttacaccatccagtctgaactgggagactacgacccagaactccatggcgtggattatgttagtgattttaaactggccccgaatcagaccaaggaacttgaagagaaggtcatggaactgcataagtcatacag

SEQ ID NO: 95 - DNA, 1500 bp, Homo Sapiens
gccgcgcggggccatttccgctgctgcttctgtgagtttttccggtgcacgcgagtgcttctgaaacgtcagctgcgctcccctaggagtgctgagcccgcggaaccgcagccatgactgaggctgatgtgaatccaaaggcctatccccttgccgatgcccacctcaccaagaagctactggacctcgttcagcagtcatgtaactataagcagcttcggaaaggagccaatgaggccaccaaaaccctcaacaggggcatctctgagttcatcgtgatggctgcagacgccgagccactggagatcattctgcacctgccgctgctgtgtgaagacaagaatgtgccctacgtgtttgtgcgctccaagcaggccctggggagagcctgtggggtctccaggcctgtcatcgcctgttctgtcaccatcaaagaaggctcgcagctgaaacagcagatccaatccattcagcagtccattgaaaggctcttagtctaaacctgtggcctctgccacgtgctccctgccagcttcccccctgaggttgtgtatcatattatctgtgttagcatgtagtattttcagctactctctattgttataaaatgtagtactaaatctggtttctggatttttgtgttgtttttgttctgttttacagggttgctatcccccttcctttcctccctccctctgccatccttcatccttttatcctccctttttggaacaagtgttcagagcagacagaagcagggtggtggcaccgttgaaaggcagaaagagccaggagaaagctgatggagccaggacagagatctggttccagctttcagccactagcttcctgttgtgtgcggggtgtggtggaattaaacagcattcattgtgtgtccctgtgcctggcacacagaatcattcatacgtgttcaagtgatcaaggggtttcatttgctcttgggggattaggtatcatttggggaggaagcatgtgttctgtgaggttgttcggctatgtccaagtgtcgtttactaatgtacccctgctgtttgcttttggtaatgtgatgttgatgttctccccctacccacaaccatgcccttgagggtagcagggcagcagcataccaaagagatgtgctgcaggactccggaggcagcctgggtgggtgagccatggggcagttgacctgggtcttgaaagagtcgggagtgacaagctcagagagcatgaactgatgctggcatgaaggattccaggaagatcatggagacctggctggtagctgtaacagagatggtggagtccaaggaaacagcctgtctctggtgaatgggactttctttggtggacacttggcaccagctctgagagcccttcccctgtgtcctgccaccatgtgggtcagatgtactctctgtcacatgaggagagtgctagttcatgtgttctccattcttgtgagcatcctaataaatctgttccattttgatgaca

SEQ ID NO: 96 - DNA, 399 bp, Homo Sapiens
gttggctatacagtgcgctttgatgatgtcacctcagaagacaccaggatcaagtttctgacagatggcatgcttctgcgtgaagcaatttcagactctttgcttcggaaatacagctgtgtcattttggatgaagctcacgaacggactatccacacagatgtgctctttggagtggtgaaagctgcacagaagaggagaaaggaactcgggaaacttcctctgaaagtgattgtgatgtcagctacgatggatgtggacctgttctctcagtatttcaatggcgcccccgtcctctacctagagggtcggcagcatccgatccaggtgttttacaccaaacagcctcagaatgattacctgcacgccgcgcttgtctccgtcttccagatccaccag

SEQ ID NO: 97 - DNA, 307 bp, Homo Sapiens
tgagttattctccccacctcagaagtaccagcttttggtgtatcatgcagattctctctttcatgataaggaatatcggaatgctgtgagtaagtataccatggctttacagcagaagaaagcgctaagtaaaacttcaaaagtgagaccttcaactggaaattctgcatctactccacaaagtcagtgtcttccatctgaaattgaagtgaaatacaaaatggctgaatgttatacaatgctaaaacaagataaagatgccattgctatacttgatgggatcccttcaagacaaagaactcccaaa

SEQ ID NO: 98 - DNA, 642 bp, Homo Sapiens
gtggaggatgcgctatcttatcttgaccaggtgaagctgcagtttggtagtcagcctcaggtctacaatgatttccttgacatcatgaaggaatttaaatctcagagcatcgacaccccaggagtgattagtcgtgtgtcccagctattcaaaggccaccccgatctgataatgggattcaacaccttcttgccccctggctacaaaattgaggtgcaaaccaatgacatggtgaatgtgacaactcctggccaggttcatcagattcccacccatggcatccagccacagcctcaaccaccaccccaacatccttcccagccttcagcccagtcagccccagctcctgcccagccagctcctcagcccccacctgccaaagtcagcaagccctcccaactgcaagcacatactccggccagtcagcagactcccccacttccaccgtatgcatccccacgttctccgccggtccagcctcacacaccagtgacaatctcgttgggaacggccccatccttgcagaacaatcaacctgtggagtttaatcatgccatcaactatgttaataagatcaagaacagatttcagggccaaccagacatctacaaagcattcctggagattttgcacacatatcag

SEQ ID NO: 99 - DNA, 370 bp, Homo Sapiens
agctaccatagaggaggcatactccaggtcaatgacaaaactagcaaaatctgcaagcaattattcacaacttggaacatttgcaccagtatgggatgtattcaaaacatctacagagaaattagcaaattgtcacttggatcttgttagaaaattacaagaattaataaaggaagttcagaagtatggagaagaacaagtaaagtctcataaaaagactaaagaagaagttgcaggaactctggaagctgtccaaaccattcagagcataactcaggccctccagaaatccaaggaaaattacaatgccaagtgtgtagaacaggagcgtttgaaaaaggaaggagctacacaaagagaaatagaaaag

SEQ ID NO: 100 - DNA, 273 bp, Homo Sapiens
ctctacgtctcctccgagagccgcttcaacaccctggccgagttggttcatcatcattcaacggtggccgacgggctcatcaccacgctccattatccagccccaaagcgcaacaagcccactgtctatggtgtgtcccccaactacgacaagtgggagatggaacgcacggacatcaccatgaagcacaagctgggcgggggccagtacggggaggtgtacgagggcgtgtggaagaaatacagcctgacggtggccgtgaagaccttgaag

SEQ ID NO: 101 - DNA, 528 bp, Homo Sapiens
gttttgagagtattttagaagggctttatggaccacggctacgaagagacctcagtttatttgaagactgtgaaccagaagagctgactgactggtctatggatgaaaaatgttcattttgtaacctacagagagaagcagtcagtgattgtataccatctcttgattcttcacagtcaacaccaacagaggagctatcatctcagggccagtccaacactgataagattgaatgccaagcagaaaattacctaaatgcactctttcgaaagaaagatcttcctcagaactgtgatcctaacattcccctagttgctcaggaattaatgaaaaagatgatacgtcaatttgcgattgagtacatttcaaaaagtggtaaaactcaagagaatagaaatggttcaattggaccaagtatagtatgtaaaagtatccaaatgaatcaagcagaaaactcccttcaggaagagcaggaaggccccttagacctcactgtgaatcgaatgcaagaacaaaatactcagcaag

SEQ ID NO: 102 - DNA, 1975 bp, Homo Sapiens
tcacagcaaactgtacaatggcagttgagtgggttttgaaaaacgtctgaattgtagtgtctacatctaccttctgccttctctctaccctttgaaaaaaaataaatggcatgtttgagaagagagctctattaaaagaggaatattgcaaatggcttcagattctatgagtagtaaacaagctaggaatcacattacaaaggggaaaaggcaacaacagcaccagcaaataaagaacagatcctcaattagtgatggtgatggagaagattcctttatttttgaagcaaatgaagcttggaaagattttcatggttctcttcttcgattttatgaaaatggagaactctgtgatgtcacactcaaggttggctcaaagctaatctcttgtcacaagctggtattggcttgtgttattccctactttagagccatgtttctttctgaaatggctgaagccaagcaaacgctgattgagattagagattttgatggtgatgcaatagaagacttggtaaagtttgtctattcttcacggctcactttgactgttgacaatgtccagcctctcttatatgcagcctgtattctgcaggttgaactggtggctagagcttgttgtgaatacatgaagttacattttcatccctccaattgcctggcagtaagagcctttgcagaaagtcacaatcgaatagacttaatggacatggcggatcagtatgcctgtgaccattttactgaagtagtggagtgtgaagactttgtaagtgtatcaccgcagcacctccataagcttttgtcctccagtgatctaaatattgaaaatgaaaagcaggtctataatgctgccatcaagtggcttcttgccaatcctcagcatcattccaaatggttggatgaaacacttgcacaggttcgcctgccattgttgccggttgattttcttatgggtgttgtggcaaaagaacagattgtcaagcaaaatctaaaatgtagagatttactggatgaagcaagaaattaccaccttcacttgagtagcagagcagtacctgactttgaatactccattcggactaccccaaggaagcatactgctggtgtgctgttttgtgtaggtggtcgaggtggatctggtgacccctttcgcagtattgaatgctattctatcaacaaaaacagttggttctttggaccagaaatgaatagtcgaaggcgacatgtgggtgtaatctctgtggaaggtaaagtgtatgcagtaggtggacatgatggaaatgaacatttagggagtatggagatgtttgatcctctcactaataaatggatgatgaaggcatcaatgaacacaaagaggcgaggaattgccttggcttccttaggaggcccaatttatgcaattggagggttagatgacaatacttgcttcaatgatgtggagagatatgacatagaatctgatcagtggagtacagtggcaccaatgaatactccccgtggaggagttggctctgttgctctagtaaaccatgtttatgcagtaggtggcaatgatggaatggcttctttatctagcgtggagagatatgatccacatctggataagtggatagaagttaaagaaatgggtcagcgaagagcaggcaatggagttagcaagcttcatggttgcttatacgtagttggtggttttgatgataattctcctctgagttcagttgagcggtatgacccccgaagcaacaagtgggattatgtggcagcacttactactcccagaggtggagtgggaatcgcaacagtgatgggcaaaatctttgcagttggtggtcataatggcaatgcatacttaaatacagtagaagcgtttgatccagtgctgaataggtgggagcttgttggatctgtgtctcactgcagagctggagcaggagtagctgtgtgttcctgtttaactagccaaattcgagat

SEQ ID NO: 103 - DNA, 220 bp, Homo Sapiens
agccgcagagcacacaacattgacctgaaggggacagtcgtgatctttgacgaagctcacaacgtggagaagatgtgtgaagaatcggcatcctttgacctgactccccatgacctggcttcaggactggacgtcatagaccaggtgctggaggagcagaccaaggcagcgcagcagggtgagccccacccggagttcagcgcggactcccccagcccag

SEQ ID NO: 104 - DNA, 292 bp, Homo Sapiens
gtgtcagcagttgagtctttggagggccccctgctccaggtggaaggactgagtgaccttcgactagagcttcacagcaagaagatgaaccttcacttggttctcatagatgaactacaccggcacctgtacatcaaatcgactagccgagttgtgcagcgtaacaaggaaaaagggaaaatcagctccctcgtgaaagatgcttctgttcctctgattgatgttacaaacctccctactcctcgaaaattccttgatacctctcactattctactgctggaagctcaagtg

SEQ ID NO: 105 - DNA, 236 bp, Homo Sapiens
acacgaagggcctgtatttccaggcatttggaggccgccgccgatgtctcagcaagagcgtggcccccttgctagcccacggctaccgccgcttcttgcccaccaaggatcacgtcttcattcttgactacgtggggaccctcttcttcctcaaaaatgccctggtctccaccctcggccagatgcagtggaagcgggcctgtcgctatgttgtgttgtgtcgtggagccaaggat

SEQ ID NO: 106 - DNA, 942 bp, Homo Sapiens
cttcttttccgacaaaacaccaaatggcggatgacgccggtgcagcgggggggcccgggggccctggtggccctgggatggggaaccgcggtggcttccgcggaggtttcggcagtggcatccggggccggggtcgcggccgtggacggggccggggccgaggccgcggagctcgcggaggcaaggccgaggataaggagtggatgcccgtcaccaagttgggccgcttggtcaaggacatgaagatcaagtccctggaggagatctatctcttctccctgcctattaaggaatcagagatcattgatttcttcctgggggcctctctcaaggatgaggttttgaagattatgccagtgcagaagcagacccgtgccggccagcgcaccaggttcaaggcatttgttgctatcggggactacaatggccacgtcggtctgggtgttaagtgctccaaggaggtggccaccgccatccgtggggccatcatcctggccaagctctccatcgtccccgtgcgcagaggctactgggggaacaagatcggcaagccccacactgtcccttgcaaggtgacaggccgctgcggctctgtgctggtacgcctcatccctgcacccaggggcactggcatcgtctccgcacctgtgcctaagaagctgctcatgatggctggtatcgatgactgctacacctcagcccggggctgcactgccaccctgggcaacttcgccaaggccacctttgatgccatttctaagacctacagctacctgacccccgacctctggaaggagactgtattcaccaagtctccctatcaggagttcactgaccacctcgtcaagacccacaccagagtctccgtgcagcggactcaggctccagctgtggctacaacatagggtttttatacaagaaaaataaagtgaattaagcgtg

SEQ ID NO: 107 - DNA, 39106 bp, Homo Sapiens
gtgtcatccttgaaggatagaataaatcagctgtcgtgttctcagtgaaaagtggaaagaagttttaaaggatctgcaaaatagaattatgagacagaggaaagaaagaaacagacaatctgatctctatcttcatcggccaggtaagatttgaagtgtttaaagaacgggattcagtttggttacaaaagagtagctgggtcccagggatgggtttttcagttaggataatctgaactacatttgagagagaatttctagcaaactgcctgtccaaggatctcatggctggccttggaaggtagtgagcttcccgttattggaagtgttccagcagacatagagaatagattctgcagtgactaaaggattggataagatgtagaactagacctcagaagttttcttccaatgatgagatgctataaattttatgaacaacatacttaaaatagctttaagacactctacacgaattcgaggctgaaataagactagttaaactataatcagtcaattgattattaaaaccaagcacagacttacacccactacatacacttatgctcacaaatgataaatgccaggatagtttggattgcctttgaagtttattatttgcaggtgtagaagctcttaagttttagtttgtagtgtttctaaaagtgaggcattctcttgaactgtctttgatgatttgatgttcatacattatgtctgctgtgaatttggaaagcactgcagaatcactttttcaaagatcagaggaatgtgggatagaataaattaagtttctggctaatatttggtattcttctttagggatatgctttcagagatctgtttctggaaggagaagggaagtcatgacattttatgtgaactcaactgggcaatgtgaggttttcctatcattagctaataatatacatttaaagtcacatggaagtgtgttgcctgtgatctgaatttttattcctaaataaattagagattaaaatctgtggtttccagttcaagggtagggtaccaaaaaaaattgtctgatgtgggtttttatgtcattaagaaaggcggcaacagctaagtattattggcttttcccttttcttggttgcactggtccttttcccttatcttgaaaggcggtagtaaatgttgactttttgcctctaattcacctcaatgattgtacattttaacatgttttaagataggtggttttagcccctaccttattttcctggtctcttgcttccactgctctggttcccaagaatcaaaattgaggccagatgtatgttaacatcaagaaactctgactggcgctcttgttctctccctccccagatattaatgcaagaagggaactaggacaccaggaacagccactgttaacattctcctataatttctgcctgttattcctctgcaagacctttatgagagctgaatgtatttttgttctgtgttttaaaattttaacatttaaaggcagtgctttcctgtcacattgtaagtctctgaaaacattgtatataaatatatagatatattgcatatttgttttcaattttaaagtgggattttcttgcacaaaggtttgtatacaattctatggccaactgacctatctatgtatccatatatatatatatatatatatatatatatatatatatatatatatattttcctaattatgaaatgagaaatgaaaatatgtaaagtgtaaactatataaacaacaaaaacaaatgagaatttgtaaaatataaaaatcatagtaacttagcatccataataaaatctctattagtgtttgtaagtaccaagtcttttcccctccatatttagacatataatttattcaattattgattagaaattataggtaaaaatcatccaaatcatgcaaattctgaaaaactagaaatagcagctgttaacagtattggtatatgtcccttcatatactcaaatacatatatatatgcccagtattattttctattaatttttaaataactttttttactataaaagtgattcatatataattcagaacccctggaaaatataaaaatgtaaacaaaaaatggactgaaatatttccactcaaacataaaccaatcatttttaccaattcttccaaatatgtacacttatgtctgtaaaccccacacttttcaaaaaataaaaatgacacaagtatatattgctttgtggctttttaaaaaatatttaggaatgtatcaaacatttttgtgttaataaatgctcaataaaatttgttttggtatgaatttttttttttaaatctcattcctgactctaaacctcactttgagatttcccatctgtgcctctgtgtaattcgtttgtagtgatggtccactttactaattatctcttcagttgcacttaatcagctgtattactcacctactgaatatctaatttgaaatactgtgtttttatatttctaggaaccctgtttctatttttattttaggaaccctgtttacatcttcttaaaagttgtaagtcatttttaatatattatcctttaatatgtcctcttttacttttaaacttattaaacatttttattttatagtctgtattggataatcccagtatctgtagactttgtaagtctgcttcgactgtttgttctttttgatgactcttgtaaatgttgacttgtttctgtatgtgctttgcgatcccaccccactccccattctcccaatgtaagctcatgtttctaggaactttttctctgggaattctttgagagcttaaggtgaatcagttcagaaagaatttgtgttttcttctgtttgatgcattaaaggcactctcaacccatgtccacatcaggttgaattattagcttgaggtttttgggtggctacaccagcagtaggaattcaggtcatccccagtttattaagggcaagtgtccggatataaattctcaggggcgactctttcctacctagatccatgtagatccatgtgtgagacagtcacattttctgaccctctccctgtgtgacttgggtcagtattttttctattagttcagctgttaagggtaggatgtcccagctttttgcagaggtctttgatctgacttccatttttcacttgatttctgcttttaaaagccaaaactcaaggtcacctgggattattgggatataatcggctgacagactccctagtttattattgttatttttttcttttccgagttgatttcttgattacttttgcttcttgggagattcatgacttttgccaacttgggtaaacatttacagaggtggaggtgtgtgcttatgtatgcatacacacacagcagttttacatgtttttgtgctagaagagttttcactgtattttgctcttagtattctgggaaaagacgtctatagtgtgacttttaaaatagcaggacatattttgtccttcaaatatattgttattaataagattttttttttttgagatggagtcttgttctattgcccaggttggagtgcagtggcacaatcttggctcactgcaacctccacctcccaggttcaagtgattctcctgtcttagcctaccgagtagctgggattacaggtgcccacgaccacaccgagctaatttttgtatttttagtagagatggggtttcaccatgttggccatgctggtcttgaactcctgacctcaggtgatctgcctgccttggcctcccaaaatgctgggattacaggtgtgagccaccgcgctcggcatcttaaattaataccacttctcgaaaccttagggttgctttcaatttcccggatatagttttgtaatgattgtcttcaataggtatgttagcacagattgctgtttcttttccaatgataaacatcttgaagtaggattgctaggtcaaagggtgtgtttgtctggcttttgatacatattgccaaaatcatccttcagggacaggatgagatttttccatactgaaacccctgatcacagtgctcgaaagtgcagcaacttcattaaactgaatattataaatttagaaagattatcaatgtgttgggggcaatggcattttatttttaaaattagattatttaatcactttaagttaggcacaattttttcttatgctcatcgatcatttgtatttcataaatttcttgtggatgtttatcaaccatttttctattgggatgctcatctaaaaatttacttgtttaagctatatatttaatacatataattttcgtttttaaactttaagaatgttaacagtttatgatgtgatataaagcattttttcaaatatgcatttgtttaatataatgaagctttgaatgtttaggtagtgcattaggccatttttatactgctgtaaagaaatacctaagactgggtaatttataaagaaaagaaatttaattggctcacaattctgcgggctctacaggaagcatggtgctggcatctgctcggcttctggggaggcctcacaaaatttacagtcatggcagtaggtgaagggggaggaggcacctcatgtggtgaaaacaggagcaaacaagcaccactgaaagagagagagaaagggagagaaggagtgagttagttggggtgaggtgacacatacttttaaatgacaatatcttgagagaactcagatcacttaccaccaagagaatgttccagaaccattcatgagggatctgtccccatgatccaaacacctcccatcaggccccaggtataacactggggattacatttcaatgtgagatttggttggagacaaatatccaaactattttaggtagttacctttatcagtcttttctttggccttatgctaaaatagcctcctctcaatctgtgactatataaacattctactgtattttcttctagatctcttaaggctttatttttcatactcaattctaatgcataaacatttgtttatgtgctataaagatttataccttatacccctgtgaatttatttgagttggcttgcctgtgtacctatttggaggtgtgatttggatcaacccaaattatttgggattttttttgtgtgttgagtctgtaacttttgaatatgtaattaccatggcatggaattggtaatatagatgtttgcacaggcaatggtgaactccaaattcatgatggaacaagcaggtgcgtgtgttaaggaagaaggaataagttaaaggatgtgccaagacagaaagttaattataatgcttaacctttagggatcatttccccaacattttgtatgtttctttttcttcctaatggccattgggaatggaatgtgtgggattgactaggaaggctcttgcatcatcaggattttcagtgacttgagaaagctgggaagattatattaccagcactttgtgtcttgtgtacagccttttgcatgcatgcatgaatgaagaaaatcatttgacaggaaatagttgtgttagaatacttgtgcttttgggcaaaccaattcttcagttcatttttcatttctagtggctatatgccttcattaattcctccttttattgttcaattaccctttattgagcactgtctatattgccaggcacactgagcattaggataaaaaaataattatagtgctggctttatcctcaggaagctcaaagtccagcagttgtctcataactgaatagggtttagtgtgatgaatgttatgacttatgtgcaaggtacagaggtgatggcatatagcagggcacaccaacacactgctgatgatgtgtgtgtggcagtgattaaggagactcctgagacatcagctggacattggaagattgaaagataaagggagttcaccttgtggataggcagtggaagaaggcattttaattgtgaaaacagaatatggaaggcatgaagtaacatgatgttttctaggaactccaggtagttttgttggtaaataatgttaagacttgctagtggatgaggcagaggctggatcaggggaggtcttggggaatgtacaaaggaatctgaagtttgcttcctccttcccaattcttctttttttttttttttttttttttttttttttgagacagggtctcactcgttgctcaggctggagtacagtggtgccatcacagtatactgcaacctccacctcctgggctcaggtaattctctcacctcagcctcccgagtagctgggactacacagtcacacaccaccacacccagctaatttttttcaccatgttgcccaggctggtctcctgagctcaagtgatccacctacctgggcctcctacagtgctgggattacaggcatgagccgtcacgcctggcccacctccctcccaattcttaacaattgaaaggatttccacaattttgttagatcattgcaggaggttaaattggagagagacagagagaaatgaaatgagattagagctagagaacttttttttttttttttttttttttgagactttgtcacccaggctagagtacagtgttgtaatcatggctaactgcagcttcgacctcttgggctcaaccaatgcttgcgctttagccttttgagtacctggaaccacaggcacacgccatcacacccagctatgtttgtttgttttttgtagagatggggtctcactttgttgcccaggcttgtctcaaattcatgggctcaagtgatcctcccactttagcctcccaaaatgctgagattacaggcatgagccaccgtgctcaaccaagaatggatttgaatctattaagttggaatgttggactacattctggaatgggatgcataattaagtatcaccagactataagtgtctgtggaattgattggtatggataagatggcctaaggagagtaagcagaagtagaataggaaagggccaagtacagagcttgggggtaagccaacatttcctagtcatcagaggaagaggatcctgcaaagaagcttgagaaaaggctatcacaaagttggataagaatcaagggagcatggtgttgtgagtagggcaaaattaaaacgagtgattaatgatgcaaaatgccaacagagggtcaagtaagatgaggacagagaaagaacctttttagaggtcagtgatcttaaggttttgagttggggtggaggaagccatggttcatcatataaaaatgtaaaagcaataagcacagtttcttcagtatcctgggccttgaaaagggaggatggggtaggggaacgagagagaaggaagaagttggggtcaagggagtactttttctttcttgaataggaaacctagcatgtttatattctgaggactgggaaatgaggagagaacaaaactgaagccacagaagagagaataattgaatgaagtaaactgcagtgcaaaacaagatgtggccaattgaagactaggatgaatgggagagatatcttgcaaagtcattgttagccaaaattggtgacggtgactatcactggaggatcaagacatgcatttcacaaatgtttatgagcacaaagaactatggcagctgtgtactgggtgaaggatggagccttgattatgccctcatggaatgattagtttttcttaagtctgagttgttgggatgaacttcattgaaatagttaatgtgactttgacactactaagagtatgaatattacatgttagttggaatagtggtccaagacatatgcaaataaattcagttatttctgagttatgaattctgagcccaattatttttaccattatatttttgtttctgtgagccatataaatctgagatacagctggaggtttgggggaataattcctaggatatgtggctgcaccatctccccttaactcccacaaagaatggtttggttttcatggtccggctccttgggatggtggaggaggaagcttctgacctggccacactgcattggctcatgggtgactgccctttctgtgattgcagccaccactttttagtccctgatccccaaataatagttcactgtacttatcttaccctccccaaagagaaggagattttccttccaaataagaaaactctttagggcatcctcagggatgcctatgtgtttgtaacacttttagcacccaacatagccctttctcttttgatttgtggttcactctattaagcaaattacattcattgtccctagagaatgggttctaagtagatttgtgtgccaccctgctggtctcactcttgcgggcttttacttgagatgcgagggtgcatgtgttgactctgtcttaccgtggagtgttctggcttgcctactaaactccagggatagggacagccataatcctgccttctctgtttgtgactgaatctctgtggtttaaatactttatataaaacttacacatttcagctgattaataacagctatgcaggaacattcaattctgaggcaattttaatgtagtcgttgataaaatacaaactatcataataaaactgtttaatttacagttgattccacaggatcaggggatttccatttcaaatgggctcctgatgatctgcctggattcccagaatttctcatctgggtatacaaattaggtttcttgtaaccaacacttcatattttatttatttattttttttgtagagatggggtctcaccatgtggaccaggctggtctcaaactcctggcctcaagcaattctcctgcctcaggcccacagactgccgggattacaagtgtgagccaccatgcctggctcccttcatattttaaagagaagttacacctacagcgctttcgacttttgatggacacatctttctagagcatagttcttgcattcctcttttactgaaagttgttaattgctttgttccattagccttagcaggaaaaatcctatctctggagttttaggtcccttggtttatcttaatttcgattaggaataagagaatacctacctattcagctctcaaacagcaatgaatattgcactgtgttcatcctcattacctcccattattagtttacattctgggacactttttctatctttaatgataagttcattcagaaatatgttcccacattgagccatacttctggttttgtgtgattaactttggcaccccatgagtagtgtctgttaaaattaaatgcttgttttgcctcaagagagctagaatacatacacatattaccgtgatttactgttaagtgtgcagattgaggtggtttagaagtttaaaattggtttccgaagggaaatttacaagaaaagtataacagatgaatatgtgattagaactgcattccttttttaagtaaattaaacagttttattatgatagcttgtattttatcaatgactacattaaaattgcctctggaattgaatgttcctgcgtagttgttattaatcagctgaaacatgtaagtgttatataaagtaaaaagagctataaaccaaggagaaagtctgaagatagaactttaaaaaaccactgttatttccaggtaaaatatttaatttccaaagaaggttagcatatgttaaaagctaaatcttgttctgcatttgttggctacatttatatgaatttttatatggaagttcctatccagggtatttattagtcattttggaagtttttatccagggtattgattagttggcatcttatatgttctttcagaagctgtatgtattatttttaaaaatagaattctaattgagcttttaatctgtctgctgaatcaggaacattacatgactttctttggaggggaaggaagccagtggttagttttaggtataaaagagatcttcaagcacggtctttttaacttcttgtttctgtattatttcaggaaatagaggcagctttccctacattagattgttagcacagaataacagtagaagggttcagaaatgaagctaaaaggaaataagagaccagcatgatgctggagttcccctaggccagtcaaattatgttcaggcctttagtccatctggatgttagttctatgtatgatatgaagttggggtgtctgattaggtatatttttccttttgccttagaaccatttttttttccagtccctttatgtccccactgtaatgtcatgtgtatcatttatgtaagactgttttgaaactttgtgtacctctgaatgtctagcctggaacaattgtcacattaccttcattacatggcttttaatatgtcttactcattggtaggtcaagtcctctacttctattctcttaatttgtcttggctattttggtccttagcatttcactggaaatttaggagcaggtttatctatagacttccttggactttgaatttaaataatcattttgattgcaaataagggcagttttgactctttcactccttacatgttatttattgttgtcttgtttcattgaattaactaggctgtctagaactgtgttgaatagtaggagtaatagtgagattcttgttattcctgatattgaagaaaatgcttctaaagagaaccctattcctttttgaggagtattatttaaaacagctcagcatttccatgtgtttgattttagcatattaaaacataggaaacatgcagtgaacaaaaggggttattatgagcttagtgtaccagctgtcttaaggttttcattgttttagaggcatttagttagatttaattctgactccaaaatgaccgacctctctggagactgtattttgtaggagagaataagagtatttccattttaatggaaaagtggcaaagcttccttccacttcgtaatttttgtggacagtattccagcattgtccaacagatttttgtgataatggaaaatgtatttctgcactgtgcacgtaggagagcaactatcatattggacagtgtgggcctagactttggagttctaaccatgccccatttactagctgcacaggttcctgcctggtttcagaaggcttattcttttgggctagcacagctcacactgaggcacgtggctgggggagtagagcagtccttgctggattgcagccatggctctccttttagcctgatgcctttgtgcaagtgagcagctggcaccattgacaagggtgatcctggtgactacctctggaggctctggaccctgactgctgaaacaggagaaaaccctggccccatgcccctaagaagtcacccgatcaaagtggagcctgttgtttccctagtcacaagtcaatactaagagatttggctagtgacaaggaaatttttcatgttagaattcttgtacccagctctttttagaactggactactgttctcccatattcaggaggccatgaaccaaccagtagattgtttattgtgcttgctgatttatgaaaaaacttctttgtacagagcttctgaaaggggtaagagaaagggagaatagcagtgttatggactgaagaatgtgtctccctgaaattcatatgttgaaatctcagtccccaaggcgatggtattaggaggtgattaggtcataagggctccgccctcatgaatgagattaggggccttataaaagagaccctacagtcggtgtgttgactcacacctgtaatcccagcactctgggaggctgaggcagaagatttgcttgagctcaggagtttgagaccaacctgggcaacacagcaagaccctgtgtctactattaataaatattttaaaaaattagcctggtgtggtggcgtgtgcgtgtagtttcagcttcttgggaggcttaggtggcagaatcacttgagcctaggagtttaagtctatagtgaggtagatcgcgccactccagcctgggtgacagcaagaccctgtcctaacatgctcttaggagtttcataaatgtgaccccagaagagctctctagccctgtttccacttatgaggctaaattgagaagatcgcagaccacaaccccgaagaggcctcatcagagcccaactttgttgtcaccctggtcttcgacttccagccttcagatctgtaagaaagaaatgtttgttgtttaagccacccagtctgtggtagctttcatagcagcctgaactgactaagacaagtggatagctgtctggttgttttagaaagcagggctgcatggtgtggggatgctgctgctactcattcctgctcattccagtgttctggaaaatgaggtagggtgggggaggtctgaaccttctaatttatttctagctgttcttttttagcaattaaagcattcgttggacaccagttgcttttaattcagttccatctgcactgtacttaatagatattactcccaggttcttataaatggctggttgttattcttggtttcagagctattttgagtttttgtcttcatttgtattttgactggaaattgggtgtggacatattggggagttggggctgacatcatctttattcagaagtcactttatgaccatgtgcgggatatattaatttcactacagcttttgatcttaataaaatgtctctgggttgagctgactgatcacgaagattttaaactttaatgtttcctaagaggattgatacagccatccagagaataacatagtatataggagttatcttttgacagatcaagttccatttccaaatgatataaaacccacctgtgtccacatccttatgccttcttttgaatctatgatttgataacttaagaaatctatctatgcattagatattgcctttccggtagagctatggtgtgatcttatgctcatgaacagaaaatgaatttaagaatcctcagacctattgtccagttcttttctttaagaagaacttgctattgactttgtcaaaactaaatctgttgccttgggcgacaccctccaaaattcagattcagatggattacactaatagctttatgctacagatcaagtcatataatgtgaaggtacaattgcagctcccttattgttgtagacgcttgccgggatgaagttctcaaacttaacatgctcttaggagtttcattaaacatgtgtgtgtatttttaatgtccaggatttataaatggtaacacctgccctcagttcttttttgcactggtccacggtgtttcagttacagtctttattgaataattcaaaagttctctcctgaaacaatatttctggcagtccttataattgaccaggtggaatgaccttcagtttggatgaaagtccaatttatttatttatttaataaaattccaaatttgccttttctgccaggtattaaaagcacttaaaactttttctaatcaacaatactgaacacttcagacacttatcagagactttagtcttgaattatgtattattttaaattattatttccagttctattaaaggccatgtgaggccactgattttttaaatggacagtttagtgagtggttcttaaagtgtggccccatgagcagcaccagcatcacttggagatttgttaataaatgcaaattcttggatccaccccagacctactgaatcagacatgcagtggggtggggctcatacatctgtgtttgaacaagccctccagtgaattctgtgtgccacatattacaatttgagaaccactggtgtaatgcagtagttaacacacctagctttcaacctagacccactttgggtgcttgttaaaatacagattcttggttctctctcgcacagcatcttatgcaccagctttggagaagacacaggactcagtgttactatattctgcagtaaagcttgctctgctcaccttatatcatattagatcacttactagcaataaatgtagaatataatacgtttgttctagtccaagtatctgtgaagaactctgatttaagataatggcatgtaatcagacatacgcttcagaattaaggagacttaaaaaatacagatgtctagtttctactaaataaaaaactctgttaaaagtctggttttgttttgtgtttaagtttcatgggtgctataccatccaagaaccatttatttttatcttcagctttcattgagagttgataggagaagaaatagccatattaatatttttgtatattttataccatttgataactcttactaggaattagtagaagaaaataacatctctctctctcaatagttaatacgcaactcttcattttcccttttgctttggttaccagagagctcagagtaaaattttgcactcagttttgaaatacctatttcccttgcttactttaatccatgactcttgttctttataatcttctgtaacctatctgaaacttatttggaagtagatggggccccaaaactaagtaaatgatggaatatctgccctttgctttgaaatgggcttcttgattgactctttcccagcttggggtcagtgtcttctttatatcatccttcctttcatagacacaatacaattgttggtagcctcactgtgaaagagttaatgtttcccaataccaggcactgacataggcagtaggaaaaatctggttcattttgtttcaaaatacaccataccacgaataagtacaaacagctgcaatttgggcttggggccaaaagaaatcagtgtggtgctatgaattccagctgagaggaggagttcatgtaacttagatcagagatcagtctctgtaacgagaccatcagaccccagaaggggaagggtccttgtatgtcacttcagttacctccatcgctaccaccaaaagaaactggggtagttctttggagaggtggccttttagaaatctaaaattatacccttggagccgcttctctgcaattggctgaacacatcacattttatcccttgcccccagcactgtattactgtaaaatggggaaatctgggaatacaaaagatttagagacttgccacctgctcattttatcgctatgacttggcttaaaacaacagtcgctgtaacctcagccctttggcacacgcttccttctcaagccccaggaaattcttggtcaggtcgcacccacagccactgagtctggttgtttcttcttctcccagattttttagggaatatcagtttagtaaaacttatttttttgtaagcggactccttttgtatcagaattcctttcctttaaaaaatatttttatatttattattattttttaagtagagatggcatcttgctatgttggccaggctggtctcgaacccctggtctcaagtgattgtcctgccttggcctcggagagtgttggagttacaggcttgagccaccacacccagcccagaattccctttctaatgtcataatgccttatctcttttttggagtggtcatcaaatcccttttcacactagttagaagttttctgtgtgatattctccattctctacatatttggactatcccactgtgtttttctggacatgtaggacagcatctttcaggcccattagcagatagaagtctttcaagtggggcctcacagctgtttttaggtctccggttggtttttaggtcatgtctcagacataggggctttgtggctactttgtggttttactatctcctcatagtttattaaaaaagaggctccttttcccttccattttctactgctttcctgataaaagacttcagccattatttttaaaagtgtccgctattatattttaaaccctttcataacaggaaaacaggagttaaccactcacataagttagagaggggaccccccttttttggtgattttcatagttactaaatcattgtgaacagagaaaagtcacttaattccctttgcttcacttcctgatttcttcaacagagtggtaacatttgagatgctattcatagaattgtatatcaaagaatgaggccagctctttcttggtgctccctcaagttggtaaaatcacgtatgtgacttctcaggaagccagagtatttacatatcctcgacccagctgggaataattcatcctctagtacaatactagttttctttagattcccctctatatttactgttttagagaggtattgccaagagggttttcaatgaggaagaatggtaatatgctgggagcaggaaaggaaatgaaattaagcgtctcattatttttcccatgctgaagtagatattaggattgtattccacaggtcttattttcttccccctccccacctcaaaacttcaggcaggatgtataagtctagacttaggttatatactgattttttttaagcttttatctccagtttaattagaggctggtgaaggaaactgggtttcttatttggctctcaaactttgaaaataaacaaaactttcatatatttgaatagcacattgtgggccaaacaacatctggaagagattagtctcactggtaggcagaagaaatttcataatgtaccatcttcaaagaacatggtacctcgtcacatttctcatgtgacatgtatcctggaaacagaagtacttagggccacattgctggagcatctgcctttgtctttgttttgtgtttaacataaatgtgtctttctaatactatgatatttctttagcttttttgaaaacacagctaaacttggtggtacgatgctgcctgcgctgttgactgtggtctagacttctcctttttcagaagacctatttgacttttggcaacgtgcctcttagagtttgtgatttgtttgagacatcttttcctgtcttctctaaaatcaggcacatatgtgtctggggtagtcatgtactttgtaacagtcacttttagccagatcttggggatgtctattcttatggttagtagtgacacatgaaccctctcttccacttaaaattattttatgtaagatgactgcttctccttctatccctgctccttcctctcctcctcctctactctgcctccctcaaacttatagaaaagttgcaagtacagtaaaaaagtgtgaagaagcttttatctccctaagtcaattaagagtgaattgtttacactgatgcgctaacatcttcaaatactctgtttttctcacaaacaagcacattcttcaacatagtcacaaagcaaccactgaaatcagtaaattcaccctattatcttactatcatctaattatcagatcccattgaaattttcccatattgtcacaacaatatccttgatagtgaaggatccagctcatcatcttgcactgtatttataattaggtcttttctaagtctactttattatggaatagtttctaacttttttcttggcttttatacccttgatctcttgattataggctgttcattgtgtagaatgttcctcagtgggtttgttacatgtttcctgttgattaaattcagatttcaaacttttggcaggaacatgacagaagtgatattctcattgcattttgtcaggtggcacacactgttgatttgctccattattggtgatgttattttgatcacttgattaaagtgatgtcttccagacttatttactgtacagttattattttgcactttatggtaagtattttctagggaagtgtttgagtctttgtaaatatcaattttttggtcaaacttttatttgtattaaaatgaacttacggattcctttttcattttcaaacttagaaataattagaccagtgagtacctttcaaactagtttccttgtcctttttacatgtctgcataattcttttaagtgctgtcttaactgtctgctcaaggtaaccaaggcatcatgtatttccctaatctgaggaacaggcatatctccaaggagcctggcacctttctgtagagaatggtattaagagacgaagatgtaagtgttaggtgtgctcattgttattgggatatcactattcccaggcttctcactgtgcaacacacacattgataactgtttttatttctatttctattgaaaacctctctccttctctcctggattacctctttctttctctcctggttgacccctcacctttcaggctttgacacaaactctgggctaccttcccatggggacatcctcttcacctcacttagactctgacactctgcgttgggctgcccttggagccactgaggaacattctacacaataaacagcctataatcaagagatcaagggtataaaagccaggaaaaaacttaaaaactattccataataatttttaaaaattagtttgtggtatgagaacacatggacacaggcaggggaacatcacacaccaggggcctgttggtgggtgaggggctggggaagggatagcattaggagaaatacctaatgtaaatgacaagttgatgggtgcagcaaaccaacatagcacatgtatacctatgtaacaaacctgcacgtggtgcacatgtaccctagaacttaaagtatattaaaaaaaaattagtttgtggttagtggtcatattttcttgtatgccagagttttccatttaaggagaatgttctttaaatattacattttaaaaagttgtaaaatagcgtttttctttaagacttatgctttcttttaattttttcattgataacatagactacatatatttaaagtatacaatttgatttgttttgacgtatgcatatactctcgaaattatcactgcaatgaagatggtgaacatatccaacactcctcagcttttctcctgttgtttgaaatccttccctcatgtcattctgctcttatactcccaggcaaccacttaattgcttttggtcactataaattagtttgcattttctacagttttatataatggaatcatacagtaagtgttgtgttttgtctggtttattttactcggcttattttgagagtgatagatagtattatacataccagtagttcatttgtttttattgctgggtagtcttctgttttatggatacataacaatttgtccattcatttatgaatggacatttgagctgtttccagtttttggctatttacagataaaactactagaagcatttgtgctcaagtctttgtatagacaactactttattttctcttgggtaaatacctaggagttaaatggcagaactggataattaacttttaaagaaactgtcagattattttccagactgattctatcttacattcccacatcagtgtgtgagagttgcagcttctttaaatcctcactgacacttggtatggttggttttttcaattttatccattctaatagtcatgtagtggtgtttcattgtggttttaatttgcatttttcctaattgctcttctatgcttatctgccatccgtatattatctttggtaaactgtcccaatcttttgcccctttttgaattttgaaatttcttatatgctttggatacaagttccttatggacatatactttgaaagtattttctctcagtttgtggcttgccatttaatttatttaacaatgtcttttaaaaagtaaatttaaatgaagttcagtttttctgtttgttctttatagatcctgcttttgatattttgactaaagaaaaaaaaaaacaagcttttaaagaatttaagttagttttatccagaagtcttactgagagctatagactgactgaggactacagcctgggaggagtctttcagaaaggttctgttggccaggagtggtggctcacgcctgtaatcccagcactttgggaggccgaggtgggcggatcacctgaggtcaggagtttgagaccagcctgaccaacatggagaaaccccatctctactaaaaatacaaatggcacatacctgtaatcccagctacttgggaggctgaggcaggagaattgctcgaacctgggaggcggaggttgcagtgagccaagatcgtgccattgcactccagcctgggcaacaagagtgaaactgtcccaaaaaacaacaacaacaacaacaacaacaacaacaacaacaacaacaaaacaaaccagaaagattctatcagactgctccaaaatagtgtttcagctcacagtttatatacaggttcagtgcatataaaatcacatcaaagtttgggtgcaagagtctatctagttatagattacagaagtgtaatcactaaccctgtcaggtgttctcttatgtgtaggaaaaggcaaggactagggccatttatcttttaaggaatacagtgactcaggcaagagctggagggtgggaggccctgttctctattctgttttgtcttcaatgcacgtttctggagagctacacgttgtcacagagtcaggggctttgggaaagtatgttgacaagcagaaatgagcaaatatgtcttcttacatttgttactttggctcacaggtatcttatctaaggaaactttgcttacgcaaagtacacaatgattttctcttatgttttcctctagaagttttatggttctaagttttatatttaggtttatgacccttttcagttaatttttgtatatggtatgaggtatgaatcagttggttttttcagtttatatttttgcttatggatatctaatagttccaccatgattttaaaattgtgacttcatatttaaaagtggagaaaatgctatactacactgtcttaatcatttattatacttttaatgtctctgacagggacaatagtaatgacccctctttcaattttattattagtcatttatatcttctcaccttttttctcgactaatctggttagagcgttagcaattgtattgatgttcttaaagaaacatcttttggatttgttttatagtttctatataattatctactattttattttttcctgcttactttgattttaattaaattatccttttctagtttttgatggtaaaagatgaggtcatttatttgagacttttcttcttttctattataggtgtttaatgctatggttttcctctttggcactggtttagtgacatcccacaaattttgatatgttttgttttcctcttcatttagttcaaaatattcaattcaaaagattttttaattacctcctttgacttctttgatttataaggtatatacagatacattacttagtttccagcatttgatgattttccagagatcattctgttattgataatttaagtgtggtcagagaacagactttttatgactcaaattcttttacatttattgagacttgttttatggcccagaatatggtctatattggtacagatttcttttgttcttgagaggaatgtgtagtctggtgttgggttgagtcttttcaaaatgtcaaattagatcaagttgactacttgtattgttcatgtctcttatatccttactgattttttctgttctgtcaattaatgactgaggcatatttatctctggctatatttgtggatttgtttatttatccagttactcgtttttacttcgtatgtttcaaaaatctgtaattagatgcatacacttttaggattgttatgtcatcttgataaagagactctatcattatgaaataacctttttttttaaaatccctgctaatatgttttgctctaaaatctactaatctactctgttagattttaatattgatacttcagctttgttttgtttagtgttagctgggtatttcgtttttttatccttttacttttaatctatttgtgtcttcatgtttaaagtatggttcatgtagggagcatatagttgtgtcttgcttttttatccagtctcataatctatgccttttaattgagttgtttagaccacttacatttaatgagtgtatatatatgtgttttcatatggttatgtttaaatctatcatcttgcttttgtctattgtcacatttgttctattttttggatttggtatactttatgatttcattttatcttcttgttggattgttagccataattctttattttatttttagtggttgattcagtgtgtgcagtaggctttaatttattgtaggcaacttttaagtgacatcgtacccctttatgtatagtacaagaacctcacaacaatacagtatacttactttactcccctgctttttaaaacttttgtaataaactctattgttattttcattatctttttagaacaactttattgaggtataatttaataccataaaagccagccatttaaaattcaatagtttttgatatattaagagttctgcagcagtggcatgtgcctgtattcccagctattcaggaggcagagacaggatcacttgagcacaggagtttaaggccagcctgggcaacagagccagtccttgtctccagatagatagatagataatgtgcaatcatcactccaatcaagctcagaaaatgtttatcattccaagagaaacctcatatttgttttcaatcactccctatttatcctcttccaaactcctgaaaaacacgaatctactttctttctcatagatttacctatcttggacatttcatgtaaacaggcttatagatttgtggccttttttgattggcttttcacttagaaaaaaatgcacaatactgcatgttgtagtatgtatctttctttctttttttgttgctgctcaataatattccattgtttaaaaataccatatttaactttatcctttcaacagttgatagacatttggattatttctactttttgatgattctgtaatgcttctatgaatattcttggagaagtttttgtgtggatatatattttcagttcttttgggcatatacttaggagtggaattgctggatcatatgaaaatgctatttaatattttgaggaactaccagattttcacagtggctgcaccttttatattcccaccagcaatgtataagggttccaatttctccacattcttaaccaacacttgttattatgtcttttaaattatagccatcctagtagttatgaagtgtgaatttggcttttgtttccctaatggctaaaggatatccagtgtcttttcatattcttgttagccatttgtctgttttcttagaaaaatatctattcagatatttaatcattttaaaattagcttgtctcttcattattgaattgcaggagttctttattctggatacaagtcccttatgatttgcaaatatttttctttctatgagttgttttcaattatatttttcagatacttatataataaaatggtttttatatttatccatgttattactattaatatttttggtatccttcatttctttatgaggatccatattttcatgtggtataattttctttctgtctgatgaacttctttttaacatttcttatggtccaacgtctgcttatgatgaattctttcaccttttgtatgtttgaaaatgtttttattttactttcacctctgaacattttttaatgtgtataaagttctagattgatagttcccccacttcccattattttaaagatgttgatcacctgtctttttgcttggattgtttctgatgagaaatctgctgtcattcttatccttgttcctctctaggtgtctttttttttctttaatccagtaactttaaagattttattgttatggttttgtaaatttgattgtgcatttttctttgtttcctgtgctcatgcttcattgagcttgttgagtctgtgggtttatagtgtttatttttagccttgttttttcactctccctttttgctttggggactccagttacgtgtacagttgacccacgaacaatgcagatccatttatatgcatatttttttccaaccaaatgcagatcagaaatatagtattcatggtatgcgaaccccacaggtatggagggccaactttatatatatatatatatatacatgtaggttccacagggctgtctgtggtgtaactgcccaatgggttctccttacctactccccaggtggaactgatttatcaagacaggagacttgcaatagcgaaagagtttaattcatgtagagccagctgaatgggtgactggagttttattactcaaatcagtctccccccaaatttggagactgaagtttttaaagaataatttcgtgagtagggggccagggagtagggagtgttattggtggcactggagatgaaattattgggagttgaaatattatataaacatatatattatataaacatatgtattatacaattgaatataatacctattatatatgcaatatattatatagaataaacataataattgaatagatatataatagttgaattgcatgtaaatgtagttttaataacaaggttgccaagtactgtattactggatgcttacattgttcctaaaattatcctatcatacatgatattgcattaagcatccttgtagcaaattctttgtcctttatatttttcccatagtataaattcctagtggtggagttacatgccttctgattcagcacactttttaggatcttgatggaaaccatgcatatacacacatttccctaatattagcattccagaaaggttacaacagtttcacctataccagaaatacatttgagcgcctattttcctactttgtatacaaacacatcagaaaaccatctttcaaggataactttcttatgttgaggcagttttaaatcacaatcattcaagcactcagtattgattttgattcattctcccaaaggtgcttgaattttaagctacttgaaaccttaaataatggaagatgtctctgttaagagaactctctgctgttgaacactgttattcagggctgaatcactttacttaccacagccacagacttctatattctgactagctatttcgacactttgtttccacccccattggtctcttttcttccctaattttcttcccttcacttcatatattcacattccttctattttaggccatttgggaattactttttttttttagaatagttaactttttaaatttttgttttatttttaatttttgtggagacagagtttcactatattgccctggctggtctcaaactcctgggctcaagcaatcctccggccttggcctctcaaagtgctggaattacaggcatgagccaccatacttggctgaaaattacttctcatgttagaaacatgcacttactcttagaatgaaatatttttcaagactcatctgtggttggaagagtctagcctgcccgaagcttggttatagaggttgtaattgtccttgttctgaaaactacggactgaaaacaccccagcttggcacagtggcgccctcagtagaaatgtatccaccctatagggtgcttctcctgtaagtggaaatcctaattcaaatttatatctgcattgctctaggtccctatgcaaatggctgagggtcatggcatgcacacacaaagaagatcaagagtttagatgactctggaaagaaacagggcagaatgaagtctgtctcttacccagagtagagttctacttttcaagaggatttttttcagtcttctttgtggaatcctcctcatttccccttaaaatgtagatctcattccttctcttcttgctctctgtcttcatgagtgatcacactcccttgagctcaattttctccaactcagaaatctgtagttccaggcctttcctgatttactacttctaagtagagattttctcccattttctaagcattgtaacacaaactagcttgaccttgaggctaaacagtttggccaatatatcctttgccttcatagtgacacttattctaagatagtaatctccataatttgaattttacaattcttggttattttggtaagtgtcaggatagcattcctctccatcaggaccacttctaaagtgaaaactatacagctgggaatacagtctttgaaattaagacagtgtttacttatataagttttggccagtttctccagcttctagaatacctgacctggcccatcatgacctgataaaatacatgtaaaaaggcgcaattgtgttaattgtgtttctctctctgcagtcagtgtgcgcaggatgggctagttgtatttcttccttggaatgcgttagctctgtgccctgtagaaaaaatgatagacttttagaccaatttgaaaataaaagatctaattgaaaagctggagaagcaaatgaaaagactgatcttatttgtggaaaaggatttatgaacatgaaatatattgtatcctgtgagtacactgaagcaagatcttagggctgacactttgaggaagagaggaaagtagccacagagagaggaaagcagcctgtagccacaggtggcgatggtattttaaaacaaaacataaggtagtcatttggtttctcaaatgcatttctgatttctttgttctgaatctgcatgtgaagcaaatgaacccatagcctccttcatcccagctgaaccatttgcttcactgtcagtgaagggggtagagtcagtggtagggataagtaacccagataaaaagtttagcatggccatgtctctgtcatcctgttttcccctttcctttttctttttgattctttaaaacggaaattctgattttatcaactttagaaatgaaacagtattgattggctcctttctcaaaaggtgaagaatgttctcctttttcttgtgattttctggagttttcatcttagcttattagttacacgtatttcaactgtatagctttttttttttcctattagttaaaactgcattcttttttttttgtcaccgttactatcacaggtattctgtgcttatctggattgacaagctgctcttgccaaaggtttcctgagcacattattttgatttgtcatatgagtagttgtattatgtgtttgaacaaatttttcagggagaaatatatcttaattataccatatgatccagcaatctcactgctaagtatatacccaaaagaaagaaaatcagcatattgaagaggtatcttcactctcatgtttattgcagcactgttcacaatagccaagatttggaagcagcctaagtgtccattaacagaaaaatggataaagaaaatgtggtacttatacacaatggagtattattcagccagaaaaggaataagatcttgtcatttgcaacaacatggatggaactggaggtcactgtgttaagtgaagtaagctagtcacagataaactttgcatgttcttatttgtaggaactaaaaaaattaattgaacatatggagacagagaatagaaggatagttacgaggggctaggaaggtgggttggttaatgggtacagaaatatagttaaatagaatgaataagatctagtatttggtagcataacagggtaaatttattgtacattttataatagttaaaagagtatagttggattattggattgtttgtaacacaaagaaaggataagtgcttgaggtgatgcgtacccatttaccatgatgtgactattacatattgcatgcctgcatcagaatatcccatataccccacaaatatatacacctactatgtacccacaaaaattaaaaataaaaatattaaaagtggcttgtttttgttattgcaggtaaacaaaggcttgattggctgtagaatttttgagtcgttccatatttctgacaaaatcttgaaatttattccttcatcttatttgttggggtgaggttaatggctgtgtccagcccatttttttcccattatgatttgatttattttccccatttgatttttttaatgggatttcttttaatttttaaagcttattaacttaaacctgttttgcacttatcatttacttcatagttttcctggaatttgtcacacttttttaaattatggtaaaatacacataacataaaatttaccctcttaaccatttaaatgtacagttcagtagtgttaagtatattcagactgttgtgcaaccaatctccagaactcttttcgtcttgtaaaactgaaaccccgtacccattacacagcaatttctcattcatccctctcccccagtccctggcaagtactactgtactttgtctctattaatttgactattagctaccttatatatgtggagtatttgtccttttttgactggcttatttcacttagcataatgtcttcagggttcatccatgtcatagcatatgccagaatatccttttgaaggctggatgataagccattgtatgtatataccaatttagtttatgtctgttcatctgttgttgaaaacttgggttgcttctacctcttggctcttgtagatagtgctgctacaaacatgggtatacaaatatctctttgaaagatcattaacagttattttggatatatacccagaggtgggattgctaaatcatatggtaatcctattttaatttttttgaggaacctctatactgttttccatagtgattgtaccatttttggtcccaccagcagtgctgggttccagtttgttcacatccttgctaatacttcttttctttttttgataatggctatcttaatgggtgtcagatgttatcttactattttgatttgcatttgcattttactgatgattagtgatgttgagcatctcttcatgtttggccgtgtgtgtgtgtgtgtgtgtgtgtgtgtgtgtgtgtatgtgtatatacatttttgtagaagtgtctactcaagtcttttgcccaatttttaagttgtttgttggtttgttgtttttgattttgagttgtaggagttaggaaagtttacttttattgctaagtaacttctgttccacttatttttgtcaagtatgccgattatgttgaatctttatgtcctgcgatttatatccataatttctttttaaatttttttatttttattttttattattatactttaagttctggggtacatgtgcacaacatgcaggtttgttacataagtatacatgtgccatgttggtttgctgcatccatcaactcgtcatttacattaggtatttctcctaatgctatccctcccctagtccatcaccccctgacaggccccagtgtgtgatgttcccctccctgtgtccatgtgttcttattgttcaactcccacttatgagtgagaatatgtggtgtttggttttctcttcttgtgttactttgctgagaatgatggtttccagtttcatccatgtccctgaaaaggacataaactcatccatttttatggctgcatagtattccatggtgtatatgtaccacattttctttattcagtctatcatttgatgtgcatttgggttggttccaagtctttgctattgtgaacaatgctgcagtaaacatatgtgtgtatgtgtctttatagtagaatgatttataatcttttaggtgtatacccagtaatgggatggctgagtcaaatggtatttctagttctagatccttgaggagttgccacactgtgttccacaatggttgaactaatttacactcctaccaacactgtaaaagcattcctattttccccacatcctctccagcatctgttgattcctgactttttaatgctcgccattctaaatgatgtgagatggtatctcattgtggttttgatttgcatttctgtaacgagcagtgatgatgaacattttttcataagtttgttggctgcataaatgtcttcttttgagaagtgtctgttcatatcctttgcctactttttgatgaggttgttttcttcttgtaaatttgtttatttgtagattctgtatgtccataatttctaattaatctagtaaattccattattgtaagcacatgtactttttcccaagccagacttccatggcaccaacttggttttctgcagtggcacttctgtcttttgtagttttaaatttttagtttctgcagtgatttttttttatttccttaaaaaattttaaaatcaaacttgtaaatgtttataatttaaaaatgtataggtagatactttatttggcaaacaatgttagctatcacttttaccatgttcactatattgttcacagtatgataagtattactccaggtgtcttaggtacattaatcaattcaattcttatagtcatatgtggcagccttactattattatacccattttacagaggagaaatctgaggcaaggagacatgacataacctgcccaaggtcatactggcaggacaaggatttgaacctaggcagttggattccctagcgtactgtacttgtaactactatgcagcactgcctctcttcctccttttcttaaaaaaagaaatgtatgtgctttatttgaattctgactcaaccaaccaaactgttaaaagttataagaaaatcaggggattgtgaacttaccgaatatttgatgatatcaagtacctctgattaattttttaactgtgatgatggtaaggtagtttccttataaaagagttcttttaaggacaaaacagctaaatatgtaaacataaagttacttacagcatatgtattatgctgagttatttacagaagaaattaaatgatgcttgggatttgttcctctatattatctttaaatatattcattttaattgatatacatttgtacatatttatgggatacagtgtgatattttgatacatgtatatacaatgtgtaatgatcaaattaaggcaattaacatatccatcacctcaaaccttcatcatttctttatacgggtaacagtcaaagtcctctcttctagctatttgaaaaaatacaataaattgttgtttgctataagtcactcttcagatctatagaacactagaacttatttttgctatctaggtataactttgtacatccattaaccaaactctccctatccctccctgtcccctaggcttcctggtatctagtaagcactatcctactctctacttctgtgggatcaacttttttagctcccacataggagcgagaacattgcagtatttatctttctgtgcctgacttacttcgcttaacatgcagtcctccaggctcacccgtgttgctgcaaatgacaggatgtatgtaatttaaagtgtcaaatagctctgaggcttgccaggaaagaggagtccttgcttgcttcctacaattttaccttccccagagctaccactttcagttgttttggcagcctcatttgatatttgcatttaatctgtaagattttttttaacttctttttgaaaatttttaaaattgtgaatgatatgttttgaaatatgtgctatgtggaattctaaggtttttatttctttttatcttactagtttggaatatttatagatgtaccatgtgcaaagtgtaccattcacatactcccatcctgaacctccttttcttagtatagttaggtcatgattttgactaggccattattcagtgttaataggatgatgaccttgttaatgctgttcatacctgagccatgtagaattctatgaatccttttcctcttctgcaaattttttgttctatttaaactaattgttcttttgatgtttgcttagttttctatgtgctttttactcattcagtcctaaatgctcttggttatccaaatctcctcttgatatactacttcttgagtctttttgcttatcacccaaggatttcccttcacaattatgccaggaattcctttttttctccctcttgtattggactcctgtttcctgtatcacatgcctgtctttttcttagtttcctccctgttttgcagaagaacatctgccaatagaattctaggttgtcaactgccttcctcattgcttcattgtcttctagctttcagggttgctgttgaaaagtccagagactttctgattcctaatcttttgcttatgagcttccctacctgctttttgatattttataggacctttttaatatccttacagttaaaaaaacacaactcatgatgacagatgctgtgtttaatgtgggcatattgaaatcaaatgaaatgggcaggtaggcactcagtgggcccttacagtctacacagttatgaccttacgttttaggaaatattttgagttagtcatttgacttcactccaccccatttcctttactcttttattctggaattctatttggctattagacctgctggactcattttgtagccttctcatttttactgttctagtcttctttacttttttttttttaactctacattttgggcacattccttaaagttatattccaatctgtctactgaattttttttgaattcttaaaaattgacatataaaattataagtatttatcatgcacaatatgatgttttgagtatatatatattgtggaatggttaagtctagctaattaacaaatgcattacctcacatagccaccatttgtttgcggtaagaacagttaacatccactcccttggcattttccaagaataccatgtatcatcattatctatagtcactgtcctacacgatggatctgaccttattcctcctaactgcaatagatctcctgtctaactgtaaatatgtatcctttgatcaacatcttcccaactccctctccccacaaccaccccagcctttggtaaccaccactttgctctctacttctgtgggaaaaacttttttagattttgaattaccactttttaagcatcctgtttttgtttgaataagtgcaatatattcttctcgatgttactattgttaaacctattcatctgctcagattccagagagaaaaatctttccgtctcaggccttagatgatgtaagactggcttcagatgctatttagctctgataggcggaagagataataagaaagaagacaagcttcagctctgaactgcctgtatctcatcttgttcctccaggtttgggcttcaggaatttcttctattttgtttatatcagactatagggctctctgaagaatgatacatagtttgaaaaatttccccattttatctaaattgttgtatatctgtaatagttttctaaagtttttagaataaatttgtcctttccaggtttcagcattgatggtttcttctttcctcctttattgcttttcattcatctattctgttttggagacgaaggctttaaaatctttacccacgtagcttcccccagctacctctgagaagtctgttttatcttttgcatcactaattttattttttgctttttaaaaatattctactttctgcttcattggttgcttttattcaagttcataattcagattttcttttctggttagcgcagactcttttctggtctcatatttctttctatggctacctgctctatgtgcaaagattctactatgtcccctggaattttaagaacatgagctagaaattttctgttaatagagttttctcttttatatagtatatctatttcttaagtaaatatttgctctgattctagaatcttccccttttctgaaccacaatgtgttctatttgcctggtggatttagatttttctcctttgcttatccttagggaagtcaatgcatattcagtattgggtattgccttgttttctttccttctttccttctttccttccttccttccttccttccttccttccttccttccttccttccttccttccttccttccctccttccttccctccctccctcccgccctccctccctccttccttccttccttccttctttccttctgttttaaaattttactttaagttccgggatacatgtgcagaacatgcagtttcttacataggtatacatgtgccatggtggtttgatgcacctatcaactcgtcatctaggttttaagccccgcgtgcattagtaaccctgtgttctttcaagaaacttgtatgagtcactatttgactcaagtccagacaaaggaaaaaggttacatttgtagtagattaatttaccaattatgaattcatattcctgtgaggcctgggaaaagagccagcatcagctccagaagtaatcaaagggaagtttgcctcaaaccaaagactttgttttaggtatttggtgtggcactcagtaattagcaaggtcattgtcacttggcatggctgcccttgccaggagccagctgggagtgtctttttcccatgtgttttctgtggtagtcatcatggccacctgtcaaccagaagtaaatgcagccagaggttacttgcttggctggcctctgaagccaggagcccttttaaaggagttagtgtgttttaaatgtaccccaagtcttcctgggcttagttccctcagcccccatcgctctaaactaggggtatcattatgggtttagtggattctcatttgcttattttctggttgtgcagttgcagtttcacatgaagaggatatcgtgttttagtccttttgtcaacctcccatgttgattctttcaagtatatatttctgaacgtttaagatctatttatagtttccagggctgattcaggatattcctctgttataatcaactggtcatttctttggtaatctggcacaaagaaataagcttatagctttcttatttggaaattgtattttatatatgtgatttgaatcataggggggatattgaggtaatttagacccagtgattgtaaaggaatggatgaatgatccaataaataaatagatgacaaagctcatcttttgcaggtgagaggcattagaagtaattttacggttttgagaagagaaatatagatgagaagagatgcctaggagaatagaaaatggagacaactaggaaagaagagaagaggcagcagtaatgctccaagggcagttggagagcctgatgtcagctgggcaaacccacattgcttcatggttttctgtggctagaacacagttttgaagctcaagcaaaggaaatggagagttaggttggtggcaggttggaagagttgcagaacaggtatccaagtacttggaagagttcagaggggtgtatttcttagtctcactctgttgcccaggctggagtgcagtggtgcaatctcggctcactgcaacttctgcctcccaggttcaagcagctctctgcctcagcctcctgagtagctgggattacaggcgcctgccgccacgcctggctaaatttttttttttttttttttgtatttttagtagacacggggtttcaccatcttggccaggctggtcttgaactcctgacctcatgatccacccacatcggcgtctcaaaatgctaggattacagacgtgaaccaccacacccagccagggatgtatttctttaaaaattattgtgcaactctctagttgacagtctggggctctggtaaggcgggggttcatctgtcaaagtgagccagcttactcagctagatactgtatggcatgattacttagggtaatgtcttttttgtcttgtttagttcccccatgatttgtgcagttttgtaaataagttgagaagtaaatagatttcatgatataattttaagacttcactcccaagggaaaactgatttatacctttgaagttatgtcatttagcccttgtatttggcaaagtaagcatacactattttataaagcgttcttgtaatgtgtgaataggttcaacaaaattagacgtgtcgcatgttttaggagaaggcagttaattttctgtcttattttcttcttgaaaatgtgaaactaacaatctgtcatgtttattaggcatctcaaggaaatgaaacatccaaatgagggttaaatggttaaaattttatatagatgagaagagcacaaggttctctgtctcagggttactctgcaggtcatctgactttcatcctactttctccattttttatctgacatttttgcttaaagattccatgtgctttccatctctttttaatctttgatcacatggaatattggcctatggaaagagtatgaatcttagcataggccctggttcagacctcagctctttcatttcctgactgtgtgactgtgggtatgttgcataacctcacaatacctttcaaagcctcagatgtctcctctgtaaaataactgaatagtatctgcctcaaactgttggggaaatgactgctccaaagctcagcatccagtggcctttcatgaaatcccagtctcgcatctttccctctgctgttgttagtaataacgtcatatgacatatttgtttggtggatctcatccatttctatttcgctttccttactctaagctaggtgcctctatttaccttttaggcaactctctatattctgaatattgaaaatattttaaggttatattgcttaatgacatagcttggtgaaatgatattggacctgagcatatagtatgcgtccttaagaatggaatcttatgtgcttgattttttgctttttagttgtattattgatatattcatttatgtcacatagagcaagataatgtgaattataaatacataagagggagaaaggaaaaatgaaagtaggagtgggcaaaaaataatgccaggaataataaatacatgagaaggagaaaggaaaaatgaaagtaggagtgagcaaaaaatagtgccaggaataagttcagtgtaaaacagtgcaccttggggttcctgtaattttcttgggtaggtttggcactctagcagtcatcttgaaaaaagaaaggtcctgtatacatatgcttttctcctgggttgtttatttactttcacagtgtgataaacaagtctcatttgttcctaaaactgggactaaggagaatatcagtactgtcacagggcagctgtggaattcggtgttgaatgtcatgacaagataacttgagaactttactacttatttgtgaatgaaaataattgctgtaatgggagaagaaaataggaaggctctacagttctaagcagccagaacctaaagtgattgaaactgtagtatcaaggatgctgcacaaaaatgtagcaattaaaaatttaagcttatagaagttgctgtcaaacctagattatttcttagaaaagcagcacttctgagctcctcatttgcccagtatccctgagctaatatagggtagcaccaagggatgtgggggacaaaggacttggattaaatgtccaactccacagttcctgctgtgcaagacttggatcttgtgacatctggttcattacttttactattatgaaaagtatcatttcccaatagtttaaaaagtgttcaggtatcatcttgcatgttggtgaaactaggaagtatatttacagacaaaagctccttaaaggcaaggttggcttagccttactttttttttttgagacggagtcttgctctgttgcccaggctggagtgcagtggcacgatctcggctcactgcaacctccgcctcctgggttctagtgattctcctgcctcagcctcctgagtaggtgtgtgccaccacgcccagctaatttttatattttaagtagagatggggtttcaccatgttggtcaggctggtctcaaattcctgacctctaatgatctgcccccactggcctcccaaagttttgagattacaggcctgagctgctgtgcccagccaataacgtcttttgaataaaagaaaagatattgttagggagtatgaacatcaccagaaaatgaaaatgtttcttaaaaataaacgaatttttaaaaattattttcttaaaatgaaaaatttttttcttctggactaaattgcaatgtggaaagagtcacatcagcctttggacagaactagggctgtgatttactacagggaagttagcatctttctgagttttcttaatgaacacgtgaataagagatttggttatactttgtgcttatttagcatcctctctttgaggatgtcaaaaggattttagaaatgttctctttctcacacaggtggacgtctgatttatgaagctccccatccacctatctgagtacctgacttctcaggactgacacctacagcatcaggtacacagcttctcctagcatgacttcgatctgatcagcaaacaagaaaatttgtctcccgtagttctggggcgtgttcaccacctacaaccacagagctgtcatggctgccatctctacttccatccctgtaatttcacagccccagttcacagccatgaatgaaccacagtgcttctacaacgagtccattgccttcttttataaccgaagtggaaagcatcttgccacagaatggaacacagtcagcaagctggtgatgggacttggaatcactgtttgtatcttcatcatgttggccaacctattggtcatggtggcaatctatgtcaaccgccgcttccattttcctatttattacctaatggctaatctggctgctgcagacttctttgctgggttggcctacttctatctcatgttcaacacaggacccaatactcggagactgactgttagcacatggctccttcgtcagggcctcattgacaccagcctgacggcatctgtggccaacttactggctattgcaatcgagaggcacattacggttttccgcatgcagctccacacacggatgagcaaccggcgggtagtggtggtcattgtggtcatctggactatggccatcgttatgggtgctatacccagtgtgggctggaactgtatctgtgatattgaaaattgttccaacatggcacccctctacagtgactcttacttagtcttctgggccattttcaacttggtgacctttgtggtaatggtggttctctatgctcacatctttggctatgttcgccagaggactatgagaatgtctcggcatagttctggaccccggcggaatcgggataccatgatgagtcttctgaagactgtggtcattgtgcttg

SEQ ID NO: 108 - DNA, 166 bp, Homo Sapiens
cgcaaagacttccccagcaacagcttttatgtggtggtggtggtgaagaccgaagaccaagcctgcgggggctccctgcctttctaccccttcgcagaagatgaaccggtcgatcaagggcaccgccagaaaaccctgtcagtgctggtgtctcaagcagtcacgt

SEQ ID NO: 109 - DNA, 87 bp, Homo Sapiens
gtccttgatataccttggagtggggacttgatcctgggggcaagggccttggagttagggacagggtctgggtattgactctgggag

SEQ ID NO: 110 - DNA, 363 bp, Homo Sapiens
gtctccttgacagtatcttgtcagttattcttcatcctagcatttctgttcgactagcagcagcttggtgtttacactgcattgccgtggcattaccctcctacctaacaccactcttggatcgttgccttgaacggcttactggacataagtcttcacctgaagcagtgactggcttcagttttgctgtagcagctttgttgggagcagtaaaacattgtcctttaggaattcctcatggaaaaggcaagattattatgacattagcagaggatttgctgtgttctgctgctcaaaacagtcgcctttcagctcagcgcacacaagctggatggttgctgatttctgctctgatgacattag

SEQ ID NO: 111 - DNA, 8827 bp, Homo Sapiens
gtatgataaagaagagtcgtatcagctgagcagtcgtgattgtgattacgtccctggttgactcatgaataggctttgtaatggtagattgttcttttggacacacagccaaaatgctaaagttcactgggaggtctgctaaggtttcttacatcaaagagttgacagagaaaggacttactaaatgaaaatattatttagtagtttctagtaaacctcaaaaatcattccaaattggaacatacactatgtttacatgtttaagttaaactgagtcatgaagctaaccgctgacttatgtatgctttcagtagtgttgcctaaatgttctcatttaaaagaagtccccataggattatgagatgtgcatatagtacgatgtcatttttcttgggaaactttcagcttacttatgcaataaggaaatttaaggttatttgagtacatctggaataatagctttagatttttgaaatttgtctttttggggtagcttttccctttttccttgacaaaatataacacaacgatcctatttcctatctgagcttagttcaagttgttatggtggcaaagttatgggttatgaaatgcacaaaaatgttggtacagatatacttagtggcccacttaagaaaataaaattggagattaagaatagagggaaggttgtggctccttggtattatgttaggagagcagaatccattatagtacactcttacagttaaacaatttggggacaaacaaggttatgtgagtgacaaagtttcataaaactagaaagcactatgttaatgcaagattactttctaaaaatattgattctcattcccttttttccttcacaataaaattgtatggaacgtttgtgtggtagaacaaggagaacttctaaagtgcatgttagtttcatcctttgaggccacatattgggtctcctgcattctatctgtttactctctataagatttaaaacagagagagtaaatagacagaaggcagaggacccaactatgtggcctcaaaagatgtggccgcaaaggtactatgaaagtatttagtagtagtattatagggaccagttactccagatttgtatgaacaataatccccagttttgatgcagcatcatgttggaggtgctaagtaggatccttagacttattagttgaatctcataatcaccaggagattaacatactattttatgaaaaatggagtgtgaaaagtccttatggactgcatagattctgaatttttataacattaatccaaacccagtaccttgtctgtaattatagtctaagcatatgtgatttgattaacacatgaattacaaatatatctacttttgggcatatattccaccttttgtactgcagaaaaattttgtgcctttgcagtgaccaaaactatagttctgtttagggtcagtaggagaagatgtttttcctcttaactgagaagtttatgtttcttggaggaaaatcctagaataaatgcaatcttccattgtatcttggaattacaatttgaaatatcttaaacagtattttttgaaagaattggttgtctactgtatgccttttccaatgaactacataaataaaggcagagtttgtcttttcctctctgaatgcctaatgcctagaacggtgcctgacccggagtagttgctcagagcacgggtgctgaatgaggaaataaagatgatactgctatctctcatatttcagatgctattttgtagtcttcggtcataattttagaattctctttttggtaagaggtttaaaaaagtgagatattatttgatttttaatgaaatgtaaacatatagcagatgcacaataaacatttattaaattaaaaaaccactgaaagaaacatgatactgcaatcctggtgctttatttactattctaatcatctgcagtcctccttttgcatctaatgagagtcattttttaaaaagctcattgacctattgatttataaaagaagacatttagcaaagctgaagagaggcccacttgaatttgaatcattttaactagcacttaaccctcggcctttacctagcggtttgagatccaaagattctttgagttcttaaacctatttcatatttgattatttattgcctaacaggatctttgatgctacatttgagcctttctggacatcgggattatggattaaaagccgttgctgtacctcctgcctggtactgtgcctgacatagaatctaggggctccacagggctggctttgtgggcatgtgacctgtgcatttgtacagggctccaagctcagaagggccctgagtttggttaaatgatctgctgtcactgtcttgaaattaatttttgagcaaggggtcttcatttctctgccttgctaaggtacaactgaataaaaaacagatggtgtgtttgggatccatccaatatatcagctgaattggaatattatttggaatcaaatatgaatggttttgaaaaagaaaatcttctccaaagtgttcaatatatcctcttggttggaacataggagactgtagctaacatcatgggaaatgtaataggtagcattaactatcttatttttaaatgtcatctgttatattttggttcatgcttatcttattgttcactattaaatatttaactatgatttttttttttttgagacggagtcttgctctgttgcccaggctggagtacagtggcacgatctcagctcactgcaacctccgcctcccggattcaagcaattccctacctcagcctcccgagtagctgggattacaggctcccacaccatgcctggctaatttttgtatttttagtagagacagagtttcaccatcttggccaggctggtcttgaactcctgacctcgtgatccacccgccttggcctcccaaagtgctgggattaccaggcatgagccaccgcgcctggcctaaatatttaactatgaaatatttatctcttattagtggtataattatttactaattactgatttattgataatagtttgttagatgttgggcaggatttatattaaaacatgtttgttttctggagagtactatgtgtttacatgaatacatgttcatataaatactatgtatttacatgctcatacctgcaaaataataggaggggacaagacagtcttttaaaaaaatctgtttttgaggctctctagggatgtacatgtcataattcttattgagagccagttctgttgcttaatcagacttacgactggaaaagaggcagtcagtatagagtgggaccagaactgtgggtttcagagctggcctgcctgggtttgagtactgtctcagttactttctagttgtatgacttttggcaagttacgtttatccccttagcctccagtttgctcacctgtaaaaccaggatacctcttaaaacagaccttatgaggcttaagtgtactaatatatgtaaagaacctggcctgtagtaagcactcaataaatgaagttcaggccactctggatcttactattttaaaattttagaagtatacttatgtggttatttatatttattaaatgcgaattgagagcttgtgttgctatccccaaagacatcatagtcaagtgaaggaactgacttgcaattcaagaattaggatattggaataagggcaaaagtagggggaagcagagggtgaaaagaagggcctggagggagccctaaaccctccttgcagaagaggtgaaatggaacattccctcagctgcctttggaaggatcaatagacactgagagcagcttggtgagagtttgagaatgcatagcgagtttggggaattttaaagagcttagtatggctgaagtttaaaggacaagtgggagattgtgaaaaatgggatagagatgtaagatgaggccagatcctgaggatgtggactagacagagaggagggaaatgaagaaagggtcacgtcttagacgccaacgaagttgagtttccccaaagagactttaagcatcaataccatatgctatggagagttaaagtaagataagaactgaaaagtgattaataattaggaaattgccagcatgtagtttgatctgtcatttgctatttagggtgattgactatttaagctttgattttttggaagtgtggagaaggcatttaaattagatcccaaagtttctgactaaaggaggattaagtcctagatttccaaaagaagtttgacaatcctttttctccctagttatttcagattaggattaactactggtttgagattaatacatggcctgacttagattgtaatcctcagtagggatagggcaatagagccttattttatcactgtaacttcagaatctgatgggatttttagttcataatgataattaaaaaaaactgaacatcagacacttttctaggccttttacatgtgttatcctcttttcttttgaggaagagtctcattgtatgaccaaggctggagtgcagtggcacaatctcggctcactgcaacctctgcctcctgggttcaagcgattctcgtgcctcagcctccccagtagctgggattacgggcgtgtgccaccacgcctggctaatttttgtatatttaatagagacaggtttcgccatgttggccaggctggtctcgaactcctggcctcaagcaatctgcctgcctcccaaagtgctgggattgcaggcatgagccaccttgcgcagcctatcctcatattttaaactttttatttttttgaggcagggctgtttgtcttcttattttatagatgaaaacattggaagggcagagaagttaaatatcttgtcacactgctagtatgttttggagcttgcatttaagctccggcagtcctaattcaaatcccagactttcctttttcatgacctgcttccttactgtagctctgtatacttttgctgaatgaatgggtgaatgaatgaataaattggggaaaagagatggatttatatatgtatattgagacaggatctccctctgtcactcactgtggattgcagtggcatgataatagctcactacagcctctacctcctgggctcacaggatcctcctacctcagtctcccaagtagctgggactacaggcatgtgctccgattcccagctaattttttaaattttttgtagaaatgggttctccctatgttgcttaggctgatctggaactcctgggctcaagtgatcctcctgcctcagcttcccaaagtgctaggattacaggcgtgagctaccatgcccagctggattaataaatttttaatccagactagtcaatctgagtataaaattttttatctccttgttggtggtaggtacctcatcccttttattttcttcagagaagtttattcttctgttacctatgtgataacccagtttctaaattcttgaagcacaggatgtattcaggaattgatttcctggaaattagggaatttccaaatgagttctccctcctgctatccaataagaatcatggggtcttggttgtcagagaggaggtctttaatacatacctgtgatctggatagtacaagagtggacaccatgggtagggctcaaagtgaagccgtgagccactcacatgtgacggaggactagaccaaggtcagctctgcatgccgaacctttggaagccaggacttcggagatacaaagataattgagtgacaaataagtagcagaatatgatagaagggatattatccttacatttgtgactttctttttagctataactctgtgacctcatgtagcagttacacaaaatgggactgtaattcttacctttggagataaaatgagtggtagaggaaatgtttgttaggcttatagaaagatccaactattgtgtctttacagtgaacagaataattaaatttgatagaaccaactttcctactgtagaaatacttttaaagagtttatgtctaatgacataaaaatatacttaggaagctctgtttttatacagaacaaagtgtttttcaacttctgttgttacatcctttgaataggttaacatttctgcctcctttgaggactaattctttgctctttcacatcagtgaccaatgtagagtttgcttacatttatttacaacttattaacattactagagaatagcacactaaatgtgaaaggaacagagtaagatccatgtcagggtatcaatttttaactcaataaacttatcagtattttaaaatgccattttattatttttgcatagataatgtatattacatcttatttcccaaagcttaataggtttgaacatggactttatttgtagaaggcaagtaagagaataattataaaagtgggtctttgagtagaataatgataaacttacccatattataaactgcattgtgacatgtgtttccttaaaagaaatgtgattctgaaaaaaatggcttgcatttgagcctgttactgtggatctagatttgcatagaagacatttatagacggttcctgatgtgagctgcagctggtaaaatacattccttacctatccaataagaatataataggatttgctttttaatttttaaagcacaattatgagtcaatattaaactataaagctctgaaactggcgaagaaaacaactatcaggaagagagcagagaatattttgtatatttcacataaagggcatttaacagtattgttttctaattttactttcatttcaatctgtttcattaaaaaagaaagttgaggtgacttgcctcaaaggtctgtataatttttgaggcttctttctgcttgtgtttttgtaactgtaaaataagaacacactgaaaatatactggaattaagaaagccagtgtgagtaaatgaaaatgtcattaaatttggtagagaaatggacagatgggtaacttgaagttaataaagacataaaaatcaaagcagaggaatgtattttaagaataaaacaatctaaattgcatgacttaaaagtaaaataataggtagggaattatgtattataaagaaacatgaatgtgttaactacaggtgttaatatttaaaaaattatagtgggaatgatttgagacttcagacttggttacatggcttttatgacatcactttttctaaattactggaacctgtgtttgtttttacattgttttgtcatgagaaaatacatttatataacttaaggctcttttcttcatttatttcaccttgtcttcaaaaaattacatgaacatgttaattccctaaaagtgaaagaacattatgttttttattggtgaataagaagttgttatggagtgctctatttttacagctgtgcttcagggtatcacgttctgaagaggtcagcttggcatgatctcatgggcatgaatcttggggagacaattctgaaacagccacaggcctgcagcgtcgcaaagccagggcagaacatccagatgctgtcgttaggaggcacgcggtctgttaaagctttagggtggaagacccaggaggatgcagctagaagaatgccgcatccacattcgtggcaactctctgggtgctgttgtaattttaactcttgagttcgtttaatagaaaaaaataaatagggctgtgagcgatggctcaggcctttaatctcagcactttgggaggccgaggcaggcggatcacctgaggtcaggactttgagaccagcctggccaaaatggcgaaacccggtctctattgataaataaataaaagtaaataaaataaatagggaaaaataaatagggaggcaaagaggaaagggtttaaggaaaaaggtattgagactgaagtagagagactgttgtagtttcatttcatggctcacattgttcttgttaaaattacagacgctccgggcatgatggctcacgtctataatcccagcactttgggaggccaaggtgggtggattgcttgaggtcagaagtttgaaaccagcctggccaacatggcaaaacctgtctactaaaaatgcaaaaatcagccaggcgtggtggtgcatgcctgtaatcccagctactcgagaggctgaagcaggagaactgcttgaacccaggaggcggagcttgcagtgagccaaggttgctccactgcgctgtagcctgggcaacagagcaagactctatctcaaaaaaaaaaaattacagaagcatattatgattttgagaaggtgtgtgaagctactttctttatctgataagagcagaatttctgtgtctaatactcagatatgattatttgggatatggattagctttgctgtctttactttggactcctctgtatcaaggttggtggtagttgagtacatgtgtgattggaagttgtttataagattcttatttaatgaaataagttaggaaatactaatgttttcccatcatttagaaatatgtgttatattttttatcatcactgttaatgagaataaaatttgttatttggaatccacatgcactcagaattgttgccacttgcagcgcatgtagtgtcacgtggatgtcattcatacctgttgcttctgctgcggtggctgtcactgtccctatgcagagctgtaccaagatctgcactcagaaaattttgaggaaaattgtttaatagggttggagtgatataacctttgttttcattattcatgtattctgtttttcatatcaagtaggttgatgatcctggagaagagagaattagtgataaaaccaacagtacatcacatacgaaact

SEQ ID NO: 112 - DNA, 264 bp, Homo Sapiens
ccagcacaggccagccagaagatgacactgagactacaggtttggaaggcggcgttgccatgccaggtgccgaagatgatgtggtgactccaggaaccagcgaagaccgctataagtctggcttgacaactctggtggcaacaagtgtcaacagtgtaacaggcattcgcatcgaggatctgccaacttcagaaagcacagtccacgcgcaagaacaaagtccaagcgccacagcctcaaacgtggccaccagtcactccacgg

SEQ ID NO: 113 - DNA, 136 bp, Homo Sapiens
ggccagccaagtctcgggatggtgaacgtactgtctattgcaacgtacacaagcatgaaccccttgtgctgttttgtgagagctgtgatactctcacctgccgagactgccagctcaatgcccacaaggaccacca

SEQ ID NO: 114 - DNA, 211 bp, Homo Sapiens
gtttggcttgagcaacaagtttgatactgaatttccctccgttctaacagggaaggtggccccagaagaatttaaaaccagcattggccgtgtgaatgcatgtttgaaaaaggctctcccggtcaatgtgaaatggctgctgtgtggttgtctctgctgctgttgcacactgggttgcagcctatggcctgttatttgtctcaacaaaaga

SEQ ID NO: 115 - DNA, 1061 bp, Homo Sapiens
ggccgcgatgggcctcttggaacattggtgtgttcatctgcattcgatgtgctggaatccacaggaatctgggggtgcacatatccagggtaaagtcagttaacctcgaccagtggactcaagaacagattcagtgcatgcaagagatgggaaatggaaaggcaaaccgactttatgaagcctatcttcctgagacctttcggcgacctcagatagacccagctgttgaaggatttattcgagacaaatatgagaagaagaaatacatggaccgaagtctggacatcaatgcctttaggaaagaaaaagatgacaagtggaaaagagggagcgaaccagttccagaaaaaaaattggaacctgttgtttttgagaaggtgaaaatgccacagaaaaaagaagacccacagctacctcggaaaagctccccgaaatccacagcgcctgtcatggatttgttgggccttgatgctcctgtggcctgctccattgcaaatagtaagaccagcaataccctagagaaggatttagatctgttggcctctgttccatccccttcttcttccggttccagaaaggttgtaggttccatgccaactgcagggagtgccggctctgttcctgaaaatctgaacctgtttccggagccagggagcaaatcagaagaaataggcaagaaacagctctctaaagactccattctttcactgtatggatcccagacgcctcaaatgcctactcaagcaatgttcatggctcccgctcagatggcatatcccacagcctaccccagcttccccggggttacacctcctaacagcataatggggagcatgatgcctccaccagtaggcatggttgctcagccaggagcttctgggatggttgcccccatggccatgcctgcaggctatatgggtggcatgcaggcatcaatgatgggtgtgccgaatggaatgatgaccacccagcaggctggctacatggcaggcatggcagctatgccccagactgtgtatggggtccagccagctcagcagctgcaatggaaccttactcag

SEQ ID NO: 116 - DNA, 322 bp, Homo Sapiens
gtaaaatctactggtggagtcactccattattcttatctgtggagatctagatcttgatttgaaagtttctgagaaaatcttcagctcagacttgagggtcaactttaccagctgaaggatctgcatttactgctcaaccacatctaatttgatgtcctctgcagatttaaaatgtgtgccttcttttccgtcaccaagtcatccctgggttactactgaacatccttctcaattccccccgacccatggatggctgttctccattgtctgtttcaccagatgtcctcaaaacaaacagacagaagaaggaagtggctaatg

SEQ ID NO: 117 - DNA, 252 bp, Homo Sapiens
gccgagcagctgcgcaggcagaaggtggaggaggacaagcggcggcggctggaggaggtgaagctgaagcgtgaggaacgcctccgcaaggtgctgcaggcccgcgagcgggtggagcagatgaaggaggagaagaagaagcagattgagcagaagtttgctcagatcgacgagaagactgagaaggccaaggaggagcggctggcagaggagaaggccaagaaaaaggcggcggccaagaagatggaggag

SEQ ID NO: 118 - DNA, 718 bp, Homo Sapiens
tggtggtctcaaggaaaacgacgtcataatcagcatcaatggacagtccgtggtctccgccaatgatgtcagcgacgtcattaaaagggaaagcaccctgaacatggtggtccgcaggggtaatgaagatatcatgatcacagtgattcccgaagaaattgacccataggcagaggcatgagctggacttcatgtttccctcaaagactctcccgtggatgacggatgaggactctgggctgctggaataggacactcaagacttttgactgccattttgtttgttcagtggagactccctggccaacagaatccttcttgatagtttgcaggcaaaacaaatgtaatgttgcagatccgcaggcagaagctctgcccttctgtatcctatgtatgcagtgtgctttttcttgccagcttgggccattcttgcttagacagtcagcatttgtctcctcctttaactgagtcatcatcttagtccaactaatgcagtcgatacaatgcgtagatagaagaagccccacgggagccaggatgggactggtcgtgtttgtgcttttctccaagtcagcacccaaaggtcaatgcacagagaccccgggtgggtgagcgctggcttctcaaacggccgaagttgcctcttttaggaatctctttggaattgggagcacgatgactctgagtttgagctattaaagtacttcttacacattgc

SEQ ID NO: 119 - DNA, 190 bp, Homo Sapiens
gaccccgagggtaagcccaagaaggccaacatccgggacaatggggatggcacgtacactgtgtcctacctgccggacatgagtggccggtacaccatcaccatcaagtatggcggtgatgagatcccctactcgcccttccgcatccatgctctgcccactggggatgccagcaagtgcctcgtcacag

SEQ ID NO: 120 - DNA, 743 bp, Homo Sapiens
agatgctgcccccacccccttaggcccgagggatcaggagctatgggaccagaggccctgtcatctttactgctgctgctcttggtggcaagtggagatgctgacatgaagggacattttgatcctggtgaggagactgaatcatgggtccctgagggccagggcttgggaggtagagagttgggggccttgacctgttacatgcctgctttttactcagccaagtgccgctatgccctgggcatgcaggaccggaccatcccagacagtgacatctctgcttccagctcctggtcagattccactgccgcccgccacagcaggtacttggcacacctggcacacttgtagctgccccgagaggagctcctgggacctctacttcccctccaacccctctgcccatgccagtgaaacccctgcaggctgagggggcaaatgaagtggggtttaaatactggagatggaggcagacctggggccagatgttctctgtgcccctcttcaccctcaggttggagagcagtgacggggatggggcctggtgccccgcagggtcggtgtttcccaaggaggaggagtacttgcaggtggatctacaacgactgcacctggtggctctggtgggcacccagggacggcatgccgggggcctgggcaaggagttctcccggagctaccggctgcgttactcccgggatggtcgccgctggatgggctggaaggaccgctggggtcaggag

SEQ ID NO: 121 - DNA, 1059 bp, Homo Sapiens
cacccccccaggcagcagcagcagttttcatctctggatgacaagccccagttcccaggggcctcggcggagtttatagataagttggaattcatccagcccaacgtcatctctggaatccccatctaccgcgtcatggaccggcaaggccagatcatcaaccccagcgaggacccccacctgccgaaggagaaggtgctgaagctctacaagagcatgacactgcttaacaccatggaccgcatcctctatgagtctcagcggcagggccggatctccttctacatgaccaactatggtgaggagggcacgcacgtggggagtgccgccgccctggacaacacggacctggtgtttggccagtaccgggaggcaggtgtgctgatgtatcgggactaccccctggaactattcatggcccagtgctatggcaacatcagtgacttgggcaaggggcgccagatgcctgtccactacggctgcaaggaacgccacttcgtcactatctcctctccactggccacgcagatccctcaggcggtgggggcggcgtacgcagccaagcgggccaatgccaacagggtcgtcatctgttacttcggcgagggggcagccagtgagggggacgcccatgccggcttcaacttcgctgccacacttgagtgccccatcatcttcttctgccggaacaatggctacgccatctccacgcccacctctgagcagtatcgcggcgatggcattgcagcacgaggccccgggtatggcatcatgtcaatccgcgtggatggtaatgatgtgtttgccgtatacaacgccacaaaggaggcccgacggcgggctgtggcagagaaccagcccttcctcatcgaggccatgacctacaggatcgggcaccacagcaccagtgacgacagttcagcgtaccgctcggtggatgaggtcaattactgggataaacaggaccaccccatctcccggctgcggcactatctgctgagccaaggctggtgggatgaggagcaggagaaggcctggaggaagcagtcccgcaggaag

SEQ ID NO: 122 - DNA, 304 bp, Homo Sapiens
atgtggccaattagatagtgctttgcatgaagctactagtcgcgtgagaacacttgaaaataagaataacttactggaaattgaagtgaatgatttgagagaacgcttcagtgcagccagcagtgcttccaaaattttgcaggaacgaattgaagaaatgagaacaagcagtaaagaaaaagacaataccatcattcgactaaaatctagactgcaagatttagaagaagcatttgagaatgcttacaaactctcagatgataaagaagctcaactaaaacaagagaacaaaatgtttcaagat

SEQ ID NO: 123 - DNA, 287 bp, Homo Sapiens
aacagaattcagccatggccccaagaaagagaggtggacgaggtatttcattcatcttttgctgtttccgaaataatgatcacccagaaatcacgtatcggctgcgaaatgatagcaactttgcgcttcagaccatggaaccagcattgcccatgccccctgtggaggagctggatgtcatgttcagtgaactggtggatgaactggacctcacagacaaacacagagaagccatgtttgcacttccagctgagaaaaaatggcaaatatactgtagcaagaaaaag

SEQ ID NO: 124 - DNA, 4715 bp, Homo Sapiens
ctggttcctccctctttgaactcatctggatctggcttatttcaaatgtccatccccacaggtgagaaagtagtttagattttagaatcaaacagtcttgatttagagtcttagttctgcctctttctagctatgagatttttaggtcaatgacttaatctttctatgtctgtatttttatacctttaaaatgaggtactgttaatacctacctcatgggatgttatgaatattaaataagtttatgcaatttgtttatcataacattgtttaataaatgttagttataattgcttttagaattatcatgtaagataatacatagttatattaattattattgtcattatgctaagattttggaaactttcattggcatacaccaagatgatgattggcagttatttcctgtctttacctgcttggttccttaatatgcacagatagaactgtttaagatttgaggcctgggacactgcattcaacttccctcaccttgcaaagtgctctggcatctgccttttgttatagactttctccccttgtcagtgtaaatactgaaggctatacctgtgcaatgctctgaatattggtgtctcccctaaattcctgtgttgaaacctaatagtattaagaaatgagacctttggggaagtgattaagttatgagtgttctaccctcacaaatggaattaatgctcttataaaagattgcagggagttgctttgtccttctgccatgtgaggacacagcaagaggctcccttttaagcagagaacatgccttcaccagacactgaatctgctggcattttgatcttggacttcccagcctctagaactatgagaaataaatttccgtttcaataaattacccagtctaagatgttttgttgtagcaccctaaatggactgagacagcgtgtcacaattacagacactgctgcactgagctgtattagtgtcttcttttaaccccagacttactttttattgattgtgatttttccatcacttcttggctttagataaaaataactaatatcatacaaatgtcatcaacaaatcagaagttctttcttatctgtcttctattctttgaccctctccttctatattcatatgctgctggctttctggaagctacttttttctcagtaaagaaggtacaaggtttaatttttagccatttcttgttctgaagcctgaaaattctgcaaggctaaaatcttgagtcaccttcattacctttacatttacattcattgttgattatttatttagctcattactgacatgaggagacagaatctgtgtttattttaaataaactctttttttctcttttgacaaatacaaatataagtatagacctatatattataattaacacctctgcatccattcctaagtactattatatatttattcttagaatatttggcttaaaagtttcaaaactgattcatgaaagtcaacgtgtatatttgaaatgcaaatgatatttatattatgataactgagaaaatgaattacctcttcacaaacttgagataatccatttttattgcctacgtcatttgaacatatccatatggtacaggataagaatagcagcttgttcagtacaagaatgactatacctagaaagacttcattttatcttgggtaaataatacatatttagagacatattttattacatatgagaaaattttagtagaacccttcatgtttgggcttctgagttattaaaagagtgaaaacaatagttgtattttctctaaatcgtcaactaattaagtcaaatgagtttgctataattacatgtataatcctcctgttctcgacaccccccatttttgcctgatatttaaagtaaaactgagacaaacagctgctggattggattatctactttagtattttattccttttttcctgagagaatgcaatctaaatttaatgcacaattcaatgtacattttagtgcctgctgtgtatggggaaatagacttggcatggggaatgctcagaagctcaagataaggcctctgtaccacaagataaagctcattgtctaatagatagacaagtaaacctctatgagatcaggtaagtaaatctcctggcagggaaaataccaaacatattctaaattcaaacttaaattatttgaggaatatacattcttattttctctgttagcattgttactcatttaattcaattccagacactgttttggggcagatattaaatcatattctggtattacagctggataaaaaggtcaacataatgccttcttactttatcaggaaagacagataattaagaagtatttatttaagataagtgtcaggacccaaaaacattaaataccaaggcagtatgctttaaatagcatggaataactatgttatttaaataaaataccattacaacaaataattacaagaaggaagagtactcattacataggacacattatggacctttcataggacaaataaaataataagatatcgctgattaagatatcattgatcaagttaagttagctatttaaggaatacttctataaaacaggctttttttcttatggtaaaattcttacagttttcaatggtacaccattactttcataataaagttaaacttttctgcttaccacaggaggccattgaggatatggcctttcttttctctagattcatctcttacaattttacaacagttttttcataccctccttgaaatctaaactactttcacattagctcacaatgttgtgtcatacctctcttcatgtctatgtgctgtttatttacgtagcgttattcatcccacttatgtttcaagacttagcactattttctctgcaaaatcatccagatacttccaatttgatttaaatttttctcttctgttcctcagcacaccttgtgctacctttatccaagcttttcatcattctcttggtttacttccatatattatatgctccttgaagtattgctgtatcttaacagactttatacttttcaaaccctggcatatagtacatactcaatatcatatggatggatggctagatgaataaatgaatggatgatgattttttcattttctatatcaaatgtataaaatagaaattagatataatgggctgagttcatcttaatttgcattttacaatgtaaatagaacctttaaaaatatccagactactctttaaaatatgttataagtctagatctaagttctagagagaattgtggagtaaactgcagattcacttcctcagtaaatatttatgtaaaaatgttaagtgataaaccctgattagatataccatatcctatcttccagtgggctcaaaatccttattactagcttttactatggagaaaatagttttcactactatttccacttgcagtcctgtgccaagggaatctatatgggaattatatatattatcactagaaaaagatgcttagatactctatattataaataaactttatttttgaataatgctagtctgtgttcactgaatctaaaaaattcatgtttggcatgcgaactcctatgcataaaaattttttctttgaaagaaaaagtatactatataatagtaagttattaaataataattaaagatattataattcatgcaataatttaaaaagtactttttatatgaacacgtttatttattttgtgtcttctagcatttttcatatcagcaagtatattaaaaggaacatattgtgtgcaatgtgtaggcccagcataggattctctatactaaaattttgcaatgatactggttaattgctagatttcaggcacctttctaacctccgttttagtgggcgtgaaagggtttatttgtaagagatattgctgctgacagaattttatctttttctcctggaatctcaacagctactcccatgttataggcatccccagaaagctcgtcaaaggatttccacccacactctgaatatctaacagggcttgacccattttttttctcttattaatatagcagcatacacatcatgaagaaagatagaataaggactagtctaagcttggaatttcaaaaagttatctacctgtatgcaaggaattgaagcggacaacaaaattagcctttattttttttaaattatgcccttaacaatcttatcacattaaggaatcacatctaatttcatgaatatagttttattttgagtggcttaaaatgtacatttgtattcttaaatgcagtgactataaatgccaaaaatagagtgcactataaatcaagaattctcctttgtactcaattgttaggacataatatactgcttaataacatagataataattaaaagaagactaaagctttgtgtagtaatcctagtagaatcctttggaagtaatcaggatagttataagacagaatatgaaaatacatgtttaaagtaattttcttgtataaatgattacatttttttgtttatgtaaaccattagctttttttgttttccttatgtaacatctaaatactcatatatagactgtggctaattttttttttaacttaaaggataaaagtccctctggaatacaacactgcattcagttttccctcttaactctctattgttgggcaagttactccaagcctcagtcacctcatcttccttatg

SEQ ID NO: 125 - DNA, 3022 bp, Homo Sapiens
gctttgttcttcctgttaagtggtgtactggtgatgaccggaagcatggccttgattgttttggattgggtacacaatgcacctggaggtggccattaattggcaccactcaaactcaaactcagtccatctgatgccagtgttgagtaaactcaactactatgaaatttcacctaatgttttcagtttcacttccttttgaagtgcagattcctcgctggttcttctgagtgcagaataagtgaacttttttgttttgttttgtttttttaagaaacttatctgtatgttagaaatggatatgaacaacaaaaccacgagtctcgggttaagggaagtgacaattttattccattccagagaatggacaaactcttaacttttatcaagccacatgcttggctgtgtcattgtttaacttggatattttatgattttacttgaatgtgcctaatggaaccatttgatgtgagaaacaattctttttaatttacagcaaaatattgaataaccattgacaaaaacactattattttttgtaccaaaaatacttaaagacctcagaagcactcttttacttttaagaaattgcttttttgaactttattcagaagcagttatcaataaattccataaaataatgtcattggtatttaaaaatgaatattaatataatgaaatggtttgcctttttgtaggcataataagccaaatacttttttacccaaaataatttttagagaaaatgatgtaatgaaaaattgtaccatgaattaggagcatagttttttccatttaaacgtcaccattacttaaaagatgattgattattgctataccaaatcagatgaactctgttcatcacttttcttctctgtccccaaacaatttggttcattcagactgaaatgtttgtgtcttcaacttattagaatggaagataatgcagatatttctgtgggaaataaaataactaattttgaggtaccaaatagtgcaattgggtaaaacagggtttattcagttgcatctgtctccagtgttgtattgacagctctgggtctttttttgggccagcccttttttgacattgcttccagcagtggaaaatgggcatttgatggcaataggccaaaattattgtgtccagagagtacactttttcaaaatgctcacctactggaagtgtgaattacttgacaatgtatggcttagttgtgttcatgttttgtctacagtagaggtctaatccacaggttacacctatgtttgatatgatataagttctctttgcgtaggccactgggtttctcatgcagtaagctttataaaaactcatttgcactggactgtcatctcattcttgtacaacgtagaattacttgtttacatccaacaaatggttagctagggaaaacagtgcaaactgagtgttagtagtcattttggtccaactgcatgtcaacccttccatttcaatcccagttagaaatgaaaataattactttgaaacttggctttaagagcacatttatcgtacgtcacagtgtatggtgaatatattattaaataatgtggtacttcgctcatcaggcataatgtctaaaatctaatatacataattccattaagtggttgaaggaagcaaataatggaattgtcaattggtcatctggctgtaaggtttgcccttgaactaaaaatgttgtttggggcaagggccagaaatgtggagacatggtttttgttacgcattcttgtattatatgtgactaaatttacaaacaagatacatgtgtaattaaagacccttatggaactggaagacgtcttgtagtgctacattgggtgaaaccgttggtccatttttgtctgtttctatgaagataaaataattgggggccatctagaaatagaaaggcagtgggaagacagattctacggcactgctttcatttaattgggctttaggcactccattcgaatgcagaacctcacctctagttgagaccaagaattggcaaatttgcatgagctcctggaaagagttgctgactttgtatctaagacctgccagggaataccaagagttgtttctacagacttttttttttttttttgtatgggagaagatactgtggcaaccaggaaggaatggaaaaaaaattcttttctctacagcaaattaatgtgaggaagctcctccaatcctctggctatttaaggttcaaaatcaagtgcctagggaaaattccaatggatgattttctgggagctatcttgtctaccttgaggttcctgaacaatgaattcccattaatgagcagtcttcagtattaaaaccactgtcttgtcacctcattttgcattactgtcttccgtggatgtttcagttacaactgtaatgttatttatagaacaacattaatccattaaagctaacctatttttcaatatttatgataatctatgtacatatattgtctgtccatatgtatttgtaaataggttgtatataatgtcaggtttgggtcttgggttcaagtgtatatattcctgtaagtttcttaactgcattttgatgaattcacattatgtaactataagaattgtcccaaaagtacctgtacagaaaattgaatattgaaaaattgacaaattgtgtacaaacactaaaaaaaacttgtttaaattgtatttgcaataaacaacatcaaattttttcatgaaatcttggtacaaattcagatctcttatttaaaatttaaataaggaatacattttcaaaatgcagtaatcaaaatgtgatctagtgtaatgaaataaaatgtgatctagtgtaatggaagacctttgagaacctgggtgtattaactttgtgtatatagtgtaaatatccccactgtactgttagaggccaacaattctagtatggcttgttggcaaagagtgctacaccgtttcaatgaaacaatgtatgtttgttttaactgaactaaaataaatacatgcttaatcctga

SEQ ID NO: 126 - DNA, 1435 bp, Homo Sapiens
aactccatccggcacaacctgtcactgcatagtcgattcatgcgggtccagaatgagggaactggcaagagctcttggtggatcatcaaccctgatggggggaagagcggaaaagccccccggcggcgggctgtctccatggacaatagcaacaagtataccaagagccgtggccgcgcagccaagaagaaggcagccctgcagacagcccccgaatcagctgacgacagtccctcccagctctccaagtggcctggcagccccacgtcacgcagcagtgatgagctggatgcgtggacggacttccgttcacgcaccaattctaacgccagcacagtcagtggccgcctgtcgcccatcatggcaagcacagagttggatgaagtccaggacgatgatgcgcctctctcgcccatgctctacagcagctcagccagcctgtcaccttcagtaagcaagccgtgcacggtggaactgccacggctgactgatatggcaggcaccatgaatctgaatgatgggctgactgaaaacctcatggacgacctgctggataacatcacgctcccgccatcccagccatcgcccactgggggactcatgcagcggagctctagcttcccgtataccaccaagggctcgggcctgggctccccaaccagctcctttaacagcacggtgttcggaccttcatctctgaactccctacgccagtctcccatgcagaccatccaagagaacaagccagctaccttctcttccatgtcacactatggtaaccagacactccaggacctgctcacttcggactcacttagccacagcgatgtcatgatgacacagtcggaccccttgatgtctcaggccagcaccgctgtgtctgcccagaattcccgccggaacgtgatgcttcgcaatgatccgatgatgtcctttgctgcccagcctaaccagggaagtttggtcaatcagaacttgctccaccaccagcaccaaacccagggcgctcttggtggcagccgtgccttgtcgaattctgtcagcaacatgggcttgagtgagtccagcagccttgggtcagccaaacaccagcagcagtctcctgtcagccagtctatgcaaaccctctcggactctctctcaggctcctccttgtactcaactagtgcaaacctgcccgtcatgggccatgagaagttccccagcgacttggacctggacatgttcaatgggagcttggaatgtgacatggagtccattatccgtagtgaactcatggatgctgatgggttggattttaactttgattccctcatctccacacagaatgttgttggtttgaacgtggggaacttcactggtgctaagcaggcctcatctcagagctgggtgccaggctgaaggatcactgaggaaggggaagtgggcaaagcag

SEQ ID NO: 127 - DNA, 1311 bp, Homo Sapiens
gctcacttcatggactcactttgcgtgcttgttaaatgtgctgtgttgctcccaagaccatgtaaagcctactgaccactaacctccctcacagcagaaactagacgtcaggttaaaatgggcaactccgacagtcagtacacccttcaaggatctaaaaatcatagcaatactattactggtgctaagcaaattccttgctccctgaaaatacgtggcattcatgcaaaagaggaaaagtcattgcatggatggggtcacggaagcaacggagcaggttacaagtccaggtccctggcccgaagctgcctttctcactttaagagtaaccagccttacgcatcgagactcggtggccccacatgcaaggtctccagaggtgttgcctactccacgcacaggacaaatgccccagggaaggatttccagggcatcagtgctgctttctcaactgagaatggcttccactctgttggccacgagctggcagataaccacatcacctccagagactgcaacggacaccttctcaactgctacgggaggaatgagagcattgcctccaccccaccgggcgaagaccgcaagagcccccgagtgctcatcaaaacgctggggaagctggatgggtgtttaagggtcgagttccacaatggtggcaaccccagcaaagtgcctgcagaggactgcagtgagccggtgcagctgctgaggtactcacctaccttagcatcggaaacctcccctgtgcctgaagccaggagggggtccagcgccgattccctgcccagccatcgcccctctcccacggactctcgcctgcggtccagcaaaggcagctccctgagttctgagtcatcctggtacgactccccttggggcaatgctggagagctgagcgaggctgagggctccttcctggcccccggcatgcctgaccccagtctccatgccagcttcccacctggcgatgccaaaaagcctttcaaccaaagctcttccctctcctccctccgggaactgtacaaagatgccaacctggggagcctctccccctcaggtatccgcctttctgatgaatacatgggcacgcatgccagcctgagcaaccgtgtctcttttgcttccgacattgatgtgccctccagagtggcacacggggaccccatccagtacagttccttcactctcccctgtcggaagcccaaagcctttgttgaggatactgcgaagaaggactccctcaaagccaggatgcgacggatcagtgactggacgggaagcctctcaaggaagaaaaggaaactccag

SEQ ID NO: 128 - DNA, 807 bp, Homo Sapiens
gcagcggaggaggaaatggcaggtcctaatcaactctgcattcgccgctggactaccaagcatgtagctgtgtggctgaaggatgaaggcttttttgaatatgtggacattttatgcaataagcaccgacttgatggaatcacattgctaacattgactgaatatgatctccggtctcctcctctggaaatcaaagtcttaggggacattaaaaggttaatgctctcagtccgaaaattgcagaaaatacatattgatgttttagaagagatgggctacaacagtgacagtcccatgggttccatgacccctttcatcagtgctcttcagagtacagactggctctgtaatggggagctttcccatgactgtgacggacccataactgacttgaattctgatcagtaccagtacatgaatggtaaaaacaaacattctgttcgaagattggacccagaatactggaagactatactgagttgtatatatgtttttatagtatttggatttacatctttcattatggttatagtccatgagcgagtgcctgacatgcagacctatccaccactcccagatatattcttagacagcgttcctagaatcccatgggcctttgccatgacggaagtatgtggcatgattctgtgctatatttggctcctggttcttcttcttcacaagcacaggtcaatacttctgcgaaggctctgtagtctgatgggaactgtattcttgcttcgctgctttaccatgtttgtgacctccctctccgtgccaggacaacacctgcagtgtactggaaag

SEQ ID NO: 129 - DNA, 356 bp, Homo Sapiens
ggttgaaaaataccggccacagaccctgaatgatctcatttctcatcaggacattctgagtaccattcagaagtttatcaatgaagaccgactgccacacttgcttctctacggtcccccagggacaggcaagacatctaccatcctagcctgtgcgaaacagctatataaagacaaagaatttggctccatggtcttggagctgaatgcttcagatgaccgaggaatagacatcattcgaggaccgatcctgagctttgctagcacaaggacaatatttaagaaaggctttaagctagtgatcttggatgaagcagacgccatgactcaggacgcccagaatgccttgagaagag

SEQ ID NO: 130 - DNA, 471 bp, Homo Sapiens
aggggtgtttgatgcttccctcaaaatggctggcttctatggattgtatacctggctgactcatactatgtttggcatcaatattgtcttcataccatcagcattagcagcaatccttggagcagtgccattcctggggacatactgggcagcagtacctgcagttcttgacctgtggctgacacaagggttaggatgcaaggccattttactgttgatttttcatctcttgccaacatactttgtagatactgcaatctactctgacatatcaggaggtggccatccttacctgacaggcttggcagtggccggtggagcatactacctaggcctggaaggagcaatcatcggtcctattcttctctgcatacttgtggttgcttccaatatctatagtgccatgctagtgagtcccacgaattcagttcccacgccaaaccagaccccatggcctgctcagcctcagcg

SEQ ID NO: 131 - DNA, 329 bp, Homo Sapiens
gagttgatggagacctccaagaagctggttcaagctccctaaacccagagctggaggaagaccctgctgaacagatgacgtcacagtcagaggcagaagccccaggattggtagacagacagacgttggaatgagagaaacagagtccggaccccaggtagggtggaactcagccctctgcaggcccagctccccgttgtacaccccaggcccaccttgatcttggagccggcggtgctggggcgccccttcttgtctccatccagcttctcggggaagagcatccggaggaaggccctgggtaggaaaggggagaggaggagttggag

SEQ ID NO: 132 - DNA, 4059 bp, Homo Sapiens
ttcaatataaaagaacaaaccgttacgagaggagctagaaggcaccttaagaggcaagtgaaccctttggacagcatcactttgaagttactctacacagaaacatctgtcttccaggagcctcccagctgcttcactccgtcctgaagtcagaacccctacctccagtcctctctgaattcaggcgtgtttgtgcatttgcttttctaactgggaacacgaagacacctgagagggttggagctggcgggaggagggcagggataaacaccccagcgccttttccccactattgtcttgttctttcttccacttcacctttgctttcttttttaacttgtatattaagttcaggggtacttgtgtaggtttcttatatggtaaacttgtgtcatgggagggggttgttgtacagattattttgtcacccaggaattaagcttctaaataattcagggggccccagcatttgcagaagttacctactggctttagaagaatttctgaaaagtttgatgtcagtcaaatcttttttttttttttaacttaatatgctggctaagatgctggctatttaaagttggaatgaatgaatccctgggcaaaaaggaagcttttaggagcacgcataagcacacaccatactctggctaaattagattttctaaaattgggctttcctaaagaaagtcatatggtagatggccataggcccagtgaaccgaattcagaaagtgttatttcatgagcaagtttcccagaaaggttcgagatgtttctgatagagcaggaatgtacttcctactacacagtactcagccctggaggctcagagcaggaagacctgacctgcacaggccccttttgttggctgggaaacttccacatccctgtgtcaccatgaaaaagagctaactttgtcttcaacgacgagttggaaaattattttaatctctgcattaaactagatgcaaaattgcctctctacctaggaatcacgctgcataatcccacattatctgcattttcagtcatgccttgaataatgatacttgagttcttgaggaggtcattgtggggctgggaagacccctagacttccccatgtagatgtggcctggaagtaagactaacgtagcagcaaagccctggcgatcatgcacactcctgagtaaggcagaggtgtgaatcattatatcaccatggctggctagcttcagaggaaccgaaaaaatgaaaacacttgcgttgctcattagcaaagggaaatacacaaatgttgagggactctttgtgtcacagccacactcttctgtttcgatctggtaagcagagcactctccatacctgcttcaagcttccagctgagagttctcttttagctgttgtacctattgctgaggatcttgatagtgggctagcaacaaggggctttagaaaataagagcatttcagcatcaactgttttaactggagtaagatccattttgtgatttgatttgatttttatttttttagagacagactttcactctgtcttgcaggctggagtgcagtgacgtgataatagctcactgcaacctcaacctcctaggctcaattgatcctcccacctcagcctcccaagttgctgagaccacaggtgcatgccaccacgcccagctagtttttgtattttttgtagagacagggtcttgcttgttgcccaggctggtctcaaactcctggcttcaggtgatccacccgccttggcctcccaaagtgctgggattgcaggcgtgagcctgggatgccaggcatgcaccaggccccattttgtgatttaattctcattgtagactgacattttctatagcagaatttttcaacagtagtactgtagttgaggggccacatttggggctggagatgggtaactctttactatgggagctctcctatttattgcagggtgcttagcagcaccctgggcctctacccactaaatgccagtagcaacccccatccccgcaaattgtgtaaaccaaaaatatatacacacattgccaaatgtcccataggacacaaaatcatatacccatacgctaacacacccatacactaacaccacacgcatacacacacacacacacacacacacacacacacacactttaagactcgctgctctggccaggcgtggtggctcacacctgtaatcacagcactttggggagctggggcaggcagattacttgagttcaggagtttaagaccagtctggccaacatggtggaaccccctctctactaaaaatgcaaaaattagctgggcatggtggtgcatacctgtagtcccagctactctggaggctgaggcaagagaatcgcttgaatctcgtaggcagagttgcagtgagccaagatcatgctactgcactccagcctgggcaacagagtgagactccatctcaaaaaaaaaagaatcactcctccataggagagaggcagtgtataaaataatcttttttctaaattggttgtgcattttaaatagattcctggctggaaacaatggcttacgtccataatcccagcactttgggaggccaaggcagggggattgcttgagctcaggagtttgaaaccagcctgggccacatggcaaaaccccgtctctactacaaatatgaaaattagccaggcatgatggcacacgcctgtaatcctagctacttgagaagctaaggtgggaggatgacttgagcccaggaagcagaggttgcagtgagctgagattggttcactgcactcctgcctggggaacagagccagactctatctcagaaaaacaaaaactaaataactaaataaataagtagattctaagattttacacacattgaatcccgtgtgttactggtttgttccctacatggagtagacaggttttctctttggccttcacggaagctcagactgttactcattctactttcttgtactcatgcattgctaatagttttcatctggttattaattcagtagcaggccgttaagcatgttcactcgtgctgagatatttcttagtgagctctcaccacatgccctgatggtgagcagcctcacctcttaaacaggattcctgcatggttgagaacccgtccacctttctcggcttgttatgatcagagcttacacttttgggaggtagaactgcatctgacatggaggaacatggtccttagagtcagacagacctggattcaaatcccacttctgcttacaagccagatagccttggactgatgattgaacctgcatgcctccgtttccccttatgaacatggggatgattttgtctacctggcagggttctgcatggccttagcgagatcacgtatgtcagacatcagtaaatggtaacttgttatctgtcagttgatagctgtaaggtatgaattaccatctttgtaggtcaaaaaccatcaaatattggcaacttcgtacaattgggcctaatatgtaaaccccaggtcaccatcacgctgtcctctttgctctatggatatccagcagatgaaagggatggttctgctttgttattttcttgttttcctgcttcttctgagggggcttcccgctggcacaatctaacaaacttcagagaggacgaagtcagtctctgctgtcatcagtgttcacagacagtagtttcaaagtgtgtaccacatgaagttgcagtcttccaaccttccagccagtgtgtatggaaataacctgaattgtattaatagcagttcttcaatgtgggcctgctgggggatgcttggttgttttccgttgtttgttccctgggtgcccgtcttgggcaagcatttctctgatgtctctatttatgtggcaggtcaccctgctgattgccttcatctgtgtgcggagctccctgtggaccaactacagcgcctacagctactttgaagtggtcaccatttgcgacttgataatgatcctcgccttttacctggtccacctcttccgcttctaccgcgtgctcacctgtatcagctggcccctgtcg

SEQ ID NO: 133 - DNA, 236 bp, Homo Sapiens
tcctcaagtaccgtaagggtgatgaagatagccttatggagaaaatttccaaactgaacatccactccatcatcaagaaatccaaccgagttagcagtcacctgaagcatctcactggctttgctcctcagataaaagatgaagtatttgaagaaacagaaaaaaacttccgaatgcaagaaagattgattaagtcttttatccgagacctgtctctctacctccagcacatccgg

SEQ ID NO: 134 - DNA, 662 bp, Homo Sapiens
ttatttctcaaactctgaggctgcggagttcacacattgaaatctgtcatctttttcagagagaaactttcggtaatgtatgtaaccttctgtaatggaagatgattgaagacaaggggcctcgtgttgctgactactttgttgtagcaggattaactgatgtttcaaagcctctagaagaagaaattcacttcaatgatgcttgtcataaagtagctaaaccaaaagaacctattacagatgtttcagttattatcaaatctcttggggaggaagtcccacaggattatatctgtattgatgttaccccaactggattgtcagctgatcttaataatggaagtcttgtggggccacagatttacctttgctatagaagaggaagagataagcctccacttacagatctgggggttttatatgactggaaagaaagattgaaacagggttgtgaaattattcagagtactccctatgggcgccccgcaaatattagtgggagtacctcatcacaaagaatttatatcacttaccgaagagcctctgaaaacatgactcagaatacgttggctgtcactgacatatgtattattatacccagtaaaggagaaagcccaccacacacgttctgcaaagtcgacaagaatctcaataacagtatg

SEQ ID NO: 135 - DNA, 268 bp, Homo Sapiens
gtcaggttgccaagaaggaagatctcatcagtgcgtttggaacagatgaccaaactgaaatctgtaagcagattttgactaaaggagaagttcaagtatcagataaagacacacacaactggagcagatgtttagggacattgcaattattgtggcagacaaatgtgtgactcctgaaacaaagagaccatacaccgtgatccttattgagagagccatgaaggacatccactatttggtgaaaaccaacaggagtacaaaacagcag

SEQ ID NO: 136 - DNA, 271 bp, Homo Sapiens
gtgaattcatcattggccgagttatcaaagccatgaataacagctggcatccggagtgcttccgctgtgacctctgccaggaagttctggcagatatcgggtttgtcaagaatgctgggagacacctgtgtcgcccctgtcataatcgtgagaaagccagaggccttgggaaatacatctgccagaaatgccatgctatcatcgatgagcagcctctgatattcaagaacgacccctaccatccagaccatttcaactgcgccaactgcgg

SEQ ID NO: 137 - DNA, 474 bp, Homo Sapiens
gtcctacagctgggctcagacatccttccccagtacaagcaagaggcaccaaagactccccctcacatcatcttacattattgtgtttttaagaccacgtgggattggatcatcttgatcttgaccttctatacagccatcttggtcccttataatgtctccttcaaaaccaggcagaataatgtggcctggctggttgttgatagcatcgtggatgttatctttttggtggacattgtgctcaattttcataccacctttgttggaccagcaggggaggtgatttctgaccccaaacttatccgcatgaactacctgaagacgtggtttgtgattgaccttctgtcctgtttgccatatgatgtcatcaacgcttttgagaacgtggatgaggttagtgcctttatgggtgatccagggaagattggttttgctgatcagattccaccaccactggaggggagagagagtcag

SEQ ID NO: 138 - DNA, 325 bp, Homo Sapiens
accgtcaacaaagatgtggacgcacaggctgaaggagaagggagccgcccatccatggacttattcagggccatctttgccagttcctcagatgaaaagtcctcatcctccgaggatgagcaaggtgacagtgaagatgatcaggcaggctctggggaggccaacttccaaagctcccaagacactgacttgggggaaacatcatctgtggctcacgctcttgtgccagcaccccaggagccgccaccttccttcccgatacaaaagatgcagatagatgaaagagaagagttcggcccgcggctgcctcccgtcttctgcccca

SEQ ID NO: 139 - DNA, 3139 bp, Homo Sapiens
gtccctgacaggctggatgaaatgagatccccatgtagcaattgccatggaaacctgtgactcccctcctatctcaaggcaggaaaatgggcagagcacatcaaagctatgtggaacgacacaacttgataatgaggtgccagagaaagttgcagggatggagcctgacagggaaaacagctccacagatgacaacctgaaaacggatgagcgcaaaagtgaagccttgctgggtttcagcgttgagaatgcagctgccactcaggttacctcagcaaaggagataccctgcaacgaatgtgccacttcttttcccagtttacagaaatacatggaacaccactgccctaatgcccgccttcctgtcctgaaggatgacaacgagagcgagatcagcgagttagaggacagtgacgtggaaaatctaacaggggagatcgtttaccagcctgatgggtcagcatatataattgaggactccaaagaaagtgggcagaatgcacagactggggcaaatagcaaactcttttctacagcgatgttcctggactccctggcatctgctggagagaagagtgatcagtctgcttctgcacctatgtcgttctacccacagatcatcaacacttttcatatcgcttcatccctcgggaaaccatttacagccgatcaggctttcccaaatacctcagcattagcaggagttggtcctgtgttgcacagtttccgtgtctatgatctccgacacaagagagagaaagactatctaaccagtgatggctcagccaaaaactcctgtgtgtccaaagatgtccctaacaatgtggacttgtccaaattcgatggttgtgttagcgatgggaaaaggaaacctgttttaatgtgtttcttgtgcaagttgtcttttggttatatcaggtcatttgtaacccatgctgtgcatgatcatcggatgaccctcaatgacgaggagcagaagctcctcagtaataaatgcgtctccgccataatacaggggattggcaaagacaaagaacctcttataagctttctggaaccaaaaaaatccacttctgtttatccccatttttctactacaaacctcataggacccgatccaaccttccgcggtttatggagcgcttttcatgttgaaaatggtgactctttgccggctggctttgccttcttaaaaggaagcgcgagcacctcgagctcagcagagcagccgctggggattacccaaatgccaaaggctgaagtgaatctgggggggctgtctagtttagtagtgaacaccccaattacctctgtctccctcagccactcatcgtctgagtctagcaagatgtcagagagcaaagaccaagagaacaactgtgaaaggccaaaagaaagcaacgttttacacccaaacggggagtgccctgtcaaaagtgaacccactgaaccgggagatgaggatgaagaagatgcgtactccaatgaacttgatgacgaggaagtattaggtgaactcaccgatagtattggtaacaaagatttccctctcttaaaccaaagcatttctcctttatcatccagtgtgctaaaatttattgaaaagggtacctcgtcctcctcggcgactgtttctgatgacacagaaaagaaaaaacagactgctgctgttagggccagtggcagtgttgctagtaactatggcatcagtggcaaggactttgcagacgcaagtgccagtaaagacagtgccacagctgctcatccaagtgaaatagcccggggagacgaagacagttcagccactcctcaccagcatggctttaccccgagtactcctggcacaccagggcctggaggagacggctcaccgggcagtggcatcgagtgtccaaagtgcgacactgtgttggggtcttcgaggtctcttggtggtcatatgactatgatgcactcgaggaactcatgcaaaaccctcaaatgtcctaaatgtaactggcactacaaatatcagcagaccctggaggcccatatgaaggagaaacaccctgagccgggtggctcttgtgtttattgtaagactggacagcctcaccccaggcttgcccggggtgagagttacacgtgtggctataaacccttccgttgtgaggtttgtaactactctaccactaccaaaggcaacctcagtattcatatgcagtcggacaagcacctgaacaatgttcagaatctccaaaatggcaatggtgagcaggtgtttggccactctgccccagcccccaacaccagcctcagtggctgcggaacaccctctccgtccaaacccaaacagaaacccacctggcggtgtgaagtttgtgattatgaaaccaatgtcgccaggaacctccgaattcatatgaccagcgaaaagcacatgcataatatgatgcttttgcagcagaacatgaagcagatccagcataatctgcacttgggcctcgccccggcggaagcagagctttatcagtactacctagcccagaacataggcctgaccggaatgaagctggaaaaccctgccgaccctcagctgatgatcaatccattccagctggatccagcgacagcagcggctttggcaccagggctcgtaaataatgagctgccgcctgaaatccggcttgccagtggtcagctaatgggtgatgacctgtccctccttactgcaggagagctgtcaccttatatcagtgacccagcgctgaagctattccagtgtgctgtttgcaacaaattcacctctgacagcctggaggccctaagtgtgcatgtgagcagtgagcgctctctccctgaagaggaatggagggcagtaattggagatatctaccagtgcaagctctgcaactacaacactcagctcaaagccaacttccagctacactgcaagactgataaacatatgcagaaatatcaactggtggctcacattaaagaagggggcaaaagcaatgagtggaggttgaagtgtattgccattggcaaccctgttcacctaaaatgtaacgcctgtgactattacaccaacagtgtggataaattacgcttgcataccaccaatcacaggcacgaggcggccctgaagctctacaag

SEQ ID NO: 140 - DNA, 383 bp, Homo Sapiens
atatcggaagaagaaatcgaaaggatcatgtctgggcaggagtttgaggaagaggccaatcactggagcaaccatggtgatagtgaccacagttcccctgggaacagggcttcggagagcaaccagccctcaccaggctccacactgtcttccagtaggtctgtggaactgaatggattcatggccttcagggaacagtacatgggaatgtctgtgcctccacattaccaatatataccgcacctttttagctattctggccactcaccacttctgccccaacaagctcgcagcctggatccccagtcatacagtctgattcaccagctgttatcagccgaggacctggaaccattgggcacgcccatgttgattgaagatgg

SEQ ID NO: 141 - DNA, 491 bp, Homo Sapiens
gctggagtctgggatgcagaacatgagtattcatacgaagaccaccagcggctatgcaggtggtctgagctcggcctatgggggcctcacaagccccggcctcagctacagcctgggctccagctttggctctggcgcgggctccagctccttcagccgcaccagctcctccagggccgtggttgtgaagaagatcgagacacgtgatgggaagctggtgtctgagtcctctgacgtcctgcccaagtgaacagctgcggcagcccctcccagcctacccctcctgcgctgccccagagcctgggaaggaggccgctatgcagggtagcactgggaacaggagacccacctgaggctcagccctagccctcagcccacctggggagtttactacctggggaccccccttgcccatgcctccagctacaaaacaattcaattgctttttttttttggtccaaaataaaacctcagctagctctgccaatgtc

SEQ ID NO: 142 - DNA, 339 bp, Homo Sapiens
gttgaatgtctccattgaatgtaagcgagtgtctggactggagccagccaccgtggattgggccttcgacctgaccaaaacgaatatgcaaaccatgtatgagcagagcgagtggggctggaaggaccgagagaaacgggaggaaatgacagatgaccgagcctggtacctcatcgcgtgggaaaacagctccgtccctgttgccttttctcacttccggtttgacgtggagtgtggggatgaagtcctgtactgctatgaagtgcagttggaaagcaaggtgcggcggaaaggcctggggaagttcctcatacagatcctgcagctcatggccaacag

SEQ ID NO: 143 - DNA, 1548 bp, Homo Sapiens
gtacttgaatctaaaacaaatatggacacacagagcaagaaaatagctgaacagtgagtatgtgaagagtaaaccaacagacaccaggattattcaccataggccagccttctagctcccaagatgttccctccctgccagtcttcattgtctctcagtattgagatgtcagcctcctttccatattacctggaataattgccaagcagtcatgccacttgaaaatgaggtaacaagtcctttgattgaactggcagtcccactccctgtcatagcatccttgtatcagtgttgtttattcatttcatggtctgttctctcaagaaataagcatcaggagtggacagtgatgtttggggggatcccaaggcacatgggaggcagatcttacctctttctgaggaggccacagagagctggatagtccctctgtctgctccctggtagctggaatgtggatctgtgactccatgcagccaactggaagcttaatgcagggaatgtgaactttgaactagttagagattattcactgtgaggcttttgcaaagggcacaggctggatgagtggccgtgagcgtccgtgttcctgagcagactgtgctgtggcccaatcctgtgcgtgtctcctcttcaggctcctgccggagcatgcttctctaatcttaccatttatcactgacccttcggtgaaactctcttctgtttaaatttaccagggtacactttgtaatacttgggaccaagaatttgaagatagtaatcagacaaatcaaacaactagtagtagtggagagacacgtagccctacaggaaggggtagaattatcattaaaaacacagacaaaccagagactgcctgaaaaaaaaaaaaagatgaactggaaagattcaaagtaggctctattggaggtgagtatggaaaacaagcttagctttttattttgaagtttccttccaggagttggatactgtgatctcttcaatcagcatgtttatatagtgaggaggaagaccagaaatgagaagaggaataagaaaatggcagcacagccttttggggtatggataagggtggtcttcctgctacagcagaggagaggggcatgtgtggcccatgtggaagagcaactgagttggtggctgactgaatttgggtccgaagcaaaaataggatatgaggataggtataagaattttattttgtgctatgtgtgtcatttggcttttaaactgtctagaaataaaatgagatgaaaacaatctgccctcatttcttggttgcttttcttaaatatcattggcccaaagcatagccaagccttttatttagaaaatcctaccttcaaaaagtgataggttacactaattatatttgtgctgttctacagattttctattttgcactggtcctttacctgctgctcgctgtgcgggcagctaaggtgagagtttgaagactatggacttttaagctgagaggctgctgtggtcatacagttttcaacacaaaacttgttctccctctagtgttccacttgcacaggg

SEQ ID NO: 144 - DNA, 269 bp, Homo Sapiens
gtggtatgcattgggtcagtggcggaacttgaagaactgtcaggagcaaagatctcagatctccacagagagagtgttgaccacctgaccattccttcacgctgtgggaagggatccttgcaccgcatctctgaagtgtttgactgttggtttgagagtggcagcatgccctatgctcaggttcattacccgtttgaaaacaagagggagtttgaggatgcttttcctgcagatttcattgccgagggcatcgaccaaaccagaggatg

SEQ ID NO: 145 - DNA, 238 bp, Homo Sapiens
gtcttcgccttgatcgtgttctcctgcatctatggtgagggctacagcaatgcccacgagtctaagcagatgtactgcgtgttcaaccgcaacgaggatgcctgccgctatggcagtgccatcggggtgctggccttcctggcctcggccttcttcttggtggtcgacgcgtatttcccccagatcagcaacgccactgaccgcaagtacctggtcattggtgacctgctcttctcag

SEQ ID NO: 146 - DNA, 362 bp, Homo Sapiens
gtactaaggtctacatcgaccccttcacttatgaagaccctaatgaggctgtgagggaatttgcaaaagagatcgatgtctcctacgtcaagattgaagaggtgattggtgcaggtgagtttggcgaggtgtgccgggggcggctcaaggccccagggaagaaggagagctgtgtggcaatcaagaccctgaagggtggctacacggagcggcagcggcgtgagtttctgagcgaggcctccatcatgggccagttcgagcaccccaatatcatccgcctggagggcgtggtcaccaacagcatgcccgtcatgattctcacagagttcatggagaacggcgccctggactccttcctgcgg

SEQ ID NO: 147 - DNA, 148 bp, Homo Sapiens
gtcgggagggccgaggcgccgcttgtgggtcccctcgctggagagggctggctgggcgaggccggctcgaatctctggctcctggcttagagcagttgctcttaaaggggaacgggccacccccggctgctccagtgggctaggctgg

SEQ ID NO: 148 - DNA, 264 bp, Homo Sapiens
ccaaggtggtgccccagcagatcacgcacacttctcctcggatccagccagactaccctgccgagaggagtagcctgattcccatctccggacatcgggcctctcccaatcctgtggccatggaaacccgaagtgacaacagaccgtctgttcccgttcagttccaatattttttgccaacttaccccccttctgcatacccactggcggcacatacctacaccccaatcaccagttccgtgtccactatccgacagtatccag

SEQ ID NO: 149 - DNA, 1521 bp, Homo Sapiens
agtaacagttacccctcaatgagcgacccctacctgtccagctattacccgccgtccattggatttccttactccctcaatgaggctccgtggtctactgcaggggaccctccgattccatacctcaccacctacggacagctcagtaacggagaccatcattttatgcacgatgctgtttttgggcagcctgggggcctggggaacaacatctatcagcacaggttcaattttttccctgaaaaccctgcgttctcagcatgggggacaagtgggtctcaaggtcagcagacccagagctccgcgtatgggagcagctacacctaccccccgagctccctgggtggcacggtggttgatgggcagccaggctttcacagcgacaccctcagcaaggcccccgggatgaacagcctggagcagggcatggttggcctgaagattggggacgtcagctcctccgccgtcaagacggtgggctctgtcgtcagcagcgtggcactgactggtgtcctttctggcaacggtgggacaaatgtgaacatgccagtttcaaagccgacctcgtgggctgccattgccagcaagcctgcaaaaccacagcctaaaatgaaaacaaagagcgggcctgtcatggggggtgggctgccccctccacccataaagcataacatggacattggcacctgggataacaaggggcctgtgccgaaggccccagtcccccagcaggcaccctctccacaggctgccccacagccccagcaggtggctcagcctctcccagcacagcccccagctttggctcaaccgcagtatcagagccctcagcagccaccccagacccgctgggttgccccacgcaacagaaacgcggcgtttgggcagagcggaggggctggcagcgatagcaactctcctggaaacgtccagcctaattctgcccccagcgtcgaatcccaccccgtccttgaaaaactgaaggctgctcacagctacaacccgaaagagtttgagtggaatctgaaaagcgggcgtgtgttcatcatcaagagctactctgaggacgacatccaccgctccattaagtactccatctggtgtagcacagagcacggcaacaagcgcctggacagcgccttccgctgcatgagcagcaaggggcccgtctacctgctcttcagcgtcaatgggagtgggcatttttgtggggtggccgagatgaagtcccccgtggactacggcaccagtgccggggtctggtctcaggacaagtggaaggggaagtttgatgtccagtggatttttgttaaggatgtacccaataaccagctccggcacatcaggctggagaataacgacaacaaaccggtcacaaactcccgggacacccaggaggtgcccttagaaaaagccaagcaagtgctgaaaattatcagttcctacaagcacacaacctccatcttcgacgactttgctcactacgagaagcgccaggaggaggaggaggtggtgcgcaag

SEQ ID NO: 150 - DNA, 153 bp, Homo Sapiens
acatagatgagtgtgaagtttctggcctgtgcaggcatggagggcgatgcgtgaacactcatgggagctttgaatgctactgtatggatggatacttgccaaggaatggacctgaacctttccacccgaccaccgatgccacatcatgcacag

SEQ ID NO: 151 - DNA, 293 bp, Homo Sapiens
tgacaaaaatgctgagtttcttccgtagaacactagggcgtcggtctatgcgtaaacatgcagagaaggaacgactccgagaagcacaacgcgccgccacacatattcctgcagctggagattctaagtccatcatcacgtgtcgggtgtcccttctggatggtactgatgttagtgtggacttgccaaaaaaagccaaaggacaagagttgtttgatcagattatgtaccacctggacctgattgaaagcgactattttggtctgagatttatggattcagcacaagtagca

SEQ ID NO: 152 - DNA, 309 bp, Homo Sapiens
ggggcgacgatggccttctgaggggctgggacaccagggtacccggcaaatttctcttcaccagcaaaagacacaccatgggtgtgtgcagcatccagagcagccctcatcgggagcacatcctggccacgggaagctatgatgaacacatcctactgtgggacacacgaaacatgaagcagccgttggcagatacgcctgtgcagggtggggtatggagaatcaagtggcaccctttccaccaccacctgctcctggccgcctgcatgcacagtggctttaagatcctcaactgccaaaaggcaatgg

SEQ ID NO: 153 - DNA, 440 bp, Homo Sapiens
ccatgcgatgatggacctactggttgaactttgccttcagaaccacctgaatccatcccaccatgcccttgaaattcggtcttcagaaacccaacaacctttgagttttaagccaaatactttgattgggaccctgaatgtgcatactgtgtttctgaaagaaaaagttcctgaagagaaggttaagcctggtccccctaaggtgcctgagaaatctgtgcgtttggtcgtgaattacctgcggacacaaaaagctgttgtgcgtgtgagccctgaggttcctctccagaatattctcccagtcatttgtgcaaagtgtgaggtcagcccagagcacgtggttctcctcagggacaacattgccggagaggagctggagctgtccaagtccctgaacgagctcgggataaaggagctctacgcgtgggacaacagaagag

SEQ ID NO: 154 - DNA, 251 bp, Homo Sapiens
gatcaagacttcaaatcccaaaagaacttcatcattaacatgacttgcagattttgctggcagcttcctgaaacagattacgagtgtaccaactccaccagctgcatgacggtgtcctgtcctcggcagcgctaccctgccaactgcacggtgcgggaccacgtccactgcttgggtaaccgtacttttcccaaaatgctatattgcaattggactggaggctataagtggtctacggctctggctctaag

SEQ ID NO: 155 - DNA, 654 bp, Homo Sapiens
atcgcttgacaatatcttattgtcggagtagtggtcctggggggcagaatgtgaacaaagtgaattccaaggcagaagtcaggttccatttggcaactgccgagtggatcgcggagcccgtgcggcagaagatagccatcacgcataaaaacaagatcaacaggttaggagagttgatcctcacctctgagagcagccgctatcagttccggaatctggcagattgcctgcagaaaattcgagacatgatcactgaggccagccagacaccgaaggagccaacaaaagaagatgttaaacttcatagaatcaggatagaaaacatgaatcgggaaaggctgagacaaaagagaattcattctgctgtaaagacaagcaggagggtcgacatggactgaaatcaccctctgcagctgggagggctcttctgggcgtccgggcagctgcagctgagaggactttcacaccataaggagatttctgtttttctttttggctgttaatgcttgtctataacattggagccatcacaagaatgttcatttggaatgaaggctgcaggcactggttgcagacgtctttataggcagtcaccatgttgtcaaaccttaataatgcacctcatgtattagtcacaataaaaatcagaactca

SEQ ID NO: 156 - DNA, 1911 bp, Homo Sapiens
gcggcgtccgtccgtactgcagagccgctgccggagggtcgttttaaagggcccgcgcgttgccgccccctcggcccgccatgctgctatccgtgccgctgctgctcggcctcctcggcctggccgtcgccgagcctgccgtctacttcaaggagcagtttctggacggagacgggtggacttcccgctggatcgaatccaaacacaagtcagattttggcaaattcgttctcagttccggcaagttctacggtgacgaggagaaagataaaggtttgcagacaagccaggatgcacgcttttatgctctgtcggccagtttcgagcctttcagcaacaaaggccagacgctggtggtgcagttcacggtgaaacatgagcagaacatcgactgtgggggcggctatgtgaagctgtttcctaatagtttggaccagacagacatgcacggagactcagaatacaacatcatgtttggtcccgacatctgtggccctggcaccaagaaggttcatgtcatcttcaactacaagggcaagaacgtgctgatcaacaaggacatccgttgcaaggatgatgagtttacacacctgtacacactgattgtgcggccagacaacacctatgaggtgaagattgacaacagccaggtggagtccggctccttggaagacgattgggacttcctgccacccaagaagataaaggatcctgatgcttcaaaaccggaagactgggatgagcgggccaagatcgatgatcccacagactccaagcctgaggactgggacaagcccgagcatatccctgaccctgatgctaagaagcccgaggactgggatgaagagatggacggagagtgggaacccccagtgattcagaaccctgagtacaagggtgagtggaagccccggcagatcgacaacccagattacaagggcacttggatccacccagaaattgacaaccccgagtattctcccgatcccagtatctatgcctatgataactttggcgtgctgggcctggacctctggcaggtcaagtctggcaccatctttgacaacttcctcatcaccaacgatgaggcatacgctgaggagtttggcaacgagacgtggggcgtaacaaaggcagcagagaaacaaatgaaggacaaacaggacgaggagcagaggcttaaggaggaggaagaagacaagaaacgcaaagaggaggaggaggcagaggacaaggaggatgatgaggacaaagatgaggatgaggaggatgaggaggacaaggaggaagatgaggaggaagatgtccccggccaggccaaggacgagctgtagagaggcctgcctccagggctggactgaggcctgagcgctcctgccgcagagctggccgcgccaaataatgtctctgtgagactcgagaactttcatttttttccaggctggttcggatttggggtggattttggttttgttcccctcctccactctcccccaccccctccccgcccttttttttttttttttttaaactggtattttatctttgattctccttcagccctcacccctggttctcatctttcttgatcaacatcttttcttgcctctgtccccttctctcatctcttagctcccctccaacctggggggcagtggtgtggagaagccacaggcctgagatttcatctgctctccttcctggagcccagaggagggcagcagaagggggtggtgtctccaaccccccagcactgaggaagaacggggctcttctcatttcacccctccctttctcccctgcccccaggactgggccacttctgggtggggcagtgggtcccagattggctcacactgagaatgtaagaactacaaacaaaatttctattaaattaaattttgtgtctcc

SEQ ID NO: 157 - DNA, 259 bp, Homo Sapiens
gtggaagatgagctcagctccccagtggtggtgttcagatttttccaggaattaccaggctcagatccggtgtttaaagccgtcccagtgcccaacatgacaccttcaggagtcggccgggagaggcactcgtgtgacgcgctgaatcgctggctgggagaacagctgaagcagctggtgcctgcaagcggcctcacagtcatggatctggaagctgagggcacgtgtttgcggttcagccctttgatgaccgcagcag

SEQ ID NO: 158 - DNA, 179 bp, Homo Sapiens
gttgtgcaccatctccaggtgcttatgatgttaaaactttagaagtattgaaaggaccagtatcctttcagaaatcacaaagatttaaacagcaaaaagaatctaaacaaaatcttaatgttgacaaagatactaccttgcctgcttcagctagaaaagttaagtcttcggaatcaaag

SEQ ID NO: 159 - DNA, 169 bp, Homo Sapiens
ctcccgcggctccagcctccgcgccctcccggccggcgcgagtgtgtctgcgcgtgagtgtgagtgtgagtgtgtgttgtgcgcgcaggagccagtgcgtcgggagcagcggctcgggcagctgctcctgcccgcaccctcccctggagcccggggaggacaaggcgag

SEQ ID NO: 160 - DNA, 117 bp, Homo Sapiens
atcctgcccggcctgtacatcggcaacttcaaagatgccagagacgcggaacaattgagcaagaacaaggtgacacatattctgtctgtccacgatagtgccaggcctatgttggag

SEQ ID NO: 161 - DNA, 104 bp, Homo Sapiens
gatgaatttttagatgtgatctactggttccgacagatcattgctgtggtcctgggtgtcatttggggagttttgccattacgagggttcttgggaatagcagg

SEQ ID NO: 162 - DNA, 2681 bp, Homo Sapiens
aggaccctggccacgaatcatttgccatctcctctaccaaagactcaaaatgtatggccctggtagtcagctgggaaagtctggcaataacagctgggctaaggaacgtggatgctcaattgcctgccaagggtctcttacttcagcccggctacatgcccccagcattggagagaggccactttcacacatgcgttggaagggtaagagactctggcaaaaataaattcgtctctctgcatttagcagtgggctcagggccatgaggaagggaattagaggaaaggaattagcaacagcccaccatgtgtctcgtctggcatgagtggacaggtgttagctctcctctgtatatgaggatctgattttcagctgagttgtcgatgttgaaccatgttccatttccacttagatttttatccaggctccttctatctaaccaacatgaacagaatgtcctgcagtagcacataggaaaattacattgggtgcagtctaggaaatcttaatcagggagggcctggtgatctgaaagcacaaagctttcagtgtctatcagatgatttagtcgaatttggagtctgaaatgacacataagacagcctaggaacctagctctgtgtcacctcccctccccttcagagtccctggtttactagggaagtggctgtggtggatgatcagctcagtgaaaacccagcctggttttgtttccccccgacaatagcccctgcatttttcctttcctaggtcccaactgttactgctgtatcactccaggccttcctcaagtctggtttactgtcaaatagcttcctagcccagtacctggctttgagcttgttcctgtgtaagctgtccagtgtgctcctgccacatttactgtcatcctaaacatcactttgatcacattgctttccatttaaatacttttgctctgcttttcccagacctggctcttacccgttaggctgctttaggctgacttttcctgtttctcactgtgtaccacttagcactaggcggtttggtagtgagttgctctgattattacgtctgtgttagaaggagattagaaggcttttgaagtcaagggtgatggctgtgtcttttgaattcctcccatagctattcttgttgaaacactctggggaatagacacaattctcagacagcagagtgttgatgtctagggaaaatgtttacaatctgaaaaattaccttggcaggctgaggacaaagccacaaaatcgtaacttgtttttcggctttcttcagcctcacctctaccagagaatttcactttggagttttcttatctgtaaaaggggaagggttgaagtacattatctgtcagctcccacccaaccctgaaaggctaattttctttaattctttcccttaatctttccattgaaattaaaactgatgagctgtttctggatcttgaattaggaatttttcatttctttgtaattctttccctgacgcattttgcagttgtctccgggtatgtgctgctaaaagaaatttggattgcaatttggctactttttaggagagcagacaaaatgccataccattctgtttttcctacttggagcatttcatttgtaaaagtaaagctttcagtttggtgcataattctaacataattctcactcttcctttgcagacacgatgaggcgagtggtacgacagagcaagtttcggcatgtatttgggcaagcggtgaaaaatgaccagtgctatgatgacatccgggtttctcgtgtgacctgggatagttccttttgtgctgtcaatcccagatttgttgccataatcatagaggcaagtgggggaggagcgttccttgtcctccctctgcacaagactggtcgaattgacaaatcttaccctacagtatgtggccacacaggaccagtgctggacatagactggtgcccacataacgatcaggtcattgccagcggttcagaggactgcacggtcatggtatggcagatcccagaaaatggactcaccctttccctgactgaacctgtggtgattttggaaggccactcaaagagagtcggcatcgtggcttggcatccaacggcccgcaatgtgcttcttagtgcaggctgtgataatgccattatcatctggaatgtgggaacaggggaagcccttataaacttggacgatatgcattcagacatgatttacaatgtgagctggaaccggaatggcagtctgatctgcacagcttccaaagacaagaaagtgagagtcattgatcccaggaaacaagagattgttgctgagaaggagaaagcacatgaaggagcaagacccatgagagccatcttcctggccgatggcaatgtcttcaccactgggttcagccgcatgagcgagcggcagctggctctctggaatccgaaaaatatgcaggaaccaattgctcttcatgagatggacactagcaatggggtgttgctgcctttctatgaccctgacaccagcatcatttacttatgtggaaagggtgacagcagtattcgctattttgagatcacggatgaatccccgtacgtccactacctcaacacattcagcagcaaggagcctcagagagggatgggttacatgcccaagaggggacttgatgttaacaaatgtgagattgccag

SEQ ID NO: 163 - DNA, 372 bp, Homo Sapiens
agttcacccagggcatgatcttacctacaatgaatggagagtcagtagacccagtggggcagccagcactgaaaactgaggagcgcaaggctaagcctgctccttcaaaaacccaggccagacctgttggagtcaaaatccccacttgtaagatcactcttaaggaaaccttcctgacgtcaccagaggagctctatagagtgtttaccacccaagagctggtgcaggcctttacccatgctcctgcaacattagaagcagacagaggtggaaagttccacatggtagatggcaacgtctctggggaatttactgatctggtccctgagaaacatattgtgatgaagtggaggtttaaatcttggccagagg

SEQ ID NO: 164 - DNA, 340 bp, Homo Sapiens
gtctctggcttccgtttcttacgctgctgttgattttttccgaccatcagctcagagactgatagaagagaaaggtgcagtggatgcattggctgcagctttagcccacatttctggtgcatcaagctttgaaccacgatctttgatcacctctgataaggggtttgtgaccatgactctggaaagcctagaggaaatacaggatgtcagctgtgcttggaaagaacttaacagaaagctgagtagtaatgcagtgtctcagattaccagaatgtgcctcctgaaaggaaatatgggtgtttgctttgatgttcctacaactgagtcagaaaggttacag

SEQ ID NO: 165 - DNA, 247 bp, Homo Sapiens
gcatctggccatctgaagaggttttggcttactactgcctcaagcacaataatatattcagggcccttgctgtgtgtgagctagggggtggcatgacatgcttggctgggctcatggttgctatttctgcagatgtcaaagaagttctgttaactgatgggaatgaaaaggccatcagaaatgtgcaagacatcatcacaaggaatcagaaggctggtgtgtttaagacccagaaaatatcaagctg

SEQ ID NO: 166 - DNA, 203 bp, Homo Sapiens
gaagtgtttggattgtgagctatttcagaactgttctcaggactcattattttaacatttgggagaaacacagccagaagatgcacacttgactgaaggaggacagggaatctgaagactccggatgacatcagagctacttttcaacagccttctcaattttctttctcagaaagcagaggctcagagcttggagacagacg

SEQ ID NO: 167 - DNA, 753 bp, Homo Sapiens
ccgatttattttgcatattcccagcgaggaaagagacaatgcaatccgagtgtgttttcagattgaacttgcccattggttttacttggatttctacatgcagaacacaccaggattacctcagtgtgggataagagactttgctaaagctgtcttcagtcattgtccgtttttgctgcctcaaggtgaagatgtggaaaaagttttggatgaatggaaggaatataaaatgggagtaccaacatatggtgcaattattcttgatgagacacttgaaaatgtactactagttcaggggtacctagcaaaatcaggctggggatttccaaaaggaaaagtaaataaagaagaagctcctcatgattgtgctgctagagaggtctttgaagaaactggttttgatatcaaagactatatttgtaaggatgattacattgaacttcgaatcaatgaccagcttgctcgtttgtacatcattccaggaattccaaaagacacaaaatttaacccaaaaactagaagagaaattcggaacattgagtggttctctattgagaaattgccttgtcatagaaatgatatgacccccaaatccaaacttggtttggcacctaacaaattttttatggccattccctttatcagaccattaagggactggctttctcgaagatttggcgattcctcagacagtgacaatggattttcctcaactggtagcacgccggctaaacccactgtggaaaaattgag

SEQ ID NO: 168 - DNA, 982 bp, Homo Sapiens
atcatggagctttgtggtgcaacaagacttggttattttggaagaagtcagttctacattgctttgaaacttgtagctgttgcccagtctggtttccctttaagagtggaaagtataaatacagtaaaggaccttcctctgccaagatttgttgcttcaaagaatgaacaggaatctcgccatgcagcctcatattcttcagattctgaaaatcaagggtcgtattctggtgtaattcccccaccacctggcagggggcaagtgaaaaagggatccgtaagccatgatacggttcagcctcgtacatctgcagatgcacaggaacctgcatccccagtagtttcaccacagcaatccccaccaacttctccacacacatggaggaagcacagccgtcatcccagcggtgggaatagtgagaggcctctcgcgggacctgggccattttggtctccctttggtgaagcacaatcaggttcttctgctggtgatgcagtgtggtcagggcattccccacctccacctcaagaaaactgggtcagttttgcagatactccaccaaccagtactcttttaaccatgcatcctgcttctgtccaggaccagacaacagtacgaactgtagcatcagctacaactgccattgaaattcgtaggcaatccagtagttatgatgatccctggaaaataacagatgaacaaagacagtattatgtaaatcagtttaaaaccattcagcctgatctaaacggatttattccaggatctgcagctaaagagttttttacaaaatcaaaacttcctattcttgaactttctcatatttgggaactctcagactttgataaagatggtgcattgacactggatgagttttgtgctgcttttcatctggtggttgctaggaagaatggctatgatttaccagaaaaacttcctgaaagcttaatgcccaaactgattgatttggaagattcagcag

SEQ ID NO: 169 - DNA, 779 bp, Homo Sapiens
gtatgtgatagtggaatgtgaagatcaagatactcagcagagagatccaaagacccatgagatgtacttgaacgtaatgagaagattcagccaagcattgttgaagggtgataagtctgtcagagttatgcgttctttgctggctgcacaacagacatttgtagatcggttggtgcatctaatgaaggcagtacaacgcgaaagtggaaatcgtaagaaaaagaatgagagactacaggcattgcttggagataatgaaaagatgaatttgtcagatgtggaacttatcccgttgcctttagaaccccaagtgaaaattagaggaataattccggaaacagctacactgtttaaaagtgcccttatgcctgcacagttgttttttaagacggaagatggaggcaaatatccagttatatttaagcatggagatgatttacgtcaagatcaacttattcttcaaatcatttcactcatggacaagctgttacggaaagaaaatctggacttgaaattgacaccttataaggtgttagccaccagtacaaaacatggcttcatgcagtttatccagtcagttcctgtggctgaagttcttgatacagagggaagcattcagaacttttttagaaaatatgcaccaagtgagaatgggccaaatgggattagtgctgaggtcatggacacttacgttaaaagctgtgctggatattgcgtgatcacctatatacttggagttggagacaggcacctggataaccttttgctaacaaaaacag

SEQ ID NO: 170 - DNA, 295 bp, Homo Sapiens
caaaagcggcgccaggaaattgaacaaaaacttgaagttcaggcagaagaagagagaaagcaggttgaaaatgaaaggagagaactgtttgaagagaggcgtgctaaacagacagaactgcggcttttggaacagaaagttgagcttgcgcagctgcaagaagaatggaatgaacataatgccaaaataattaaatatataagaactaagacaaagccccatttgttttatattcctggaagaatgtgtccagctacccaaaaactaatagaagagtcacagagaaaaatgaacg

SEQ ID NO: 171 - DNA, 693 bp, Homo Sapiens
cccttacgtcgtcctgactcatctgatgaccgttatgtaatgacaaaacatgccaccatttatccaactgaagaggagttacaggcagttcagaaaattgtttctattactgaacgtgctttaaaactcgtttcagacagtttgtctgaacatgagaagaacaagaacaaagagggagatgataagaaagagggaggtaaagacagagctttgaaaggagttttgcgagtgggagtattggcaaaaggattacttctccgaggagatagaaatgtcaaccttgttttgctgtgctcagagaaaccttcaaagacattattaagccgtattgcagaaaacctacccaaacagcttgctgttataagccctgagaagtatgacataaaatgtgctgtatctgaagcggcaataattttgaattcatgtgtggaacccaaaatgcaagtcactatcacactgacatctccaattattcgagaagagaacatgagggaaggagatgtaacctcgggtatggtgaaagacccaccggacgtcttggacaggcaaaaatgccttgacgctctggctgctctacgccacgctaagtggttccaggctagagctaatggtctgcagtcctgtgtgattatcatacgcattcttcgagacctctgtcagcgagttccaacttggtctgattttccaagctgg

SEQ ID NO: 172 - DNA, 298 bp, Homo Sapiens
aactgtcagcacaacaccgcgggagagcactgtgaacgctgccaggagggctactatggcaacgccgtccacggatcctgcagggcctgcccatgtcctcacactaacagctttgccactggctgtgtggtgaatgggggagacgtgcggtgctcctgcaaagctgggtacacaggaacacagtgtgaaaggtgtgcaccgggatatttcgggaatccccagaaattcggaggtagctgccaaccatgcagttgtaacagcaatggccagctgggcagctgtcatcccctgactggag

SEQ ID NO: 173 - DNA, 1583 bp, Homo Sapiens
gcatgtgctgataagttgaaaagagaaatagagctgtatggttgcccccctgattttccagatgaagaagaggaagaggaagaaaccagtgttaaaacagaagatataataattaaggataaagctaaaggaaaaaagagtaaagctgctgctaaagctggatcttctaaataccagtggggcattatgaaatcccttggcctgtctgatgaagagatagtaaaattttctgaagcagaacattggcttgattatttcccgccactggctattcaggatttaaaaagaatgggtttgaaggtagactggcgtcgttccttcatcaccactgatgttaatccttactatgattcatttgtcagatggcaatttttaacattaagagaaagaaacaaaattaaatttgggaagcggtatacaatttactctccgaaagatggacagccttgcatggatcatgatagacaaactggagagggtgttggacctcaggaatatactttactcaaattgaaggtgcttgagccatacccatctaaattaagtggcctgaaaggtaaaaatattttcttggtggctgctactctcagacctgagaccatgtttgggcagacaaattgttgggttcgtcctgatatgaagtacattggatttgagacggtgaatggtgatatattcatctgtacccaaaaagcagccaggaatatgtcataccagggctttaccaaagacaatggcgtggtgcctgttgttaaggaattaatgggggaggaaattcttggtgcatcactttctgcacctttaacatcatacaaggtgatctatgttctcccaatgctaactattaaggaggataaaggcactggtgtggttacaagtgttccttccgactcccctgatgatattgctgccctcagagacttgaagaaaaagcaagccttacgagcaaaatatggaattagagatgacatggtcttgccatttgagccggtgccagtcattgaaatcccaggttttggaaatctttctgctgtaaccatttgtgatgagttgaaaattcagagccagaatgaccgggaaaaacttgcagaagcaaaggagaagatatatctaaaaggattttatgagggtatcatgttggtggatggatttaaaggacagaaggttcaagatgtaaagaagactattcagaaaaagatgattgacgctggagatgcacttatttacatggaaccagagaaacaagtgatgtccaggtcgtcagatgaatgtgttgtggctctgtgtgaccagtggtacttggattatggagaagagaattggaagaaacagacatctcagtgcttgaagaacctggaaacattctgtgaggagaccaggaggaattttgaagccaccttaggttggctacaagaacatgcttgctcaagaacttatggtctaggcactcacctgccttgggatgagcagtggctgattgaatcactttctgactccactatttacatggcattttacacagttgcacacctattgcaggggggtaacttgcatggacaggcagagtctccgctgggcattag

SEQ ID NO: 174 - DNA, 371 bp, Homo Sapiens
gattcactccttctccagttgcacagccacacccttcagctggccttaatgttgactttgaatctgtgtttggaaataaatctacaaatgttattgtagattctgggggctttgatgaactaggtggacttctcaaaccaacagtggcctctcagaaccagaaccttcctgttgccaaactcccacctagcaagttagtatctgatgacttggattcatctttagccaaccttgtgggcaatcttggcatcggaaatggaaccactaagaatgatgtaaattggagtcaaccaggtgaaaagaagttaactgggggatctaactggcaaccaaaggttgcaccaacaaccgcttggaatgctgcaacaatg

SEQ ID NO: 175 - DNA, 576 bp, Homo Sapiens
gcatgggactcgctgtgctggagaagtggcagccgctgcaaacaattcgcactgcacagtcggaattgctttcaacgccaagatcggaggagtgcgaatgctggacggagatgtcacggacatggttgaagcaaaatcagttagcttcaacccccagcacgtgcacatttacagcgccagctggggcccggatgatgatggcaagactgtggacggaccagcccccctcacccggcaagcctttgaaaacggcgttagaatggggcggagaggcctcggctctgtgtttgtttgggcatctggaaatggtggaaggagcaaagaccactgctcctgtgatggctacaccaacagcatctacaccatctccatcagcagcactgcagaaagcggaaagaaaccttggtacctggaagagtgttcatccacgctggccacaacctacagcagcggggagtcctacgataagaaaatcatcactacagatctgaggcagcgttgcacggacaaccacactgggacgtcagcctcagcccccatggctgcaggcatcattgcgctggccctggaagccaa

SEQ ID NO: 176 - DNA, 379 bp, Homo Sapiens
agggaagtgtaacatctcccatttttctgacatatttgctgctagagagtttaaagcccgagtggattcatttttctacatattaggatataaccctgagacaaggaggctgaacagtacccagggggagattcgtgtcggtcctagtcatcaggccaaacttccagatctgcaaccatttccttctccagatggtgatacagtgacccaacatgaggaactggtctggatgcctggagttaacgactgtgacctccttatgtacttgagggcagcaaggagcatggcggcatttgcaggaatgtgtgatggaggctctacagaggacggctgtgtcgcagcctctcgggatgacaccactctgaatgcactgaacaca

SEQ ID NO: 177 - DNA, 516 bp, Homo Sapiens
ctgaatgcaatatccatacatctccttctccgggaattcaagtaaggcatgtctacaccccctctacaacaaagcatttctcacccataaaacagtcaaccactttaaccaacaaacacagaggaaatgaggtctctaccacacctctgttagcaaattctttgtctgttcaccagttggctgctcagggagagatgctctatctggctactcgtatcgaacaagaaaatgttatcaatcacacggatgaagaaggatttactcctctgatgtgggctgcagcacacgggcaaatagctgtggtagagttcctacttcagaatggtgctgatccccaacttttaggaaaaggtcgagaaagtgcactgtcgttggcctgtagtaaaggctacacagatattgtcaaaatgctgcttgattgtggagttgatgtaaatgaatatgattggaatggaggaacacctctgctttatgctgtacatggaaatcatgtgaaatgtgtaaagatgctcttag

SEQ ID NO: 178 - DNA, 181 bp, Homo Sapiens
agaagatggcgaattagaagatggtgaaatagacgatgcaggatttgaagaaatacaagaaaaagaagcaaaagagaatgaaaagcagaaaagtgagaaagcctacagaaaatcaagaaaaaaacataagaaagagagagagaagaaaaaatccaaaaggagaaaacgtgagaaacataag

SEQ ID NO: 179 - DNA, 602 bp, Homo Sapiens
gaggatagcaaacaagctcaatttttagccttggcagtagtatactttatctctgttcttatggtctccaagtacagagacattttggaaccccaaaatgaaaggcatagccagtcatgtacagaaactggcagtgaaaatgagaatgtatcactctctgaaatcacaccagcagcattcagcactttaactacggcatcagtggaagaatctgaaagcacatcatctgctcgaaggagggactcaggcattggggaagaaacagccactggtttaggaagccatgtggaagtaactcctcacacagcacctcctggtgtcagtgcaggcccagatgcaatcagcgaggtgctatctactctttctttagaagtcaataagtctccggaaaccaaaaatgatagaggaaatgacttggacactaaggctacaccgtcagtttcagtttcaaaaaacgtcaatgtgaaagacattctccgaagcttggttaacataccagcagatggagtcacagtggatcctgcccttctgccaccagcctgccttggagcccttggtgatctatctgtggaacaacccgtgcagttcagatcttttgacag

SEQ ID NO: 180 - DNA, 175 bp, Homo Sapiens
tgtggcaaagcgctccctggcctttcaaagcaagaggactgctgtggaactgtgggtacctcctggggctttaacaaatgccagaaatgccccaagaaaccatcttatcatggatacaaccaaatgatggaatgcctaccgggttataagcgggttaacaacaccttttgccaag

SEQ ID NO: 181 - DNA, 1571 bp, Homo Sapiens
gtttgatgattcctgtcttttgtgtcgtggagcagttggacggctctcttgaatatgacaacagagaagaacacgccgagtttgtcctggtgcggaaagatgtgctttttagccagctggtggagactgcgctcctggccctggggtattctcacagctctgcggcccaggcccaaggaataatcaagctgggaaggtggaaccctctccccctcagttatgtgacagatgcacccgacgcgacagtggccgacatgctacaagatgtctatcatgttgtgacgttgaaaatccaattacaaagttgttcaaagttggaagacttgcctgcggagcagtggaaccatgccacagtccgcaatgccttaaaggaactgctcaaagagatgaaccagagcacattagccaaagaatgccctctctcccagagtatgatttcatccattgtaaatagcacatattatgccaatgtgtcagcaaccaagtgccaggagtttgggagatggtataaaaagtacaagaagattaaagtggaaagagtggaacgagaaaacctttcagactattgtgttctgggccagcgtccaatgcatttaccaaatatgaaccagctggcatccctggggaaaaccaacgaacagtctcctcacagccaaattcaccacagtactccaatccgaaaccaagtgcccgcattacagcccatcatgagccctggtcttctttctccccagcttagtccacaacttgtaaggcaacaaatagccatggcccatctgataaaccaacagattgccgttagccggctcctggctcaccagcatcctcaagccatcaaccagcagttcctgaaccatccacccatccccagagcagttaagccagagccaaccaactcttccgtggaagtctctccagatatctaccagcaagtcagagatgagctgaagagggccagtgtgtcccaagctgtctttgcaagagtggcattcaaccgcacacagggattgttgtctgagattctgcgtaaggaagaagaccctcggacagcctctcagtctcttctagtaaacctgagggccatgcagaatttcctcaatctgccagaagtggagcgagatcgcatctaccaggatgagagggagcggagcatgaatcccaatgtgagcatggtctcctcggcctccagcagtcccagctcctcccgaacccctcaggccaaaacctcgacaccgacaacagacctccctattaaggtggacggcgccaacatcaacatcacagctgccatttatgacgagatccaacaggagatgaaaagggccaaggtgtctcaagccctgtttgccaaagtggctgcaaataaaagtcagggctggctgtgtgaactgctccgctggaaggagaacccaagcccagaaaaccgcaccctctgggaaaacctctgtaccatccgtcgcttcctgaaccttccccagcatgagagggatgtcatctatgaggaggagtcaaggcatcaccacagcgaacgcatgcaacacgtggtccagcttccccctgagccggtgcag

SEQ ID NO: 182 - DNA, 1703 bp, Homo Sapiens
atccagtccttctgtggaacttctgaacatcttttattagtggaaatattttctacacaatgaagtcaacaacttaatttaaaccagtgtttgtgcggttctgattcatctgctgtggttcccgaagcttgagatctaaggagtacagggtcttttgtgatgacaatatgactaatagtaaaggaagatctattaccgataaaacaagtggtggtccaagtagtggaggaggttttgtagattggactttacgtttaaacacaattcaatccgacaagtttttaaatttactcttgagtatggttccagtgatttaccagaaaaaccaagaagacaggcacaaaaaagcaaacggcatttggcaagatggattatcaactgcagtacagacttttagtaatagatctgagcaacacatggagtatcacagtttctcagagcagtcttttcatgccaataatgggcacgcatcatcaagctgcagccaaaagtatgatgactatgccaattataattactgtgatggaagggagacttcagaaaccactgccatgttacaagatgaagatatatctagtgatggtgatgaagatgctattgtagaagtgaccccaaaattaccaaaggaatccagtggcatcatggcattgcaaatacttgtgccctttttgctagctggttttggaacagtttcagctggcatggtactggatatagtacagcactgggaggtgttcagaaaagttacagaagttttcattttagtccctgcacttcttggtctcaaagggaacttggaaatgacattggcatccagattatccactgcagtaaatattgggaagatggattcacccattgaaaagtggaacctaataattggcaacttggctttaaagcaggttcaggcaacagtagtgggttttctagcagctgtggcagcaattatattgggctggattccagaaggaaaatattaccttgatcattccatacttctgtgctctagcagtgtggcaactgccttcattgcatctcttctgcagggaataataatggttggggttatcgttggttcaaagaagactggtataaatcctgataatgttgctacacccattgctgctagttttggcgaccttataactcttgccatattggcttggataagtcagggcttatactcctgtcttgagacctattactacatttctccattagttggtgtatttttcttggctctaacccctatttggattataatagctgccaaacatccagccacaagaacagttctccactcaggctgggagcctgtcataacagctatggttataagtagcattgggggccttattctggacacaactgtatcagacccaaacttggttgggattgttgtttacacgccagttattaatggtattggtggtaatttggtggccattcaggctagcaggatttctacctacctccatttacatagcattccaggagaattgcctgatgaacccaaaggttgttactacccatttagaactttctttggtccaggagtaaataataagtctgctcaagttctactgcttttagtgattcctggacatttaattttcctctacactattcatttgatgaaaagtggtcatacttctttaactataatcttcatagtagtgtatttatttggcgctgtgttacag

SEQ ID NO: 183 - DNA, 660 bp, Homo Sapiens
ttcgatcctttggtacagaagacagaccgacagatcgtccaataccacctcgagatgaagtctttgaatacattatattccgtgggagtgacattaaagaccttactgtttgtgagccaccaaaaccacagtgttctttgcctcaagacccagctattgttcagtcctcactaggctcatcgacttcttcattccagtccatgggttcttatggacctttcggcaggatgcccacatacagtcagttcagtccgagttccttagttgggcagcagtttggtgctgttggtgttgctggaagctctttgacatcctttggaacagaaacatcaaacagtggtaccttaccccaaagtagtgcggttggttctgcctttacacaggatacaagatctctaaaaacacagttatctcaaggtcgctcaagccctcagttagaccctttgagaaaaagcccaaccatggaacaagcagtgcagaccgcctcagcccacttacctgctccagcagctgttgggagaaggagtcctgtatcaaccaggcctttgccatctgccagccaaaaggcaggagagaatcaggagcacaggcgagctgaagtacacaaagtttcaaggccagaaaatgagcaactcagaaatgataacaagagacaagtag

SEQ ID NO: 184 - DNA, 963 bp, Homo Sapiens
gttggtactaagaagtgcctttcctgacgtctctgctgcttggaaccgcttctagagcagtctctgcttttgccttgcttgctgccagctagactgtgacgacagcacatccaccctccacctctagcccagacacccccatttctacttataatcaagagaaaagctctaagtatctggcattgccctaggctgctttagtgttaaaagaaaagtttgctgaaaaagtaagatatcttctgccaggaaatcaaggaggaaaaaaaaaatcattttctcgattttgctctaaactgctgcatctgtctatgccaaactaatcaataccgattgcaccaccaaactccattgcaaattcagctgtgaggagattccctttcagacaactttgctgaaagcagcttggaaattcggtgtcgaagggtctgccacgttttcatgcttgcattttgggctccaaattggcactgggaaggggttactgagagcacaaggctgataccaggccctacttttaaacgttcatctacttacaatcctagtatttctctaaaaaccaaaacctctttgaattaacagtttcatgctgtgaatttctagtgggagatcttttccttgatattgacgacacaattttccatgtacttttaaagcagggagtggggaaaagtattttgaggggacattttcatcatcagttcagctttttttttttggttgttgctcttttttgggggggttgggtttgttggtttcactgaaacatttaactacctgtaaaatctaaacatggctgttagtgtcacaccaattcgggacacaaaatggctaacactggaagtatgtagagagttccagagggggacttgctcacggccagacacggaatgtaaatttgcacatccttcgaaaagctgccaagttgaaaatggacgagtaatcgcctgctttgattcattgaaa

SEQ ID NO: 185 - DNA, 970 bp, Homo Sapiens
ctttttaaagatgacatcaggtatctgttgacaatggacaaactatggcggaaaaggaaacctccagttccgttggactgggctgaagtacaaagtcaaggagaagaaacgaatgcatcagatcaacagaatgaaccccagttaggcctgaaagaccagcaggttctagatgtaaagagctatgcacgtcttttttcaaagagcatcgagactttgagagttcatttagcagaaaagggggatggagctgagctcatatgggataaggatgacccatctgcaatggattttgtcacctctgctgcaaacctcaggatgcatattttcagtatgaatatgaagagtagatttgatatcaaatcaatggcagggaacattattcctgctattgctactactaatgcagtaattgctgggttgatagtattggaaggattgaagattttatcaggaaaaatagaccagtgcagaacaatttttttgaataaacaaccaaacccaagaaagaagcttcttgtgccttgtgcactggatcctcccaaccccaattgttatgtatgtgccagcaagccagaggtgactgtgcggctgaatgtccataaagtgactgttctcaccttacaagacaagatagtgaaagaaaaatttgctatggtagcaccagatgtccaaattgaagatgggaaaggaacaatcctaatatcttccgaagagggagagacggaagctaataatcacaagaagttgtcagaatttggaattagaaatggcagccggcttcaagcagatgacttcctccaggactatactttattgatcaacatccttcatagtgaagacctaggaaaggacgttgaatttgaagttgttggtgatgccccggaaaaagtggggcccaaacaagctgaagatgctgccaaaagcataaccaatggcagtgatgatggagctcagccctccacctccacag

SEQ ID NO: 186 - DNA, 320 bp, Homo Sapiens
gaacaggaaatatagtggtcattatgattagctatccaaaaggaagagaaattttggagctggtgcaaaaaggaattccagtaacgatgaccataggggttggcacccggcatgtacaggagttcatcagcggtcagtctgtggtgtttgtggccattgccttcatcaccatgatgattatctcgttagcctggctaatattttactatatacagcgtttcctatatactggctctcagattggaagtcagagccatagaaaagaaactaagaaagttattggccagcttctacttcatactgtaaagcatggagaaaag

SEQ ID NO: 187 - DNA, 1094 bp, Homo Sapiens
gggttccttgtggaggctcacccagacaatgcctgcagccccattgccccaccacccccagccccggtcaatgggtcagtctttattgcgctgcttcgaagattcgactgcaactttgacctcaaggtcctaaatgcccagaaggctggatatggtgccgctgtagtacacaatgtgaattccaatgaacttctgaacatggtgtggaatagtgaggaaatccagcagcagatctggatcccgtctgtatttattggggagagaagctccgagtacctgcgtgccctctttgtctacgagaagggggctcgggtgcttctggttccagacaataccttccccttgggctattacctcatccctttcacagggattgtgggactgctggttttggccatgggagcagtaatgatagctcgttgtatccagcaccggaaacggctccagcggaatcgacttaccaaagagcaactgaaacagattcctacacatgactatcagaagggagaccagtatgatgtctgtgccatttgcctggatgaatatgaggatggggacaagctgcgggtactcccctgtgctcatgcctaccacagccgctgcgtggacccctggctcactcagacccggaagacctgccccatttgcaagcagcctgttcatcggggtcctggggacgaagaccaagaggaagaaactcaagggcaagaggagggtgatgaaggggagccaagggaccaccctgcctcagaaaggaccccacttttgggttctagccccactcttcccacctcctttggttccttagccccagctccccttgtttttcctgggccttcaacagatcccccactgtcccctccctcttcccctgttatcctggtctaataaccccccacacatacacctctggtgacctatttgcacagaccgtcgtcttccctccagtcttctgagggataggggacattccatcccaagcttctcccttacccacacctatccttttgaggggctttggggtggagctggggcaagcagagggactgggtcttcacttcttgggctaataaaattgtttctttgtggactaa

SEQ ID NO: 188 - DNA, 378 bp, Homo Sapiens
taccctcaacaacaacaacatcagtcgcatcctggtcaccagcttcaaccacatgccgaagatccgaactctgcgcctccactccaaccacctgtactgcgactgccacctggcctggctctcggattggctgcgacagcgacggacagttggccagttcacactctgcatggctcctgtgcatttgaggggcttcaacgtggcggatgtgcagaagaaggagtacgtgtgcccagccccccactcggagcccccatcctgcaatgccaactccatctcctgcccttcgccctgcacgtgcagcaataacatcgtggactgtcgaggaaagggcttgatggagattcctgccaacttgccggagggcatcgtcgaaat

SEQ ID NO: 189 - DNA, 900 bp, Homo Sapiens
tgctctgggcaccacagtctgccccttcaggctcctggacactctctcctgtggcctttgtgatgtggctatccctgctctcccactgcctcacaggcactgccttctctgccccctccttttgtctgtcatggtgggtctttagctaagttcttcttgggcttcccacaccacacgaccccaccccccaggcaagctgcttgtcccctggggatcttggggatcccacagaccattggcctttgctggcaactcttcctcaacttgtttcctccccccgtactgactcattcattctttccccaactgtttgccaaggacctgcctttcactaggccctgagctcagatgcagaagtgagctgaaaaaccatttctgccttgagtttttcagtccagcaaaggaaacagagattaattagacccctaactaggttctcaacagaggaccattttacccccatagtacattttgaagtgtctgcgagacatttttgatcatcacaattgggatccgccatggtagttcactcttgtgggatattgaggtagggggatcatttgaggccaggagttcaagaccagcctaggcaacatagcaagaccccccatctctacaaaaagttaaaagattagccaggcatgttggtgcacacctgtagtcccagctactcaggaggctgaggcaggaggatcccttgagcccaggacgtcaaggctgcagtgcactatggttgcaccactgcactccagcctgggcaacagagtgagactcccaactccaaacagacaattggaggcggataggttttcctggtgtcaagcgggtggaagccagggatgctgttcaatgtcccacagtgcaaggaggcctcctccctagagaatgatctggccccaa

SEQ ID NO: 190 - DNA, 1024 bp, Homo Sapiens
acttaattcagtgcacaaatgagatgaatgtgaacatcccacagttggcagacagtttatttgaaagaactactaatagtagttgggtggtggtcttcaaatctctcattacaactcatcatttgatggtgtatggaaatgagcgttttattcagtatttggcttcaagaaacacgttgtttaacttaagcaattttttggataaaagtggattgcaaggatatgacatgtctacatttattaggcggtatagtagatatttaaatgagaaagcagtttcatacagacaagttgcatttgatttcacaaaagtgaagagaggggctgatggagttatgagaacaatgaacacagaaaaactcctaaaaactgtaccaattattcagaatcagatggatgcacttcttgattttaatgttaatagcaatgaacttacaaatggggtaataaatgctgccttcatgctcctgttcaaagatgccattagactgtttgcagcatacaatgaaggaattattaatttgttggaaaaatattttgatatgaaaaagaaccaatgcaaagaaggtcttgacatctataagaagttcctaactaggatgacaagaatctcagagttcctcaaagttgcagagcaagttggaattgacagaggtgatataccagacctttcacaggcccctagcagtcttcttgatgctttggaacaacatttagcttccttggaaggaaagaaaatcaaagattctacagctgcaagcagggcaactacactttccaatgcagtgtcttccctggcaagcactggtctatctctgaccaaagtggatgaaagggaaaagcaggcagcattagaggaagaacaggcacgtttgaaagctttaaaggaacagcgcctaaaagaacttgcaaagaaacctcatacctctttaacaactgcagcctctcctgtatccacctcagcaggagggataatgactgcaccagccattgacatattttctacccctagttcttctaacag

SEQ ID NO: 191 - DNA, 234 bp, Homo Sapiens
acatttgaagacagtgatttgttccatttgattggtgttatctgtggcttagcaatttataattgtaccattgtggacctccattttcctttggctttatataagaaactactgaaaaagaagccatccttggatgatttgaaagaactaatgcctgatgttgggagaagcatgcaacagttactggattatccagaagatgacatagaggaaacattttgtcttaattttacg

SEQ ID NO: 192 - DNA, 235 bp, Homo Sapiens
acttattgacagtgccaaagctcggtactggacacaacgagggacctgggtctacgataacgcgcttttgctcctcctgaagtgtctttggtccaacgttgttccagagtgtaccatggcttccagtaacactgtgttgatgcggttggtagcctccgcatattctattgctcaaaaggcaggaatgatagtcagacgtgttattgctgaaggagacctgggtattgtggagaag

SEQ ID NO: 193 - DNA, 199 bp, Homo Sapiens
aacatctccccatggattctggcggtattgactcttcagattcagagtctgatatcctgttgggcattctggacaacttggacccagtcatgttcttcaaatgcccttccccagagcctgccagcctggaggagctcccagaggtctacccagaaggacccagttccttaccagcctccctttctctgtcagtggggac

SEQ ID NO: 194 - DNA, 224 bp, Homo Sapiens
atatcaaacaaagaattactctgtacaaagccagaacacatatatcaaagtaatcctgaagtatcagaacaaaataataggctgtaacagaggaggaaatgattttgaatagcctctctctgtgttaccataataagctaatcctggccccaatggttcgggtagggactcttccaatgaggctgctggccctggattatggagcggacattgtttactgtgag

SEQ ID NO: 195 - DNA, 292 bp, Homo Sapiens
ttatttgctggtataaaggatggagaaagcctgcagctctttgaacaaagttctcgctctgcgcataaacaagagacacacaccatggaggccgtgaagcttgtagagtttgatcttaagcaaacgcttaccaggctcatggcacatctttttggagatgagctggagataagatgggtagactgctacttcccttttacacatccttcctttgagatggagatcaactttcatggagaatggctggaagttcttggctgcggggtgatggaacaacaactggtcaattcag

SEQ ID NO: 196 - DNA, 264 bp, Homo Sapiens
gtgtcagttttcaacctttgagcagtgtcaagagtggctgaagagactgaacaacgcaatccgaccacctgctaaaatagaagatctcttctcatttgcataccatgcttggtgcatggaggtctatgccagtgaaaaagagcaacatggagacctgtgcagaccaggggagcatgtaacttcaaggtttaaaaacgaggtggagaggatgggttttgatatgaacaacgcctggaggatttccaacatcaatgagaagtacaa

SEQ ID NO: 197 - DNA, 658 bp, Homo Sapiens
ttgcagatctgatctcttctgaacacctcatcgtgtctccatccctgggaatctgaccctagcaactggaccactttgttcttggaattttgggtgtcctcttttctcacctttcccttttcccttttccccttccccctcctgagaactccggaagactgtagagattgtcatggagtccagggaaaccttaagcagctcccggcaaagagggggcgagtcagacttcctgccggtctcctcagccaagcccccagctgctcctggctgtgcaggagaacctttgctctccactccaggacctgggaaggggatcccggtgggcggagagcgcatggagccagaggaggaggatgaactaggctcagggcgggatgtggattccaactccaacgcggacagtgagaaatgggtggcaggagatggtttggaagagcaggaattttctatcaaggaggcaaacttcacagagggaagtctgaagctaaagattcagaccacaaagcgggctaagaaacccccaaagaatttggagaactatatatgtccacctgagatcaagatcaccatcaagcagtctggggaccagaaggtgtcccgtgctggaaaaaatagcaaagccacgaaggaggaagaaagaagccactccaaaaagaag

SEQ ID NO: 198 - DNA, 271 bp, Homo Sapiens
acaacagttgatatttctacttggtgtgagcagtttgcaactttttgttcagagcaactggacggggccccctgttgacttacaccctcaggactttttgtcatctgttttgttccagcaattcagtgaggttaaaggactggatgcatttgttctgagcctgctcactctagatggtgaatcaatctacagcctgacctcgaagcctatactactgttattagcacgcattatcctagtgaatgtaagacataaactgacagctattcag

SEQ ID NO: 199 - DNA, 574 bp, Homo Sapiens
gctgacagagtacttcttggtggtcctggtagcttttattggcaaggtcagcttatttcggatcaagtggcagaaatcgtatctaaatacgaccccaatgtttacagcatcaagtataataaccaattagcaactcggactgcacaagctatttttgatgacagctatttgggttattctgtggctgtcggagatttcaatggtgatggcatagatgactttgtttcaggagttccaagagcagcaaggactttgggaatggtttatatttatgatgggaagaacatgtcctccttatacaattttactggcgagcagatggctgcatatttcggattttctgtagctgccactgacattaatggagatgattatgcagatgtgtttattggagcacctctcttcatggatcgtggctctgatggcaaactccaagaggtggggcaggtctcagtgtctctacagagagcttcaggagacttccagacgacaaagctgaatggatttgaggtctttgcacggtttggcagtgccatagctcctttgggagatctggaccaggatggtttcaatg

SEQ ID NO: 200 - DNA, 3648 bp, Homo Sapiens
tctcctaaagagcccgaacagctgaggaagctcttcattggagggttgagctttgaaacaactgatgagagcctgaggagccattttgagcaatggggaacgctcacggactgtgtggtaatgagagatccaaacaccaagcgctccaggggctttgggtttgtcacatatgccactgtggaggaggtggatgcagctatgaatgcaaggccacacaaggtggatggaagagttgtggaaccaaagagagctgtctccagagaagattctcaaagaccaggtgcccacttaactgtgaaaaagatatttgttggtggcattaaagaagacactgaagaacatcacctaagagattattttgaacagtatggaaaaattgaagtgattgaaatcatgactgaccgaggcagtggcaagaaaaggggctttgcctttgtaacctttgacgaccatgactccgtggataagattgtcattcagaaataccatactgtgaatggccacaactgtgaagttagaaaagccctgtcaaagcaagagatggctagtgcttcatccagccaaagaggtcgaagtggttctggaaactttggtggtggtcgtggaggtggtttcggtgggaatgacaacttcggtcgtggaggaaacttcagtggtcgtggtggctttggtggcagccgtggtggtggtggatatggtggcagtggggatggctataatggatttggtaatgatggtggttatggaggaggcggccctggttactctggaggaagcagaggctatggaagtggtggacagggttatggaaaccagggcagtggctatggcgggagtggcagctatgacagctataacaacggaggcggaggcggctttggcggtggtagtggaagcaattttggaggtggtggaagctacaatgattttgggaattacaacaatcagtcttcaaattttggacccatgaagggaggaaattttggaggcagaagctctggcccctatggcggtggaggccaatactttgcaaaaccacgaaaccaaggtggctatggcggttccagcagcagcagtagctatggcagtggcagaagattttaattaggaaacaaagcttagcaggagaggagagccagagaagtgacagggaagctacaggttacaacagatttgtgaactcagccaagcacagtggtggcagggcctagctgctacaaagaagacatgttttagacaaatactcatgtgtatgggcaaaaaactcgaggactgtatttgtgactaattgtataacaggttattttagtttctgttctgtggaaagtgtaaagcattccaacaaagggttttaatgtagatttttttttttgcaccccatgctgttgattgctaaatgtaacagtctgatcgtgacgctgaataaatgtcttttttttaatgtgctgtgtaaagttagtctactcttaagccatcttggtaaatttccccaacagtgtgaagttagaattccttcagggtgatgccaggttctatttggaatttatatacaacctgcttgggtggagaagccattgtcttcggaaaccttggtgtagttgaactgatagttactgttgtgacctgaagttcaccattaaaagggattacccaagcaaaatcatggaatggttataaaagtgattgttggcacatcctatgcaatatatctaaattgaataatggtaccagataaaattatagatgggaatgaagcttgtgtatccattatcatgtgtaatcaataaacgatttaattctcttgaatgaaatgacaactgtatggatttgggactggcagagatttggactttccctacccactccccctgataataatgttgaatgcttctatcacaattcaagttcaaagctctgccagggaatagaaactagctgctggctaatgccgctccataaatccgcagatttgaagtgtctgggaggccttttaaaaataactaatatcagaaagcattttaatgaacgtaaagataggcttacattaaaggaaaactgcagtttgactgggttctggttgggtgggccttcagaggttctatcagttaactatactaattaatttggagattcaaaccataccaatagaaactaaatttttctacataatttcatgttaactgcagtttccctttatggcacaaggggtcacacaacctacctaaaatgttaattgtatagaaaatgatttctaaacctttaacagttaatatccaataatgtgttatttgtacatagattcttttgagccttacctttggtgctctctcaatttcagtgctattgtacctagtagcatagagatttgtctatcaataggactgtatattccatcccatgggtgtgttggaagtttggggaggaggagggtggtgggaggtgggtgggaggtggtggtgggtgggaaagcatgggtgatagttccatgatactggctgagtttgcaatagcaggtggaaccctaactattgagggagtttgcagatacctcaggattcgtgacaatgcctttaaagatccaggagagatgttcagctactaggaactgctagcaagtatagcgcgaatggctccgagctcagacactctacagctgagagtagacacttgtggtatgtggagtacagataagccaggggcaggccacggcacgctccatgaaagctaggagggagtgaaatatcagtgatcatcgcaaggaaggaggcagacaagagtaaggcacacctgactcttaggactagcagtcagaaccaggaggaaaggttttattgctatgcgggtaggtaagaacagattttacttacatccatatagttacttaaagtccagttttctgttaaacatttttcttaatatattgagccaaaactagtccagttaagctgaacttggtttttctggagatgaattgttttaaattgacaccctattgatggctcccagttgaaggaagtgagcacattatttgtactgtgaatataaatttttgcccttttatttatcttcctttgacccatttccttaaaataatggctcaaagtaatagacttccccaaatggtggggggatgggtgggttattaatgggaggtatggggggtttagcttgagatgggacttggtcttagagctagttctaaaggttgtttacttttctagggaggagtctgctactagtcttatcagctcttaaaaacagaaactcatctgtccaagttcgtggcagaaaggtaagtttttacaaattagtgctcagcaaaagaatgccctgcgttcccaaagtaaaagaatgacaagctgtaccttaaaccaaaacacttcgtaatctcatccaattgcaaaaagagttattagccaaccaggtattcccagtagtgacagtggatataactgtgtagtcattcacctctgcttatatgaatactttacaacctcttttgccttttgcaggaacgtccttgtgaagacctttatctgagccactgtacttcgttatcactgccatgcagtttacatgagctgttctgcagctcaaattccattttgtgaatgggtttttttttttaataaactgtatttaacttag

SEQ ID NO: 201 - DNA, 182 bp, Homo Sapiens
ggacaatggtgacagatccaggcaccgagctccacgcaatgcccggatgtctgcaccttcgcttggacgctttgtcccaaggcgtttcttgctgcctgagtacttgccttatgctgggatttttcatgaacgtggacagcctggcttggctactcactcttctgttaacagggtcctggcag

SEQ ID NO: 202 - DNA, 357 bp, Homo Sapiens
cccagcagaccgggttatcccagtccgaggtccagcgaaggattttatcccagcccacagcacatggtacaaaccaatcattaccaggtttctggctaccctggttcacatggaatcacagccatggctggcagcatctatccaggtcaggcatctcttttggaccaaacagattcatggaatcatagacctcaggagatagcaatgtggcagcccaatgtggaggactctacagtattggacctgcgagggattgggcaagtgttgccaacccatctgatggaagagcgtctaatccgacagcaacaggaaatggaagaagatcagcgctggctggaaaaagaggaaagatttctg

SEQ ID NO: 203 - DNA, 198 bp, Homo Sapiens
agtgaagagttccacatttatacccagtattgcactaactatccaaggtccgtggctgtgctaacagagtgtatgaggaacaagatactggccaaattcttcagggagcgtcaggaaactctgaaacactcgctgcctctggggtcctatctcttgaaaccagttcagcggattctcaagtatcatctccttctgcat

SEQ ID NO: 204 - DNA, 503 bp, Homo Sapiens
gttgtgacaaagacctttcatggtgcaggcttggttgttccagtagataaaaatgatgttgggtaccgagagctccctgaaacagatgctgacctcaagagaatttgcaagacaatagttgaggctgcaagtgatgaggagagactaaaagcttttgctcccattcaggaaatgatgacttttgtgcagtttgctaatgatgaatgtgattatggcatggggcttgaattgggaatggatctcttttgctatggctcacattattttcataaagttgctggccagcttttacctcttgcatataatctgttgaagaggaatctgtttgcagaaattattgaggagcatctggcaaacagaagtcaagagaacatagaccaacttgctgcatgagtaaggtggctttgattggtgtacagtatttcaaaggactagtattaaacttgtgatttttgttttgtttttaaggaatacaaaaaataaacatttactaaaaacgttta

SEQ ID NO: 205 - DNA, 237 bp, Homo Sapiens
gttttccagcttgcagctcatctggtgtactggggcaaggccatcatcatctacccgctgtgtgagaacaacgtctacatgctgtctcccaatgccagcgtatgtctgtactccccgctggccgagcagttctcccaccagttcccatctcatgacctgccgtccgttcttgccaagttctccttgccggtctccttgtcagaatttaggaatcccctggcccccgctgtgcaggag

SEQ ID NO: 206 - DNA, 3135 bp, Homo Sapiens
agttctcaactaatgaaaaatcattgcattatcttgagagcaatagcaatgatcatgccaacagtaattcagactgtgtcataagttgggtcacacagtccctattttcaaactttgtggatgtctgtcaactggcagtgaattgtattcaggagaaaagaagtctaaagtaagtagtccaagtgtatgatgtcaagaatctgcccatacttggtagtcccttgctactgtacaataatagtggtgcacatttgaaggactgagatgccactaactctctgctccaccattttggggtcacagtgcatatcagtggagggaagcaagtgtttagagggatcctggctttggaagacatttgtgatgatgctggttgaaagcaaaagcatacagcactcagtattaccaggtggtctccaatctgaatcctggttgataagatacaactttgctcaaaatttgctttttatggggatgtatgtggtggcttatgtctttaatcctggcactttgggaggctgaggcgggcggatcacgaggtcaggagatcgagaccagccagtctggccaacatggtgaaaacctgtctgtactgaaaatataaaaattagccaggtgtggtgacatgtgcctgtaatcctagctactggggaggctgagccaggagaatcacttgaactcaggagaaagaagttgcagcgagccgagattgtgcaactgcactccagcctgggcaacctagtgagactccgtctaaaaaaatttttttttttgcttcttaatcctattttacgtaatacaggggaaaagttgagtagaaaattccagaagtgcctttcttcccattgcagaagacagaggacccctgcacctgacttgcagttgtcttccactgtgagtctctcaggaacccagaagcctgaccttagagattacacaatacttcgtagctcagtgtgtgtgccaggaagtgtcagggtttgagcttccaattcttaaattccgcttcatctcaggtacactcctgctttgtctctgactgtttttcctgctaggccaatatattaggagatgtcaaagggctgagagtgtaccagcccaacttagctgacctttcttagagccactgcttatcaaccaactcaagacctgacacaggacattccctggctgatcaaaaaataatgataagagctttaagggaggggcacaataaaagggtaaatctcaaagcaggtggctgtactttgggcttgctcaggacataatgtaactgtcctgggtctggaaataactcagatggactcacagagcagacccagatacaaagcagacatctgtaggggcttttactaccccattcatgttcatatatgccatagtaatgaatgtattataggatgtgttcttgtttgtttttctttctatttttctctgtctttgtcaaggaatcccatatttgcaaagtcccttgcccttactgcattagaaatgggcattagaaatgggactgaatgttgggtccatctagcatcctggggccttgattataaatacagtttgtaaaatggattacagaagcaaatgaaagattgtttaccggcatcttatttggtggggaggaaatgtaattcagtacagtagtagcccaggttttgtggagacagaggtacattggggaaagctggggctaggagtttcttattgggacctttagtatatgggtgaattttgtcagggtctggtatgcggaaagattcatagaagtagttgtcagcagacaaaagagatttgtcagcaaactaaaaatctcataccctgagacagtgatggaggaaagaaacttgtcaggaaggttttctagaagatgctgcattctgcagtggacattttcacagctgaaaaataagtggcaacatcctaagtactcataatatcaaggtggtaaaatcctgctctgcttttgagcaagatgatataagtgaatcaatacattcatgctattaaggttataggtttttacagctttcacttgcatgaaaaaacatgttgtctatgcttctgcagttctcagggcagtgaacctaagccaggagctgcttaggcattaaggcttctatacccagaatcgtgtgctaagctttctctccaagtcagcacaacaaagcaaaccagagcatcgtaccaacaacctgaaaggtcacctgactccttccttctcactcccattctgtacttctgcatacgtgggcaggcaagtgaatggtaagtaagcctgcaaacagcctttctttgtatagggaatgacaggcatttcatcagcaactccaggatggcaggccgttatcctgaaacctatcagaacagctcctgcttggcttccaaaatatcacaattttggaaatgtacaaaggttagttttcacagtccttgaagcctctttccaccctggccatagcccatgtcagcagtaacctatgtctttgttttacatatcatgttgttttactcacctggtgcctccagtgccctatagggtccctgagctgtcctgatactctttcttagatcctgtcaaaacactcacttcaacaaattctgcccttggactctgatgggaggttctgataaagaaaattattgaaaaccgcattatttaaatgtggttatatttcagctggacctggaaataaagacgaaatactgcttcgtgactgtgctttaaggaatttttgcttctaggagatgattgtgagccaggcagaggtgaaggagcaggagcaaatagccaaggcacagttcacagtgctggattttcttgtggatatagctgcattgttgttccttctgccttgtccaactgcttcctggtatccctaccctcaccttttcgttatcttttcatctctctaatcattgagaaaaagagtacaaaaagccagattttgcagttccctattttgtattccaggttgaagagtgctctggctgtagagggtccccacgatctgtcatctagtaactcaccagatacataagatattttgcctgctatgaagccattggagaatcaggtccagctgtgtcctacagatttataccctcactttggatgcactgtgaatcttgaaacaa

SEQ ID NO: 207 - DNA, 360 bp, Homo Sapiens
ccgtcctggtgcgcggggacagtgaggcttggctcctttatcagtgacaccggtccctgggtcttgctggggtgagaagaaccaaaacattgtgggtgatgagctaagcgggactgctaaggttcacctgtgcgtttccgcagagataaccttaatggacatggcaaggctgtgtgacttggaggcagctttttccattgacgttgctctccctaaaatgcttcgtattccaaaatatttctagaaagaccttgtaggataaggttgataaagagtccattgtatacgtttccgggtagttaaattagggaacagttttctctgccccgcccgtggttcacaggtagataactgtctcgt

SEQ ID NO: 208 - DNA, 1324 bp, Homo Sapiens
gaacttgctactaccagcaccatgccctaccaatatccagcactgaccccggagcagaagaaggagctgtctgacatcgctcaccgcatcgtggcacctggcaagggcatcctggctgcagatgagtccactgggagcattgccaagcggctgcagtccattggcaccgagaacaccgaggagaaccggcgcttctaccgccagctgctgctgacagctgacgaccgcgtgaacccctgcattgggggtgtcatcctcttccatgagacactctaccagaaggcggatgatgggcgtcccttcccccaagttatcaaatccaagggcggtgttgtgggcatcaaggtagacaagggcgtggtccccctggcagggacaaatggcgagactaccacccaagggttggatgggctgtctgagcgctgtgcccagtacaagaaggacggagctgacttcgccaagtggcgttgtgtgctgaagattggggaacacaccccctcagccctcgccatcatggaaaatgccaatgttctggcccgttatgccagtatctgccagcagaatggcattgtgcccatcgtggagcctgagatcctccctgatggggaccatgacttgaagcgctgccagtatgtgaccgagaaggtgctggctgctgtctacaaggctctgagtgaccaccacatctacctggaaggcaccttgctgaagcccaacatggtcaccccaggccatgcttgcactcagaagttttctcatgaggagattgccatggcgaccgtcacagcgctgcgccgcacagtgccccccgctgtcactgggatcaccttcctgtctggaggccagagtgaggaggaggcgtccatcaacctcaatgccattaacaagtgccccctgctgaagccctgggccctgaccttctcctacggccgagccctgcaggcctctgccctgaaggcctggggcgggaagaaggagaacctgaaggctgcgcaggaggagtatgtcaagcgagccctggccaacagccttgcctgtcaaggaaagtacactccgagcggtcaggctggggctgctgccagcgagtccctcttcgtctctaaccacgcctattaagcggaggtgttcccaggctgcccccaacactccaggccctgccccctcccactcttgaagaggaggccgcctcctcggggctccaggctggcttgcccgcgctctttcttccctcgtgacagtggtgtgtggtgtcgtctgtgaatgctaagtccatcaccctttccggcacactgccaaataaacagctatttaagggggagtcggc

SEQ ID NO: 209 - DNA, 487 bp, Homo Sapiens
atggagttttaacattgaatgcggagaacactaattatgcctatcaagttccaaacttccataaatgtgaaatctgtctactatcttttccaaaagaatcccagtttcaacgccacatgagggatcacgagcgaaatgacaagccacatcgatgtgaccagtgcccccaaacatttaatgttgaattcaacctgacacttcataaatgcacccacagcggggaagatcctacctgccctgtgtgtaacaagaaattctccagagtggctagtctcaaagcgcatattatgctacatgaaaaggaagagaatctcatctgttctgagtgtggggatgagtttactctgcagagtcagctggccgtgcacatggaggagcaccgccaggagctggctggaacccggcagcatgcctgcaaggcctgcaagaaagagttcgagacctcctcggagctgaaggaacacatgaagactcattacaaaattag

SEQ ID NO: 210 - DNA, 308 bp, Homo Sapiens
acatgaagccattattatatcaggaaccgaaatggccaagcatatccagaaagaaatacagcgaggtgtggaatcatgggtttcccttggaaacagaagacctcacctcagtataattttagtgggagataacccagcaagccatacatatgtcaggaataagataagagctgcctctgctgtaggtatttgtagtgagctcattctaaaacctaaggatgtttctcaggaagaacttttggacgtaactgatcaattgaatatggacccaagagtcagcggtatattagttcagttaccactaccag

SEQ ID NO: 211 - DNA, 153 bp, Homo Sapiens
aattccctgaccaggagcgcacccctccccaacgactcccaggcccgcagtggagctcgttttcacacttcctacgacaaaagatacatcatcaagactattaccagtgaagacgtggccgaaatgcacaacatcctgaagaaataccaccag

SEQ ID NO: 212 - DNA, 389 bp, Homo Sapiens
aaatctcccagcaaaaccagctgaagaagctcagaagcacagacagcagtatgaagaaatggtggttcaggccaaaaagcgagagctgaaagaagcccagcgaaggaagaagcagctggaagaaagatgcagagtcgaggaaagcattggaaacgctgtgctcacctggaataatgagatcttacctaactgggaaacaatgtggtgctctagaaaagttcgagatttatggtggcagggaatccctccaagtgtgagaggcaaagtctggagcttagccattggcaacgagttaaatatcacccacgagctctttgacatctgtcttgcccgagccaaggagaggtggcggtcccttagcacaggaggctctgaagtggagaacgaag

SEQ ID NO: 213 - DNA, 854 bp, Homo Sapiens
caactgaacgaggaagggatgctgttggcttcagagatcaaacacctgccccgaagactcctaataggtcaagagagagagacccagacaagcaaactcaaaataaagagaaaaggaaacgaagaagctccctctcaccaccctcttctgcctatgagcggggaacaaaaaggccagatgacagatatgatacaccaacttctaaaaagaaagtacgaattaaagaccgcaataaactttctacagaggaacgccggaagttgtttgagcaagaggtggctcaacgggaggctcagaaacaacagcaacagatgcagaacctgggaatgacatcaccactgccctatgactctcttggttataatgccccgcatcatccctttgctggttacccaccaggttatcccatgcaggcctatgtggatcccagcaaccctaatgctggaaaggtgctcctgcccacacccagcatggacccagtgtgttctcctgctccttatgatcatgctcagcccttggtgggacattctacagaacccctttctgcccctccaccagtaccagtggtgccacatgtggcagctcctgtggaagtttccagttcccagtatgtggcccagagtgatggtgtagtacaccaagactccagcgttgctgtcttgccagtgccggcccccggcccagttcagggacagaattatagtgtttgggattcaaaccaacagtctgtcagtgtacagcagcagtactctcctgcacagtctcaagcaaccatatattatcaaggacagacatgtccaacagtctatggtgtgacatcaccttattcacagacaactccaccaattgtacag

SEQ ID NO: 214 - DNA, 277 bp, Homo Sapiens
cccatcgatttgagtgcatgcactgttgcacttcacattttccagctgaatgaagatggccccagcagtgaaaatctggaggaagagacagaaaacataattgcagcaaatcactgggttctacctgcagctgaattccatgggctttgggacagcttggtatacgatgtggaagtcaaatcccatctcctcgattatgtgatgacaactttactgttttcagacaagaacgtcaacagcaacctcatcacctggaaccgggtggtgctgctccacg

SEQ ID NO: 215 - DNA, 1041 bp, Homo Sapiens
atccctgggtatcagtatgtgtcacctgaaggcaattgtatagaacatgttcagcctacttctgcctttattcagcaacccttcattgatgaagatcctgataaggaaaagaaaataaaggaacttgagatgcttcttatgtcagctgagaatgaagttagaagaaagcgaattccatcacagcctggaagtttttctagctggtctggtagtttcctcatggatgataacatgtctaatactctaaatagccttgacgagcacactagtgagttttacagtatggatgaaaatcagcctgtgtctgctcagcagaattcacccacaaagttcctggccgtggaggcaaacgctgtgttatcctctttgcagaccatcccagaatttgcagagactctagaacttattgaatctgatcctgtagcatggagtgacgttaccagttttgatatttctgatgctgctgcttctcctatcaaatccaccccagttaaattaatgagaattcagcacaatgaaggagccatggaatgccaatttaacgtcagtcttgtacttgaagggaaaaaaaacacttgtaatggtggcaacagtgaagctgttcctttaacatccccaaatatagccaagtttagcactccaccagccatcctcagaaagaagagaaaaatgcgagtgggtcattccccaggcagcgaacttagggatggctcattgaacgatggtggtaatatggcgctaaaacatacaccactgaaaacactaccattttctccttcacagtttttcaacacatgtcctggtaatgaacaacttaatatagaaaatccttcatttacatcaacccctatttgtgggcagaaagctctcattacaactcctcttcataaggaaacaactcccaaagatcaaaaggaaaatgtagggtttagaacacctactattagaagatctatactgggtaccacaccaagaactcctactccttttaagaatgcgcttgctgctcaggagaaaaaatatggacctcttaaaattgtg

SEQ ID NO: 216 - DNA, 171 bp, Homo Sapiens
tgcacttggaggctcctcagctcttcatatcccagcttttccaggaggaggatgtctcattgattacgttcctcaagtatgccacctgctcaccaacaaggtgcagtacgtgattcaagggtatcacaaaagaagagagtatattgctgcttttctcagtcactttggcac

SEQ ID NO: 217 - DNA, 7173 bp, Homo Sapiens
gctgaactcacgggaatcaaatggcgtaggtacaattttggagggcatggggactgtggacccataatttcagccccagcccaagatgatccaattctgttaagtttcatccgctgtctgcaagctaacctgctttgtgtatggcgtcgtgatgtcaaaccagattgcaaagagttatggatattctggtggggagatgaacccaacctagtgggtgtaatacatcatgaactgcagggtaaggtttctgtttttttcttaaaaattatttgcatcatatttttttgaacattgataaatttcaaacagattattaagacttgtctaaaagaacgatcttaagtgtttcatggatgtctctctcaaaaggaacattctcacccctcttcctcccatccctgtttttttttcctaactcttgtccttttccagttatgctgagaatcagaaccttgtgaaccgctgctgttaaatgagagtattgtgtctcctgggtgcggtggggtggaaaccaggctaagatccactgtttttaaatgatagtgaattgtgagtaactaacaagacagtgaattgaacagttactgtttttttccttggactgtggttgttatggttcactgccagctgaactctttggtggaaaattggagagtgatacagaaatttagttttgagcctaattggacaaaattgtgagattacattgaatagtattttgaggaaagatgaatatatgggatctggtctgaattgggtgtagcaaattttcttttgcttttatttctgtttttcatgtaatttggtgattccaggaaacattggctccggtgttgcctgatttttagcttcagattgcatctgatatctggaaactgaattaatgtaagagtttcatttttctgtgctgctttatatatttaccacagttttatgagcaggaacttttatcagttgtctttgaatttctggtaaaaatttttacctttttatctaagtgtttctatttagtatgcaaagggattttctcaagcaagactaattctaatctttagttagggtattaactctttttgacttagtctcttagaaccatcttttattatctccaaactatattgtgttaatctgtgtctgaaaaatttcagatcgtgtttgaaatgttttgttacttgatgtttttaatgttttttttttttaaaggaaggagtaaaactaactgtaatgtatgttcgaggctctgttctaaagcttttacatttgcctgaaggatttcctgccaacttttgcttgacaccttgctagtttttcttggcagccattttttatctgtgatatctcctgccacagcagccagagggcggtaaaccacaatggactaattgaactgtttactttatggtccatttgtctgttcagctcaaacctttatcctgatattttttagggtatttggtctataaataagagaaccattgtgggtgacattgcttgcttagctgggtaatcagcaaagtcccagaacttaacattcatctcaaagcctgttctatgtagttagcagttaacttaggtaaaattagagtttcagaacatctggattctgtttatcagaattttaaaaataagatgtcacctagaagataattacaacatttttgtttgtccattaaaatatgaaaacatatttttactttttttccctatgtagtagagaggtataaagctttttaaaaagtgtctttcttatctttcatgattgtttctgactgtcatcttctgtttctctgtattaatctcttacgttttagctttttggtctgctctttgttagtgtggtacatataattttgtaatgatgaggaaaccaaacccaactgctaatgagggttatttagttgataatatagaataaatttatttatctatgccaacggagaaaatcgtcagtgttcttagaaatagcttattaaattattgtgttatttttcatgggctttgttactgagttaacaaaaaggagtagggcatttaaattttgggatagttgctttagattgcatgtgtaaattaatatcttaagtattttttggtaaagagttaagcaaagattgaattatttttagatatttttaaaattacagaaacactacatttaagttagaaaattaggaaaacagtcatcatatggagagaacaaagaaataaaaatactgaagtctgaaagagttttagatgttttatgtgtaaatgaaattggtaccatacagtttttctaattttctaattaaatgtagtattgtgaatattttctcttgggaatgattacttttctttggagtttgatagtggtataaactgtttatattttgtattctatcaggaaagtattacagtaatggaataattataatttcattgagtgcgtctcctctttttaatatgaattttgagcaagtttttagattcatttatatgaagtgtccatttttgtaggtaaaagtagttgaaaaaggtaatgtatggttcaacctaataacagaaaatatctagccttaattagaaaaataagtcataaagacaactttgtttcaaataaggggaaatacgatagcagtcatgaaaaactgaaggatacaaagagaatattttaattggtagccatacaaagtggagatattttagaatctatttcattctctaatatatttgagtcattttattttgggtaaaataaattataggcggagcatggtggctcacgctggttatcccaacactttgggaggctgagggggcccaaatgcttgaggccaggaatttgagaccagcctgaccaatatggtgaaaccctgtgtttacaaaaaataaaaaaaaaaaattagccaggtgtggtgacgtgcacctgtaattccaactactcgggtgactggggctggaggatcacttaaacccaggaggccaaggttgcagtgagcagagataatgccactgcactccatcctgggcaacaaagtgacacctgtctcaaaaaataaaataaataaatagtagcagaatccttttagcaggctttaatgttaagcatgagattaccattatttgtaacattttttaaatagggacattttactgtttcttacattgctgcttatttatgttatttggcatttcctggctggtcacggtggctcacgcctgtaatccctgcactttgggaggcctaggcaggaggatcacctgagttcaggagttggagaccagcctggccaacatagtgaaaccccctctctagtaaaaataaacaaaaattagctgggcatggtggtgggtgcctgtaatcccagctactggagaggctgagacaggacagtcgctggaaaccgggaggcagaggttacagtgggctgagatcacgccacagcactctgctgggcaacacagcaagactccatctcaataaataaataaataaataaataaataaatagcagaatcctgttagctggcttaaattaagcatgacattaccattatttaacattttttttaatagaggcattttattgtttcttaaattgctttttagttatttggcatttccctttgtcttcatagcgtactccaaatagcgtcctaccaagaaacctcacggtgagatgtaataccaaagcaaccatcttgcatttaaatgtataaagtatcccataaaatgagaataatcctacagggtacacacacaagttagttttatgatattatactttttgtttaattgcttgacatatctctgaaatagtctgagagaaggctataacacatttataatctgtccttgtcacttggataaacacttgggttcctgtctggggaattgttaatgcgtgtggtgtagtgattcagagcacgggctctgggttgcttgggtttgaatgcttgcatagtcactagctgtgtattcttgatctaattatttaacttaaactgtgcctcagttttctcactgtgaattaggttatgaggttaagaaaaattaaaagtacttagagcagcatatatagcagttaatctataattgtaactattgctactgctactgtgcaaagacctacaggctgcgaatgaattgtggcaagaacaggttacctggggcttagggtaggagtgaaatagtctcccctctaaaaggctgagtaggtgggtcatagtatggagtacacagaaccaaggataagacatttagacttaatttgatagattttgaggaattaaacatttctgatcaggaaagcgatataattaggaagattaacttagtaataatacaggattaatgtgtacagggaaaccataatattgtgattttaaaattctaaattggaatataaacttaaatattctgttcaggatacagatgtatttaaattaaaaatactaatgattatatactataattcatattaatagtctttaacacttttattatacctttgctgttttggcttgactaaattctacattttaatttaagttttagagattaatacagtgtgctgagtggacttctcctaatctaccaaacaaatacttcttacttttttttttttttttttttcctttagacagagtcttgctctgttgcccaggctggagtgcagtgatgcaatcttggctcactgcaacctctgcctcccgggttcaaatgattctcctgcctcagccttctgagtagctgggattgcaggtgtatgccaccacacctggccaatttttgtatttttagtagagatggggtttcaccatgttggccaggttgatctcaaactcccaacctcaatcgatccgcccgcctcggcctcccaaagtgctgggattacaggcgtgagccactgggcctggcaacaaatgctttttaaataaacacaatattctcgccaattcactgcctgttagttagtggtgggaaagacatgcaatctggaaaacccaaaatagtacagagagcaagcacagaactgccaattgcttgccccaaagcattacaaatctctggtccagaaggagaagtatcagattcagcctagtatgtgcttaagaatgtttgtcgaatcagttaatgaaacgtattttatgaggttctaatgtagcgcagtattgcactcccggcagattgacttataataaatttgtgaaaatttttaatgatttgataatgcgaggatattgaagcctagttaaaataaagcatattatttctggttttaattcccaaggggatagaaatagccaatttttggggtatattttctaccacaaatatttagcactaaatatgcactgtctacaattttaaaatttatgttgtctctttttttgattattagacatataaattaaggatacatctgtacaactctaaaaatgattgtccctataacagtatcagaagtcttctgaattggttttattgtcagtgcaggggtagattggcagttattaccctttgttgtgaatttagagacataaatattgctttttctatttttaagagtgctgccacctgtgccaggaggtgtgggcgattgtaaaaacattagacataagagagcctggaaaaatggatctgttttctgacagtgaccacggtgtaagataatttaagatactagtactattgtttattgaactcattgcaagtgggcattttgtaggagtgtgggaaggctggatttttttagttttgtaatctgtatctatagattacagtctacaattccaaatggtgggattgataaaacctttgtttagataactccaagcaagaacctagcttctccactcctcctagctttttatttttaaatatatttacacacttatatgcatatgcctctttttccacctaaactatttgaggcaattttcagacttcacggacctttgcccctaaatacttcagcatttaagtcctgtgaatgagaatgtgaagcactcttttacgtaaccagagcacttttggcatgctcaagaaatttaatatcgatataattaaattatctaatatttagtccatattcaaatttcccaattgtcttagcaataccttttatagttttctctctctctgatccagaatgcaatcaggatcacacatttttagatggcttgtgtcctctcatttagaatattcctgaagttgtcttggggatcaatgatattgatgttatttgaagggagcagtttagttgtcctgtaaaatatcccacattttgaatttgtctaattgttttcttacagttagagtcaggataaacatatttggcaagaatactgagtaggtgatgttgtgtcctttccacggcatcacatcagaaacaggtcatgtcagtttgtctcaatattgatggtactgagcttgatcccttgattaaggtagtgtctgccagatttctccagtgtaaagatatcctttccttttgtaattgttaagtaaatttgggatgaaactttgagattgtgtgaatatctctttcctcaaaagagcctaattttgagcctgtttaaaccaaccgaaaaattccggaatcacctgcttttagaaaatcagttgagatcacatgaagactgcatttttaagtcacagcagagttcaagaggagaagagaataaaatattgaagatctaccaaaggcaaattataatggcaatgtcacactatgaaaatgaatagcccctattgcttagcatacctaaaaagagaagtgttgacattgaattttccaaaatcatagacaataatgtatgtaggaatatattactgtgtatcttagagtctgtgaactttcaattgatttttgatggtttctggttatccttaactaaatggtaaaaataatttcatcggtattatacatgaatttattatcagttgaaattaaaaaattgctttctggaaaagtaatgactattttatatataaattgacaggaataagattatctcttgtcattttgaaaatttctttcaaggagaaaatctgatctttgactcattttcactctgtttttctatctttttttttagtggtattaaatttgtgagtcattgaaattaatccctgactgaattggaaaggaattgtatttgcagtatttggatttatttatttttcag

SEQ ID NO: 218 - DNA, 419 bp, Homo Sapiens
gcatgagccatgagcccaagtccccttcgctagggatgctttccaccgcgaccaggaccaccgccaccgtcaaccccctcaccccctcgccgctcaatggcgccctggtgcccagcggcagccccgccaccagcagcgcgctgtcggcccaggccgcgccatcctccagctttgccgccgcgctgcgcaagctcgccaaacaggcggaggagcccagagggtcctcactgagcagcgagtcgtcccccgtgtcctctccggccaccaaccacagctcccccgccagcacacccaagcgcgtgcccatgggccctatcatcgtcccccctgggggccacagcgtgcccagcaccccccccgtggtgaccatcgctccaaccaaaaccgtgaatggtgtctggaggagtgagagccggcag

SEQ ID NO: 219 - DNA, 277 bp, Homo Sapiens
ccttaaattggaatgtgagaaactggcaagtgaaaagacagaaatgcagaggcactatgtgatgtattatgaaatgtcatatggattaaacattgaaatgcacaaacagactgaaatcgccaagagattgaatacgatttgtgcacaagtcatcccatttctgtctcaggaacatcaacaacaggtggcccaggctgttgaacgtgccaaacaggtgaccatggcagaattgaatgccatcatcggggtacgtggcctaccaggtctacctcctaca

SEQ ID NO: 220 - DNA, 371 bp, Homo Sapiens
atcttctgtctacatggtggtctctcgccatctatagatacactggatcatatcagagcacttgatcgcctacaagaagttccccatgagggtccaatgtgtgacttgctgtggtcagatccagatgaccgtggtggttggggtatatctcctcgaggagctggttacacctttgggcaagatatttctgagacatttaatcatgccaatggcctcacgttggtgtctagagctcaccagctagtgatggagggatataactggtgccatgaccggaatgtagtaacgattttcagtgctccaaactattgttatcgttgtggtaaccaagctgcaatcatggaacttgacgatactctaaaatactcttt

SEQ ID NO: 221 - DNA, 977 bp, Homo Sapiens
agcagctcttaaagctttggattcggctgtcgtggagataacgggcaccagaaaaacttgtaggagtagcaacagctatttggtcggatggaggccagaagccaagaataatcgaaacacgctgccagcgcccattagatggatgggaaatgagtgtaactcattgggaaagatattagacttcaaatcacctaaggcaaaccctacttcctggggcgcgggcgcgggcgctggcgttaggagacgtcactcccgcgcataactgacatggggccctcttggtcggcgtttccgggcgggtccttccgacggccgccgcggtgattccatcactcggctttcttcccggcctgcctcgcgcccgtagccgggctgggccagaacagcccaagatggccgacttcgatgatcgtgtgtcggatgaggagaaggtacgcatagctgctaaattcatcactcatgcacccccaggggaatttaatgaagtattcaatgacgttcggctactacttaataatgacaatctcctcagggaaggggcagcacatgcatttgcccagtataacatggatcagttcacgcctgtgaagatagaaggatatgaagatcaggtcttaattacagagcacggtgacctgggtaatagcagatttttagatccaagaaacaaaatttcctttaaatttgaccacttacggaaagaagcaagtgacccccagccagaagaagcagatggaggtctgaagtcttggagagaatcctgtgacagtgctttaagagcctatgtgaaagaccattattccaacggcttctgtactgtttatgctaaaactatcgatgggcaacagactattattgcatgtattgaaagccaccagtttcagcctaaaaacttctggaatggtcgttggagatcagagtggaagttcaccatcacaccacctacagcccaggtggttggcgtgcttaagattcag

SEQ ID NO: 222 - DNA, 2334 bp, Homo Sapiens
tcctgggtcaagctgagtatctccacgtttaggtgcacaaatactattttgtgaagatcttaaatttgtggtataagaacttgaaggaaatagaactgatttgttacttctaaaagatttgtaagactccagggcctcccagccgtgaataatctgatggttcctgaaatgactggggaagcagacgcttcgtatggcagttgaagagtgtgtgtctatgtgcatttaaaaccttctttctgtacttacacattcacacgggaagacaggctcattcttgtgcacacttgagagttttacaactgatgaaaattaatttaagaatcagatggagcaacttgacaccagtgggctcaggagcccggggagaaaaatacatcactaatggccagttttccatatggtctgcacgggtaaagaaagtctgcaaaaagaagaaaaaaaaatttgcgcagattaaaccacaaaaatattctccagtttaaagaaggaactaagtgagaaggtgactgagaaagaagtgtgatttcaaacattgcagcggctcacacagtgtttgttgcactttatttttcagtgggtttggtgatttggacggattaaaattctagactgaaaagtaactcctactgtggttatggctagaggaaagcaagttcaagtatgatgggacaagtttgaataatgaactgattcctttgcctatcttaattaactgtatttgagaaattttaatttattattcccccccttttttcctgcatctataggataatattgtaaaatagcaattgaaaccaataattattaaataaatatcaaggaaaatccaagcaaagctttctttttgttggactagtggtgtggtgtttggagacagtctctgaatgtgaacaggaaagcacccatcagcaaaacactatcactctctagggagacagctgggggaatctgactctggcttctgcttttgttttaagggattaacttccctgtcaagtccaagaagacttgcgtatgagaagattacctgatggacttaattctaagattagcttttttcatcaagatggaaaaagatctttaggagcagaaaaggggagtgctaactgggggagcgagaagggagacgagcaaaagaaacaaaatcttgccacgtggctctgttttgtcagcaagaggatttaagactcacccagggcaaacactgggaccactgtaagagcgctggaacattctgcctcttgagtgaaggggccttctttctagcctctatggcactgaggggtgcgccggctggtggaggagcagtccgatggagccctgcgttccccggggacacagggccaagctttgaggtggaaagtttctggttctgaaacaacaaggagagagtctgtttttcttcctaaaatttggactcttgtctgcacaaactctggtctgttttgcacggtttgtgtgcctttttttccctttatgcaatctttttcagctttagcagcagaaatttgtctagttcaggaaacatgctagagggtggcttcagaaggaagatgatcctgtgtattctgtctctgcatccgaacttttgaagagaaaaattcgagctagagggattcttaaagccttaagttacttgaaatctatgtatttgcaaccctttgtctctggaatcatattacactaaactggaatctcaggctgaatgagaataaccaagtggagtaaaaagaagaaaaccgtttcttgatcaccacttaattaacgatgctctttctccaaaggatcagcacgttcttcctctgagaacttgaaaatacaaatggaccccatgtttttttaagcattaccttttcttagaagactgccatcatcttttatagaggaattttttcactatgcattcggtggatctttataaaatactgaccttctaattagattcaggtcagtcttaattaaagggggaaaaaagcaacgcaagccaaccacaaaaacacatataccaatgaaagaaattggtttaaatttcacagcattaacattactttttaagtaaaacagttcattgaagaaagtatgtatgcagcagtggaacatgggcctgtgctttgcagcgattccaacatcctgtgcctgtcctggaaggggcgtgtccccaagagtgagaaggagaagcctgtgtgcaggagacgctactatgaggaaggctggctggccacgggcaacgggcgaggagtggttggggtgactttcacctctagtcactgtcgcagggacaggagtactccacagaggataaatttcaacctccggggccacaatagcgag

SEQ ID NO: 223 - DNA, 354 bp, Homo Sapiens
gtaatcagatcctgtcccttgggagcctctcaaaagatcagatttatccaatgaaactcaagggcatctccatctgctattcagctctcaagtctgccttgtgtggaaattatgtcagctttggcgtcttcaagttgtatggggacaaccattttgacaatgtactccaggcttttgtcaaaatgctgctgtcagtgtcccacagtgacttgctacaataccggaaactgagccagtcttattatccactcctggaatgtctcactcaggaccatatgagcttcatcatcaacttagagcctcctgtactcatgtatgttctcacatctatctcagagggactcactactcttg

SEQ ID NO: 224 - DNA, 2056 bp, Homo Sapiens
gttttggttcctttgtggacaaaacggtgctgccctttgtgagcacagtaccctccaaactgcgccacccctgccccacccggctggagcgctgccagtcaccattcagctttcaccatgtgctgtccctgacgggggacgcacaagccttcgagcgggaggtggggcgccagagtgtgtccggcaatctggactcgcctgaaggtggcttcgatgccattctgcaggctgcactctgccaggagcagattggctggagaaatgtgtcccggctgctggtgttcacttcagacgacacattccatacagctggggacgggaagttgggcggcattttcatgcccagtgatgggcactgccacttggacagcaatggcctctacagtcgcagcacagagtttgactacccttctgtgggtcaggtagcccaggccctctctgcagcaaatatccagcccatctttgctgtcaccagtgccgcactgcctgtctaccaggagctgagtaaactgattcctaagtctgcagttggggagctgagtgaggactccagcaacgtggtacagctcatcatggatgcttataatagcctgtcttccaccgtgacccttgaacactcttcactccctcctggggtccacatttcttacgaatcccagtgtgagggtcctgagaagagggagggtaaggctgaggatcgaggacagtgcaaccacgtccgaatcaaccagacggtgactttctgggtttctctccaagccacccactgcctcccagagccccatctcctgaggctccgggcccttggcttctcagaggagctgattgtggagttgcacacgctgtgtgactgtaattgcagtgacacccagccccaggctccccactgcagtgatggccagggacacctacaatgtggtgtatgcagctgtgcccctggccgcctaggtcggctctgtgagtgctctgtggcagagctgtcctccccagacctggaatctgggtgccgggctcccaatggcacagggcccctgtgcagtggaaagggtcactgtcaatgtggacgctgcagctgcagtggacagagctctgggcatctgtgcgagtgtgacgatgccagctgtgagcgacatgagggcatcctctgcggaggctttggtcgctgccaatgtggagtatgtcactgtcatgccaaccgcacgggcagagcatgcgaatgcagtggggacatggacagttgcatcagtcccgagggagggctctgcagtgggcatggacgctgcaaatgcaaccgctgccagtgcttggacggctactatggtgctctatgcgaccaatgcccaggctgcaagacaccatgcgagagacaccgggactgtgcagagtgtggggccttcaggactggcccactggccaccaactgcagtacagcttgtgcccataccaatgtgaccctggccttggcccctatcttggatgatggctggtgcaaagagcggaccctggacaaccagctgttcttcttcttggtggaggatgacgccagaggcacggtcgtgctcagagtgagaccccaagaaaagggagcagaccacacgcaggccattgtgctgggctgcgtagggggcatcgtggcagtggggctggggctggtcctggcttaccggctctcggtggaaatctatgaccgccgggaatacagtcgctttgagaaggagcagcaacaactcaactggaagcaggacagtaatcctctctacaaaagtgccatcacgaccaccatcaatcctcgctttcaagaggcagacagtcccactctctgaaggagggagggacacttacccaaggctcttctccttggaggacagtgggaactggagggtgagaggaagggtgggtctgtaagaccttggtaggggactaattcactggcgaggtgcggccaccaccctacttcattttcagagtgacacccaagagggctgcttcccatgcctgcaaccttgcatccatctgggctaccccacccaagtatacaataaagtcttacctcaga

SEQ ID NO: 225 - DNA, 80 bp, Homo Sapiens
gaagtgtttggattgtgagctatttcagaactgttctcaggactcattattttaacatttgggagaaacacagccagaag

SEQ ID NO: 226 - DNA, 204 bp, Homo Sapiens
agtgaagatacacaacagcaaatcatcagggagactttccatttggtatctaagagagatgaaaatgtttgtaatttcctagaaggaggattattaattggaggatctgacaacaaactgatttatagacattatgcaacgttatattttgtcttctgtgtggattcttcagaaagtgaacttggcattttagatctaattcaa

SEQ ID NO: 227 - DNA, 898 bp, Homo Sapiens
ctccaggacacgcgcgccccgagcctgggaggcatgctgaagccaggcggccggcaggatgagtgtgaaagaggcaggcagctcgggccgccgggagcaggcggcctaccacctgcacatctacccccagctgtccaccaccgagagccaggcctcgtgccgcgtgactgccaccaaggacagcaccacctcggacgtcatcaaggacgccattgccagcctgcggctggacggcaccaaatgttatgtgctggtggaggtcaaagagtcgggaggcgaggaatgggtgctggacgccaacgactcgcctgtgcaccgggtgctgctatggccccggcgggcacaggacgagcaccctcaggaggatggctactacttcctgctgcaggagcgcaacgcagatggaaccatcaagtacgtgcatatgcagctggtggcgcaggccacagccacccggcgcctagtggagcgtggcctcctgccacggcagcaggcggactttgatgacctgtgtaacctccccgagctaaccgagggcaacctcctgaagaacctcaagcaccgcttcctgcaacaaaagatctacacgtacgcggggagcatcctggtggccatcaacccctttaagttcctgcccatctacaaccccaagtacgtgaagatgtatgagaaccagcagctgggcaagctggagccacacgtcttcgcgctggccgacgtggcctactacaccatgctcaggaagcgcgtgaaccagtgcatcgtgatctcgggtgagagcggctccggcaagacccagagcaccaacttcctcatccactgcctcaccgccctcagccagaagggctacgccagcggcgtcgagaggaccatcctgggtgctggccctgtgctggag

SEQ ID NO: 228 - DNA, 299 bp, Homo Sapiens
gccgctgcgcggccctgcgcatgctgctggcagatcagggccagagctggaaggaggaggtggtgaccgtggagacgtggcaggagggctcactcaaagcctcctgcctatacgggcagctccccaagttccaggacggagacctcaccctgtaccagtccaataccatcctgcgtcacctgggccgcacccttgggctctatgggaaggaccagcaggaggcagccctggtggacatggtgaatgacggcgtggaggacctccgctgcaaatacatctccctcatctacaccaactat

SEQ ID NO: 229 - DNA, 249 bp, Homo Sapiens
gttctcatcagctcccccagccatgcagcaatttttgtgggtcaggccctgctttgcagaagcagcttgtggttgtctaatgggctaggggaagacaggcttgcttaattcagcccaggtctaaagcaatctgctccactgagctttcacaggaagaaatcagctgagattttgaactcttgtttctgtttccaaactcaactagaaatggaggtatttattgattgacctcctaaaacatcaacacag

SEQ ID NO: 230 - DNA, 981 bp, Homo Sapiens
gtcgtgatgtctcgggagtcggatgttgaggctcagcagtctcatggcagcagtgcctgttcacagccccatggcagcgttacccagtcccaaggctcctcctcacagtcccagggcatatccagctcctctaccagcacgatgccaaactccagccagtcctctcactccagctctgggacactgagctccttagagacagtgtccactcaggaactctattctattcctgaggaccaagaacctgaggaccaagaacctgaggagcctacccctgccccctgggctcgattatgggcccttcaggatggatttgccaatcttgagacagagtctggccatgttacccaatctgatcttgaactcctgctgtcatctgatcctcctgcctcagcctcccaaagtgctgggataagaggtgtgaggcaccatccccggccagtttgcagtctaaaatgtgtgaatgacaactactggtttgggagggacaaaagctgtgaatattgctttgatgaaccactgctgaaaagaacagataaataccgaacatacagcaagaaacactttcggattttcagggaagtgggtcctaaaaactcttacattgcatacatagaagatcacagtggcaatggaacctttgtaaatacagagcttgtagggaaaggaaaacgccgtcctttgaataacaattctgaaattgcactgtcactaagcagaaataaagtttttgtcttttttgatctgactgtagatgatcagtcagtttatcctaaggcattaagagatgaatacatcatgtcaaaaactcttggaagtggtgcctgtggagaggtaaagctggctttcgagaggaaaacatgtaagaaagtagccataaagatcatcagcaaaaggaagtttgctattggttcagcaagagaggcagacccagctctcaatgttgaaacagaaatagaaattttgaaaaagctaaatcat

SEQ ID NO: 231 - DNA, 5178 bp, Homo Sapiens
gtctgcattacatttactgaatttggaaaaatgcaaaatatttgtaactttcttgttgaaaagctagatagctctgttgtcatcagcccaccccagttctatcatactccaggttctgttgagaatcttcgacggcaagcctgtcttgttgctgttgagaatgcgtggcgcaaagctcaagaagtctgtaaccttgttggccaaaccttaggaaaacctttactaatcaaagaagaagaaacaaaagaatgggaaggccaaatagatgatcaccagtcatccagactctcaagttcattaactgtacaacaaaaaatcaaaagtgcaacaatacatgctgcttcaaaagtatttataacttttgaggtaaagggaaaagagaagagaaaaaagcacctttgaaattccaaacaaattatattgtacttgtatctttttacctatttttatactttttataatgtttacgtttgtcctgaatatatatatatatatatatatatatatatatatggtataggagataagctatttctttccaaaatctataaatctttgaatatagtcccacagaatacttatgttcactttctatttttaaaagactactctgaaaaacactcttggataataaatgtattatgtgcatacttatatactgacagttcatacaagcacatgtgtaatatttctattataaggacagatattgacccttgcatttcataatttttaaagatatttaagctaaaattttctcagccacatttttatataaaaccaaattttatgtaaaaactaggcttcaataagttagctaaattttttaaattgtaagacaaaatttttatggaaaagaattcctatatcccatcaaacctaataattttccacaactaaaaatgagtataaatacataattttaagtgcatcctgtcaggtctgatatttaattttactttagttagaagctaagtagttaccctgttacttcttcatggatgctaacagaagacttgagattcccagacagaaacaaaggactattactcatggcacaacaaggagcatgagcatcagcacattctcattgcttccccttgtcccaggtcccatgggggcaaaatgatatggccatatgcaatatggctacatatacagcaggcttttgttgtaggtaaagaatactgagcttggagggttcactgctttttatccatgaacagaagcaaacctgctctttgcctcatcccttatagatgctcactcaaaactcaatactgagaaatggcccaggtaaagaacagccaaggtctttcatgcttgccacacccagcaatatgtttaggaaggtgaagtctactgagggctatctcacatctaacagtttctttactaggaccagagaccattttggtaagatgcagataacaaccccaaccttaacaacttcacaacatgaatagaaatagtgataaataattcatagctttttacaaaagaataatgaggtataataggaaaatgttttgatgccaaaaataaaagagttaatgtttggatcggactccaacagggctccttagagtgatacaaatacaaagcaaacgatatgtgtaatttaaaatttcctagtgcacatgtttttttatttttctttttcgagatggagtctttctctgtcgcccaagctggagtgcagtggtacgatctcagctcactgcaacctctgcctcctgaatttaagcgattctcctgcctcagcctcccaagtagctgggattacaggcgtgagccactgcatctggccctgtacgcacatttttaaaaataaaactttaaaaaagaaacagttgaaactaattttaataacattttatttaaccaaatatatcaaaaatattatttcagcatgtaatcagtattttttaaattgagatatttgttttatcttcaaaattcagtgtatatttttacataatagcacatctcaattcacatgctaaattttcatcagaaatattttatctgtatttaaatttcctaaaatgtacagttgaaaaaaagagattcacatacccaggttgttctagcatgcctaaaaaatcttgtaactggatcaagtatcagtttttaaattagttaaaattaaataaaatttaaaatttagttcctcagtcacactagccatatttcaagtacttgaaggccacaaatggcttgagactaccttgctgaacagaccaaggtcaaacactgagataataatgattcttaaggccatttgaaagttaacagcaagtatgtattactgctatctacagtaagaattacattttatctacaggcaatcataagccatgtctgttatgcagcataggctttccattctctatacatctaggtcatagagtttttccattgataaatctggatgtttatataccaacagtactttcttacaacatattccagtatatagtgtccagcttcacccactttttaaagtggccctgaaacaattttattcatcttattggagtgttgctgtaggggaagagtagaagctaagaagagtttgagttcaacaccattatatccaaatcctgaccttactactggaatgtaagctccttgaaagtggagatcttgtctgtcttgttcacagttgtgttcccagccccagaggtagtctcagggccaatatcaagtatgctctcaaatatttgctgggaaaatttactagctggataaaaggcaagttactttaatctctttaatcttcctttgtccttacaaatgaaagagactaatagtacttaaccttgcagtgttattataaggattaaagttgataaatatatgatcatgagaacaattgctagcacataacggtcactctgaaaatagtagctgatatatcagctgcattgtttgattattgttggctcaattaaaagttggcttttccaaatcctccattaggaagaatttttaaatagccatattgaaaataaagttgtcagaaaaccaatgtaacatatgttgaatttttattaccaaaaagaagttactatccagttgtacagaaaagtagaatgaggttgtgcatcaagaaaaaaattgttcgttctcaacaaaaatttaatcacatgggtagtttttttttttttagctatgggttcatgctgtatttggttgcatcatatttgataaagaagcttccaacttagcatgaaatacctaaagacaattttttttttaagttagtaggcactttttagaatttccaggaacttttaaagatgttgtctccaaaaaaaacaaaaaaatcctgtaaagtatacaatcctacatttttctatagaagagcttagggaagaaatcattcaaagattggaaagacttatgtgtagtattctgtatactaaagggtatcaaatttagttgtcagttgatagcaatttaatttcttgatacatggcatacactggaatcttatgatgaattatttaggggacctgtgccacaaagtctactagaataaacctatcctaaacttaatcctgttagaatttccttcttttgaactctgtttaaaaaaaaaaaaatctttccagaaagtaggaagttataatttctattttctgaaagacaaacaaaaattctaaataggatatttgaaacagacccactgtatcctaaatggaagtgctattaaaaggaaatgctattaaaataagatttcatatattactgataattcataatgaattttaggattccattcaattacatactaaaaattacattttttatagtggcaatgtacttttatcagtattcttatttgacggtcaatttgagttcagtgtaaacaaataggacttagaaaaataatttgtaactcaagatgctgtacctcatcctataattgttgttttgtaatattccttctcaggaatatgggctaaggatatctcaactctgtcagatttgttgataagaatttaatagtaaggagttcttatagcctataaaaagcatgaattatgagatcaaatgtgggagttactatatgattatctgcattacaaaatgaaagcaaatgctttatttattccaacaaaaaaagacctatataaattagtttaagctaacatttagacatttactattacatatatggtatttgcaaccaaatcactgagtttgttgttgtcgttgtttgaggctgttgctctgtcacccaggatggagtgcaagtagctcaatcatagcttactgcagcctaaaaactcctgggctgaagaaatcctctcacctcagccttttaagtagctgaaacattaggtgcacaccaccaggcctggctaattttttattagtgataaggtcttgctatgttgcccaggctggtctcaaactcctggcttcaagcagtcctgcctcagcttcccagagtgctggactacaggcttgagccactgcacccagccccaaatagcttctactcataacctagtgtggcctaaatttatttaaaatgcccgggtaggccaggcacgttggctcacgcctgtaatcccagcacttgggaggccgaggtaggcagatcacttgaggtcaggagttcgagaccaacctggccaatatattgaaaccctgtctcttctaaaaatacagaagttactcaggcgtggtggcgtgtgactgtaatctcagctaccggggaggctgaagcagaagaaacgctcgaacctgggaggcagagcttacagtgagccgagatcgcaccactgcacttcagcctgggtgacaaagtgagactccatctacatacatacatacatacatacatacatacatacatacatacatacatacataaaatgcccagtatcttacaagactgtagttcacagtgggtaattcaaatcagacactgctcttcaagagaggtaatattaatagaaatctttcaagaaggattgttttctactattaaaacaataaaactcttataaacctgtttatcagaaggatatttctgtttcagcaactcctggaatccttcttcaacatcccaaccaacaattactcccagatagccatgtcacctgtgaattatcatgaatcccacatcaaatgaacaaatactgcctctggactctgaat

SEQ ID NO: 232 - DNA, 273 bp, Homo Sapiens
gccagaacacaaagcgtgaatccggaagaaaagttcaatctggaaacatcaatgctgccaagactattgcagatatcatccgaacatgtttgggacccaagtccatgatgaagatgcttttggacccaatgggaggcattgtgatgaccaatgatggcaatgccattcttcgagagattcaagtccagcatccagcggccaagtccatgatcgaaattagccggacccaggatgaagaggttggagatgggaccacatcagtaattattcttg

SEQ ID NO: 233 - DNA, 4474 bp, Homo Sapiens
gagtgtttctcaggagctatcagaaactatcctcaccatggtagccaattgcagtaatgttatgaataaggccagacaaccaccacctggagttatgccaaaaggacgtcctcctagtgctagcagcttagatgccatttctcctgttcagattgaccctcttgctggaatgacatctcttagtataggtggttcagctgcccctcacacccagagtatgcagggttttcctccaaatttgggttctgcattcagtacccctcagtcaccagcaaaagcatttccacccctttcaacccccaatcagaccactgcattcagtggtattggaggactttcatcacagcttccagtaggtggtcttggcacaggcagcctgactggtataggaactggtgctcttggactccctgcagtgaataacgacccttttgtacagaggaaactgggcacctctggactgaatcagcctacattccagcagagtaagatgaaaccttcggacttgtctcaggtgtggccagaggcaaaccagcactttagtaaagagatagatgatgaagcaaacagctatttccagcgaatatataatcatccaccacatccaaccatgtctgttgatgaggtattagaaatgctgcagagatttaaagactctactataaagagggaacgagaagtatttaactgtatgctaaggaacttgtttgaagaatatcgtttttttccccagtatcctgataaagagttacatataacagcctgcctatttggtggtataattgagaaaggactggtcacttacatggcactaggtctggctctacgatatgttcttgaagccttacgcaagccttttggatccaaaatgtattatttcgggattgctgcactagatagatttaaaaacagattgaaggactatccccagtattgtcagcatttggcttctatcagtcactttatgcaatttccacatcatttacaggagtatattgagtatggacagcagtctagagatcctcctgtgaaaatgcaaggctctatcacaacccctggaagtattgcactggctcaggcccaggctcaggcccaggttccagcaaaagctcctcttgctggtcaagttagcactatggtaaccacctcaacaactaccactgttgctaaaacggttacggtcaccaggccaactggagtcagctttaagaaagatgtgccaccttctattaatactacaaatatagatacgttgcttgtggccacagatcaaactgagagaattgtggagcccccagaaaatatccaggagaaaattgcttttattttcaataatctctcacagtcaaatatgacacaaaaggttgaagagctaaaggaaacggtgaaagaagaatttatgccttgggtttcacagtatctggttatgaagagagtcagtattgagccaaactttcatagcctgtattcaaacttccttgacacgctgaagaatcctgaatttaacaagatggttctgaatgagacctacagaaacattaaagtgctcctgacctctgataaagctgcagccaatttctcagatcgttctttgctgaagaacttgggacattggctaggaatgatcacattagctaaaaacaaacccatcttacacactgacttggatgtgaaatcattgctgctagaggcttatgttaaaggacaacaagaattgctctatgtagtgccctttgttgccaaagtcttagaatctagcattcgtagtgtggtttttaggccaccaaacccttggacaatggcaattatgaatgtattagctgagctacatcaggagcatgacttaaagttaaacttgaagtttgaaatcgaggttctctgcaagaaccttgcattagacatcaatgagctaaaacctggaaacctcctaaaggataaagatcgcctgaagaatttagatgagcaactctctgctccaaagaaagatgtcaagcagccagaagaactccctcccatcacaaccacaacaacttctactacaccagctaccaacaccacttgtacagccacggttccaccacagccacagtacagctaccacgacatcaatgtctattcccttgcgggcttggcaccacacattactctgaatccaacaattcccttgtttcaggcccatccacagttgaagcagtgtgtgcgtcaggcaattgaacgggctgtccaggagctggtccatcctgtggtggatcgatcaattaagattgccatgactacttgtgagcaaatagtcaggaaggattttgccctggattcggaggaatctcgaatgcgaatagcagctcatcacatgatgcgtaacttgacagctggaatggctatgattacatgcagggaacctttgctcatgagcatatctaccaacttaaaaaacagttttgcctcagcccttcgtactgcttccccacaacaaagagaaatgatggatcaggcagctgctcaattagctcaggacaattgtgagttggcttgctgttttattcagaagactgcagtagaaaaagcaggccctgagatggacaagagattagcaactgaatttgagctgagaaaacatgctaggcaagaaggacgcagatactgtgatcctgttgttttaacatatcaagctgaacggatgccagagcaaatcaggctgaaagttggtggtgtggacccaaagcagttggctgtttacgaagagtttgcacgcaatgttcctggcttcttacctacaaatgacttaagtcagcccacgggatttttagcccagcccatgaagcaagcttgggcaacagatgatgtagctcagatttatgataagtgtattacagaactggagcaacatctacatgccatcccaccaactttggccatgaaccctcaagctcaggctcttcgaagtctcttggaggttgtagttttatctcgaaactctcgggatgccatagctgctcttggattgctccaaaaggctgtagagggcttactagatgccacaagtggtgctgatgctgaccttctgctgcgctacagggaatgccacctcttggtcctaaaagctctgcaggatggccgggcatatgggtctccatggtgcaacaaacagatcacaaggtgcctaattgaatgtcgagatgaatataaatataatgtggaggctgtggagctgctaattcgcaatcatttggttaatatgcagcagtatgatcttcacctagcgcagtcaatggagaatggcttaaactacatggctgtggcatttgctatgcagttagtaaaaatcctgctggtggatgaaaggagtgttgctcatgttactgaggcagatctgttccacaccattgaaaccctcatgaggattaatgctcattccagaggcaatgctccagaaggattgccccagctgatggaagtagtgcgatccaactatgaagcaatgattgatcgtgctcatggaggcccaaactttatgatgcattctgggatctctcaagcctcagagtatgatgaccctccaggcctgagggagaaggcagagtatcttctgagggaatgggtgaatctctaccattcagcagcagctggccgcgacagtaccaaagctttctctgcatttgttggacagatgcaccagcaaggaatactgaagaccgatgatctcataacaaggttctttcgtctgtgtactgaaatgtgtgttgaaatcagttaccgtgctcaggctgagcagcagcacaatcctgctgccaatcccaccatgatccgagccaagtgctatcacaacctggatgcctttgttcgactcattgcactgctcgtgaaacactcaggggaggccaccaacactgtcacaaagattaatctgctgaacaaggtccttggtatagtagtgggagttctccttcaggatcatgatgttcgtcagagtgaatttcagcaacttccctaccatcgaatttttatcatgcttctcttggaactcaatgcacctgagcatgtgttggaaaccattaatttccagacacttacagctttctgcaatacattccacatcttgaggcctaccaaagctcctggctttgtatatgcctggcttgaactgatttcccatcggatatttattgcaagaatgctggcacatacgccacagcagaaggggtggcctatgtatgcacagctactgattgatttattcaaatatttagcgcctttccttagaaatgtggaactcaccaaacctatgcaaatcctctacaagggcactttaagagtgctgctggttcttttgcatgatttcccagagttcctttgtgattaccattatgggttctgtgatgtgatcccacctaattgtatccagttaagaaatttgatcctgagtgcctttccaagaaacatgaggctccccgacccattcactcctaatctaaag

SEQ ID NO: 234 - DNA, 180 bp, Homo Sapiens
gtcggttgatgcgctgtgtccgctgtcctgtggcataccacgccaatgacttttgcctggctgctgggtcaaagatccttgcatctaatagtatcatctgccctaatcactttacccctaggcggggctgccgaaatcatgagcatgttaatgttagctggtgctttgtgtgctcagaag

SEQ ID NO: 235 - DNA, 878 bp, Homo Sapiens
ggttttgtctccacaattatccgtttacttgacagcccctcaacatgcattagagcaaaagccttcctggttcttctatatattttgatttataaccgtgagatgttgctgctcagttgccaagcaagactggtgatgtacatcgagagagacagcagaaagaccactccaggcaaggagcagcaaagtggcaatgaatacctgtccaaatgcctggatcttctcatctgtcacattgtgcaggagctgccacgaatcctgggtgacattcttaactccttggctaatgtttctggacgtaaacacccatcaacagttcaagtgaaacagctgaagttgtgtctccccctgatgcctgtagtgcttcacctcgtaacttcacaggtatttcgacctcaagttgtgacagaagagtttcttttcagctatggaactattcttagtcatattaaatctgtagactcaggagaaacgaacatagatggagccataggactgacagcatcagaagaatttatcaagatcacattgtcagcttttgaagcaataatacagtatcctattttattgaaagactatcgctccacggttgttgactatatactgccacccttggtgtccttggttcaaagccaaaatgtggagtggagactctttagcttgcggttgctctcagaaaccacatctctactcgtgaaccaggagtttggggatggcaaggagaaggccagtgttgattctgacagcaatcttctggctctcattcgagatgtcttacttccccagtatgagcacattcttttagaacctgacccagtaccagcatatgctctgaaactgctagtcgcgatgactgaacacaacccaactttcacaag

SEQ ID NO: 236 - DNA, 5130 bp, Homo Sapiens
aacgtggttctcctcaagatttagttactaaagacatcactgacacatcaattggggcttattggacatctgctccaggaatggttcgcggttacagggtctcatggaaatcactttatgatgatgttgacactggagagaaaaatctgcctgaagatgcaattcatacgatgatagaaaatctgcagccagagaccaaatacagaatttcagtatttgccacttacagcagtggagaaggagaacctttgactggagatgccacaactgaattatctcaagattccaaaaccctgaaagtagatgaagaaacagaaaacacaatgagagttacatggaaaccagcaccagggaaagtcgtcaactaccgtgttgtctatcgccctcatgggagagggaagcaaatggttgctaaggtgccccccacagtcacttcgacagtgttaaagcgacttcagccacagaccacatatgacatcacagttcttcctatttacaagatgggagaaggaaagcttaggcaaggatcaggaacaacagcttctcggtttaagtctcctagaaacctcaaaacatctgacccaaccatgtcaagcttccgagtgacttgggagcctgcccctggggaagtgaagggttataaagtcacattccaccctacgggggatgacagaagactgggggagttagtggttggaccctatgacaacacagttgttttggaggaacttagggctggtaccacctataaagtaaatgtttttggaatgtttgatggaggagaaagctcaccacttgttggacaagaaatgacaaccctttccgacacaactgttatgccaattttatcttctgggatggagtgtctcaccagagctgaggcagacattgtgttgctggtggatggatcatggagcatcggccgggcaaattttagaaccgtgaggagtttcatttctcgtattgtggaagtctttgacattggccccaaaagagtacaaattgctcttgctcagtatagtggggatcccagaacagagtggcagttaaatgcacacagagacaagaagagcttgttgcaagctgtggcaaacttgccgtacaaaggaggcaatactctcacaggcatggctttgaatttcattcgccaacagaacttcaggacccaagctggcatgagacctcgagctcgaaaaattggtgtgctcattactgatggaaaatcacaagacgatgttgaagcaccttcaaagaaactcaaggatgagggagtggagctgtttgctattggtattaaaaatgctgatgaagtcgaattaaagatgattgcaactgatcctgatgatacccatgcatacaatgtggcagattttgagtcactctccaggatagtggatgatctcaccattaatttgtgtaacagtgtcaaaggtccaggtgatttggaagcaccttctaacttagttatttctgagcgaacccatcgttcttttagagtgagctggacaccaccttctgacagtgtggatcgatataaggtggaatactatccagtttctggagggaaacgtcaagaattttatgtgagtcgaatggaaactagcacagtgctgaaagatctgaaacctgaaactgaatatgttgtcaatgtgtattctgtggtagaagatgaatatagtgagcctctgaaggggacagaaaaaaccttgccagtgcctgtagtcagcctgaatatttatgatgttggccctaccaccatgcatgtgcagtggcagcctgtgggaggagctactggctacatcttgtcatacaaacctgttaaggacacagagccaacaagacccaaagaggtgcgtttggggccaacagtgaatgacatgcagctgactgaccttgttcccaacacggagtatgcagtcacagtccaggctgtcctgcacgacctcactagtgaacctgtcactgttcgggaagtcaccttgcctttacccagacctcaggatctgaaactcagagatgtgactcacagcactatgaatgtcttttgggaacctgtgcctggaaaagtgcgtaaatatattgttcgatacaaaacaccagaagaggatgtcaaagaggtagaggtggacagatcagagaccagcacttccctcaaagacctcttctcacagaccttgtacacagtcagcgtttctgcagtacatgacgagggggagtctcctccagtgactgctcaagaaactacccgacccgtgccagccccaacaaacttaaagattactgaagtaacatcagagggtttcagagggacttgggatcatggagcttcagatgtgtctctctacagaataacttgggcaccttttggaagctcagataagatggagaccatcttaaatggagatgaaaacactttggtgttcgaaaacctgaaccccaacaccatctatgaagtttccattactgccatctatcctgatgagtcagaaagtgatgacctgattggcagtgagcgcacactgcctatcttaacaacacaagctcccaaaagtggcccacgaaaccttcaagtgtacaatgcaacatctaacagcctgactgttaagtgggatcctgctagtggtcgtgtgcagaaatataggatcacttatcagccttccacaggggaaggcaatgagcaaacgaccacaataggaggacggcagaacagtgtggtcctgcagaaactgaagccagacactccttacactatcaccgtatcctctctgtatcctgatggtgaaggaggtcggatgacgggaagaggcaagaccaaacctctaaacactgtaaggaacctgagagtgtatgacccttctaccagcaccttgaatgtccgctgggaccatgcagagggaaatcctcgtcagtacaagctcttctatgcaccagcagcaggtggtccagaggaactggtaccaatccccgggaataccaattatgccattcttaggaatctgcagccagatacctcatacactgtgactgtagttcccgtttatactgaaggtgatgggggacgcacatcagatactggaaggacattgatgagaggactggcaagaaatgtccaagtatacaatcctacacctaacagcctcgatgttcgctgggaccctgctccaggacctgtgctgcaatatcgcgttgtgtattctcctgtggatggcacaagaccctcagaatctatagtagtgccaggaaacacgcgcatggtgcatctggagcggctgattccggacacactctattccgtgaaccttgtggctctgtactcggatggagagggaaatcccagccctgcccagggccgaacgctaccacgcagtggaccaaggaacctgagagtctttggtgaaacaaccaatagcctctcggtagcctgggatcatgctgatgggccagttcagcagtacaggatcatctattctcccactgttggtgatccaattgatgaatataccacagtcccaggcagaagaaacaatgtaatactgcagcccctgcaacctgacactccatataaaattactgttattgctgtttatgaagatggagatggtggccatctaacaggaaatggaagaactgtgggactccttcctcctcagaacatacacatctctgacgaatggtatacaagattcagggtgtcctgggatccttcaccttctccagttcttggatataaaatagtatataagccagtgggttccaatgagcccatggaagcctttgttggagaaatgacatcatataccttacacaatctcaatcccagcaccacctacgatgtgaatgtttatgctcaatatgattctggactcagtgtccccttgacagatcaaggcactacattatatttaaatgtaacagatctgaaaacttaccagattgggtgggatacattctgtgtcaaatggtcacctcaccgggcagccacctcctacaggctaaaactaagccctgcagatggaacaaggggacaagaaattacagtgcgtggatcagaaaccagtcactgcttcactggcctttcaccagacactgattatggtgtcactgtttttgtgcagacaccaaatctcgagggaccaggagtctctgttaaagaacataccactgtgaaaccaacagaagcccctacagagccacccacacctcctccccctcccaccattccaccagcccgggatgtatgcaaaggggccaaggcagatattgtgttcttgactgatgcctcctggagcattggggacgataattttaacaaagttgtaaaattcatcttcaatactgtgggaggctttgatgaaatcagtcctgctgggattcaggtttcatttgtgcaatacagcgatgaggtcaagtctgagttcaagctgaacacgtacaatgacaaggccctagcccttggggccctccagaatattaggtacagaggaggaaacacaagaacaggcaaggccctcacgtttatcaaggagaaagtcttgacttgggagagcggcatgaggaagaatgtccctaaggtgttggttgtggtcacggacggtcggtcccaggatgaggtcaagaaggcggctttggtcatccagcagtcagggttcagtgtctttgtagttggtgtggctgatgtcgactacaatgagcttgccaacattgccagcaaaccaagtgaacggcacgtgttcattgtggacgactttgaatcttttgagaagatcgaagacaatcttattacatttgtttgtgaaactgccacttcaagttgtcctctcatttatttggatggctacacctcaccaggttttaaaatgcttgaagcatacaacctgacagaaaagaattttgcttctgtacaaggagtatctttggagtcagggtctttccccagctactcagcatacaggattcagaagaatgcgtttgtgaatcagcctacagcagacctacacccaaatggactccctccttcatacacgattatattattattcagacttctcccagaaactcccagtgacccttttgcaatttggcaaatcacagacagagactacaaaccacaagttggagtgattgcagatc

SEQ ID NO: 237 - DNA, 447 bp, Homo Sapiens
atgagagtccctacactaaatccgccagccagacaaagccgcctgatggagcgttggctgtgaggagacagagcatcccagaggaattcaagggctccacagtcgtcgagctgatgaagaaggaaggcactaccctgggtctgacggtatcgggaggaattgataaggatggcaagccaagagtatctaatctgcggcaaggaggaattgctgctagaagtgaccagctggatgtgggtgactacatcaaagcagtgaatggaatcaacctggccaaattccgccatgacgagatcatcagcttgctgaagaatgtgggagaaagagtggttcttgaagtagagtacgagcttccaccggtctctgtgcaaggatcaagtgttattttccgaacagtggaggtcacattacataaagaaggcaatacctttggttttgtaattcgag

SEQ ID NO: 238 - DNA, 867 bp, Homo Sapiens
aaactttgtctgcgaacactctttaatggaccagatcaggatttgagcggaagaacgaatgtaactttaaggcaggaaagacaaattttattcttcataaagtgatgagcatataataattccaggcacatggcaatagaggccctctaaataaggaataaataacctcttagacaggtgggagattatgatcagagtaaaaggtaattacacattttatttccagaaagtcaggggtctataaattgacagtgattagagtaatactttttcacatttccaaagtttgcatgttaactttaaatgcttacaatcttagagtggtaggcaatgttttacactattgaccttatatagggaagggagggggtgcctgtggggttttaaagaattttcctttgcagaggcatttcatccttcatgaagccattcaggattttgaattgcatatgagtgcttggctcttccttctgttctagtgagtgtatgagaccttgcagtgagtttatcagcatactcaaaatttttttcctggaatttggagggatgggaggagggggtggggcttacttgttgtagctttttttttttttacagacttcacagagaatgcagttgtcttgacttcaggtctgtctgttctgttggcaagtaaatgcagtactgttctgatcccgctgctattagaatgcattgtgaaacgactggagtatgattaaaagttgtgttccccaatgcttggagtagtgattgttgaaggaaaaaatccagctgagtgataaaggctgagtgttgaggaaatttctgcagttttaagcagtcgtatttgtgattgaagctgagtacattttgctggtgtatttttag

SEQ ID NO: 239 - DNA, 206 bp, Homo Sapiens
gaattttcaacgtccggttcatctaatacagacactggtaaagttactgggaccttggagaccaaatacaagtggtgtgagtatggtctgactttcacagaaaagtggaacactgataacactctgggaacagaaatcgcaattgaagaccagatttgtcaaggtttgaaactgacatttgatactaccttctcaccaaacacagg

SEQ ID NO: 240 - DNA, 1553 bp, Homo Sapiens
gaattgtgcgggcgcggggaattatgggggtgggggctgggagggtctctcaaagtggggctccgagcccgggaggagcaggcaggtgagggttagggaggggatgcggcgggctggtttgatggctgtgccacactcttctgagcttggggcctgggggacggaggggttggttattggttcaggaggtctggggcaaggaagaggctgtagaatgtggtgggacctcgaccactcctggaaaggaggctgctttgagggggtgggaccgaggcctgatgagaggaggatggtctcctggggaaggatttgggccagggtccctgatgttggggttgtgggggatagtgttagattcagagtgtggaaggaaaaaacgcttggggtttctgggtgattacagttggtgcttttggggagggtctcccctttggagaagaggctggaggtgggaggccctggtaaaagattcaatttcccccagtggccttagaggctctaaaaatggcttccctaaggcctcggaggctgtcctctccacaaaaatcccagcacccttcgctcagcttataatcaggcaaagccacagggcctgaagccccaggttcaaatcctcctttggcctttggctttctctgtgaccttgggcaaatgcctttgcccctctggcctgtttcctgttctgcaaaatgttgaggggaatcttccctacttcctggagtatttgtaaacattaaataataattagctctaataatggctaacactatttgagcctgatgccaagggcttgatgcatgggttaatttctcccaacaagctggggttgttgttacacccactgtatagatgaggaaactgaggcctggagcccttgagttacacagaaagtgatcggcagtgtggggaatcagtacagtggagactttgaggggctgtgtaggattctgacccacatctctgccccccacttccagggccaattcctgtccccccagcagcatggcatcgtgtgctgaaccctctgagccctctgccccactgcccgccggggtcccaccgctcgaggacttcgaggtactggatggggttgaggatgcagagggtgaggaggaagaggaggaggaagaggaggaagaggatgacctgagtgagctgccaccgctggaggacatgggacaacccccggcggaggaggctgagcagcctggggccctggcccgagagttccttgctgccatggagcccgagcccgccccagccccggccccagaagagtggctggacattctggggaacgggctgttgaggaagaagacgctggtcccagggccgccaggttcgagccgcccggtcaagggccaggtggtcaccgtacatctgcagacgtcgctggagaatggcacacgggtgcaggaggagccggagctggtgttcactctgggtgactgtgacgtcatccaggccctggatctcagtgtcccactcatggacgtgggggagacggccatggtcactgctgactccaagtactgctacggcccccaaggcag

SEQ ID NO: 241 - DNA, 1135 bp, Homo Sapiens
atcttgagccagatgattgtgcatccatttacatctttaatgtagatccacctccatctactttaaccacaccactttgcttaccacatcatggattaccgtctcactcttctgttttgtcaccatcgtttcagctccaaagtcacaaaaactatgaaggaacttgtgagattcctgaatctaaatatagcccattaggtggtcccaaaccctttgagtgcccaagtattcaaattacatctatctctcctaactgtcatcaagaattagatgcacatgaagatgacctacagataaatgacccagaacgggaatttttggaaaggccttctagagatcatctctatcttcctcttgagccatcctaccgggagtcttctcttagtcctagtcctgccagcagcatctcttctaggagttggttctctgatgcatcttcttgtgaatcgctttcacatatttatgatgatgtggactcagagttgaatgaagctgcagcccgatttacccttggatcccctctgacttctcctggtggctctccagggggctgccctggagaagaaacttggcatcaacagtatggacttggacactcattatcacccaggcaatctccttgccactctcctagatccagtgtcactgatgagaattggctgagccccaggccagcctcaggaccctcatcaaggcccacatccccctgtgggaaacggaggcactccagtgctgaagtttgttatgctgggtccctttcaccccatcactcacctgttccttcacctggtcactcccccaggggaagtgtgacagaagatacgtggctcaatgcttctgtccatggtgggtcaggccttggccctgcagtttttccatttcagtactgtgtagagactgacatccctctcaaaacaaggaaaacttctgaagatcaagctgccatactaccaggaaaattagagctgtgttcagatgaccaagggagtttatcaccagcccgggagacttcaatagatgatggccttggatctcagtatcctttaaagaaagattcatgtggtgatcagtttctttcagttccttcaccctttacctggagcaaaccaaagcctggccacacccctatatttcg

SEQ ID NO: 242 - DNA, 100 bp, Artificial Sequence
  A synthetic sequence
tttgaattttcagaggaaaagacagtgagagagcaagagcagattgagaagcagaagagtgagtcgcaaaaagagaaaatgacagaaatggagcaccctg

SEQ ID NO: 243 - DNA, 100 bp, Artificial Sequence
  A synthetic sequence
ggaaaagaaaccaagtctccttgaatcagtttatcccaatgtcttctggttttctatgttggctacagtcaagataacctattactaaggtaggttgtgt

SEQ ID NO: 244 - DNA, 83 bp, Artificial Sequence
  A synthetic sequence
ctgtaatgacgcaaccaaccttaatatacagccagcctgtcatgagacctccaaacccctttggccctgtatcaggagcacag

SEQ ID NO: 245 - DNA, 82 bp, Artificial Sequence
  A synthetic sequence
gcaccccctgtaatggcctatcctgctactacaccaacaggcatgataggatatggaattcctccacaaatgggaagtgttc

SEQ ID NO: 246 - DNA, 100 bp, Artificial Sequence
  A synthetic sequence
gcgaggagtggttggggtgactttcacctctagtcactgtcgcagggacaggagtactccacagaggataaatttcaacctccggggccacaatagcgag

SEQ ID NO: 247 - DNA, 100 bp, Artificial Sequence
  A synthetic sequence
tcctgggtcaagctgagtatctccacgtttaggtgcacaaatactattttgtgaagatcttaaatttgtggtataagaacttgaaggaaatagaactgat

SEQ ID NO: 248 - DNA, 100 bp, Artificial Sequence
  A synthetic sequence
ggctctcccggtcaatgtgaaatggctgctgtgtggttgtctctgctgctgttgcacactgggttgcagcctatggcctgttatttgtctcaacaaaaga

SEQ ID NO: 249 - DNA, 100 bp, Artificial Sequence
  A synthetic sequence
gtttggcttgagcaacaagtttgatactgaatttccctccgttctaacagggaaggtggccccagaagaatttaaaaccagcattggccgtgtgaatgca

SEQ ID NO: 250 - DNA, 100 bp, Artificial Sequence
  A synthetic sequence
tccccagcatgagagggatgtcatctatgaggaggagtcaaggcatcaccacagcgaacgcatgcaacacgtggtccagcttccccctgagccggtgcag

SEQ ID NO: 251 - DNA, 100 bp, Artificial Sequence
  A synthetic sequence
gtttgatgattcctgtcttttgtgtcgtggagcagttggacggctctcttgaatatgacaacagagaagaacacgccgagtttgtcctggtgcggaaaga

SEQ ID NO: 252 - DNA, 100 bp, Artificial Sequence
  A synthetic sequence
gagtagtaatgcagtgtctcagattaccagaatgtgcctcctgaaaggaaatatgggtgtttgctttgatgttcctacaactgagtcagaaaggttacag

SEQ ID NO: 253 - DNA, 100 bp, Artificial Sequence
  A synthetic sequence
gtctctggcttccgtttcttacgctgctgttgattttttccgaccatcagctcagagactgatagaagagaaaggtgcagtggatgcattggctgcagct

SEQ ID NO: 254 - DNA, 100 bp, Artificial Sequence
  A synthetic sequence
gagcatggcggcatttgcaggaatgtgtgatggaggctctacagaggacggctgtgtcgcagcctctcgggatgacaccactctgaatgcactgaacaca

SEQ ID NO: 255 - DNA, 100 bp, Artificial Sequence
  A synthetic sequence
agggaagtgtaacatctcccatttttctgacatatttgctgctagagagtttaaagcccgagtggattcatttttctacatattaggatataaccctgag

SEQ ID NO: 256 - DNA, 98 bp, Artificial Sequence
  A synthetic sequence
tcgcaccaatagtggtattagttcagcaagtggtggaagcacggagcccacgactcccgatagtgagagacctgctcaagctctcttaagagattatg

SEQ ID NO: 257 - DNA, 97 bp, Artificial Sequence
  A synthetic sequence
gggcaatgttaaatattgttcaagattcagcacttttggaagccattggttgccagatggagatgggtggtggagaaaataacctgaagagtcatag

SEQ ID NO: 258 - DNA, 100 bp, Artificial Sequence
  A synthetic sequence
tacccagtcttccatcaactggctcaatatccatgtcttccaacgtctccagtgtgctgatcttctgacattcaggtcttccagtgtctgcaatatccag

SEQ ID NO: 259 - DNA, 100 bp, Artificial Sequence
  A synthetic sequence
ggtttccgatggcacctgtgtcaaggtcttccaacaactccgggtcttccagcgacttcaagtcttccaataatctcaaggtcttccagataatcctgag

SEQ ID NO: 260 - DNA, 100 bp, Artificial Sequence
  A synthetic sequence
catcccccagctgaagaagaagtatgatgtggacacactggacatggtcttcctcgaccactggaaggaccggtacctgccggacacgcttctcttggag

SEQ ID NO: 261 - DNA, 100 bp, Artificial Sequence
  A synthetic sequence
aaataacatctgctttgctgccgagctcagaggagaccccagacccctcccgcagccagagggctggagcctgctcagaggtgctttgaagaggatccct

SEQ ID NO: 262 - DNA, 100 bp, Artificial Sequence
  A synthetic sequence
tgccgggggcctgggcaaggagttctcccggagctaccggctgcgttactcccgggatggtcgccgctggatgggctggaaggaccgctggggtcaggag

SEQ ID NO: 263 - DNA, 100 bp, Artificial Sequence
  A synthetic sequence
agatgctgcccccacccccttaggcccgagggatcaggagctatgggaccagaggccctgtcatctttactgctgctgctcttggtggcaagtggagatg

SEQ ID NO: 264 - DNA, 100 bp, Artificial Sequence
  A synthetic sequence
tgagcaaggattctggcttttaagactaaggaaccaaactgatctaaaggctctggatttgccttctttcagtctgacactgactaacaacctcgacaag

SEQ ID NO: 265 - DNA, 100 bp, Artificial Sequence
  A synthetic sequence
gcctgtctccactaactctggaccctaaaacagctcacccaaatctggtgctctccaaaagccaaaccagcgtctggcatggtgacattaagaagataat

SEQ ID NO: 266 - DNA, 69 bp, Artificial Sequence
  A synthetic sequence
tctaaagcagctcaaaagaaagcagaagccactggagaaaaacggccaagaggcagacctaggaaatgg

SEQ ID NO: 267 - DNA, 69 bp, Artificial Sequence
  A synthetic sequence
gaaccaaccggtgagccctctcctaagagacccaggggaagacccaaaggcagcaaaaacaagagtccc

SEQ ID NO: 268 - DNA, 100 bp, Artificial Sequence
  A synthetic sequence
caggccacagacaatgagcctgttgagatgctacactcaggcaccctggtgcccagccgggaactccccatcgcgttttgccagcgataatacgcctcag

SEQ ID NO: 269 - DNA, 100 bp, Artificial Sequence
  A synthetic sequence
gacaggaggaaacgcagcgccagcagcatctcatctaccctccttgacacctccccgtggctccagccagaccctagaggtcagccttgcggaccaacag

SEQ ID NO: 270 - DNA, 59 bp, Artificial Sequence
  A synthetic sequence
gcaagaacaaggtgacacatattctgtctgtccacgatagtgccaggcctatgttggag

SEQ ID NO: 271 - DNA, 58 bp, Artificial Sequence
  A synthetic sequence
atcctgcccggcctgtacatcggcaacttcaaagatgccagagacgcggaacaattga

SEQ ID NO: 272 - DNA, 100 bp, Artificial Sequence
  A synthetic sequence
acacctggcaaatttgattttttttttttttccagagacggggtctcgcaacattgcccagacttcctttgtgttagttaataaagctttctcaactgcc

SEQ ID NO: 273 - DNA, 100 bp, Artificial Sequence
  A synthetic sequence
ccccccggtatgagattgtcatcatcactgtggtagcagccgcagtcataatgggcactgcaggcctcagcacgtacctctataaccgccagcggaagat

SEQ ID NO: 274 - DNA, 100 bp, Artificial Sequence
  A synthetic sequence
tagaatttgtttcatctattttattttattgaatactgtctgtatctttggttatcctgtttgaagaaaaaggacaaataaaacatggccagcaaataca

SEQ ID NO: 275 - DNA, 100 bp, Artificial Sequence
  A synthetic sequence
cccctcttcctcgccctctctcgcgggtcggggttacatggcggcgactgcggcaaagcgagagcctcggagacgccgctgccgccagcacagccggaga

SEQ ID NO: 276 - DNA, 100 bp, Artificial Sequence
  A synthetic sequence
ggaacactgataacactctgggaacagaaatcgcaattgaagaccagatttgtcaaggtttgaaactgacatttgatactaccttctcaccaaacacagg

SEQ ID NO: 277 - DNA, 100 bp, Artificial Sequence
  A synthetic sequence
gaattttcaacgtccggttcatctaatacagacactggtaaagttactgggaccttggagaccaaatacaagtggtgtgagtatggtctgactttcacag

SEQ ID NO: 278 - DNA, 100 bp, Artificial Sequence
  A synthetic sequence
agtatcctttaaagaaagattcatgtggtgatcagtttctttcagttccttcaccctttacctggagcaaaccaaagcctggccacacccctatatttcg

SEQ ID NO: 279 - DNA, 100 bp, Artificial Sequence
  A synthetic sequence
atcttgagccagatgattgtgcatccatttacatctttaatgtagatccacctccatctactttaaccacaccactttgcttaccacatcatggattacc

SEQ ID NO: 280 - DNA, 100 bp, Artificial Sequence
  A synthetic sequence
ccagctgagtgataaaggctgagtgttgaggaaatttctgcagttttaagcagtcgtatttgtgattgaagctgagtacattttgctggtgtatttttag

SEQ ID NO: 281 - DNA, 100 bp, Artificial Sequence
  A synthetic sequence
aaactttgtctgcgaacactctttaatggaccagatcaggatttgagcggaagaacgaatgtaactttaaggcaggaaagacaaattttattcttcataa

SEQ ID NO: 282 - DNA, 100 bp, Artificial Sequence
  A synthetic sequence
acgtcatccaggccctggatctcagtgtcccactcatggacgtgggggagacggccatggtcactgctgactccaagtactgctacggcccccaaggcag

SEQ ID NO: 283 - DNA, 100 bp, Artificial Sequence
  A synthetic sequence
gaattgtgcgggcgcggggaattatgggggtgggggctgggagggtctctcaaagtggggctccgagcccgggaggagcaggcaggtgagggttagggag

SEQ ID NO: 284 - DNA, 100 bp, Artificial Sequence
  A synthetic sequence
ctggcaatggtgttgctggcaccaatatcataataggcataattgctggcaccattttagtgctggccctcatattaggaataactgcgtggggttataa

SEQ ID NO: 285 - DNA, 100 bp, Artificial Sequence
  A synthetic sequence
gtgaatgtaaatgcgaggacacgtggtccgggtgcataatgggagacactggctattatcttcctaaaaagttcacccagtgtaatattgaagagtatca

SEQ ID NO: 286 - DNA, 100 bp, Artificial Sequence
  A synthetic sequence
gagcaccaacttcctcatccactgcctcaccgccctcagccagaagggctacgccagcggcgtcgagaggaccatcctgggtgctggccctgtgctggag

SEQ ID NO: 287 - DNA, 100 bp, Artificial Sequence
  A synthetic sequence
ctccaggacacgcgcgccccgagcctgggaggcatgctgaagccaggcggccggcaggatgagtgtgaaagaggcaggcagctcgggccgccgggagcag

SEQ ID NO: 288 - DNA, 20 bp, Artificial Sequence
  A synthetic sequence
gttgtctctgctgctgttgc

SEQ ID NO: 289 - DNA, 20 bp, Artificial Sequence
  A synthetic sequence
ccaccttccctgttagaacg

SEQ ID NO: 290 - DNA, 20 bp, Artificial Sequence
  A synthetic sequence
aatgtgaaatggctgctgtg

SEQ ID NO: 291 - DNA, 20 bp, Artificial Sequence
  A synthetic sequence
ccaccttccctgttagaacg

SEQ ID NO: 292 - DNA, 20 bp, Artificial Sequence
  A synthetic sequence
tctcccggtcaatgtgaaat

SEQ ID NO: 293 - DNA, 20 bp, Artificial Sequence
  A synthetic sequence
ccaccttccctgttagaacg

SEQ ID NO: 294 - DNA, 20 bp, Artificial Sequence
  A synthetic sequence
ggttgtctctgctgctgttg

SEQ ID NO: 295 - DNA, 20 bp, Artificial Sequence
  A synthetic sequence
ccaccttccctgttagaacg

SEQ ID NO: 296 - DNA, 21 bp, Artificial Sequence
  A synthetic sequence
ccggtcaatgtgaaatggctg

SEQ ID NO: 297 - DNA, 20 bp, Artificial Sequence
  A synthetic sequence
tgttgctcaagccaaactct

SEQ ID NO: 298 - DNA, 20 bp, Artificial Sequence
  A synthetic sequence
ccggtcaatgtgaaatggct

SEQ ID NO: 299 - DNA, 22 bp, Artificial Sequence
  A synthetic sequence
acttgttgctcaagccaaactc

SEQ ID NO: 300 - DNA, 19 bp, Artificial Sequence
  A synthetic sequence
tctgctgctgttgcacact

SEQ ID NO: 301 - DNA, 22 bp, Artificial Sequence
  A synthetic sequence
ttaaattcttctggggccacct

SEQ ID NO: 302 - DNA, 21 bp, Artificial Sequence
  A synthetic sequence
atgtgaaatggctgctgtgtg

SEQ ID NO: 303 - DNA, 22 bp, Artificial Sequence
  A synthetic sequence
cttgttgctcaagccaaactct

SEQ ID NO: 304 - DNA, 21 bp, Artificial Sequence
  A synthetic sequence
ggtcaatgtgaaatggctgct

SEQ ID NO: 305 - DNA, 21 bp, Artificial Sequence
  A synthetic sequence
ttgttgctcaagccaaactct

SEQ ID NO: 306 - DNA, 20 bp, Artificial Sequence
  A synthetic sequence
gcagaagagtgagtcgcaaa

SEQ ID NO: 307 - DNA, 22 bp, Artificial Sequence
  A synthetic sequence
gccaacatagaaaaccagaaga

SEQ ID NO: 308 - DNA, 20 bp, Artificial Sequence
  A synthetic sequence
gcagaagagtgagtcgcaaa

SEQ ID NO: 309 - DNA, 23 bp, Artificial Sequence
  A synthetic sequence
gaaaaccagaagacattgggata

SEQ ID NO: 310 - DNA, 20 bp, Artificial Sequence
  A synthetic sequence
gcagaagagtgagtcgcaaa

SEQ ID NO: 311 - DNA, 23 bp, Artificial Sequence
  A synthetic sequence
agccaacatagaaaaccagaaga

SEQ ID NO: 312 - DNA, 21 bp, Artificial Sequence
  A synthetic sequence
agcagaagagtgagtcgcaaa

SEQ ID NO: 313 - DNA, 23 bp, Artificial Sequence
  A synthetic sequence
ccagaagacattgggataaactg

SEQ ID NO: 314 - DNA, 20 bp, Artificial Sequence
  A synthetic sequence
gcagaagagtgagtcgcaaa

SEQ ID NO: 315 - DNA, 23 bp, Artificial Sequence
  A synthetic sequence
gccaacatagaaaaccagaagac

SEQ ID NO: 316 - DNA, 20 bp, Artificial Sequence
  A synthetic sequence
aaatggagcaccctgggaaa

SEQ ID NO: 317 - DNA, 25 bp, Artificial Sequence
  A synthetic sequence
gtaataggttatcttgactgtagcc

SEQ ID NO: 318 - DNA, 20 bp, Artificial Sequence
  A synthetic sequence
gacagaaatggagcaccctg

SEQ ID NO: 319 - DNA, 23 bp, Artificial Sequence
  A synthetic sequence
aataggttatcttgactgtagcc

SEQ ID NO: 320 - DNA, 20 bp, Artificial Sequence
  A synthetic sequence
acatccccaccctgaatgtc

SEQ ID NO: 321 - DNA, 21 bp, Artificial Sequence
  A synthetic sequence
ttttcccagggtgctccattt

SEQ ID NO: 322 - DNA, 21 bp, Artificial Sequence
  A synthetic sequence
ggagcaccctgggaaaagaaa

SEQ ID NO: 323 - DNA, 22 bp, Artificial Sequence
  A synthetic sequence
acctgtccactcagatgcaaaa

SEQ ID NO: 324 - DNA, 21 bp, Artificial Sequence
  A synthetic sequence
tacatccccaccctgaatgtc

SEQ ID NO: 325 - DNA, 22 bp, Artificial Sequence
  A synthetic sequence
acttggtttcttttcccagggt

SEQ ID NO: 326 - DNA, 21 bp, Artificial Sequence
  A synthetic sequence
atccccaccctgaatgtcctt

SEQ ID NO: 327 - DNA, 21 bp, Artificial Sequence
  A synthetic sequence
tggtttcttttcccagggtgc

SEQ ID NO: 328 - DNA, 21 bp, Artificial Sequence
  A synthetic sequence
gaaatggagcaccctgggaaa

SEQ ID NO: 329 - DNA, 22 bp, Artificial Sequence
  A synthetic sequence
gtgtcatctaggacctgtccac

SEQ ID NO: 330 - DNA, 20 bp, Artificial Sequence
  A synthetic sequence
acatggtcttcctcgaccac

SEQ ID NO: 331 - DNA, 20 bp, Artificial Sequence
  A synthetic sequence
cttcaaagcacctctgagca

SEQ ID NO: 332 - DNA, 20 bp, Artificial Sequence
  A synthetic sequence
atggtcttcctcgaccactg

SEQ ID NO: 333 - DNA, 20 bp, Artificial Sequence
  A synthetic sequence
cttcaaagcacctctgagca

SEQ ID NO: 334 - DNA, 20 bp, Artificial Sequence
  A synthetic sequence
tcttcctcgaccactggaag

SEQ ID NO: 335 - DNA, 20 bp, Artificial Sequence
  A synthetic sequence
cttcaaagcacctctgagca

SEQ ID NO: 336 - DNA, 20 bp, Artificial Sequence
  A synthetic sequence
gtggacacactggacatggt

SEQ ID NO: 337 - DNA, 21 bp, Artificial Sequence
  A synthetic sequence
gggatcctcttcaaagcacct

SEQ ID NO: 338 - DNA, 20 bp, Artificial Sequence
  A synthetic sequence
ttcctcgaccactggaagga

SEQ ID NO: 339 - DNA, 21 bp, Artificial Sequence
  A synthetic sequence
tcttcaaagcacctctgagca

SEQ ID NO: 340 - DNA, 20 bp, Artificial Sequence
  A synthetic sequence
atggtcttcctcgaccactg

SEQ ID NO: 341 - DNA, 20 bp, Artificial Sequence
  A synthetic sequence
gggatcctcttcaaagcacc

SEQ ID NO: 342 - DNA, 19 bp, Artificial Sequence
  A synthetic sequence
ccggacacgcttctcttgg

SEQ ID NO: 343 - DNA, 21 bp, Artificial Sequence
  A synthetic sequence
tcaaagcacctctgagcaggc

SEQ ID NO: 344 - DNA, 24 bp, Artificial Sequence
  A synthetic sequence
cacgcttctcttggagaaataaca

SEQ ID NO: 345 - DNA, 21 bp, Artificial Sequence
  A synthetic sequence
agggatcctcttcaaagcacc

SEQ ID NO: 346 - DNA, 20 bp, Artificial Sequence
  A synthetic sequence
agcctgtcatgagacctcca

SEQ ID NO: 347 - DNA, 20 bp, Artificial Sequence
  A synthetic sequence
tcctatcatgcctgttggtg

SEQ ID NO: 348 - DNA, 20 bp, Artificial Sequence
  A synthetic sequence
tgtaatgacgcaaccaacct

SEQ ID NO: 349 - DNA, 20 bp, Artificial Sequence
  A synthetic sequence
gcaggataggccattacagg

SEQ ID NO: 350 - DNA, 20 bp, Artificial Sequence
  A synthetic sequence
tgtaatgacgcaaccaacct

SEQ ID NO: 351 - DNA, 20 bp, Artificial Sequence
  A synthetic sequence
tcctatcatgcctgttggtg

SEQ ID NO: 352 - DNA, 20 bp, Artificial Sequence
  A synthetic sequence
tgagacctccaaaccccttt

SEQ ID NO: 353 - DNA, 20 bp, Artificial Sequence
  A synthetic sequence
acttcccatttgtggaggaa

SEQ ID NO: 354 - DNA, 20 bp, Artificial Sequence
  A synthetic sequence
aacccctttggccctgtatc

SEQ ID NO: 355 - DNA, 22 bp, Artificial Sequence
  A synthetic sequence
acacttcccatttgtggaggaa

SEQ ID NO: 356 - DNA, 21 bp, Artificial Sequence
  A synthetic sequence
ctgtaatgacgcaaccaacct

SEQ ID NO: 357 - DNA, 21 bp, Artificial Sequence
  A synthetic sequence
tgcctgttggtgtagtagcag

SEQ ID NO: 358 - DNA, 20 bp, Artificial Sequence
  A synthetic sequence
tgtaatgacgcaaccaacct

SEQ ID NO: 359 - DNA, 20 bp, Artificial Sequence
  A synthetic sequence
gcctgttggtgtagtagcag

SEQ ID NO: 360 - DNA, 20 bp, Artificial Sequence
  A synthetic sequence
aggagtcaaggcatcaccac

SEQ ID NO: 361 - DNA, 20 bp, Artificial Sequence
  A synthetic sequence
ttcaagagagccgtccaact

SEQ ID NO: 362 - DNA, 20 bp, Artificial Sequence
  A synthetic sequence
aggagtcaaggcatcaccac

SEQ ID NO: 363 - DNA, 20 bp, Artificial Sequence
  A synthetic sequence
ctgctccacgacacaaaaga

SEQ ID NO: 364 - DNA, 20 bp, Artificial Sequence
  A synthetic sequence
tgaggaggagtcaaggcatc

SEQ ID NO: 365 - DNA, 20 bp, Artificial Sequence
  A synthetic sequence
ttcaagagagccgtccaact

SEQ ID NO: 366 - DNA, 21 bp, Artificial Sequence
  A synthetic sequence
gaggaggagtcaaggcatcac

SEQ ID NO: 367 - DNA, 20 bp, Artificial Sequence
  A synthetic sequence
acaaactcggcgtgttcttc

SEQ ID NO: 368 - DNA, 20 bp, Artificial Sequence
  A synthetic sequence
caaggcatcaccacagcgaa

SEQ ID NO: 369 - DNA, 20 bp, Artificial Sequence
  A synthetic sequence
ttcaagagagccgtccaact

SEQ ID NO: 370 - DNA, 19 bp, Artificial Sequence
  A synthetic sequence
atcaccacagcgaacgcat

SEQ ID NO: 371 - DNA, 21 bp, Artificial Sequence
  A synthetic sequence
caaactcggcgtgttcttctc

SEQ ID NO: 372 - DNA, 21 bp, Artificial Sequence
  A synthetic sequence
tgaggaggagtcaaggcatca

SEQ ID NO: 373 - DNA, 20 bp, Artificial Sequence
  A synthetic sequence
gtccaactgctccacgacac

SEQ ID NO: 374 - DNA, 20 bp, Artificial Sequence
  A synthetic sequence
catcaccacagcgaacgcat

SEQ ID NO: 375 - DNA, 21 bp, Artificial Sequence
  A synthetic sequence
ttcaagagagccgtccaactg

SEQ ID NO: 376 - DNA, 20 bp, Artificial Sequence
  A synthetic sequence
ggagtggttggggtgacttt

SEQ ID NO: 377 - DNA, 20 bp, Artificial Sequence
  A synthetic sequence
tcaactgccatacgaagcgt

SEQ ID NO: 378 - DNA, 20 bp, Artificial Sequence
  A synthetic sequence
tttgcaggaatgtgtgatgg

SEQ ID NO: 379 - DNA, 20 bp, Artificial Sequence
  A synthetic sequence
atgaatccactcgggcttta

SEQ ID NO: 380 - DNA, 20 bp, Artificial Sequence
  A synthetic sequence
gcatttgcaggaatgtgtga

SEQ ID NO: 381 - DNA, 20 bp, Artificial Sequence
  A synthetic sequence
atgaatccactcgggcttta

SEQ ID NO: 382 - DNA, 20 bp, Artificial Sequence
  A synthetic sequence
ctctcgggatgacaccactc

SEQ ID NO: 383 - DNA, 20 bp, Artificial Sequence
  A synthetic sequence
atgaatccactcgggcttta

SEQ ID NO: 384 - DNA, 20 bp, Artificial Sequence
  A synthetic sequence
tgcaggaatgtgtgatggag

SEQ ID NO: 385 - DNA, 20 bp, Artificial Sequence
  A synthetic sequence
atgaatccactcgggcttta

SEQ ID NO: 386 - DNA, 20 bp, Artificial Sequence
  A synthetic sequence
gcggcatttgcaggaatgtg

SEQ ID NO: 387 - DNA, 22 bp, Artificial Sequence
  A synthetic sequence
acacttcccttgtgttcagtgc

SEQ ID NO: 388 - DNA, 21 bp, Artificial Sequence
  A synthetic sequence
ggcatttgcaggaatgtgtga

SEQ ID NO: 389 - DNA, 22 bp, Artificial Sequence
  A synthetic sequence
acacttcccttgtgttcagtgc

SEQ ID NO: 390 - DNA, 20 bp, Artificial Sequence
  A synthetic sequence
gttcagcaagtggtggaagc

SEQ ID NO: 391 - DNA, 20 bp, Artificial Sequence
  A synthetic sequence
ttttctccaccacccatctc

SEQ ID NO: 392 - DNA, 19 bp, Artificial Sequence
  A synthetic sequence
ttcagcaagtggtggaagc

SEQ ID NO: 393 - DNA, 20 bp, Artificial Sequence
  A synthetic sequence
ttttctccaccacccatctc

SEQ ID NO: 394 - DNA, 20 bp, Artificial Sequence
  A synthetic sequence
gttcagcaagtggtggaagc

SEQ ID NO: 395 - DNA, 20 bp, Artificial Sequence
  A synthetic sequence
atctccatctggcaaccaat

SEQ ID NO: 396 - DNA, 19 bp, Artificial Sequence
  A synthetic sequence
cagcaagtggtggaagcac

SEQ ID NO: 397 - DNA, 20 bp, Artificial Sequence
  A synthetic sequence
ttttctccaccacccatctc

SEQ ID NO: 398 - DNA, 23 bp, Artificial Sequence
  A synthetic sequence
gtggtattagttcagcaagtggt

SEQ ID NO: 399 - DNA, 25 bp, Artificial Sequence
  A synthetic sequence
atttaacattgccccataatctctt

SEQ ID NO: 400 - DNA, 23 bp, Artificial Sequence
  A synthetic sequence
agtggtattagttcagcaagtgg

SEQ ID NO: 401 - DNA, 25 bp, Artificial Sequence
  A synthetic sequence
atttaacattgccccataatctctt

SEQ ID NO: 402 - DNA, 24 bp, Artificial Sequence
  A synthetic sequence
agtggtattagttcagcaagtggt

SEQ ID NO: 403 - DNA, 25 bp, Artificial Sequence
  A synthetic sequence
atttaacattgccccataatctctt

SEQ ID NO: 404 - DNA, 25 bp, Artificial Sequence
  A synthetic sequence
gcaccaatagtggtattagttcagc

SEQ ID NO: 405 - DNA, 25 bp, Artificial Sequence
  A synthetic sequence
atttaacattgccccataatctctt

SEQ ID NO: 406 - DNA, 20 bp, Artificial Sequence
  A synthetic sequence
atgtgcctcctgaaaggaaa

SEQ ID NO: 407 - DNA, 20 bp, Artificial Sequence
  A synthetic sequence
caacagcagcgtaagaaacg

SEQ ID NO: 408 - DNA, 20 bp, Artificial Sequence
  A synthetic sequence
tgtgcctcctgaaaggaaat

SEQ ID NO: 409 - DNA, 20 bp, Artificial Sequence
  A synthetic sequence
aatgcatccactgcaccttt

SEQ ID NO: 410 - DNA, 20 bp, Artificial Sequence
  A synthetic sequence
tgtgcctcctgaaaggaaat

SEQ ID NO: 411 - DNA, 20 bp, Artificial Sequence
  A synthetic sequence
caacagcagcgtaagaaacg

SEQ ID NO: 412 - DNA, 22 bp, Artificial Sequence
  A synthetic sequence
agaaaggttacaggtctctggc

SEQ ID NO: 413 - DNA, 20 bp, Artificial Sequence
  A synthetic sequence
caatgcatccactgcacctt

SEQ ID NO: 414 - DNA, 22 bp, Artificial Sequence
  A synthetic sequence
aaaggttacaggtctctggctt

SEQ ID NO: 415 - DNA, 20 bp, Artificial Sequence
  A synthetic sequence
caatgcatccactgcacctt

SEQ ID NO: 416 - DNA, 22 bp, Artificial Sequence
  A synthetic sequence
aaaggttacaggtctctggctt

SEQ ID NO: 417 - DNA, 21 bp, Artificial Sequence
  A synthetic sequence
atgcatccactgcacctttct

SEQ ID NO: 418 - DNA, 20 bp, Artificial Sequence
  A synthetic sequence
aggagtcaaggcatcaccac

SEQ ID NO: 419 - DNA, 20 bp, Artificial Sequence
  A synthetic sequence
ttcaagagagccgtccaact

SEQ ID NO: 420 - DNA, 20 bp, Artificial Sequence
  A synthetic sequence
aggagtcaaggcatcaccac

SEQ ID NO: 421 - DNA, 20 bp, Artificial Sequence
  A synthetic sequence
ctgctccacgacacaaaaga

SEQ ID NO: 422 - DNA, 20 bp, Artificial Sequence
  A synthetic sequence
tgaggaggagtcaaggcatc

SEQ ID NO: 423 - DNA, 20 bp, Artificial Sequence
  A synthetic sequence
ttcaagagagccgtccaact

SEQ ID NO: 424 - DNA, 21 bp, Artificial Sequence
  A synthetic sequence
gaggaggagtcaaggcatcac

SEQ ID NO: 425 - DNA, 20 bp, Artificial Sequence
  A synthetic sequence
acaaactcggcgtgttcttc

SEQ ID NO: 426 - DNA, 20 bp, Artificial Sequence
  A synthetic sequence
caaggcatcaccacagcgaa

SEQ ID NO: 427 - DNA, 20 bp, Artificial Sequence
  A synthetic sequence
ttcaagagagccgtccaact

SEQ ID NO: 428 - DNA, 19 bp, Artificial Sequence
  A synthetic sequence
atcaccacagcgaacgcat

SEQ ID NO: 429 - DNA, 21 bp, Artificial Sequence
  A synthetic sequence
caaactcggcgtgttcttctc

SEQ ID NO: 430 - DNA, 21 bp, Artificial Sequence
  A synthetic sequence
tgaggaggagtcaaggcatca

SEQ ID NO: 431 - DNA, 20 bp, Artificial Sequence
  A synthetic sequence
gtccaactgctccacgacac

SEQ ID NO: 432 - DNA, 20 bp, Artificial Sequence
  A synthetic sequence
catcaccacagcgaacgcat

SEQ ID NO: 433 - DNA, 21 bp, Artificial Sequence
  A synthetic sequence
ttcaagagagccgtccaactg

SEQ ID NO: 434 - DNA, 20 bp, Homo sapiens
ttgattcataaacgaattct

SEQ ID NO: 435 - DNA, 20 bp, Homo sapiens
actgtagtcgttactgaata

SEQ ID NO: 436 - DNA, 120 bp, Homo sapiens
attgtagctcaagatgaagaacttgagaattattgttacaccaatttcctagaattcgtttatgaatcaacagaaaaatactccagtaagcttttcttatgtcagtaaagtagtttgaat

SEQ ID NO: 437 - DNA, 120 bp, Homo sapiens
tagaagagttactttgagtgttcttaacaagtacccgtttaagaaataatactgtagtcgttactgaataaggcttgaattttatattataaatataattagtaatatcagtaaagatgg

SEQ ID NO: 438 - DNA, 300 bp, Homo sapiens
attttattacttttttctcagggttatcttaacattcttccagttttccacatccatagtaagttttagcatccatcactgtatgtagtacttttaaagaattgtagctcaagatgaagaacttgagaattattgttacaccaatttcctggcttgaattttatattataaatataattagtaatatcagtaaagatggtaagctatccagaccaactttcagtgagatatttacctcagaatttgtcttttgattctcatagccataaatataaatcctaggagatgtaaatatattac

SEQ ID NO: 439 - DNA, 18009 bp, Homo sapiens
ggaacaacctatcttcagcactcgagctcatgtcttccaaattgacccaaacacaaagaagaactgggtacccaccagcaagcatgcagttactgtgtcttatttctatgacagcacaagaaatgtgtataggataatcagtttagatggctcaaaggtaagctacgtttactttgaatgatttggctggttttgcttttttcagtatgacattttagttctattcagaacattttatcttcattaatataaaatgtttccttttgagttcttaaaacttttaagaaacaaaatggattattaaggtagcaacatgttttgtccactaaatgactaaccactttattacattgttacatgccaaaatattctacaaatttgaggtcattccatttatttaatgttcattgcatatctgttatgtgtcaggcattatgccaggtactaggaatatagtggtggacagaatagatatggtctagaattctaggagctctaattcttttcaaatatactgctacagactagtagcagtttaaagtccctatatcctatgaagtttgtcagtgtcaatactaaagctgacatttcttctctcaagagaagtataaggacaaaacctattttatttttaggagaggcaattagtgtaacaggagcaaggactgcctgggttcagatctcaactccaccatctactgagtgttttctctttgggcagattacttctctgagcctcattgttctcaactcagtactggtggttgtaaggattaaatgagttaatatctgcaaagcactgaaatcagtgcttagaggacagtgtgcccttcagaagagttggttggggatcccttttttttttttttttttgagacggagtcttgctctgtcacccagcctggagtacagctcagctcactgcaacctctgcctcctgggttcaagtgattttcttgcctcagcctccccagtagctggtattacaggcgtgcgcctccatgcctggataatttttgtatttttagtagagacagggtttcaccatgttggccaggctggtcttgaactcctgacctctcgatctgtctgcctcagcctcccaaagtgctgggattacaggcatgagctaccgcgctgggacgggggatcctttaatcatcaatgaacttcatggaactcacttagagaaagaaattccaaagaaaaatcagactctaagattctaggtttgaagtgtgtagaaagatgaggtcttcagtttcttaaagccatgaagcctttccactttcaggaattttagcaattaaatgaagaaggggttaagtgcacacactgtagagccagactgactggtttgaatttttgtttctactacctgcttgccatctatattagtccattctcatacttctatgaagaaatacctgagactgggtaatttataaaggaaagaggtttacttgactcacagttccacatggctggggaagcctcaggaaacttacaatcgtggcagaaggtacgtcttcacagggcggcaggagagagagtgagagcaagcaggggaaatgccagatgcgtataaaaccatcagatctcgtgagagtcactatcacaagaacagcattggggaaactgccccccatgattcaattacctccaccgggttccacccttgacacatggggattacaattcaaggtgagatttggatggggacacagagccaaaccatatcaccatctgatcttgagcaaagtatttagcttctgcatgcagggctgggcactgaggtttgtgcctgtaattctagcactttgggaggccgaggcatgaggatcacttgagaccaggagtttgagaccagcctgggtaacacagtgagttcccatctcaaaaaaaaaaaaaattagctaggtgtattggtgcatgcctgtagtcctagctactcaggagactggggctggagctaaggaggtcaaagctgcagtgagctatgattatatccagagcgagaccctgtctcttaaggggaaaaaaaaactgtatgcctcagttttctcatctgtaaaaaggaatgaataaaaatacctacatcattccaccatagggttgttgtgaaaagtaagtaaatttaggcaaaagcacagtgtatatagtaatcactatacaaacactcactgctattgttgttaatactattatcaggcgattattgcctagtgttttatggggaataaaagataatttaatgctaaactttttgtctgttttttctgtgtactcattccttatctccttaactaggttaactagatgataagaccctaagcgctatttgcactttgtgtcttgtacataagtaactaatatgtatttacttgaataattgactttttcatttttacttggaggatttaaaggacttgagaaatagaaatagttcatttccgtgaagctttatttagtgaagaataattttgtcatattttgtaactgatttagtgaagtttgacagatactagatattataacctgagactctagagtgtttatttcttttcccttatttgaaccttgaattctctgtaatctgttttgtttctgggcccaaacgacagataacgtgcctcctttattagaaaggagggttaacagaggtaacagaaaaagataacaatcctcctttattagaaaggagagttaaatgttgtatacaaaaaaaggtcaactgaatagtcccatgtcggagaaaagaaaccatatggaagtgattaaatagatttcatttgctaacttaattttcaccagattaaaatactttgagttattttaaaggaaggaattttagtgattaacacataatcctcatgtgttcttcccactgaagcaatatgaaatgttgctcttgcatttttccatttctgaatatacgttgctgattgtaattattagaatcataaatgactttggaaaagctttttgtatttttttaacaaaatctatcttctgtagcagagttatattttctatcacattttccattttctaattacttttctaaactgatgctattaactgctgcagttcctacaggagtggttgtttttttaagtcagcaatagtggtttatttgactcagctaaacaagtgttttctccatctcacccccgacttctgctcttcatgattgagttggaaaccccttaggtgccctgcctagaaaatgtgcactcgttctcacttagtgcctctactgtgcatatgaagtttgctttaaggttttctgattattctacagagacctcagcatggtcaagggccaggatcagggcactgtgtgatccatacatgaggtagtaatgacactgcatttctacctcttttccttaaaaggactagaactccagggttgagagacatatttattttcagaactaaaatttttcttttaaatgcattctactcaaagaagctaagaaataaagctttttaaaagtgtgggatcatctatcattttaaggttttcttcattaaagaacaagtaatacagagacaagggaaaggttcagaatggagattaaaataaaactcataaaagcggaaagaagttaatttgaaatagttaaaatgagggggcttagtgaccaactaccagcacgttggtcagttttcttagatgcctataaaacctgttgttgactcacttttgagttattagggtcacgtataaaatgcccttgtcagaaaacacagagatcccagttcagatgtgccccctcaggaccagcaaatctaaggcagtgcctaccagtcttgatttcttgcgtttcactctgtcacatcctggtctttatagtacttactactgtctgagattacattggtcagctatttatttacttgtatgtttactgactccttttattctagactgtaatttccaattatattgcccctcttattgttgtagctcaaatttttagaactgtgcttggcatttaaaaggtactcaattaaagttgaataaacgaatgattccttaaccatttgttgataaagtccttcctatttagtctttcatttgatgacttcatttgatgccattcaagttagagcagttattagccacattttacacatcccaaatgatatgctaagaaaagcagtttttgaactgaagggtaaggaaactgtataagtatgaatatgtaaattttgtctgaggtaaaaacacttaattgagcataagggctctcatataacagtggacacatcttctgtctgagagaaaggaagggaccactctgttttactggttttttttttagtaggacaggtgtcatgcattcatttttatgacttctccaatacctgcatagctcttaaatatttacttttatactgttgttcacatggtaaacaaatttagttttgttcagaaaatgctcacatggtaagcaaatttagttcagaaaatgcttgatgtatcattccacatttaagagttcagcagatgactgcttatggatttgttaacatctgtctattactattggaatcactttctaatcttaaatttggagtgcgttagactaaagaaatgattcctcacttttatggtattagcagagagaatgagttaatattttatgataattgaaatttattatggtgatcaccaaagttaatatttagtaacatgctaaaatactagttttctaagaaattatgtgtgaaacaaaaatgatagcaattctcaatatcatgaatctataccactttatgagaaaagaaatgagtatgcagttgactcatgacactggtttgaattatgcaggttcacttatacgtggatcaaaaaatacagtatttgtaggatgtgaaaaccaagtattagagagttgacctcgtatatgcaggttctgcagtgctgactatgggacttgagtatgcataggtttttagtatacataggggtcccggaaccaatctcccaagtctagcaaggaacacctctatatttagaggtttgttttttccttgagtaaagggaaaaagtactaaacgattgcaagtcttttctgtatcataatgtatttcaggtaaatcagcttgaagaaaactcaattatggattttagaaaatcagtgtcggccgggcgcagtggctcacgcctgtaatcccagcactttgggaggccgaggcgggcagatcaggagatcgagaccatcctggctaacatggtgaaaccccgtctctactaaaaaaaatacaaaaaagttagtagggcgtggtagtgggcgcctgtagtcccagctactcaggaggctgaggcaggagaatggcgtgaacccgggagttggagcttgcagttagccgagatcacgccactgcactccagcctgggcgacagagcaagactccatctaaaaaaaaaaaaaaaaaaaaaaaaaaaagtgtcatattttctatgcatattttcatttttgaaagattctttggagggtggtaccctacttttaaataaaccactattaaggcctaccttcttctagtgattatcatccaaaattaataaatattgattctaaacattcaaaaggaaaaaactgaccatattaaaaactctttgacactttttggatcataggtaaacttatttgagtgtaaaatgttgaaatttttgatagagtgtcatcatatttatattttagttaacatatttccacactatcacttataatgccagaaactatttctgtaactatctttatgtatttaacgttgtggcttattgataatcttaagcttttatgtaactgaatggtcttgtatttaaatgcctgtttgctgattttttgctggttgctcatacttgctttattttttaggcaataataaatagtaccatcaccccaaacatgacatttactaaaacatctcagaagtttggccagtgggctgatagccgggcaaacaccgtttatggattgggattctcctctgagcatcatctttcgaaagtgagttaaatcataaaatttgaatgaaaatcttcatcttcaagtcgtatatcttaaatatataataccaaatggagagtataaaatataggagaaatttatttaatattctcgaaacagcagtagtgacttcagagcaagaagtaagagatcattagattctgctgtcacttggaaaatgtacacattaaaaacagtcaccattggttgagaaggatttatgtggttaattgtctgtgtgataggaattaaaaggtggggattatcacttcagaatggatttttgtcagagattagtaccctcaattggagatggtctacaagataggcagataccaagctcttttgaattgtgaaaactcaagctgacagtgatatagatttcagcattttgatgtgacagcaggtacatataaattactgtgtttaggggaaattgtgctttatctggatgtggaatactccgagctctgatgcagtgcagactgagatactatagacatcatgaggaatttttagataatactcaccatgtgaccagaaaggccaataaaatgcaaagcatcaccaggctaaatattggaaataagacagttctcctgtcaataagcaaaagccatgtaatgtttataccagcagcaccttgggttcttgtggatgttacacagaaaagaatatataagaaaaaacaatctcagtatactaagtgatcaaagagaaaagtaaaactcacttttctcttcagagcttgtgcctgccagacctaaagatttattcttaaaacctctgtttgaagcattcagttaaaagaataaccatatattttaaatactttacatatgatttggtatatgcaggatctgatactggaactcaggctagagagttgaaattatccaagtgcagagaaatcaggctcttgtagatatacagtgtttatatttacattaaattttaagctgtttttctttacagaaattattctgtattcaaattcttaaactgggttctttttagaaatcttacacttttatgaatatatattgaatctattttattttcttctaattataaacatttcttaacactgttttcaagtgtttttgttatcttttggatatgaattatagtttaaatctcaattttaagggtattttaactttcgccttggtgattataaatcattttctgtaggatgattaaaacaaatattaaggctgggcacagtggctcatgcctttaatcccagcactttgagaggccaaggagggcagatcatttgaggccaggagttcaagaccggcctagtcaacatggcaaaaccccgtttctactaaaaatacaaaaaaattagctgggtgtggtggcacacacctgtaatcccagctacttgggaggctgagacatgagagtcatttgaacccaggaggcagaggctgcagtgagccaggattgcgacactgcactctagcctgggtgacagagcaagactctgtctcaaaaaacaacaacaacaaacaaatatcattttggtttctcttgtacacatcttattctttaaatgtatttcttgaactgtggtaaaataagagttttacattcaaagtatctcagcataactaggatacaggtttccattgaacattgcccttccataaacagttctgttgagaaattatatgtttattctaatacttttgccatcattcagaacctttttagaactttgtgtttttttttattgcctattaaagactatagcaaaaatctataaatatatcttgctgaatttcttaacttgacaatacaagaactaaatcatggattattttctcaaacttgccttaaatgctttttgggctgagtaattttgtctgtaaattaagttcttcgttacatctttcaagttactgagcagctagctaatgtagtaaaggaatagtttattaacttctgtcagttctactcttttcccttgtttgaacttttctctgagaatggctttttttttttaattttttttcttctttaaagttagcatatgcttttgaccagccttactggtggtaaatctagtatttgagggtaggttgaatattgggaaacagctcttaagctcttggagccaattctagcttatatactcacagaacttggctccaagagcttcagatcagtctcttcagaatgttttgatccaaagctgacaaaatcataatttatttttcaggctaccttgtagactaattttggtgttttgcagactgtaggaggctcaaaggcatttataatattttacataaacaaagcaagtacataaacattctagcttgttttatcagttaaaagccatgagtaagtcaaaacattataagggtttggagaaggcaattctagacaaagacatctcaaaataagggaaagtaccatcgttgaaaaaaaatgtgtggtctcttaagattgaatggtatgagcaaaattccagctatgcttggattgttagagtgcttactaagctgtattttgttattatgggaaatctaagctgtcacttttttattttgctgaaaactaaggatatacaaattagcaatagtgtagatttttacgaagttgagaaggcttttaaagattaactattgtggaatgttaaattagtgtaagaaaaaccttgtatactatttcagattacataaagagaagttttgttttgatttcatttccaccaatgtgagtgagacccttgtagcttcgtattcagatgttgtactgtttggagatactctgttcaattggagaagtatagtagcacaaattcactttcctcagatacacattataggtagaagtgacaaggtttaaggtaagaaggaaaagttgtgataatgatggctttaattttgaccctcctccatctcgccaggattagagtatcacaggcctttccttccaaaatttttcttcccaagatggaaatgaaatcagagttcgtttatgagggtttgagaaaggaactataatttcatgttttaattaccagctctaacagatattttaagttgcatagggatttggggtttttacatgtagttattatgtagtcttaacttattctcactgataatagtgatagactagcttccacttccaaaacataaatagatattctttgtggccaggatgttttatagtttcctatgagttcctaagaattagcatttcatgtacaataatttttgttttgaaattgttatccatcttaaagaccactgagtattattcctttctgctgctgtacatgagctatttaggaatcttcggaaataaatttttagttatgtcagttagtgaaaatagtaaatattactatttgtattactgaaaatgcattgtgtattaagagagtttatattttagtaaaatttaaaagcattttctaaatactgtgatgtttacaagtggatacttgtgaaattcagttttctcaatgataaacatagcacagtacgcttatgtttactgattctttcccctaccttggtctcagtaaattatttctgaacaaccaggagtcctttaggtaagctagctgactctgaagtctgacttagctatatcatgatgttttgaataagatttgtcaaaatacttcatagtgcacagacaactgtttggaattaggtttcatttcctgtcattttcatattagaagctataattggtttggaggctatattgcttgtgtagagtcagttattcagaaaatgaataactttactaaggtaaaagtgggcactgtgacctatttttatttggttaatgtatgtagtctctatacattcagtttgcagaaaagtttcaggaatttaaagaagctgctcgactagcaaaggaaaaatcacaagagaagatggaacttaccagtacaccttcacaggtgggtatatcatttctattcttaattatgaaccttgttcatttcagataatatccttcatgccagaattaaatctccaaagtacacacagaaatgcaccctttagtgtggtggctcacacctgtaatcccaggactttgataggcttaggtgggaggattacttgagcccgggatttcaagactagcctgagcaacatagggagacctcatctctacaaattaaaaaaaaaaaaaaaaaaaaaaaaattagccaagtgtggtggtgcaagcctgtggtctcagctacttgggaagctgaggtaggaggatcacttgactgggaggtcgaggctgcagtgagctataattgtgccactgtgctccagcctgggcaacagcaagaccctgtctcaaaaagaaaaagagagagagagaaatgcaccctttacttaatttgtactggtggaaaaaggtttttctatatgccaatgcctacaagaatatttgtaatcaaaatttcagtttagtaattcaattcaaattatttaatgtgcttgtaacttagcaaaagtacataaagatcatgtgatagtcaaatgtaaccacaaggggagtcataggaaaagttcaggaaaacaagtttattatacttgctggtcctagaaacagggaaagtcttaaggtggtcaagagacagaaagcaggagtgaggggaaagccgaggtagagctttattggaatttccattggaaaggtaaagcaggggaggggaaatagtttaggattggctgggttgaataatttcagggggctctaaactgtaggggtggtccctcattgtctggtaactggccctgggatggattaagttagaggaatattgtctcctggatcagatgtatggatcagatagaggaggtatggctctggattggctagtttgcatatcagaggcatgctctggactgggctcttgctgtccttaagaattagctagccctagaggggagaagcagtctctttgcagccagaaaatttttttaaaagatgtcagaatgtcataatatacataaagttaaaatacatgcacgtgcgtgcacacacgcacaaacaatagggtttgttttacatacttttattatttatttatttatttttgagctagagtcatgctctgtcacccagaccggagtgcagtggcacaatctcgggtcgctgcaacctctgcctcccaggttcaagtgattctcatgcctcagcctcccaagtagctgggattataggcgtgcaccaccccacctggctaatttttgcattttattagtggtggtgttttatcatgttggccaggctggtctccctggcctcaagtgatccgcctgccttggcctcccaaaatgctgagattacaggcgttagccacagccccagcctgttttacatacttttaaagtatacttaaatctgacagttggaacatctttatgcttagcactgagacaatggttataggttttacaaaagtattactttttcttttaacaaaggaccttttacttgtccatcataatcttccttcagttggaagttatatctccaagtaaagttgattaaatagttacattttggtacttttataatacagtggttttactgtatgattaatatagaatgccactattttcagctttttaatagtaggcttttctaaaattaatttttattttaattgtatcctactagtaggattcagcttttggctgttctcctctgtagtaattgtttctgaactgtactcagtagcttttatggaaaggaagaatcagatgttacaaatgtttctctggaataatgtcagtagtagcagatttacatggaactttcttgaagaaaataggaattgccttggaatttaataactaaccaaatgattggaacagtttagaaaattatttattctttatctccctctccacttctatcacccagctctatttggaaaactactggtatagagagttgattttttttttaataagaataaaggagagctctgttaaatatgcttaaagtgcatattcccttagactaaaggtgtactctggattgaagccaaagctccattatacgtctgtacatgcaatgacctggaataactttgatgatcatttgaaacttctcttgaatctgtcttctctaagtgctgcgaaggtgtagtgatacttttaaacataaaaccccatttaaaattaagtggcaatacatattgggagctctagttcctctggtgagtgattccagtgttataagaagatccaacaaacagcacattcagtaaaagagtactcaaaggattttaataaaagtgtgatggattaagctttgccattgtacttagttcccgtttctctaatcgtaacttccattttagttgaattggtcatgaaccattataagttgctgctaacaccttgttcctctttggtggaaggaatgactttaagcctctgctttgattaaaggaatatataaaagatgcttatatttgaaaaatactatgaaatataaagaataaggaaataagttcatcactatttgagagccaaaatatatggaccttaatttatcttatcaaaacatttgaagcctaattgtcaatttaatatacagaagccagagagaatcattttttaaaaagcatttatcagccaggcatggtatgcacctatagtcccagctactcatgaggctaaggtgggaggactgcttgagcccaggggtttgaggctgcagtgagctacaatcacgtcactgcactgcagcctgggtgacaatatctaatgtgtctgttttagtttgaattgtatgaccattggccatacctaccctacttgattctcagtggtttaacccaatctgctgaattaatgctgcctgaagaatgtaacgtaactctggaatttagcaaaagaacggttaagtggggtaattgataataggttaaacacgaaaatcgatagtagtagaaacttggttggtcaaagcttttgtttttatttgagggaggtgtaagagactggttaaaagtttgagtcagacaaacctagatataaattacttttctgttgccatgtaatacacttggttgtttaacacaagtgtgtttatacatttataacatgagtattacattacttatagaatcattgatcatcatcaataagtatggtaacgacacagtgtcaggtcatggttaataatgggagttctagaatctgaatttgaaactgggctccaacccttaatagttttctgagcttaggcaagtctctgtttttcttcctctataaaatgagaatgagaacaatatgtccttaataggaatattgtgagaattaaatgaggattagaaagtacttagcatatagtactaaatgcctaataaatgttaacaactgatgtggttgtaatggggaggatgatgatgtaaggccttaacatactgtctggcactaacagttatctttaaaaattcagatgccttaattgtatatttatacaatatgtatttgaaaaatactatgcgtcagaattctgcatcagaattccctgaaagacttgctaaaactcagatcggtgagctccactcccagactgtctgatttagtaggtctaagatggaacccaaggatttgcatttctaacaagttttctaggagatgctgaagctattcctatgttagatattatattatattgtagaatattatatatgcctaattcagttgtgagtattattttaattttttagccatttacattttagtcatttcaaagtatttagcctggctttcattaaaattaccactctagttttgcattcaaaatcagtagtttacatagtacttaattcagttttataattatataaatagaattggcataaacaggtttgggacacatacagctgactcttggtaagcatttgatgcagctggtggttgcaaaatattcaagcagagtaacaattaacccactaaccctaagcatcaatcagccaatatactttacatagcgaaggaagagcataaattaatccagaaagtcagattataagaacttctgtggttgtcacaaaatggatttcagaaatggtggctctggatggtgactaactataggaaaaatgtatgactgaggatatgaggatcttgtttgagagctcaacctcatcttcctttatgtatcgttttgctgtcttctttccattctcactgccatgggatctcaagactattagctcctccccagtttttctgtcaggccctctgctgagaaaacagtggtgttctcatctttgtttaagtataaaactggaatggactgagtcttccatgaatgtttgttaaatggtaaagagagggccttgttttccttaagccaacaaggttgaaactgtaatgggaatttaatttcaggttttattggcatgaaatggaaggtggtagtggtatttggtatagtgggtcatggcagtgctgctgcgtacctccccagcccatccttccaatacacagtaggtcactgaaaccaccctaaaggcttgtcctagagtgagggtcaaggtcaacttttttccatgtgttctagaatatttctcattttttggctttcatagccaaaatttcagttgtgggttttattttaattttcacttaagttacccacttcacaaatgggtaggaagagagaattcttaagaagcatttcctgaatctcacatctgtgctcaggtgccttttttataataggttgcatctgttagttttgatttccttgatactctctcagatttgttcttccaaaaataaattttttcttcagtgttactgtcctttttcttactgtagattatttgttttggtgggagttatagggagagtttctcagaaaaaaaatgagatgtaagtggttttattttctcaaagcaaggatataaagtcaatagaaaagattatttcatctgtttttatttcattttattttttgcttttttttttcatttttcttatcagctttgggtatacacacacacacacacaccccaaaaaagaataactgaagtaggatgtttaggaaattacacacaatttagattaaaaaagaaatagatgcttagaatatgttaaataaaaaacatgtttcaagtactatttttagcatatgcaaaatttctggaaggttttgcagaatacttttggctacctctgcggtagttggagggaaagagggtttcattttccttttgtgccctcatgtactgttggaattttttgttgtttctcatcagtaaatactaattttatatttttaaaactgactttaaaaaaacccaagttctcgatcggttcagaataaaaaattaaattaaaaaaataaaataccatgctttgggtgggctttttgttttttactttaggtataatgacctaggataactatgcctttacttaaatatagctaatattcaagttttctgaagaaaaacttgctgctttgagatgttggaattgctacaacctctatccctttctttctccccttcttaacttgcccctgggaacagtctttctactttagtagcattaacatttgtagcctccccaagttccatgcctcaaaccctactccctcttatattaccctcaaagtgaccggagcatttatcccagaagatggtaacaaccatgagtcaaactgctactgctcttcctctctctcttttgatcctaatgtacttttgcacactcttgtcaaccagttccagcaaatccctgagttaatccttaattgctaaccctagttatgccctttaagtttaataaggtaatttatttttatttttattttttattttgagatggagtctcactctgttgcccaggctggagtgcagtgatctcggctcgctggaacctccacctcccaggttcaagcgattcttccacctcagcctcccgaatagctgggactacaggcacctgccatcatgcccagctaattttgtttgtatttttgtagagattgggtttcatcatgtcagccaggctggtcttgaactcctgacctcaggtgatccgcctgccttggcctcccaaggattacaggcgtgaggcaccacgtcccggccggtaacttgtttctgttatcatttctactgaattgaatcttcaccaatttaactgtttagcaggttgcacattcaacctttcattattttagtttggcttggcctgtcttggagagaaataagcctgatttgttaataccagagatgattattttatgatatcttactggctggttaaattttttctgactaattaatatggtccataactgacctagaagtagtcactttgtgattgctattcccattattcacttaataggatttgactttgttgtaaaacatttttatattgtcacattatactgtagattttattcgtgcattgactgtcagctaaaggaaacagctgatttaggagaaatatttaataaaaatgaaattaaaggtcaaaattagaagttttactgaaaattgaaaaatagttaagtgataatgtggaattaagaaagtatccttttgatctttaacagcttgtatagtttatagcattatggaaccattagttaaaatacttaaatgtgaaataaaattcatacagcagtataaaaccagaagtctcaaaaacaaaagtctcaccacgttaatatttacatattttttctagcacttcatgtgtaacattctaaatgtgaaggatataaatataacccagttactgttttacattctgatttttaaaaattatatgctacattgctcatatttgtcttatttttattagaggaaatcttgtattttttttaagttgacaaattatgatcattcctaaccactcccctgttggacacttatttttttctcccaaatatagacagatatttcaattgtaacatttttttgcccagggatcataagaggtccaccaagctgctctttaacagtctggttttagcacctcccctagttaataggtctatcaaatgactcctcattctgatttccaatatcaaatttggataatctgatggtggcagacataagtgtcagcttgagcttcattttaaattcaatttatttaaaatgacataaattttaatgttatttaaataaatttactttgaaaggaaaatcttaatgaaaagtagaaataagacaggaaattttgtaaagaaaagtaatcccactatgagtaccagacaagataagttgaaatgcactaacatcattatagataataggttatagaggcatttcttcttgcttttctaagtagctctatagggattgaaaagtagggcagataaaacgtgttgcaaatttcattcacccctttgccctccccctctcctcatccacttcttctcctgtaccccttccattatctacttctttcaccaaaatcacatcttcttgcatttttgtgcatcttttcttctgaagtatagtaattctttgaatatcttttattctgaccaattagttaaataaaccaaacatttttaaattattcgaaactgtttctaaattgtgttatatttccgtttctaaattgtatgtttcagagagttttcttgtgagtagcccagcttcaaaaaactttttctcctagcttgccatgaataaaaaataaaaatttccatgttttatgtttttaagtctttgactcaaatttcagttgaaatttggttcatcagtaagcatacatcaaaggcagttttaacctcaagatgatattgtgtactttaacaaaagtgtaactattttttatcccccacccctttttttaaaggaatccgcaggcggggatcttcagtctcctttaacaccggaaagtatcaacgggacagatgatgaaagaacacctgatgtgacacagaactcagagccaagggctgaaccaactcagaatgcattgccattttcacatag

SEQ ID NO: 440 - DNA, 522 bp, Homo sapiens
ggaacaacctatcttcagcactcgagctcatgtcttccaaattgacccaaacacaaagaagaactgggtacccaccagcaagcatgcagttactgtgtcttatttctatgacagcacaagaaatgtgtataggataatcagtttagatggctcaaaggcaataataaatagtaccatcaccccaaacatgacatttactaaaacatctcagaagtttggccagtgggctgatagccgggcaaacaccgtttatggattgggattctcctctgagcatcatctttcgaaatttgcagaaaagtttcaggaatttaaagaagctgctcgactagcaaaggaaaaatcacaagagaagatggaacttaccagtacaccttcacaggaatccgcaggcggggatcttcagtctcctttaacaccggaaagtatcaacgggacagatgatgaaagaacacctgatgtgacacagaactcagagccaagggctgaaccaactcagaatgcattgccattttcacatag

SEQ ID NO: 441 - DNA, 20 bp, Mus musculus
aagcttaggatgtgtggaac

SEQ ID NO: 442 - DNA, 20 bp, Mus musculus
gttagaatagaacttgtcac

SEQ ID NO: 443 - DNA, 120 bp, Mus musculus
acatttcaaggagtgatcactctttggtgtacttctttagacgctgtcagaagcttaggatgtgtggaacaggaggactgtgactgtaaatgcttactaatcgctttgagagagtgttat

SEQ ID NO: 444 - DNA, 120 bp, Mus musculus
aacaaaaactaaaaatggagctttacttctgaactctcagttctggcccagtgacaagttctattctaacaccaggactacattgccttcattgttttaacttggcattaatatttaaaa

SEQ ID NO: 445 - DNA, 300 bp, Mus musculus
tgggttcgtatatactttcagttaagagctgtagtactgttttatgtcctcgtcatagctatgcaataatagtctcatcagatacatttcaaggagtgatcactctttggtgtacttctttagacgctgtcagaagcttaggatgtgtggacaagttctattctaacaccaggactacattgccttcattgttttaacttggcattaatatttaaaattgggaagtatgatttgtataaccttttccttttttttttttaagatcgagtcttgagtccttacaatttctaagcaaagttt

SEQ ID NO: 446 - DNA, 522 bp, Mus musculus
ggagcaacctatcttcagcactcgagctcatgtcttccagattgacccgaacacaaagaagaactgggtacccaccagcaagcatgcagttactgtatcttatttttatgacagcacaagaaatgtgtataggataatcagtttagatggctcaaaggcaataataaatagcaccatcacaccaaacatgacatttactaaaacatctcaaaagtttggccaatgggctgatagccgggcaaacactgtttatggactgggattctcctctgagcatcatctttcaaaattcgcagaaaagtttcaggaatttaaggaagctgctcggcttgcaaaggagaagtcgcaggagaagatggagctgaccagtaccccttcacaggaatcagcaggaggagatcttcagtctcctttgacaccagaaagtatcaatgggacagacgatgagagaacacccgatgtgacacagaactcagagccaagggctgagccaactcagaatgcattgccatttccacatag